(19) United States
(12) Patent Application Publication
(10) Pub. No.: US 2025/0375389 A1
(43) Pub. Date: Dec. 11, 2025

(54) LIPID NANOPARTICLE COMPOSITIONS COMPRISING PHOSPHOLIPID DERIVATIVES AND RELATED USES

(71) Applicant: ModernaTX, Inc., Cambridge, MA (US)

(72) Inventors: Jaclyn MILTON, Cambridge, MA (US); Mohindra SEEPERSAUD, Cambridge, MA (US); Karthik RAMACHANDRAN, Cambridge, MA (US); Russell B. UMSTEAD, Cambridge, MA (US); Calvin Loishiye MARAMBO, Cambridge, MA (US); Mark CORNEBISE, Cambridge, MA (US)

(21) Appl. No.: 19/099,092

(22) PCT Filed: Jul. 28, 2023
(86) PCT No.: PCT/US2023/071259

§ 371 Date: Jan. 28, 2025

Related U.S. Application Data
(60) Provisional application No. 63/393,793, filed on Jul. 29, 2022.

(51) Int. Cl. A61K 9/51

(52) U.S. Cl. CPC A61K 9/5123; A61K 9/5192

(57) ABSTRACT

The present disclosure provides lipid assemblies suitable for delivery of therapeutic agents to hematopoietic stem and progenitor cells (HSPCs), wherein the lipid assemblies comprise a phosphatidylserine phospholipid. The present disclosure also provides therapeutic and diagnostic uses related to the lipid assemblies.

### CROSS-REFERENCE TO RELATED APPLICATIONS

The application claims the benefit of and priority to U.S. Provisional Application Ser. No. 63/393,793 filed Jul. 29, 2022, the contents of which are incorporated by reference in their entirety for all purposes.

### BACKGROUND

The effective targeted delivery of biologically active substances such as small molecule drugs, proteins, and nucleic acids represents a continuing medical challenge. In particular, the delivery of nucleic acids to cells is made difficult by the relative instability and low cell permeability of such species. Thus, there exists a need to develop methods and compositions to facilitate the delivery of therapeutics and prophylactics such as nucleic acids to cells.

Lipid assemblies (e.g., lipid nanoparticles, liposomes, and lipoplexes) have proven effective as transport vehicles into cells and/or intracellular compartments for biologically active substances such as small molecule drugs, proteins, and nucleic acids. Though a variety of such lipid-containing nanoparticles have been demonstrated, improvements in safety, efficacy, and specificity are still needed.

### SUMMARY

The present disclosure provides a lipid assembly comprising a phosphatidylserine phospholipid, an ionizable lipid, and a structural lipid, wherein the phosphatidylserine phospholipid is of Formula (PL-I):

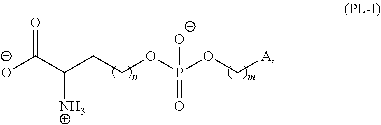

or a salt thereof, wherein:
n is 0, 1, 2, 3, 4, 5, 6, 7, 8, 9, or 10;
m is 0, 1, 2, 3, 4, 5, 6, 7, 8, 9, or 10;
A is of the formula:

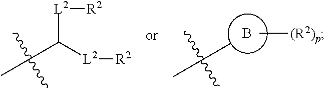

each instance of L² is independently a bond or an optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently cholesterol, optionally substituted cycloalkyl, optionally substituted C₁₋₃₀ alkyl, optionally substituted C₁₋₃₀ alkenyl, or optionally substituted C₁₋₃₀ alkynyl; optionally wherein one or more methylene units of the optionally substituted C₁₋₃₀ alkyl, optionally substituted C₁₋₃₀ alkenyl, or optionally substituted C₁₋₃₀ alkynyl are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen-protecting group;
Ring B is optionally substituted carbocyclyl, optionally substituted heterocyclyl, optionally substituted aryl, or optionally substituted heteroaryl; and
p is 1 or 2.

The present disclosure also provides populations of lipid assemblies comprising a phosphatidylserine phospholipid, an ionizable lipid, and a structural lipid, wherein the phosphatidylserine phospholipid is of Formula (PL-I):

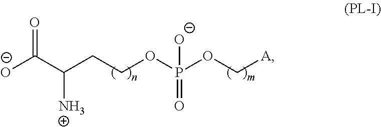

or a salt thereof, wherein:
n is 0, 1, 2, 3, 4, 5, 6, 7, 8, 9, or 10;
m is 0, 1, 2, 3, 4, 5, 6, 7, 8, 9, or 10;
A is of the formula:

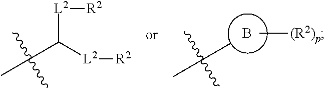

each instance of L² is independently a bond or an optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently cholesterol, optionally substituted cycloalkyl, optionally substituted C₁₋₃₀ alkyl, optionally substituted C₁₋₃₀ alkenyl, or optionally substituted C₁₋₃₀ alkynyl; optionally wherein one or more methylene units of the optionally substituted C₁₋₃₀ alkyl, optionally substituted C₁₋₃₀ alkenyl, or optionally substituted C₁₋₃₀ alkynyl are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen-protecting group;
Ring B is optionally substituted carbocyclyl, optionally substituted heterocyclyl, optionally substituted aryl, or optionally substituted heteroaryl; and
p is 1 or 2.

The present disclosure also provides methods of preparing the lipid assembly and the population of lipid assemblies described herein.

The present disclosure also provides pharmaceutical compositions comprising the lipid assembly, pharmaceutical compositions comprising the population of lipid assemblies described herein, and methods of preparing the pharmaceutical compositions.

The present disclosure also provides methods of delivering a therapeutic agent to a hematopoietic stem and progenitor cell (HSPC) in a subject, comprising administering to the subject a lipid assembly or a population of lipid assemblies described herein.

The present disclosure also provides lipid assemblies, populations of lipid assemblies, and pharmaceutical composition for use in delivering therapeutic agents to hematopoietic stem and progenitor cells (HSPC) in subjects.

The present disclosure also provides uses of lipid assemblies, populations of lipid assemblies, and pharmaceutical composition in the manufacture of medicaments for delivering therapeutic agents to hematopoietic stem and progenitor cells (HSPC) in subjects.

The present disclosure also provides lipid assemblies, populations of lipid assemblies, and pharmaceutical compositions for use in treating or preventing diseases or disorders in subjects.

The present disclosure also provides uses of lipid assemblies, populations of lipid assemblies, and pharmaceutical compositions in the manufacture of a medicaments for treating or preventing diseases and disorders in subjects.

The present disclosure also provides processes of preparing nanoparticles, the processes comprising contacting an aqueous stream comprising a phosphatidylserine lipid with an organic stream comprising an ionizable lipid and a structural lipid.

The present disclosure also provides nanoparticles prepared by the processes of preparing nanoparticles provided herein.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the disclosure will be apparent from the following detailed description and claims.

### DETAILED DESCRIPTION

### Lipid Assemblies of the Present Disclosure

The present disclosure provides lipid assemblies and populations of lipid assemblies comprising a phosphatidylserine phospholipid, an ionizable lipid, and a structural lipid. Lipid assemblies as disclosed herein have surprisingly been found to exhibit consistently high transfection and high protein expression in hematopoietic stem and progenitor cells (HSPCs), including in bone marrow HSPCs and splenic HSPCs.

In some embodiments, the present disclosure provides a lipid assembly comprising a phosphatidylserine phospholipid, an ionizable lipid, and a structural lipid, wherein the phosphatidylserine phospholipid is of Formula (PL-I):

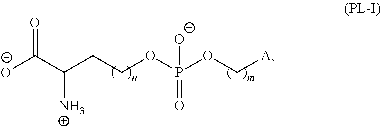

or a salt thereof, wherein:
n is 0, 1, 2, 3, 4, 5, 6, 7, 8, 9, or 10;
m is 0, 1, 2, 3, 4, 5, 6, 7, 8, 9, or 10;
A is of the formula:

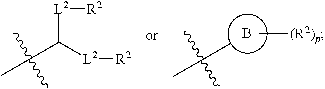

each instance of L² is independently a bond or an optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently cholesterol, optionally substituted cycloalkyl, optionally substituted C₁₋₃₀ alkyl, optionally substituted C₁₋₃₀ alkenyl, or optionally substituted C₁₋₃₀ alkynyl; optionally wherein one or more methylene units of the optionally substituted C₁₋₃₀ alkyl, optionally substituted C₁₋₃₀ alkenyl, or optionally substituted C₁₋₃₀ alkynyl are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen-protecting group;
Ring B is optionally substituted carbocyclyl, optionally substituted heterocyclyl, optionally substituted aryl, or optionally substituted heteroaryl; and
p is 1 or 2.

In some embodiments, the present disclosure provides a population of lipid assemblies comprising a phosphatidylserine phospholipid, an ionizable lipid, and a structural lipid, wherein the phosphatidylserine phospholipid is of Formula (PL-I):

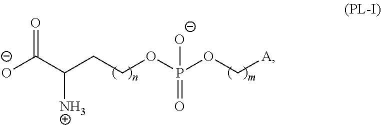

or a salt thereof, wherein:
n is 0, 1, 2, 3, 4, 5, 6, 7, 8, 9, or 10;
m is 0, 1, 2, 3, 4, 5, 6, 7, 8, 9, or 10;
A is of the formula:

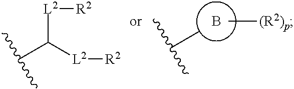

each instance of L² is independently a bond or an optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently cholesterol, optionally substituted cycloalkyl, optionally substituted C₁₋₃₀ alkyl, optionally substituted C₁₋₃₀ alkenyl, or optionally substituted C₁₋₃₀ alkynyl; optionally wherein one or more methylene units of the optionally substituted C₁₋₃₀ alkyl, optionally substituted C₁₋₃₀ alkenyl, or optionally substituted C₁₋₃₀ alkynyl are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen-protecting group;
Ring B is optionally substituted carbocyclyl, optionally substituted heterocyclyl, optionally substituted aryl, or optionally substituted heteroaryl; and
p is 1 or 2.

In some embodiments, n of Formula PL-1 is 0. In some embodiments, m of Formula PL-1 is 1. In some embodiments, n of Formula PL-1 is 0 and m of Formula PL-1 is 1.

In some embodiments, the phosphatidylserine phospholipid is of Formula (PL-Ia):

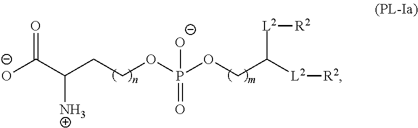

or a salt thereof. In some embodiments, n of Formula PL-Ia is 0. In some embodiments, m of Formula PL-Ia is 1. In some embodiments, n of Formula PL-Ia is 0 and m of Formula PL-Ia is 1.

In some embodiments, the phosphatidylserine phospholipid is of Formula (PL-Ib):

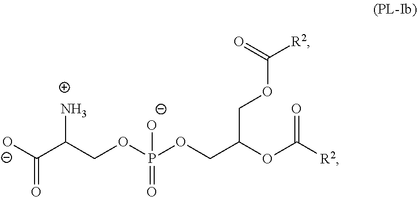

or a salt thereof.

In some embodiments, each instance of R² is independently C₁₋₃₀, C₉₋₂₁, C₁₁₋₁₉, or C₁₃₋₁₇ alkyl or C₁₋₃₀, C₉₋₂₁, C₁₁₋₁₉, or C₁₃₋₁₇ alkenyl. In some embodiments, each instance of R² is independently C₁₁₋₁₉ alkyl. In some embodiments, each instance of R² is independently C₁₃₋₁₇ alkyl. In some embodiments, one R² is C₁₋₃₀, C₉₋₂₁, C₁₁₋₁₉, or C₁₃₋₁₇ alkyl and a second R² is C₁₋₃₀, C₉₋₂₁, C₁₁₋₁₉, or C₁₃₋₁₇ alkenyl. In some embodiments, one R² is C₁₋₃₀ alkyl and a second R² is C₁₋₃₀ alkenyl. In some embodiments, one R² is C₁₁₋₁₉ alkyl and a second R² is C₁₁₋₁₉ alkenyl. In some embodiments, one R² is C₁₃₋₁₇ alkyl and a second R² is C₁₃₋₁₇ alkenyl. In some embodiments, each instance of R² is C₁₃ alkyl. In some embodiments, each instance of R² is C₁₇ alkyl. In some embodiments, each instance of R² is C₁₇ alkenyl. In some embodiments, one instance of R² is C₁₇ alkyl and a second instance of R² is C₁₇ alkenyl.

In some embodiments, the phosphatidylserine phospholipid is

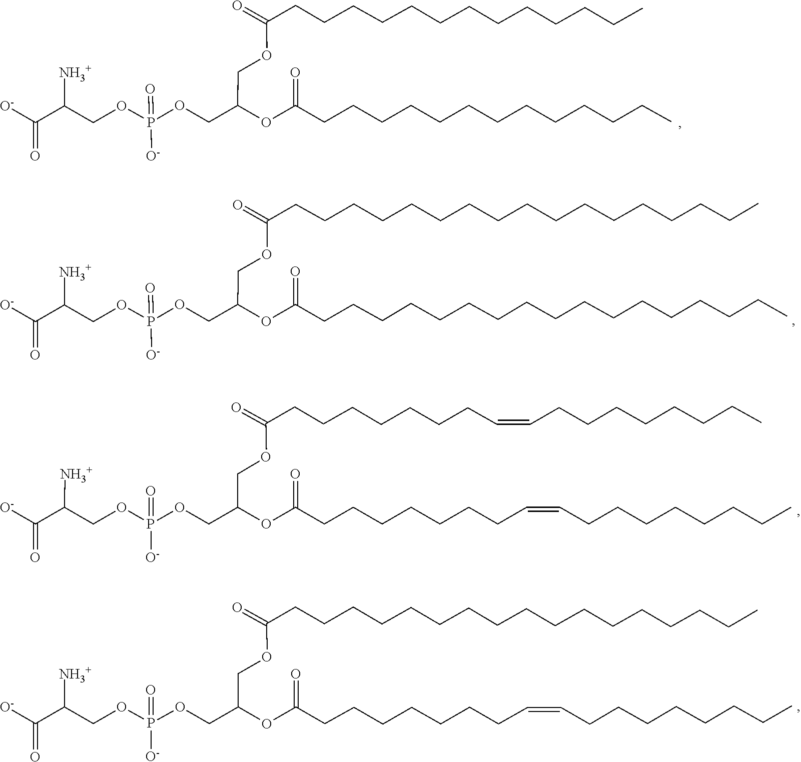 [SMILES: CCCCCCCCCCCCCC(=O)OCC(COP(=O)([O-])OCC([NH3+])C(=O)[O-])OC(=O)CCCCCCCCCCCCC ; CCCCCCCCCCCCCCCCCC(=O)OCC(COP(=O)([O-])OCC([NH3+])C(=O)[O-])OC(=O)CCCCCCCCCCCCCCCCC ; CCCCCCCC/C=C\CCCCCCCC(=O)OCC(COP(=O)([O-])OCC([NH3+])C(=O)[O-])OC(=O)CCCCCCC/C=C\CCCCCCCC ; CCCCCCCC/C=C\CCCCCCCC(=O)OC(COC(=O)CCCCCCCCCCCCCCCCC)COP(=O)([O-])OCC([NH3+])C(=O)[O-]]

or a salt of any of the foregoing.

In some embodiments, the population of lipid assemblies comprises between about 1 mol % to about 20 mol %, about 1 mol % to about 15 mol %, about 1 mol % to about 10 mol %, about 1 mol % to about 8 mol %, about 1 mol % to about 5 mol %, or about 2 mol % to about 5 mol % of the phosphatidylserine phospholipid. In some embodiments, the population of lipid assemblies comprises between about 1 mol % to about 10 mol % of the phosphatidylserine phospholipid. In some embodiments, the population of lipid assemblies comprises between about 1 mol % to about 20 mol % of the phosphatidylserine phospholipid. In some embodiments, the population of lipid assemblies comprises between about 1 mol % to about 15 mol % of the phosphatidylserine phospholipid. In some embodiments, the population of lipid assemblies comprises between about 1 mol % to about 5 mol % of the phosphatidylserine phospholipid. In some embodiments, the population of lipid assemblies comprises between about 2 mol % to about 5 mol % of the phosphatidylserine phospholipid.

In some embodiments, the phosphatidylserine lipid is DMPS. In some embodiments, the population of lipid assemblies comprises about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, or about 20 mol % of the phosphatidylserine phospholipid. In some embodiments, the population of lipid assemblies comprises about 5 mol % of the phosphatidylserine phospholipid (e.g., DMPS). In some embodiments, the population of lipid assemblies comprises about 6 mol % of the phosphatidylserine phospholipid (e.g., DMPS). In some embodiments, the population of lipid assemblies comprises about 7 mol % of the phosphatidylserine phospholipid (e.g., DMPS). In some embodiments, the population of lipid assemblies comprises about 8 mol % of the phosphatidylserine phospholipid (e.g., DMPS). In some embodiments, the population of lipid assemblies comprises about 9 mol % of the phosphatidylserine phospholipid (e.g., DMPS). In some embodiments, the population of lipid assemblies comprises about 10 mol % of the phosphatidylserine phospholipid (e.g., DMPS).

In some embodiments, the ionizable lipid is compound 301, compound 22, or I-18. In some embodiments, the ionizable lipid is compound 301 or compound 22.

In some embodiments, the ionizable lipid is compound 301. Compound 301 is a compound of the formula

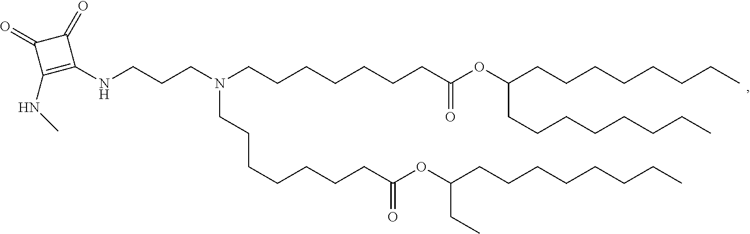 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or a salt thereof.

In some embodiments, the ionizable lipid is compound 22. Compound 22 is a compound of the formula

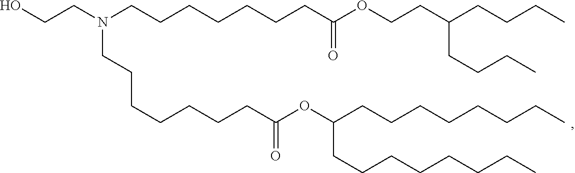 [SMILES: CCCCCCCCC(CCCCCCCC)OC(=O)CCCCCCCN(CCO)CCCCCCCC(=O)OCCC(CCCC)CCCC]

or a salt thereof.

In some embodiments, the ionizable lipid is I-18. I-18 is a compound of the formula

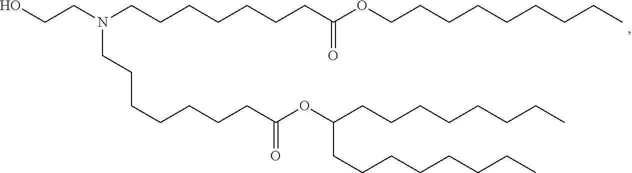 [SMILES: CCCCCCCCCOC(=O)CCCCCCCN(CCO)CCCCCCCC(=O)OC(CCCCCCCC)CCCCCCCC]

or a salt thereof.

In some embodiments, the population of lipid assemblies comprises about 30 mol % to about 50 mol %, about 30 mol % to about 45 mol %, about 30 mol % to about 40 mol %, about 35 mol % to about 45 mol %, or about 35 mol % to about 40 mol % of the ionizable lipid. In some embodiments, the population of lipid assemblies comprises about 30 mol %, about 35 mol %, about 40 mol %, about 45 mol %, or about 50 mol % of the ionizable lipid. In some embodiments, the population of lipid assemblies comprises about 30 mol % to about 50 mol % of the ionizable lipid (e.g., compound 301 or compound 22). In some embodiments, the population of lipid assemblies comprises about 35 mol % to about 45 mol % of the ionizable lipid (e.g., compound 301 or compound 22).

In some embodiments, the population of lipid assemblies comprises about 30 mol % to about 50 mol %, about 30 mol % to about 45 mol %, about 30 mol % to about 40 mol %, about 35 mol % to about 45 mol %, or about 35 mol % to about 40 mol % of compound 301. In some embodiments, the population of lipid assemblies comprises about 30 mol %, about 35 mol %, about 40 mol %, about 45 mol %, or about 50 mol % of compound 301.

In some embodiments, the population of lipid assemblies comprises about 30 mol % to about 50 mol %, about 30 mol % to about 45 mol %, about 30 mol % to about 40 mol %, about 35 mol % to about 45 mol %, or about 35 mol % to about 40 mol % of compound 22. In some embodiments, the population of lipid assemblies comprises about 30 mol %, about 35 mol %, about 40 mol %, about 45 mol %, or about 50 mol % of compound 22.

In some embodiments, the structural lipid is cholesterol.

In some embodiments, the population of lipid assemblies comprises about 15 mol % to about 50 mol %, about 20 mol % to about 50 mol %, about 25 mol % to about 50 mol %, about 30 mol % to about 50 mol %, about 35 mol % to about 50 mol %, about 40 mol % to about 50 mol %, or about 45 mol % to about 50 mol % of the structural lipid (e.g., cholesterol). In some embodiments, the population of lipid assemblies comprises about 15 mol % to about 45 mol %, about 20 mol % to about 45 mol %, about 25 mol % to about 45 mol %, about 30 mol % to about 45 mol %, about 35 mol % to about 45 mol %, or about 40 mol % to about 45 mol % of the structural lipid (e.g., cholesterol). In some embodiments the population of lipid assemblies comprises about 15 mol % to about 40 mol %, about 20 mol % to about 40 mol %, about 25 mol % to about 40 mol %, about 30 mol % to about 40 mol %, or about 35 mol % to about 40 mol % of the structural lipid (e.g., cholesterol). In some embodiments, the population of lipid assemblies comprises about 20 mol % to about 45 mol % of the structural lipid (e.g., cholesterol). In some embodiments, the population of lipid assemblies comprises about 20 mol % to about 40 mol % of the structural lipid (e.g., cholesterol). In some embodiments, the population of lipid assemblies comprises about 30 mol % to about 40 mol % of the structural lipid (e.g., cholesterol).

In some embodiments, the population of lipid assemblies comprises about 15 mol %, about 20 mol %, about 25 mol %, about 30 mol %, about 35 mol %, about 40 mol %, about 45 mol %, or about 50 mol % of the structural lipid (e.g., cholesterol). In some embodiments, the population of lipid assemblies comprises about 35 mol % of the structural lipid (e.g., cholesterol). In some embodiments, the population of lipid assemblies comprises about 40 mol % of the structural lipid (e.g., cholesterol). In some embodiments, the population of lipid assemblies comprises about 45 mol % of the structural lipid (e.g., cholesterol).

In some embodiments, the population of lipid assemblies comprises a second phospholipid. In some embodiments, it comprises about 10 mol % to about 30 mol %, about 15 mol % to about 25 mol %, or about 15 mol % to about 20 mol % of the second phospholipid. In some embodiments, it comprises about 10 mol %, about 15 mol %, about 18 mol %, about 20 mol %, about 22 mol %, about 25 mol %, or about 30 mol % of the second phospholipid. In some embodiments, the second phospholipid is DSPC, DOPE, DOPC, POPE, or POPC.

In some embodiments, the population of lipid assemblies comprises a second phospholipid that is DSPC, wherein the population of lipid assemblies comprises about 10 mol % to about 30 mol %, about 10 mol % to about 25 mol %, about 10 mol % to about 20 mol %, about 15 mol % to about 25 mol %, or about 15 mol % to about 20 mol % of the DSPC. In some embodiments, the population of lipid assemblies comprises about 10 mol % to about 25 mol % of the DSPC. In some embodiments, the population of lipid assemblies comprises about 10 mol % to about 20 mol % of the DSPC. In some embodiments, the population of lipid assemblies comprises about 15 mol % to about 25 mol % of the DSPC. In some embodiments, the population of lipid assemblies comprises about 15 mol % to about 20 mol % of the DSPC.

In some embodiments, the population of lipid assemblies comprises a second phospholipid that is DSPC, wherein the population of lipid assemblies comprises about 10 mol %, about 15 mol %, about 18 mol %, about 20 mol %, about 22 mol %, about 25 mol %, or about 30 mol % of the DSPC. In some embodiments, the population of lipid assemblies comprises about 20 mol % of the DSPC. In some embodiments, the population of lipid assemblies comprises about 22 mol % of the DSPC. In some embodiments, the population of lipid assemblies comprises about 25 mol % of the DSPC.

In some embodiments, the population of lipid assemblies is free of PEG lipid.

In some embodiments, the population of lipid assemblies further comprises a PEG lipid. In some embodiments, the population of lipid assemblies comprises about 0.5 mol % to about 10 mol %, about 0.5 mol % to about 5 mol %, about 1 mol % to about 5 mol %, or about 1 mol % to about 3 mol % of the PEG lipid.

In some embodiments, the PEG lipid is PL-02. PL-02 refers to a polymer of the formula:

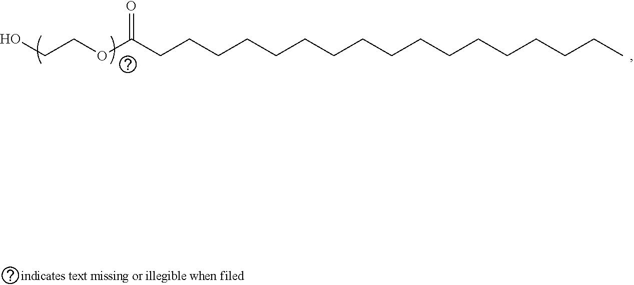

or a salt thereof, wherein r is 45. As one of ordinary skill in the art would understand, the number of repeating units indicated in the structure of a polymer refers to the average number of repeating units (a.k.a., average degree of polymerization). E.g., in some embodiments, r is an integer from about 35 to about 55.

In some embodiments, the population of lipid assemblies comprises about 1 mol % to about 5 mol % of the PEG lipid (e.g., PL-02). In some embodiments, the population of lipid assemblies comprises about 3 mol % to about 5 mol % of the PEG lipid (e.g., PL-02).

In some embodiments, the population of lipid assemblies comprises about 0.5 mol %, about 1 mol %, about 2 mol %, about 3 mol %, about 4 mol %, or about 5 mol % of the PEG lipid (e.g., PL-02). In some embodiments, the population of lipid assemblies comprises about 3 mol % of the PEG lipid (e.g., PL-02). In some embodiments, the population of lipid assemblies comprises about 4 mol % of the PEG lipid (e.g., PL-02). In some embodiments, the population of lipid assemblies comprises about 5 mol % of the PEG lipid (e.g., PL-02).

In some embodiments, the population of lipid assemblies has a pH value lower than the pKa value of the ionizable lipid. In some embodiments, it has a pH value of about 4.0±2.0, about 4.0±1.5, about 4.0±1.4, about 4.0±1.3, about 4.0±1.2, about 4.0±1.1, about 4.0±1.0, about 4.0±0.9, about 4.0±0.8, about 4.0±0.7, about 4.0±0.6, about 4.0±0.5, about 4.0±0.4, about 4.0±0.3, about 4.0±0.2, or about 4.0±0.1. In some embodiments, it has a pH value of about 5.0±2.0, about 5.0±1.5, about 5.0±1.4, about 5.0±1.3, about 5.0±1.2, about 5.0±1.1, about 5.0±1.0, about 5.0±0.9, about 5.0±0.8, about 5.0±0.7, about 5.0±0.6, about 5.0±0.5, about 5.0±0.4, about 5.0±0.3, about 5.0±0.2, or about 5.0±0.1.

In some embodiments, the population of lipid assemblies has a pH value higher than the pKa value of the ionizable lipid. In some embodiments, it has a pH value of about 8.0±2.0, about 8.0±1.5, about 8.0±1.4, about 8.0±1.3, about 8.0±1.2, about 8.0±1.1, about 8.0±1.0, about 8.0±0.9, about 8.0±0.8, about 8.0±0.7, about 8.0±0.6, about 8.0±0.5, about 8.0±0.4, about 8.0±0.3, about 8.0±0.2, or about 8.0±0.1. In some embodiments, the population of lipid assemblies has a pH value of about 9.0±3.0, about 9.0±2.0, about 9.0±1.5, about 9.0±1.4, about 9.0±1.3, about 9.0±1.2, about 9.0±1.1, about 9.0±1.0, about 9.0±0.9, about 9.0±0.8, about 9.0±0.7, about 9.0±0.6, about 9.0±0.5, about 9.0±0.4, about 9.0±0.3, about 9.0±0.2, or about 9.0±0.1. In some embodiments, the population of lipid assemblies has a pH value of about 12.0±2.0, about 12.0±1.5, about 12.0±1.4, about 12.0±1.3, about 12.0±1.2, about 12.0±1.1, about 12.0±1.0, about 12.0±0.9, about 12.0±0.8, about 12.0±0.7, about 12.0±0.6, about 12.0±0.5, about 12.0±0.4, about 12.0±0.3, about 12.0±0.2, or about 12.0±0.1.

In some embodiments, the population of lipid assemblies has a zeta potential between about −40 mV to about −5 mV, about −30 mV to about −5 mV, about −20 mV to about −5 mV, about −40 mV to about −10 mV, about −30 mV to about −10 mv, or about −20 mV to about −10 mV when measured in 0.1N PBS at pH 7.5. In some embodiments, the population of lipid assemblies has a zeta potential between about −20 mV to about −10 mV when measured in 0.1N PBS at pH 7.5.

In some embodiments, the population of lipid assemblies is free of therapeutic agent.

In some embodiments, the population of lipid assemblies further comprises a therapeutic agent. In some embodiments, the therapeutic agent is an RNA.

Embodiments of the present disclosure are directed to pharmaceutical compositions comprising the population of lipid assemblies described herein and one or more pharmaceutically acceptable carriers or excipients. Some embodiments are directed to methods of preparing the pharmaceutical compositions.

Embodiments of the present disclosure are directed to a method of preparing the population of lipid assemblies as described herein.

Embodiments of the present disclosure are directed to methods of delivering a therapeutic agent to a hematopoietic stem and progenitor cell (HSPC) in a subject, comprising administering to the subject the population of lipid assemblies or the pharmaceutical compositions as described herein.

Embodiments of the present disclosure are directed to a population of lipid assemblies or pharmaceutical compositions as described herein for use in delivering a therapeutic agent to a hematopoietic stem and progenitor cell (HSPC) in a subject.

Embodiments of the present disclosure are directed to use of the population of lipid assemblies or the pharmaceutical compositions as described herein in the manufacture of a medicament for delivering a therapeutic agent to a hematopoietic stem and progenitor cell (HSPC) in a subject.

Embodiments of the present disclosure are directed to methods of treating or preventing a disease or disorder, the method comprising administering to a subject in need thereof the population of lipid assemblies or the pharmaceutical composition as described herein. In some embodiments, the disease or disorder is a spleen disease or a spleen disorder. In some embodiments, the disease or disorder is a bone marrow disease or a bone marrow disorder.

Embodiments of the present disclosure are directed to the population of lipid assemblies or pharmaceutical compositions as described herein for use in treating or preventing a disease or disorder in a subject. In some embodiments, the disease or disorder is a spleen disease or a spleen disorder. In some embodiments, the disease or disorder is a bone marrow disease or a bone marrow disorder.

Embodiments of the present disclosure are directed to use of the population of lipid assemblies or the pharmaceutical composition as described herein in the manufacture of a medicament for treating or preventing a disease or disorder in a subject. In some embodiments, the disease or disorder is a spleen disease or a spleen disorder. In some embodiments, the disease or disorder is a bone marrow disease or a bone marrow disorder.

In some embodiments, the subject is human.

Embodiments of the present disclosure are directed to a process of preparing a nanoparticle, the process comprising contacting an aqueous stream comprising a phosphatidylserine lipid with an organic stream comprising an ionizable lipid and a structural lipid, thereby obtaining a mixed product.

In some embodiments, organic stream further comprises a PEG lipid.

In some embodiments, the pH of the aqueous stream is lower than the pKa of the ionizable lipid.

In some embodiments, the process further comprises subjecting the mixed product to an in-line dilution. In some embodiments, the in-line dilution lowers an organic solvent content of the mixed product. In some embodiments, the in-line dilution raises the pH of the mixed product above the pKa of the ionizable lipid.

In some embodiments, the phosphatidylserine phospholipid is DMPS.

In some embodiments, the ionizable lipid is Compound 22 or Compound 301.

Embodiments of the present disclosure are directed to a nanoparticle prepared by the process of preparing a nanoparticle disclosed herein.

### Phospholipids

Phospholipids may assemble into one or more lipid bilayers. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of one or more elements to a target tissue.

Particular phospholipids can facilitate cellular uptake and/or fusion. For example, an anionic phospholipid (e.g., phospholipids comprising phosphatidyl glycerol or phosphatidylserine) can interact with one or more receptors of a cell (e.g., receptors of a cellular or intracellular membrane). Cellular uptake of a lipid-containing composition (e.g., LNPs) can allow for subcellular trafficking of the lipid-containing composition (e.g., entry into endosomes or transport to the endoplasmic reticulum). Anionic phospholipids can also facilitate fusion to a membrane. Interaction of an anionic phospholipid with a receptor of a cell can bring elements of a lipid-containing composition into contact with a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds are replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipids, such as sphingomyelin.

### Anionic Phospholipids

In some embodiments, a phospholipid useful or potentially useful in the present invention is an anionic phospholipid. In some embodiments, the anionic phospholipid is a phosphatidylserine phospholipid (i.e., a phospholipid with a phosphatidylserine headgroup). A phosphatidylserine phospholipid useful or potentially useful in the present invention is a compound of Formula (PL-I):

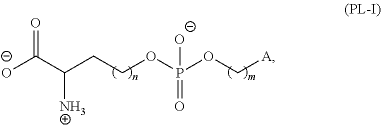

or a salt thereof, wherein:
n is 0, 1, 2, 3, 4, 5, 6, 7, 8, 9, or 10;
m is 0, 1, 2, 3, 4, 5, 6, 7, 8, 9, or 10;
A is of the formula:

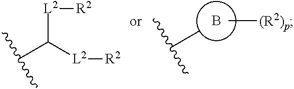

each instance of L² is independently a bond or an optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently cholesterol, optionally substituted cycloalkyl, optionally substituted C₁₋₃₀ alkyl, optionally substituted C₁₋₃₀ alkenyl, or optionally substituted C₁₋₃₀ alkynyl; optionally wherein one or more methylene units of the optionally substituted C₁₋₃₀ alkyl, optionally substituted C₁₋₃₀ alkenyl, or optionally substituted C₁₋₃₀ alkynyl are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen-protecting group;
Ring B is optionally substituted carbocyclyl, optionally substituted heterocyclyl, optionally substituted aryl, or optionally substituted heteroaryl; and
p is 1 or 2.

### Anionic Phospholipid Tail Modifications

In some embodiments, a phospholipid useful or potentially useful in the present invention comprises a modified tail. In some embodiments, a phospholipid useful or potentially useful in the present invention is DMPS, DMPG, an analog of DMPS with a modified tail, or an analog of DMPG with a modified tail. For example, in some embodiments A is

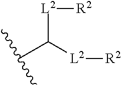

In some embodiments, the phospholipid is of Formula (PL-Ia):

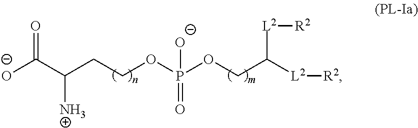

or a salt thereof.

As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in some embodiments, at least one instance of R² is optionally substituted with C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In some embodiments, A is

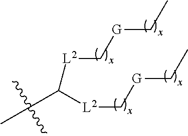

wherein:
each x is independently an integer between 0-30, inclusive, and
each G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In some embodiments, the compound of Formula (PL-I) is of Formula (PL-I-c):

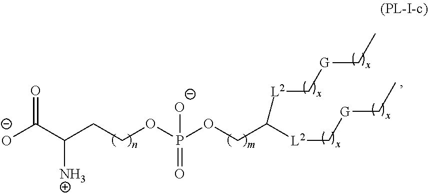

or a salt thereof, wherein:
each x is independently an integer between 0-30, inclusive, and each G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In some embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in some embodiments, n is 0, 1, 3, 4, 5, 6, 7, 8, 9, or 10.

In some embodiments, the phospholipid is of Formula (PL-Ib):

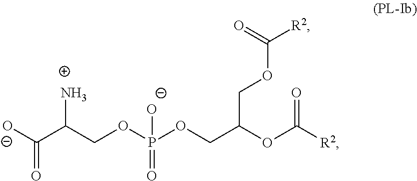

or a salt thereof.

In some embodiments, the phospholipid is

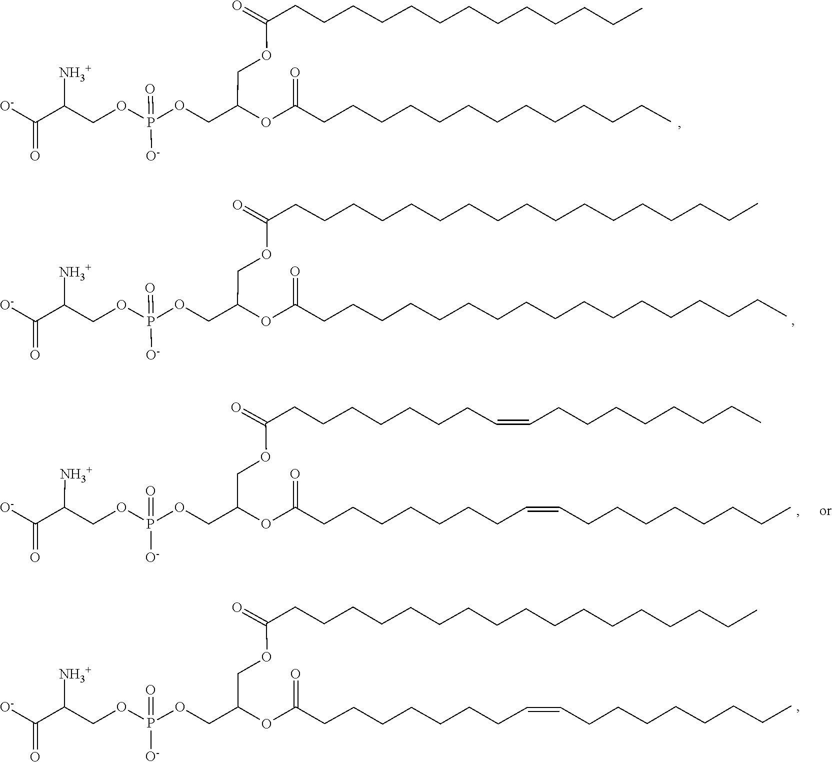 [SMILES: CCCCCCCCCCCCCC(=O)OCC(COP(=O)([O-])OCC([NH3+])C(=O)[O-])OC(=O)CCCCCCCCCCCCC ; CCCCCCCCCCCCCCCCCC(=O)OCC(COP(=O)([O-])OCC([NH3+])C(=O)[O-])OC(=O)CCCCCCCCCCCCCCCCC ; CCCCCCCC/C=C\CCCCCCCC(=O)OCC(COP(=O)([O-])OCC([NH3+])C(=O)[O-])OC(=O)CCCCCCC/C=C\CCCCCCCC ; CCCCCCCC/C=C\CCCCCCCC(=O)OC(COC(=O)CCCCCCCCCCCCCCCCC)COP(=O)([O-])OCC([NH3+])C(=O)[O-]]

or a salt thereof.

### Optional Phospholipids:

In some embodiments, a phospholipid useful or potentially useful in the present invention is an analog or variant of DSPC. In some embodiments, a phospholipid useful or potentially useful in the present invention is a compound of Formula (PL-II):

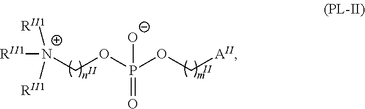

or a salt thereof, wherein:
each R^{II1} is independently optionally substituted alkyl; optionally two R^{II1} are joined together with the intervening atoms to form optionally substituted monocyclic carbocyclyl or optionally substituted monocyclic heterocyclyl; or optionally three R^{II1} are joined together with the intervening atoms to form optionally substituted bicyclic carbocyclyl or optionally substitute bicyclic heterocyclyl;
n^{II} is 1, 2, 3, 4, 5, 6, 7, 8, 9, or 10;
m^{II} is 0, 1, 2, 3, 4, 5, 6, 7, 8, 9, or 10;
A^{II} is of the formula:

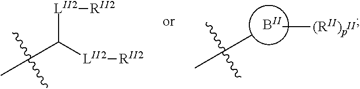

each instance of L^{II2} is independently a bond or optionally substituted C₁₋₆ alkylene, wherein one methylene unit of the optionally substituted C₁₋₆ alkylene is optionally replaced with —O—, —N(R^{IIN})—, —S—, —C(O)—, —C(O)N(R^{IIN})—, —NR^{IIN}C(O)—, —C(O)O—, —OC(O)—, —OC(O)O—, —OC(O)N(R^{IIN})—, —NR^{IIN}C(O)O—, or —NR^{IIN}C(O)N(R^{IIN})—;
each instance of R^{II2} is independently optionally substituted C₁₋₃₀ alkyl, optionally substituted C₁₋₃₀ alkenyl, or optionally substituted C₁₋₃₀ alkynyl; optionally wherein one or more methylene units of R^{II2} are independently replaced with optionally substituted carbocyclylene, optionally substituted heterocyclylene, optionally substituted arylene, optionally substituted heteroarylene, —N(R^{IIN})—, —O—, —S—, —C(O)—, —C(O)N(R^{IIN})—, —NR^{IIN}C(O)—, —NR^{IIN}C(O)N(R^{IIN})—, —C(O)O—, —OC(O)—, —OC(O)O—, —OC(O)N(R^{IIN})—, —NR^{IIN}C(O)O—, —C(O)S—, —SC(O)—, —C(═NR^{IIN})—, —C(═NR^{IIN})N(R^{IIN})—, —NR^{IIN}C(═NR^{IIN})—, —NR^{IIN}C(═NR^{IIN})N(R^{IIN})—, —C(S)—, —C(S)N(R^{IIN})—, —NR^{IIN}C(S)—, —NR^{IIN}C(S)N(R^{IIN})—, —S(O)—, —OS(O)—, —S(O)O—, —OS(O)O—, —OS(O)₂—, —S(O)₂O—, —OS(O)₂O—, —N(R^{IIN}) S(O)—, —S(O)N(R^{IIN})—, —N(R^{IIN})S(O)N(R^{IIN})—, —OS(O)N(R^{IIN})—, —N(R^{IIN})S(O)O—, —S(O)₂—, —N(R^{IIN})S(O)₂—, —S(O)₂N(R^{IIN})—, —N(R^{IIN})S(O)₂N(R^{IIN})—, —OS(O)₂N(R^{IIN})—, or —N(R^{IIN})S(O)₂O—;
each instance of R^{IIN} is independently hydrogen, optionally substituted alkyl, or a nitrogen-protecting group;
Ring B^{II} is optionally substituted carbocyclyl, optionally substituted heterocyclyl, optionally substituted aryl, or optionally substituted heteroaryl; and
p^{II} is 1 or 2;
provided that the compound is not of the formula:

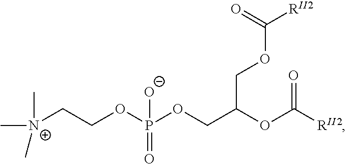

wherein each instance of R^{II2} is independently unsubstituted alkyl, unsubstituted alkenyl, or unsubstituted alkynyl.

In some embodiments, the phospholipids may be one or more of the phospholipids described in U.S. Application No. 62/520,530.

### (i) Phospholipid Head Modifications

In some embodiments, a phospholipid useful or potentially useful in the present invention comprises a modified phospholipid head (e.g., a modified choline group). In some embodiments, a phospholipid with a modified head is DSPC, or analog thereof, with a modified quaternary amine. For example, in embodiments of Formula (PL-II), at least one of R^{II1} is not methyl. In some embodiments, at least one of R^{II1} is not hydrogen or methyl. In some embodiments, the compound of Formula (PL-II) is of one of the following formulae:

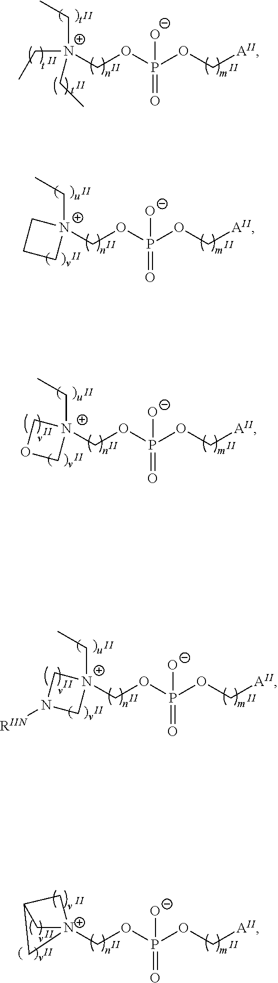

or a salt thereof, wherein:
each t^{II} is independently 1, 2, 3, 4, 5, 6, 7, 8, 9, or 10;
each u^{II} is independently 0, 1, 2, 3, 4, 5, 6, 7, 8, 9, or 10; and
each v^{II} is independently 1, 2, or 3.

In some embodiments, a compound of Formula (PL-II) is of Formula (PL-I-a):

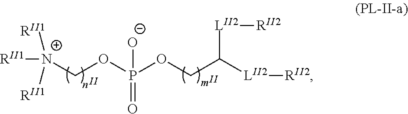

or a salt thereof.

In some embodiments, a phospholipid useful or potentially useful in the present invention comprises a cyclic moiety in place of the glyceride moiety. In some embodiments, a phospholipid useful in the present invention is DSPC, or analog thereof, with a cyclic moiety in place of the glyceride moiety. In some embodiments, the compound of Formula (PL-II) is of Formula (PL-II-b):

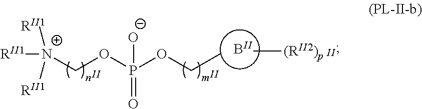

or a salt thereof.

### (ii) Phospholipid Tail Modifications

In some embodiments, a phospholipid useful or potentially useful in the present invention comprises a modified tail. In some embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in some embodiments, the compound of (PL-II) is of Formula (PL-II-a), or a salt thereof, wherein at least one instance of R^{II2} or each instance of R^{II2} is optionally substituted C₁₋₃₀ alkyl, wherein one or more methylene units of R^{II2} are independently replaced with optionally substituted carbocyclylene, optionally substituted heterocyclylene, optionally substituted arylene, optionally substituted heteroarylene, —N(R^{IIN})—, —O—, —S—, —C(O)—, —C(O)N(R^{IIN})—, —NR^{IIN}C(O)—, —NR^{IIN}C(O)N(R^{IIN})—, —C(O)O—, —OC(O)—, —OC(O)O—, —OC(O)N(R^{IIN})—, —NR^{IIN}C(O)O—, —C(O)S—, —SC(O)—, —C(═NR^{IIN})—, C(═NR^{IIN})N(R^{IIN})—, —NR^{IIN}C(═NR^{IIN})—, —NR^{IIN}C(═NR^{IIN})N(R^{IIN})—, —C(S)—, —C(S)N(R^{IIN})—, —NR^{IIN}C(S)—, —NR^{IIN}C(S)N(R^{IIN})—, —S(O)—, —OS(O)—, —S(O)O—, —OS(O)O—, —OS(O)₂—, —S(O)₂O—, —OS(O)₂O—, —N(R^{IIN}) S(O)—, —S(O)N(R^{IIN})—, —N(R^{IIN})S(O)N(R^{IIN})—, —OS(O)N(R^{IIN})—, —N(R^{IIN})S(O)O—, —S(O)₂—, —N(R^{IIN}) S(O)₂—, —S(O)₂N(R^{IIN})—, —N(R^{IIN})S(O)₂N(R^{IIN})—, —OS(O)₂N(R^{IIN})—, or —N(R^{IIN})S(O)₂O—.

In some embodiments, the compound of Formula (PL-II) is of Formula (PL-II-c):

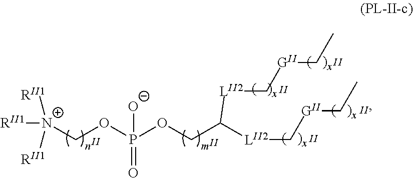

or a salt thereof, wherein:
each x^{II} is independently an integer between 0-30, inclusive, and
each G^{II} is independently selected from the group consisting of optionally substituted carbocyclylene, optionally substituted heterocyclylene, optionally substituted arylene, optionally substituted heteroarylene, —N(R^{IIN})—, —O—, —S—, —C(O)—, —C(O)N(R^{IIN})—, —NR^{IIN}C(O)—, —NR^{IIN}C(O)N(R^{IIN})—, —C(O)O—, —OC(O)—, —OC(O)O—, —OC(O)N(R^{IIN})—, —NR^{IIN}C(O)O—, —C(O)S—, —SC(O)—, —C(═NR^{IIN})—, —C(═NR^{IIN})N(R^{IIN})—, —NR^{IIN}C(═NR^{IIN})—, —NR^{IIN}C(═NR^{IIN})N(R^{IIN})—, —C(S)—, —C(S)N(R^{IIN})—, —NR^{IIN}C(S)—, —NR^{IIN}C(S)N(R^{IIN})—, —S(O)—, —OS(O)—, —S(O)O—, —OS(O)O—, —OS(O)₂—, —S(O)₂O—, —OS(O)₂O—, —N(R^{IIN})S(O)—, —S(O)N(R^{IIN})—, —N(R^{IIN})S(O)N(R^{IIN})—, —OS(O)N(R^{IIN})—, —N(R^{IIN})S(O)O—, —S(O)₂—, —N(R^{IIN})S(O)₂—, —S(O)₂N(R^{IIN})—, —N(R^{IIN})S(O)₂N(R^{IIN})—, —OS(O)₂N(R^{IIN})—, or —N(R^{IIN})S(O)₂O—. Each possibility represents a separate embodiment of the present invention.

In some embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in some embodiments, a phospholipid useful or potentially useful in the present invention is a compound of Formula (PL-II), wherein n″ is 1, 3, 4, 5, 6, 7, 8, 9, or 10. For example, in some embodiments, a compound of Formula (PL-II) is of one of the following formulae:

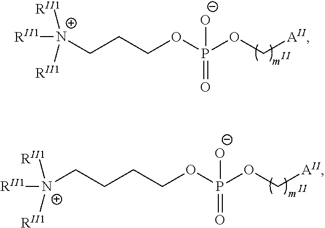

or a salt thereof.

### Alternative Lipids

In some embodiments, an alternative lipid is used in place of a phospholipid of the present disclosure. Non-limiting examples of such alternative lipids include the following:

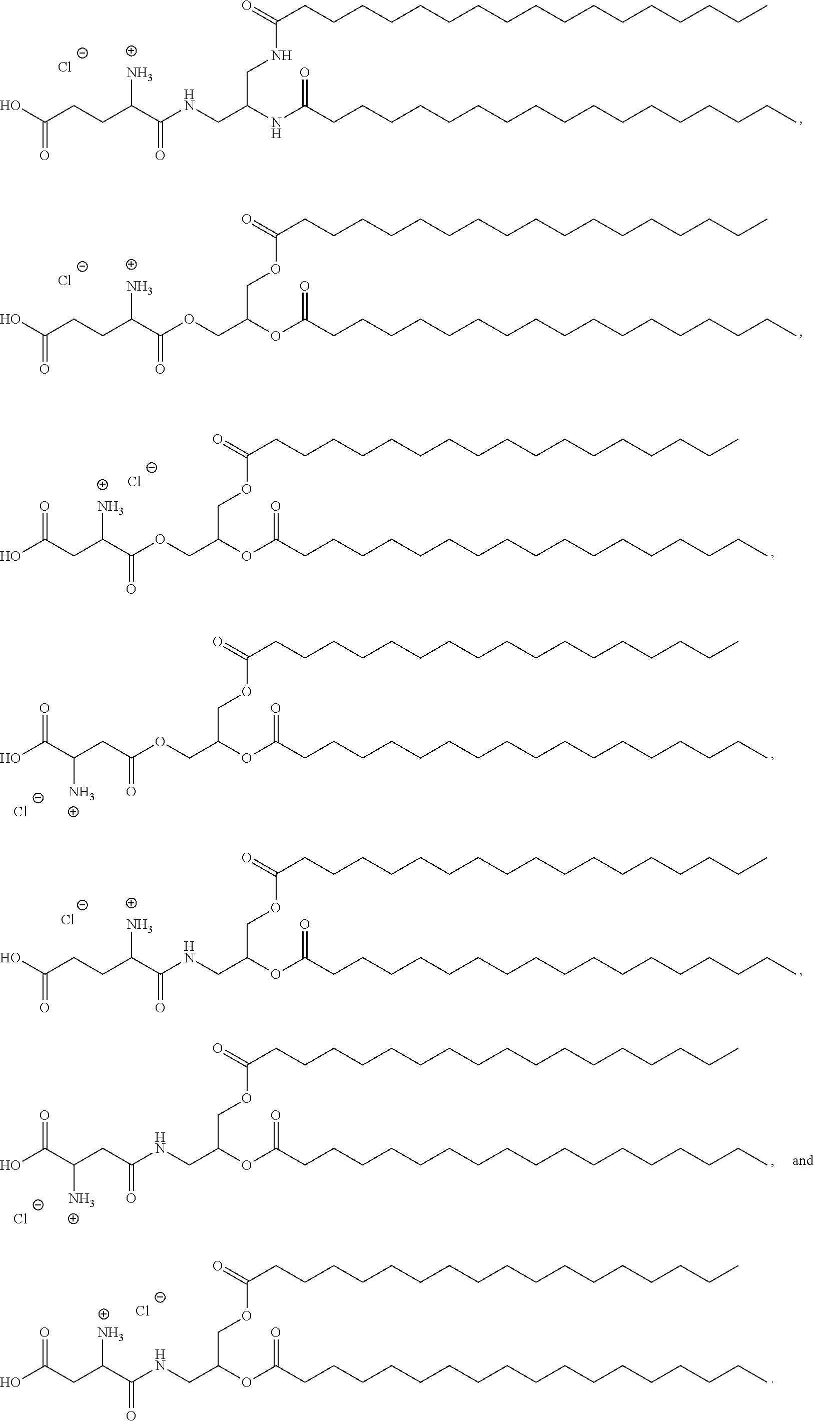

In other embodiments, a therapeutic and/or prophylactic is a protein, for example, a protein needed to augment or replace a naturally occurring protein of interest. Such proteins or polypeptides may be naturally occurring, or may be modified using methods known in the art, e.g., to increase half life. Exemplary proteins are intracellular, transmembrane, or secreted.

### Ionizable Lipids

In some embodiments, the ionizable lipid is a compound of Formula (IL-IA):

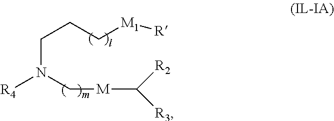

or a salt or isomer thereof, wherein
l is selected from 1, 2, 3, 4, and 5;
m is selected from 5, 6, 7, 8, and 9;
M¹ is a bond or M′;
R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, or heteroaryl, and each n is selected from 1, 2, 3, 4, or 5;
M and M′ are independently selected from —C(O)O—, —OC(O)—, —C(O)N(R′)—, —P(O)(OR′)O—, —S—S—, an aryl group, and a heteroaryl group; and
R₂ and R₃ are both C₁₋₁₄ alkyl or C₂₋₁₄ alkenyl;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H; and
R′ is a C₁₋₁₈ alkyl or C₂₋₁₈ alkenyl.

In some embodiments, the ionizable lipid is a compound of Formula (IL-IB):

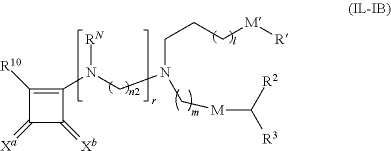

### or its N-oxide,

or a salt or isomer thereof, wherein
l is selected from 1, 2, 3, 4, and 5;
m is selected from 5, 6, 7, 8, and 9;
R^{a′} and R^{b′} are independently selected from the group consisting of C₁₋₁₄ alkyl and C₂₋₁₄ alkenyl; and
R² and R³ are independently selected from the group consisting of C₁₋₁₄ alkyl and C₂₋₁₄ alkenyl;
M and M′ are independently selected from —C(O)O— and —OC(O)—;
R^{N} is H, or C₁₋₃ alkyl;
X^{a} and X^{b} are each independently O or S;
R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O(CH₂)_{q1}N(R)₂, —NH(CH₂)ₛ₁OR, —N((CH₂)ₛOR)₂, —N(R)-carbocycle, —N(R)-heterocycle, —N(R)-aryl, —N(R)-heteroaryl, —N(R)(CH₂)ₜ₁-carbocycle, —N(R)(CH₂)ₜ₁-heterocycle, —N(R)(CH₂)ₜ₁-aryl, —N(R)(CH₂)ₜ₁-heteroaryl, a carbocycle, a heterocycle, aryl and heteroaryl;
each R is independently selected from the group consisting of C₁₋₁₂ alkyl, C₂₋₁₂ alkenyl, and H;
m is selected from 5, 6, 7, 8, 9, 10, 11, 12, and 13;
n2 is selected from 1, 2, 3, 4, 5, 6, 7, 8, 9, and 10;
r is 0 or 1;
t¹ is selected from 1, 2, 3, 4, and 5;
p¹ is selected from 1, 2, 3, 4, and 5;
q¹ is selected from 1, 2, 3, 4, and 5; and
s¹ is selected from 1, 2, 3, and 4.

In some embodiments, the ionizable lipid is a compound of Formula (IL-IC):

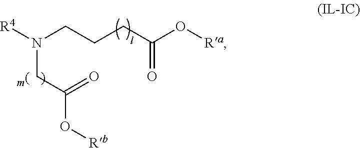

or its N-oxide, or a salt or isomer thereof,
wherein R′^{a} is R′^{branched} or R′^{cyclic}, wherein
R′^{branched} is

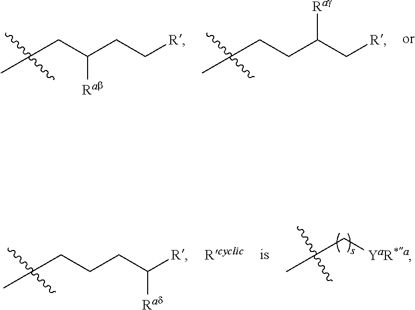

and

### R′^{b} is

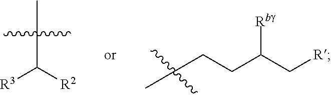

wherein

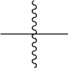

denotes a point of attachment;
wherein R^{aγ}, R^{aγ}, and R^{aγ} are each C₁₋₁₂ alkyl or C₂₋₁₂ alkenyl;
R^{bγ} is H, C₁₋₁₂ alkyl or C₂₋₁₂ alkenyl;
R² and R³ are each independently selected from the group consisting of C₁₋₁₄ alkyl and C₂₋₁₄ alkenyl;
R⁴ is —(CH₂)ₙOH or

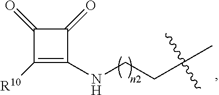

wherein

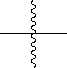

denotes a point of attachment;
each R′ independently is a C₁₋₁₂ alkyl or C₂₋₁₂ alkenyl;
R¹⁰ is N(R)₂; each R is independently selected from the group consisting of C₁₋₆ alkyl, C₂₋₃ alkenyl, and H; and n and n2 are each selected from the group consisting of 1, 2, 3, 4, and 5;
Y^{a} is a C₃₋₆ carbocycle;
R*″^{a} is selected from the group consisting of C₁₋₁₅ alkyl and C₂₋₁₅ alkenyl;
l is selected from 1, 2, 3, 4, and 5;
m is selected from 5, 6, 7, 8, and 9; and
s is 2 or 3.

In some embodiments, the ionizable lipid is a compound selected from Table IL-1.

TABLE IL-1

| Cpd. | |
|---|---|
| # | Structure |
| I-1 | 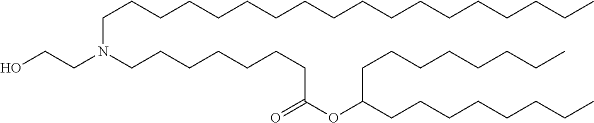 [SMILES: CCCCCCCCCCCCCCCCCCN(CCO)CCCCCCCC(=O)OC(CCCCCCCC)CCCCCCCC] |
| I-2 | 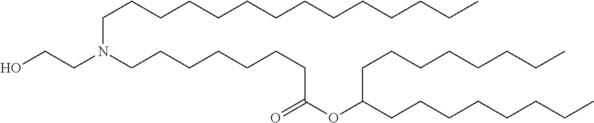 [SMILES: CCCCCCCCCCCCCCN(CCO)CCCCCCCC(=O)OC(CCCCCCCC)CCCCCCCC] |
| I-3 | 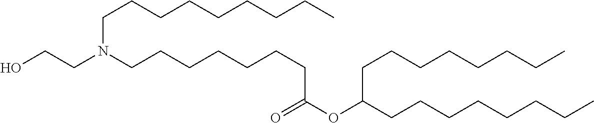 [SMILES: CCCCCCCCCN(CCO)CCCCCCCC(=O)OC(CCCCCCCC)CCCCCCCC] |
| I-4 | 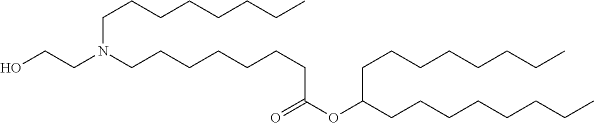 [SMILES: CCCCCCCCC(CCCCCCCC)OC(=O)CCCCCCCN(CCO)CCCCCCCC] |
| I-5 | 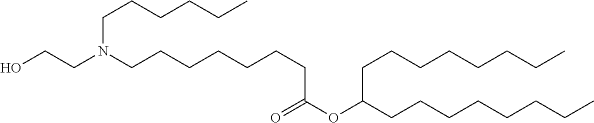 [SMILES: CCCCCCCCC(CCCCCCCC)OC(=O)CCCCCCCN(CCO)CCCCCC] |
| I-6 | 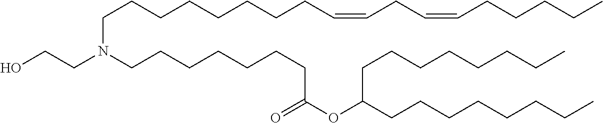 [SMILES: CCCCC/C=C\C/C=C\CCCCCCCCN(CCO)CCCCCCCC(=O)OC(CCCCCCCC)CCCCCCCC] |
| I-7 | 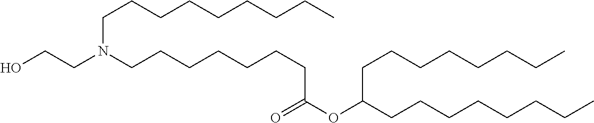 [SMILES: CCCCCCCCCN(CCO)CCCCCCCC(=O)OC(CCCCCCCC)CCCCCCCC] |
| I-8 | 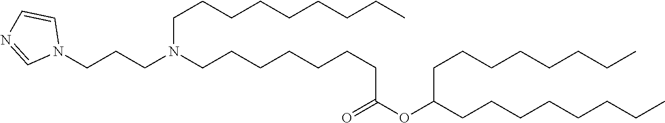 [SMILES: CCCCCCCCCN(CCCCCCCC(=O)OC(CCCCCCCC)CCCCCCCC)CCCn1ccnc1] |
| I-9 | 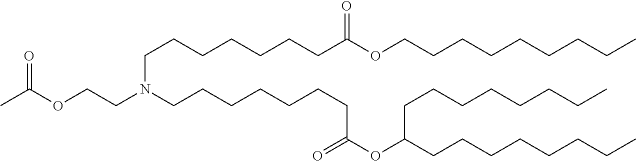 [SMILES: CCCCCCCCCOC(=O)CCCCCCCN(CCCCCCCC(=O)OC(CCCCCCCC)CCCCCCCC)CCOC(C)=O] |
| I-10 | 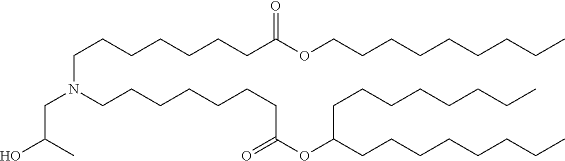 [SMILES: CCCCCCCCCOC(=O)CCCCCCCN(CCCCCCCC(=O)OC(CCCCCCCC)CCCCCCCC)CC(C)O] |
| I-11 | 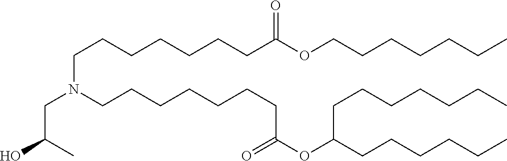 [SMILES: CCCCCCCOC(=O)CCCCCCCN(CCCCCCCC(=O)OC(CCCCCC)CCCCCCC)C[C@@H](C)O] |
| I-12 | 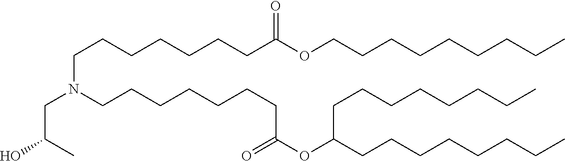 [SMILES: CCCCCCCCCOC(=O)CCCCCCCN(CCCCCCCC(=O)OC(CCCCCCCC)CCCCCCCC)C[C@H](C)O] |
| I-13 | 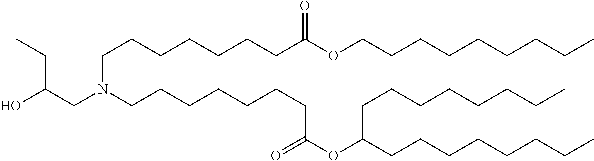 [SMILES: CCCCCCCCCOC(=O)CCCCCCCN(CCCCCCCC(=O)OC(CCCCCCCC)CCCCCCCC)CC(O)CC] |
| I-14 | 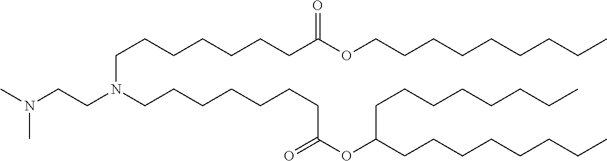 [SMILES: CCCCCCCCCOC(=O)CCCCCCCN(CCCCCCCC(=O)OC(CCCCCCCC)CCCCCCCC)CCN(C)C] |
| I-15 | 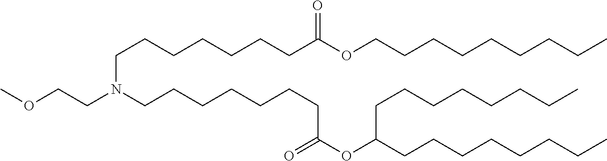 [SMILES: CCCCCCCCCOC(=O)CCCCCCCN(CCCCCCCC(=O)OC(CCCCCCCC)CCCCCCCC)CCOC] |
| I-16 | 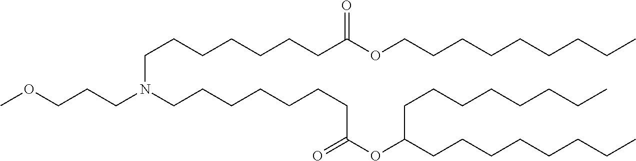 [SMILES: CCCCCCCCCOC(=O)CCCCCCCN(CCCCCCCC(=O)OC(CCCCCCCC)CCCCCCCC)CCCOC] |
| I-17 | 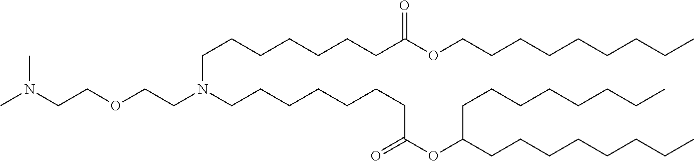 [SMILES: CCCCCCCCCOC(=O)CCCCCCCN(CCCCCCCC(=O)OC(CCCCCCCC)CCCCCCCC)CCOCCN(C)C] |
| I-18 | 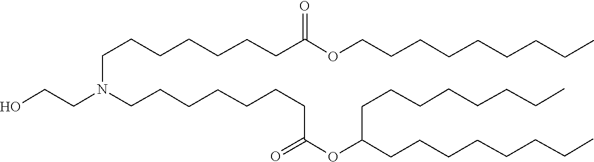 [SMILES: CCCCCCCCCOC(=O)CCCCCCCN(CCO)CCCCCCCC(=O)OC(CCCCCCCC)CCCCCCCC] |
| I-19 | 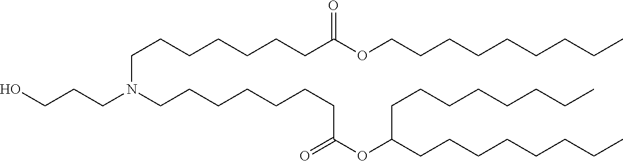 [SMILES: CCCCCCCCCOC(=O)CCCCCCCN(CCCO)CCCCCCCC(=O)OC(CCCCCCCC)CCCCCCCC] |
| I-20 | 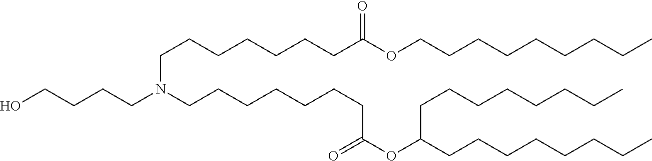 [SMILES: CCCCCCCCCOC(=O)CCCCCCCN(CCCCO)CCCCCCCC(=O)OC(CCCCCCCC)CCCCCCCC] |
| I-21 | 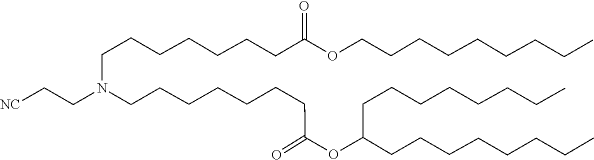 [SMILES: CCCCCCCCCOC(=O)CCCCCCCN(CCC#N)CCCCCCCC(=O)OC(CCCCCCCC)CCCCCCCC] |
| I-22 | 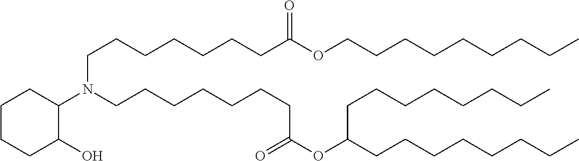 [SMILES: CCCCCCCCCOC(=O)CCCCCCCN(CCCCCCCC(=O)OC(CCCCCCCC)CCCCCCCC)C1CCCCC1O] |
| I-23 | 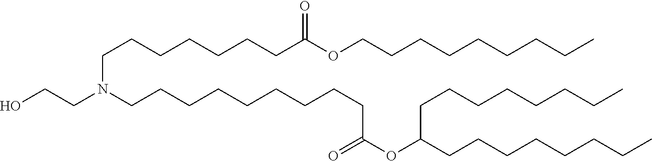 [SMILES: CCCCCCCCCOC(=O)CCCCCCCN(CCO)CCCCCCCCCC(=O)OC(CCCCCCCC)CCCCCCCC] |
| I-24 | 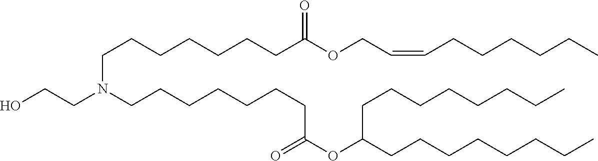 [SMILES: CCCCCC/C=C\COC(=O)CCCCCCCN(CCO)CCCCCCCC(=O)OC(CCCCCCCC)CCCCCCCC] |
| I-25 | 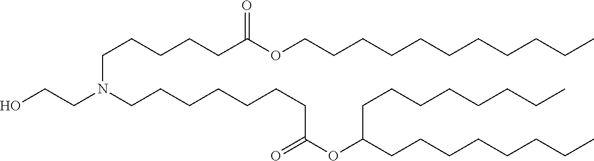 [SMILES: CCCCCCCCCCCOC(=O)CCCCCN(CCO)CCCCCCCC(=O)OC(CCCCCCCC)CCCCCCCC] |
| I-26 | 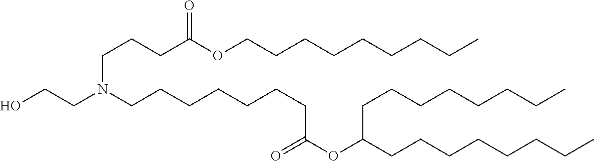 [SMILES: CCCCCCCCCOC(=O)CCCN(CCO)CCCCCCCC(=O)OC(CCCCCCCC)CCCCCCCC] |
| I-27 | 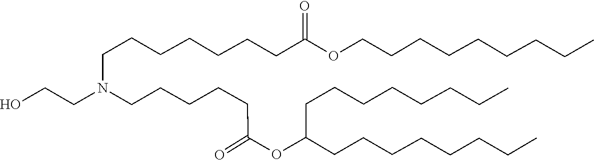 [SMILES: CCCCCCCCCOC(=O)CCCCCCCN(CCO)CCCCCC(=O)OC(CCCCCCCC)CCCCCCCC] |
| I-28 | 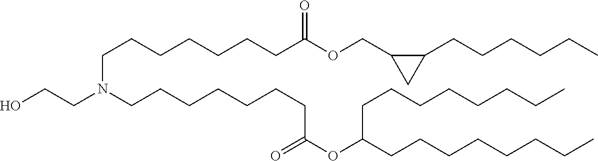 [SMILES: CCCCCCCCC(CCCCCCCC)OC(=O)CCCCCCCN(CCO)CCCCCCCC(=O)OCC1CC1CCCCCC] |
| I-29 | 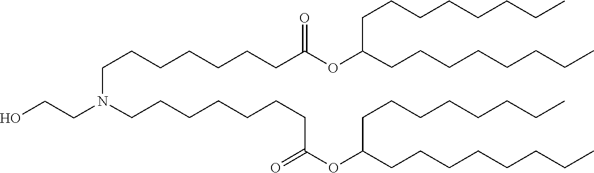 [SMILES: CCCCCCCCC(CCCCCCCC)OC(=O)CCCCCCCN(CCO)CCCCCCCC(=O)OC(CCCCCCCC)CCCCCCCC] |
| I-30 | 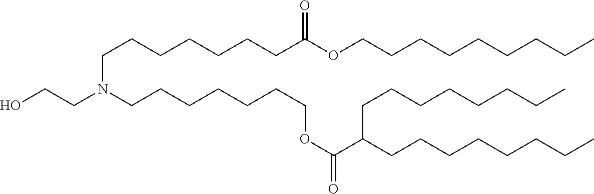 [SMILES: CCCCCCCCCOC(=O)CCCCCCCN(CCO)CCCCCCCOC(=O)C(CCCCCCCC)CCCCCCCC] |
| I-31 | 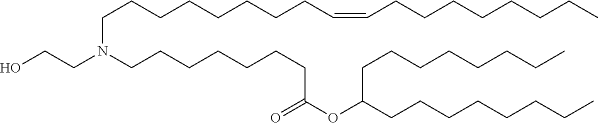 [SMILES: CCCCCCCC/C=C\CCCCCCCCN(CCO)CCCCCCCC(=O)OC(CCCCCCCC)CCCCCCCC] |
| I-32 | 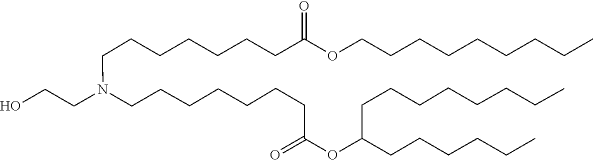 [SMILES: CCCCCCCCCOC(=O)CCCCCCCN(CCO)CCCCCCCC(=O)OC(CCCCCC)CCCCCCCC] |
| I-33 | 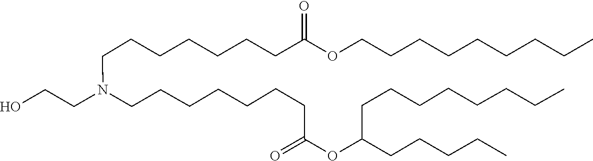 [SMILES: CCCCCCCCCOC(=O)CCCCCCCN(CCO)CCCCCCCC(=O)OC(CCCCC)CCCCCCCC] |
| I-34 | 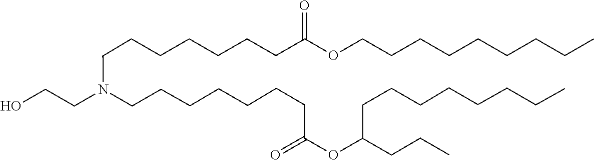 [SMILES: CCCCCCCCCOC(=O)CCCCCCCN(CCO)CCCCCCCC(=O)OC(CCC)CCCCCCCC] |
| I-35 | 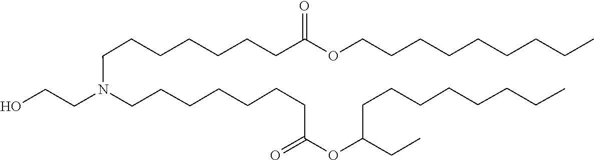 [SMILES: CCCCCCCCCOC(=O)CCCCCCCN(CCO)CCCCCCCC(=O)OC(CC)CCCCCCCC] |
| I-36 | 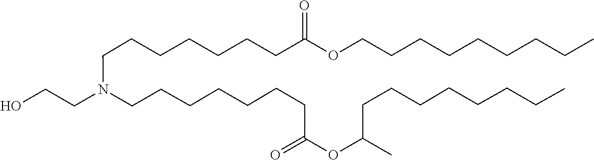 [SMILES: CCCCCCCCCOC(=O)CCCCCCCN(CCO)CCCCCCCC(=O)OC(C)CCCCCCCC] |
| I-37 | 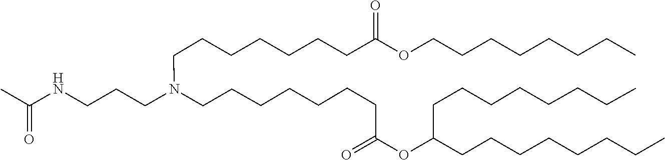 [SMILES: CCCCCCCCOC(=O)CCCCCCCN(CCCCCCCC(=O)OC(CCCCCCCC)CCCCCCCC)CCCNC(C)=O] |
| I-38 | 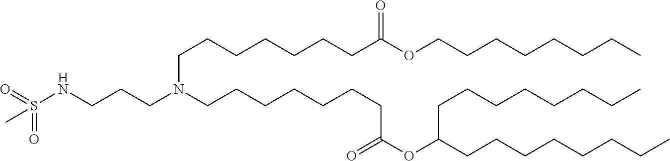 [SMILES: CCCCCCCCOC(=O)CCCCCCCN(CCCCCCCC(=O)OC(CCCCCCCC)CCCCCCCC)CCCNS(C)(=O)=O] |
| I-39 | 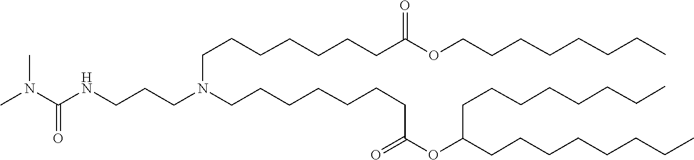 [SMILES: CCCCCCCCOC(=O)CCCCCCCN(CCCCCCCC(=O)OC(CCCCCCCC)CCCCCCCC)CCCNC(=O)N(C)C] |
| I-40 | 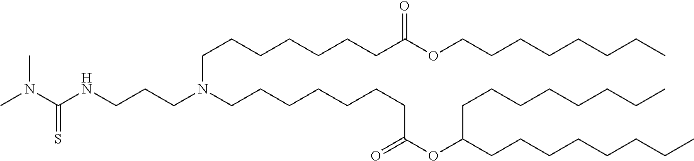 [SMILES: CCCCCCCCOC(=O)CCCCCCCN(CCCCCCCC(=O)OC(CCCCCCCC)CCCCCCCC)CCCNC(=S)N(C)C] |
| I-41 | 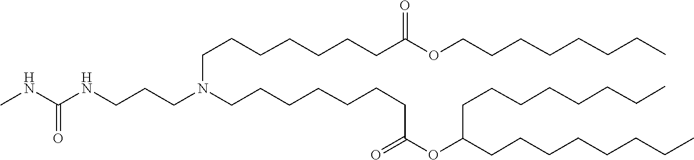 [SMILES: CCCCCCCCOC(=O)CCCCCCCN(CCCCCCCC(=O)OC(CCCCCCCC)CCCCCCCC)CCCNC(=O)NC] |
| I-42 | 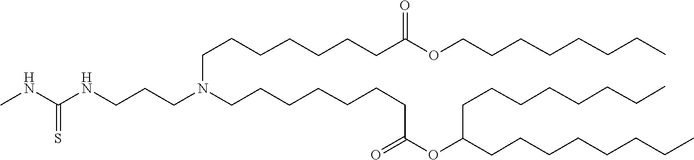 [SMILES: CCCCCCCCOC(=O)CCCCCCCN(CCCCCCCC(=O)OC(CCCCCCCC)CCCCCCCC)CCCNC(=S)NC] |
| I-43 | 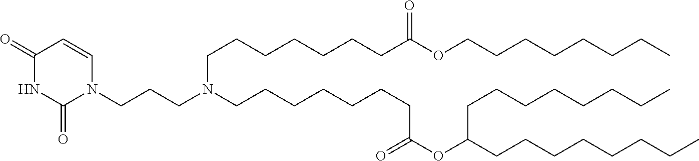 [SMILES: CCCCCCCCOC(=O)CCCCCCCN(CCCCCCCC(=O)OC(CCCCCCCC)CCCCCCCC)CCCn1ccc(=O)[nH]c1=O] |
| I-44 | 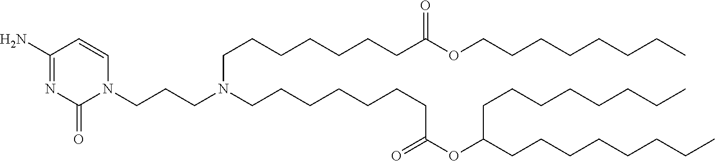 [SMILES: CCCCCCCCOC(=O)CCCCCCCN(CCCCCCCC(=O)OC(CCCCCCCC)CCCCCCCC)CCCn1ccc(N)nc1=O] |
| I-45 | 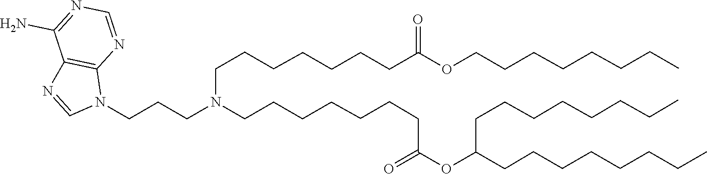 [SMILES: CCCCCCCCOC(=O)CCCCCCCN(CCCCCCCC(=O)OC(CCCCCCCC)CCCCCCCC)CCCn1cnc2c(N)ncnc21] |
| I-46 | 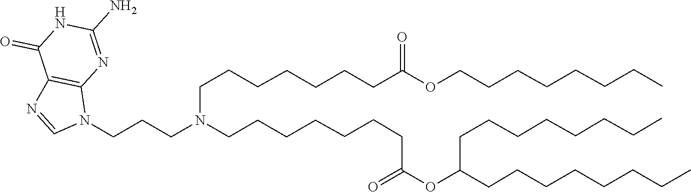 [SMILES: CCCCCCCCOC(=O)CCCCCCCN(CCCCCCCC(=O)OC(CCCCCCCC)CCCCCCCC)CCCn1cnc2c(=O)[nH]c(N)nc21] |
| I-47 | 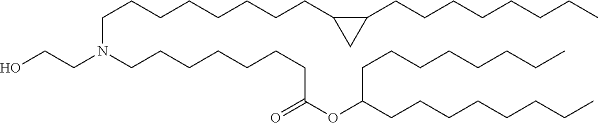 [SMILES: CCCCCCCCC(CCCCCCCC)OC(=O)CCCCCCCN(CCO)CCCCCCCCC1CC1CCCCCCCC] |
| I-48 | 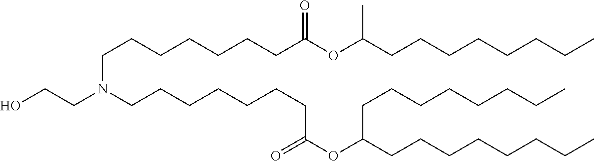 [SMILES: CCCCCCCCC(C)OC(=O)CCCCCCCN(CCO)CCCCCCCC(=O)OC(CCCCCCCC)CCCCCCCC] |
| I-49 | 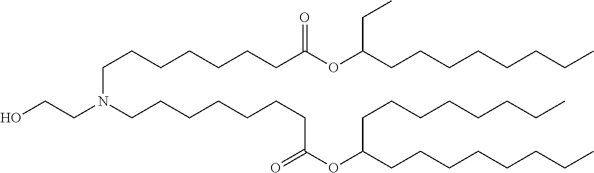 [SMILES: CCCCCCCCC(CC)OC(=O)CCCCCCCN(CCO)CCCCCCCC(=O)OC(CCCCCCCC)CCCCCCCC] |
| I-50 | 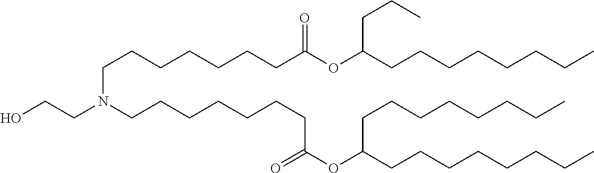 [SMILES: CCCCCCCCC(CCC)OC(=O)CCCCCCCN(CCO)CCCCCCCC(=O)OC(CCCCCCCC)CCCCCCCC] |
| I-51 | 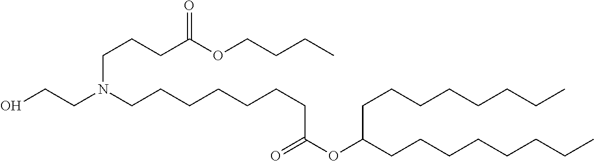 [SMILES: CCCCCCCCC(CCCCCCCC)OC(=O)CCCCCCCN(CCO)CCCC(=O)OCCCC] |
| I-52 | 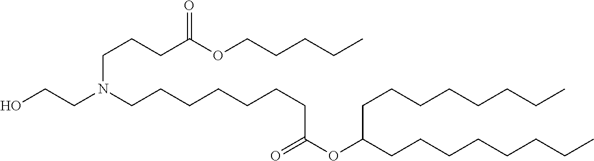 [SMILES: CCCCCCCCC(CCCCCCCC)OC(=O)CCCCCCCN(CCO)CCCC(=O)OCCCCC] |
| I-53 | 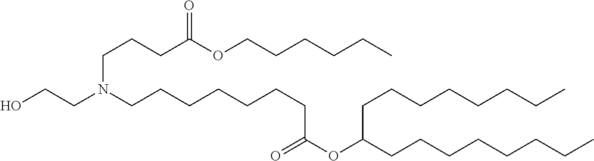 [SMILES: CCCCCCCCC(CCCCCCCC)OC(=O)CCCCCCCN(CCO)CCCC(=O)OCCCCCC] |
| I-54 | 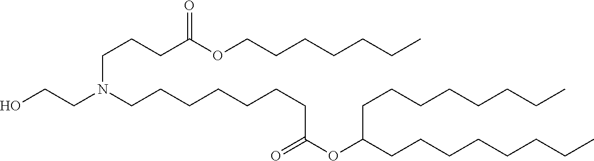 [SMILES: CCCCCCCCC(CCCCCCCC)OC(=O)CCCCCCCN(CCO)CCCC(=O)OCCCCCCC] |
| I-55 | 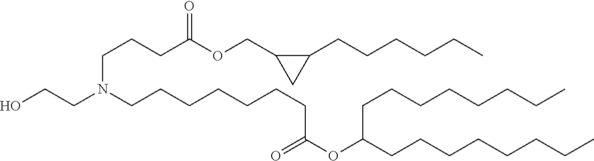 [SMILES: CCCCCCCCC(CCCCCCCC)OC(=O)CCCCCCCN(CCO)CCCC(=O)OCC1CC1CCCCCC] |
| I-56 | 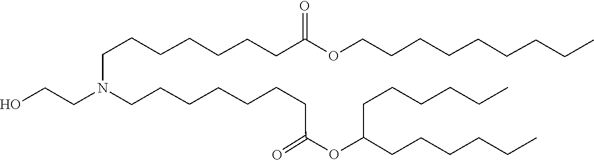 [SMILES: CCCCCCCCCOC(=O)CCCCCCCN(CCO)CCCCCCCC(=O)OC(CCCCCC)CCCCCC] |
| I-57 | 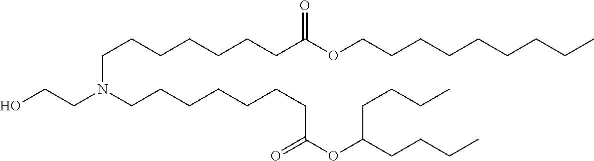 [SMILES: CCCCCCCCCOC(=O)CCCCCCCN(CCO)CCCCCCCC(=O)OC(CCCC)CCCC] |
| I-58 | 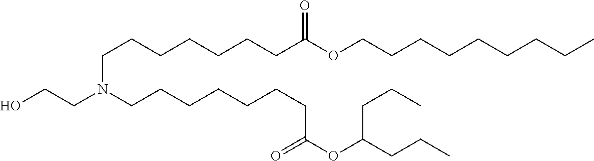 [SMILES: CCCCCCCCCOC(=O)CCCCCCCN(CCO)CCCCCCCC(=O)OC(CCC)CCC] |
| I-59 | 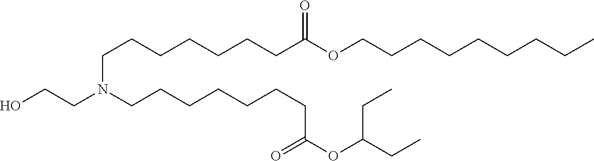 [SMILES: CCCCCCCCCOC(=O)CCCCCCCN(CCO)CCCCCCCC(=O)OC(CC)CC] |
| I-60 | 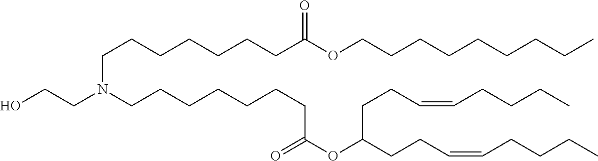 [SMILES: CCCC/C=C\CCC(CC/C=C\CCCC)OC(=O)CCCCCCCN(CCO)CCCCCCCC(=O)OCCCCCCCCC] |
| I-61 | 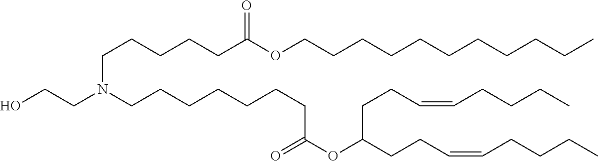 [SMILES: CCCC/C=C\CCC(CC/C=C\CCCC)OC(=O)CCCCCCCN(CCO)CCCCCC(=O)OCCCCCCCCCCC] |
| I-62 | 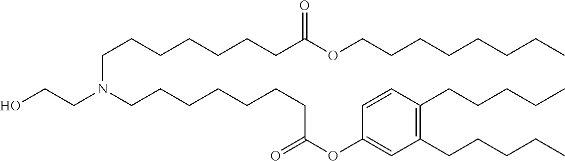 [SMILES: CCCCCCCCOC(=O)CCCCCCCN(CCO)CCCCCCCC(=O)Oc1ccc(CCCCC)c(CCCCC)c1] |
| I-63 | 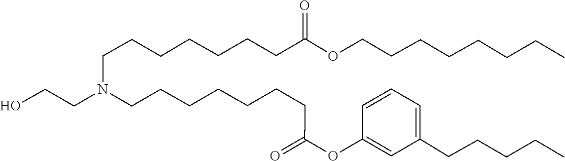 [SMILES: CCCCCCCCOC(=O)CCCCCCCN(CCO)CCCCCCCC(=O)Oc1cccc(CCCCC)c1] |
| I-64 | 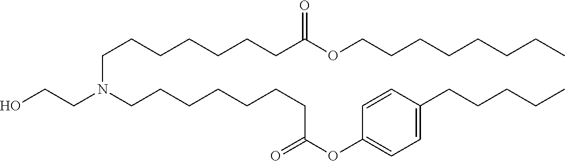 [SMILES: CCCCCCCCOC(=O)CCCCCCCN(CCO)CCCCCCCC(=O)Oc1ccc(CCCCC)cc1] |
| I-65 | 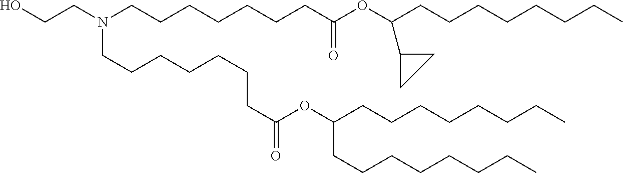 [SMILES: CCCCCCCCC(CCCCCCCC)OC(=O)CCCCCCCN(CCO)CCCCCCCC(=O)OC(CCCCCCCC)C1CC1] |
| I-66 | 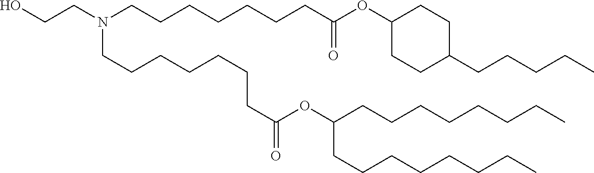 [SMILES: CCCCCCCCC(CCCCCCCC)OC(=O)CCCCCCCN(CCO)CCCCCCCC(=O)OC1CCC(CCCCC)CC1] |
| I-67 | 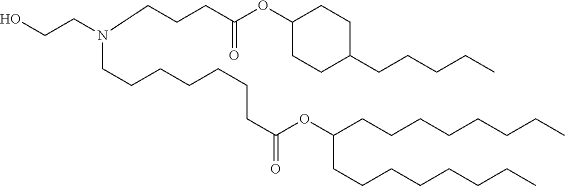 [SMILES: CCCCCCCCC(CCCCCCCC)OC(=O)CCCCCCCN(CCO)CCCC(=O)OC1CCC(CCCCC)CC1] |
| I-68 | 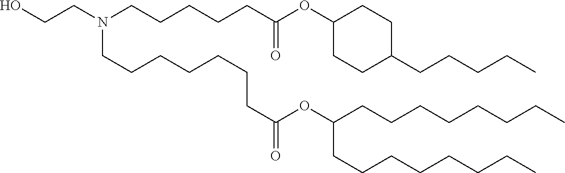 [SMILES: CCCCCCCCC(CCCCCCCC)OC(=O)CCCCCCCN(CCO)CCCCCC(=O)OC1CCC(CCCCC)CC1] |
| I-69 | 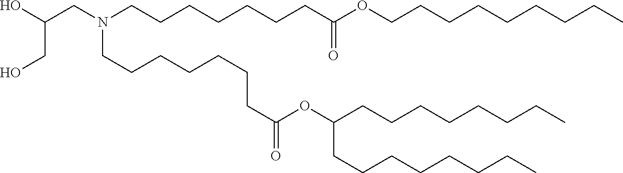 [SMILES: CCCCCCCCCOC(=O)CCCCCCCN(CCCCCCCC(=O)OC(CCCCCCCC)CCCCCCCC)CC(O)CO] |
| I-70 | 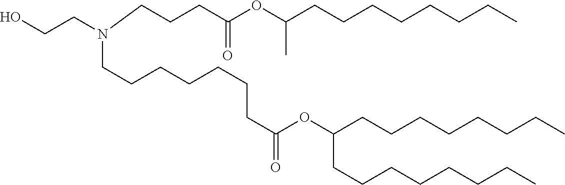 [SMILES: CCCCCCCCC(C)OC(=O)CCCN(CCO)CCCCCCCC(=O)OC(CCCCCCCC)CCCCCCCC] |
| I-71 | 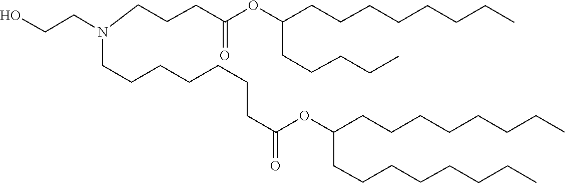 [SMILES: CCCCCCCCC(CCCCCCCC)OC(=O)CCCCCCCN(CCO)CCCC(=O)OC(CCCCC)CCCCCCCC] |
| I-72 | 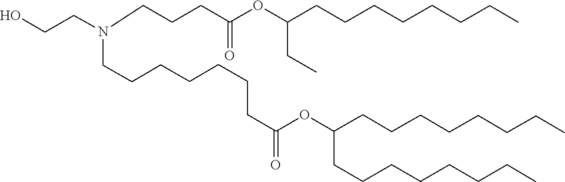 [SMILES: CCCCCCCCC(CC)OC(=O)CCCN(CCO)CCCCCCCC(=O)OC(CCCCCCCC)CCCCCCCC] |
| I-73 | 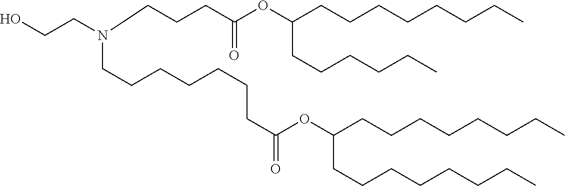 [SMILES: CCCCCCCCC(CCCCCCCC)OC(=O)CCCCCCCN(CCO)CCCC(=O)OC(CCCCCC)CCCCCCCC] |
| I-74 | 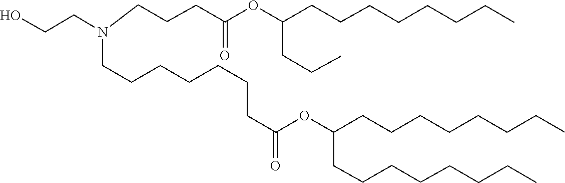 [SMILES: CCCCCCCCC(CCC)OC(=O)CCCN(CCO)CCCCCCCC(=O)OC(CCCCCCCC)CCCCCCCC] |
| I-75 | 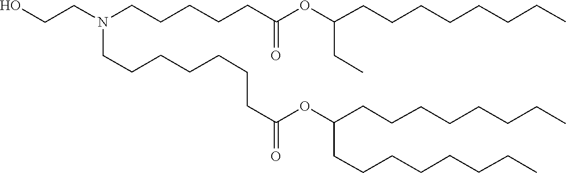 [SMILES: CCCCCCCCC(CC)OC(=O)CCCCCN(CCO)CCCCCCCC(=O)OC(CCCCCCCC)CCCCCCCC] |
| I-76 | 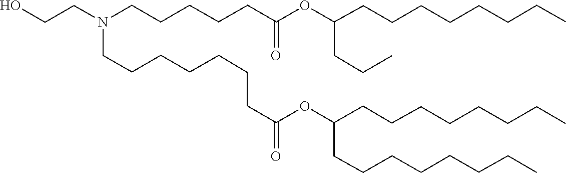 [SMILES: CCCCCCCCC(CCC)OC(=O)CCCCCN(CCO)CCCCCCCC(=O)OC(CCCCCCCC)CCCCCCCC] |
| I-77 | 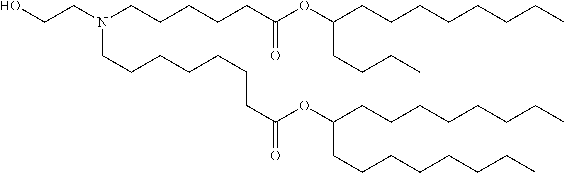 [SMILES: CCCCCCCCC(CCCC)OC(=O)CCCCCN(CCO)CCCCCCCC(=O)OC(CCCCCCCC)CCCCCCCC] |
| I-78 | 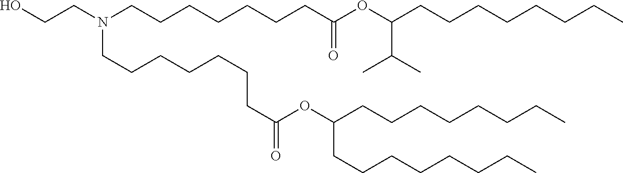 [SMILES: CCCCCCCCC(CCCCCCCC)OC(=O)CCCCCCCN(CCO)CCCCCCCC(=O)OC(CCCCCCCC)C(C)C] |
| I-79 | 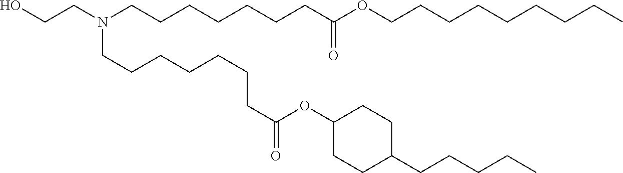 [SMILES: CCCCCCCCCOC(=O)CCCCCCCN(CCO)CCCCCCCC(=O)OC1CCC(CCCCC)CC1] |
| I-80 | 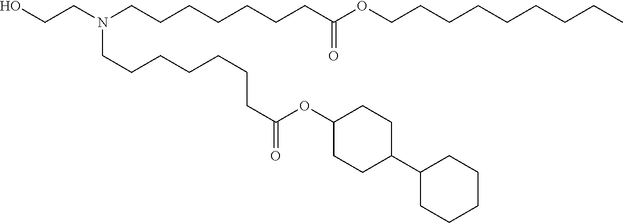 [SMILES: CCCCCCCCCOC(=O)CCCCCCCN(CCO)CCCCCCCC(=O)OC1CCC(C2CCCCC2)CC1] |
| I-81 | 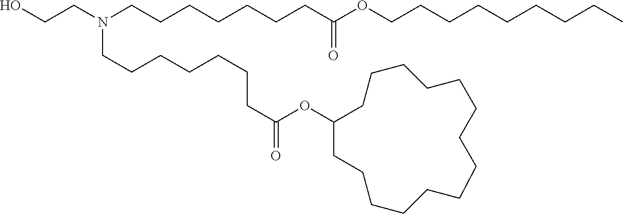 [SMILES: CCCCCCCCCOC(=O)CCCCCCCN(CCO)CCCCCCCC(=O)OC1CCCCCCCCCCCCCC1] |
| I-82 | 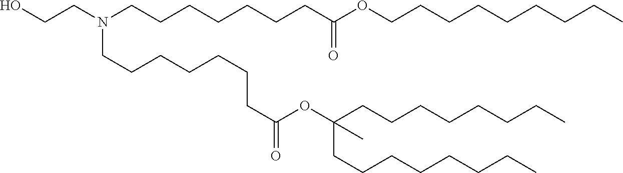 [SMILES: CCCCCCCCCOC(=O)CCCCCCCN(CCO)CCCCCCCC(=O)OC(C)(CCCCCCCC)CCCCCCCC] |
| I-83 | 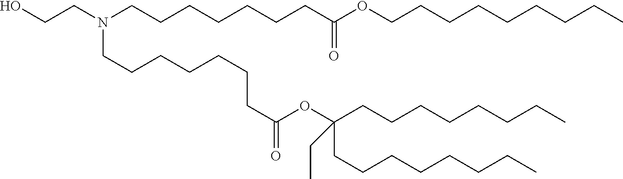 [SMILES: CCCCCCCCCOC(=O)CCCCCCCN(CCO)CCCCCCCC(=O)OC(CC)(CCCCCCCC)CCCCCCCC] |
| I-84 | 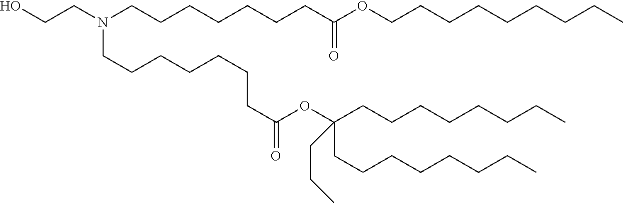 [SMILES: CCCCCCCCCOC(=O)CCCCCCCN(CCO)CCCCCCCC(=O)OC(CCC)(CCCCCCCC)CCCCCCCC] |
| I-85 | 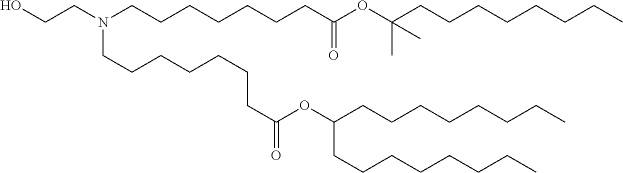 [SMILES: CCCCCCCCC(CCCCCCCC)OC(=O)CCCCCCCN(CCO)CCCCCCCC(=O)OC(C)(C)CCCCCCCC] |
| I-86 | 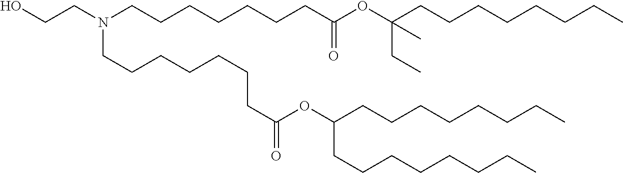 [SMILES: CCCCCCCCC(CCCCCCCC)OC(=O)CCCCCCCN(CCO)CCCCCCCC(=O)OC(C)(CC)CCCCCCCC] |
| I-87 | 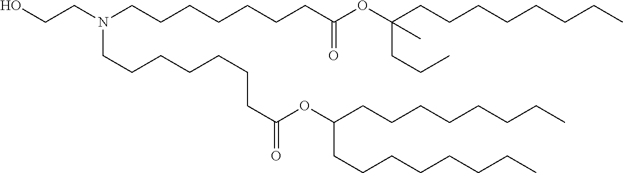 [SMILES: CCCCCCCCC(CCCCCCCC)OC(=O)CCCCCCCN(CCO)CCCCCCCC(=O)OC(C)(CCC)CCCCCCCC] |
| I-88 | 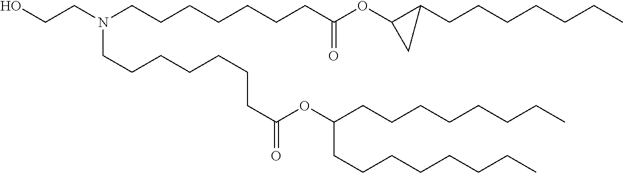 [SMILES: CCCCCCCCC(CCCCCCCC)OC(=O)CCCCCCCN(CCO)CCCCCCCC(=O)OC1CC1CCCCCCC] |
| I-89 | 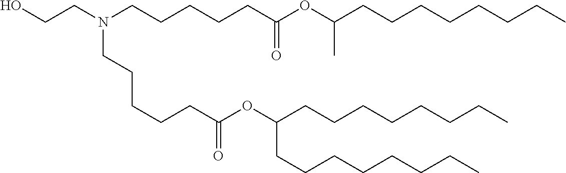 [SMILES: CCCCCCCCC(C)OC(=O)CCCCCN(CCO)CCCCCC(=O)OC(CCCCCCCC)CCCCCCCC] |
| I-90 | 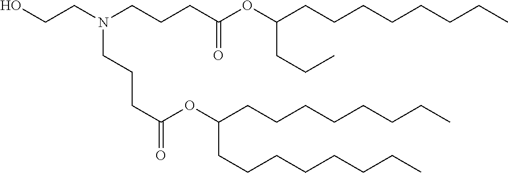 [SMILES: CCCCCCCCC(CCC)OC(=O)CCCN(CCO)CCCC(=O)OC(CCCCCCCC)CCCCCCCC] |
| I-91 | 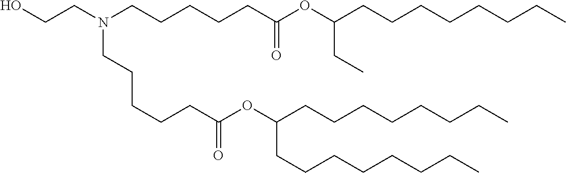 [SMILES: CCCCCCCCC(CC)OC(=O)CCCCCN(CCO)CCCCCC(=O)OC(CCCCCCCC)CCCCCCCC] |
| I-92 | 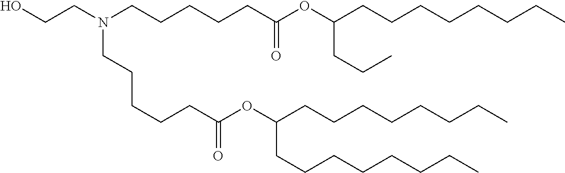 [SMILES: CCCCCCCCC(CCC)OC(=O)CCCCCN(CCO)CCCCCC(=O)OC(CCCCCCCC)CCCCCCCC] |
| I-93 | 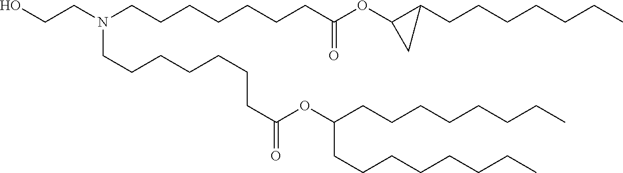 [SMILES: CCCCCCCCC(CCCCCCCC)OC(=O)CCCCCCCN(CCO)CCCCCCCC(=O)OC1CC1CCCCCCC] |
| I-94 | 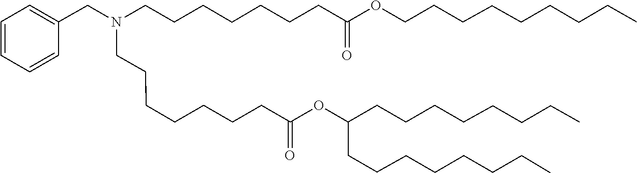 [SMILES: CCCCCCCCCOC(=O)CCCCCCCN(CCCCCCCC(=O)OC(CCCCCCCC)CCCCCCCC)Cc1ccccc1] |
| I-95 | 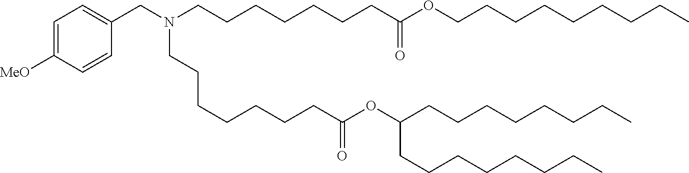 [SMILES: CCCCCCCCCOC(=O)CCCCCCCN(CCCCCCCC(=O)OC(CCCCCCCC)CCCCCCCC)Cc1ccc(OC)cc1] |
| I-96 | 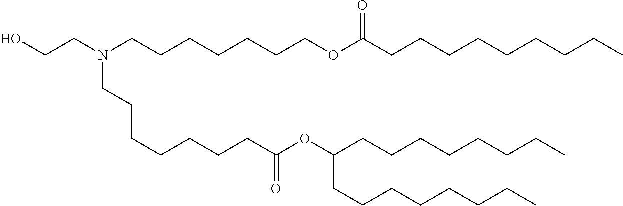 [SMILES: CCCCCCCCCC(=O)OCCCCCCCN(CCO)CCCCCCCC(=O)OC(CCCCCCCC)CCCCCCCC] |
| I-97 | 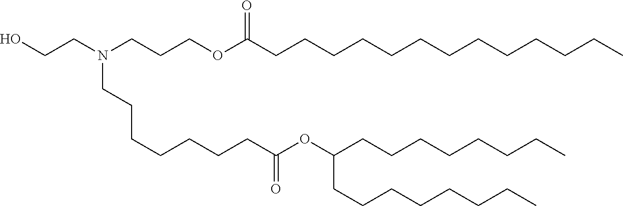 [SMILES: CCCCCCCCCCCCCC(=O)OCCCN(CCO)CCCCCCCC(=O)OC(CCCCCCCC)CCCCCCCC] |
| I-98 | 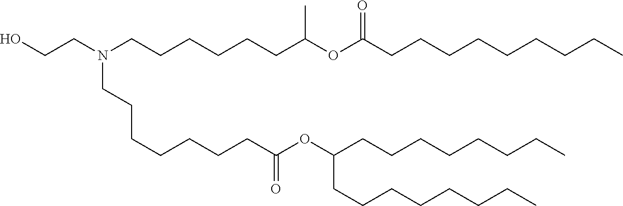 [SMILES: CCCCCCCCCC(=O)OC(C)CCCCCCN(CCO)CCCCCCCC(=O)OC(CCCCCCCC)CCCCCCCC] |
| I-99 | 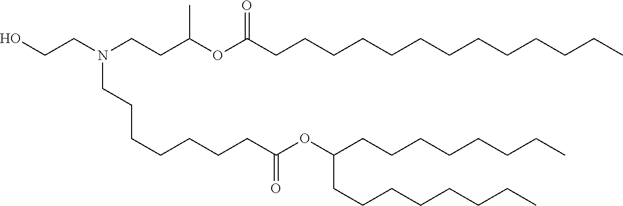 [SMILES: CCCCCCCCCCCCCC(=O)OC(C)CCN(CCO)CCCCCCCC(=O)OC(CCCCCCCC)CCCCCCCC] |
| I-100 | 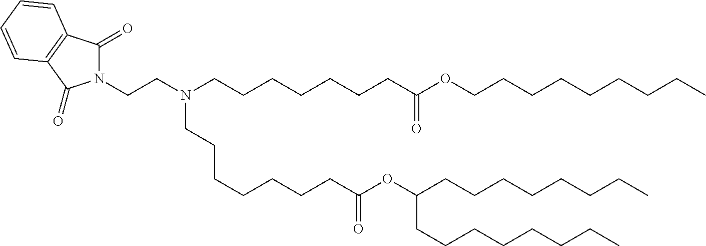 [SMILES: CCCCCCCCCOC(=O)CCCCCCCN(CCCCCCCC(=O)OC(CCCCCCCC)CCCCCCCC)CCN1C(=O)c2ccccc2C1=O] |
| I-101 | 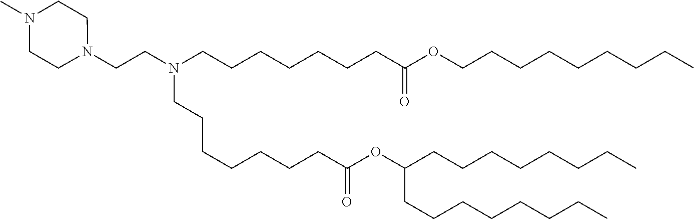 [SMILES: CCCCCCCCCOC(=O)CCCCCCCN(CCCCCCCC(=O)OC(CCCCCCCC)CCCCCCCC)CCN1CCN(C)CC1] |
| I-102 | 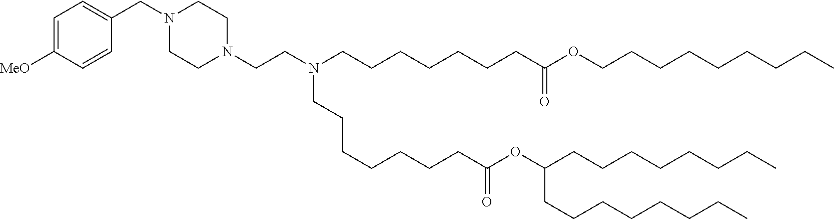 [SMILES: CCCCCCCCCOC(=O)CCCCCCCN(CCCCCCCC(=O)OC(CCCCCCCC)CCCCCCCC)CCN1CCN(Cc2ccc(OC)cc2)CC1] |
| I-103 | 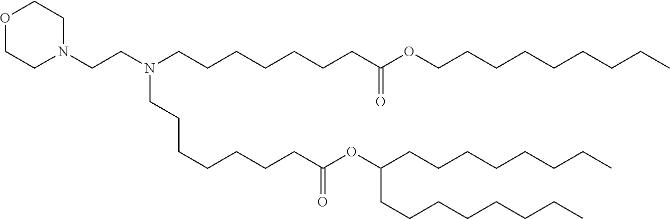 [SMILES: CCCCCCCCCOC(=O)CCCCCCCN(CCCCCCCC(=O)OC(CCCCCCCC)CCCCCCCC)CCN1CCOCC1] |
| I-104 | 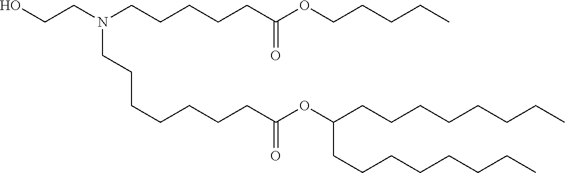 [SMILES: CCCCCCCCC(CCCCCCCC)OC(=O)CCCCCCCN(CCO)CCCCCC(=O)OCCCCC] |
| I-105 | 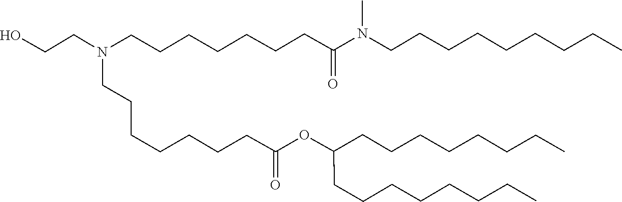 [SMILES: CCCCCCCCCN(C)C(=O)CCCCCCCN(CCO)CCCCCCCC(=O)OC(CCCCCCCC)CCCCCCCC] |
| I-106 | 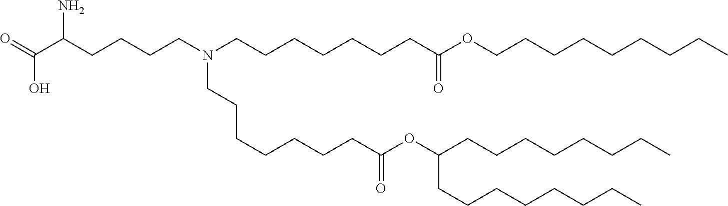 [SMILES: CCCCCCCCCOC(=O)CCCCCCCN(CCCCCCCC(=O)OC(CCCCCCCC)CCCCCCCC)CCCCC(N)C(=O)O] |
| I-107 | 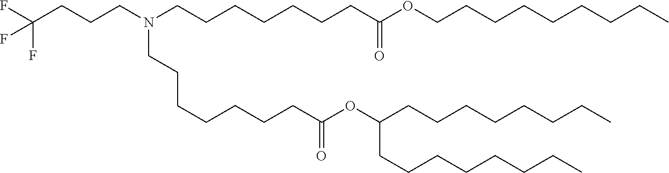 [SMILES: CCCCCCCCCOC(=O)CCCCCCCN(CCCCCCCC(=O)OC(CCCCCCCC)CCCCCCCC)CCCC(F)(F)F] |
| I-108 | 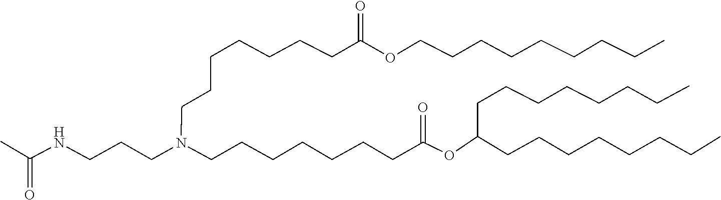 [SMILES: CCCCCCCCCOC(=O)CCCCCCCN(CCCCCCCC(=O)OC(CCCCCCCC)CCCCCCCC)CCCNC(C)=O] |
| I-109 | 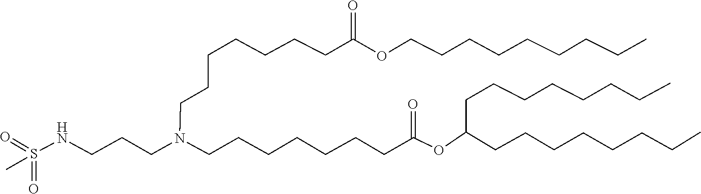 [SMILES: CCCCCCCCCOC(=O)CCCCCCCN(CCCCCCCC(=O)OC(CCCCCCCC)CCCCCCCC)CCCNS(C)(=O)=O] |
| I-110 | 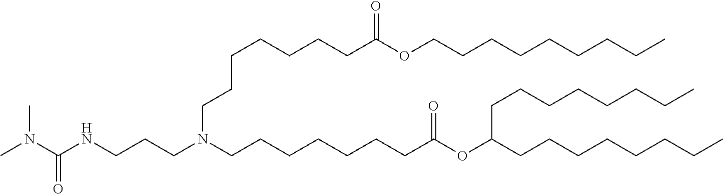 [SMILES: CCCCCCCCCOC(=O)CCCCCCCN(CCCCCCCC(=O)OC(CCCCCCCC)CCCCCCCC)CCCNC(=O)N(C)C] |
| I-111 | 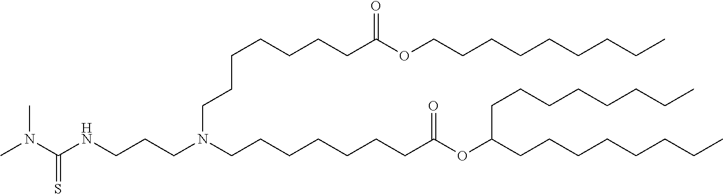 [SMILES: CCCCCCCCCOC(=O)CCCCCCCN(CCCCCCCC(=O)OC(CCCCCCCC)CCCCCCCC)CCCNC(=S)N(C)C] |
| I-112 | 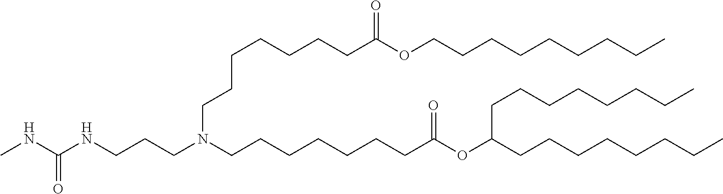 [SMILES: CCCCCCCCCOC(=O)CCCCCCCN(CCCCCCCC(=O)OC(CCCCCCCC)CCCCCCCC)CCCNC(=O)NC] |
| I-113 | 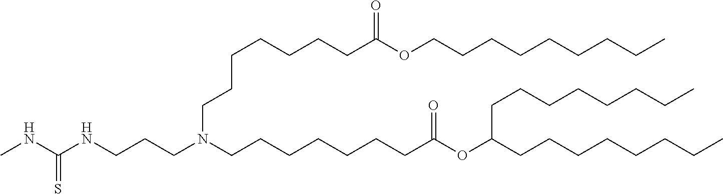 [SMILES: CCCCCCCCCOC(=O)CCCCCCCN(CCCCCCCC(=O)OC(CCCCCCCC)CCCCCCCC)CCCNC(=S)NC] |
| I-114 | 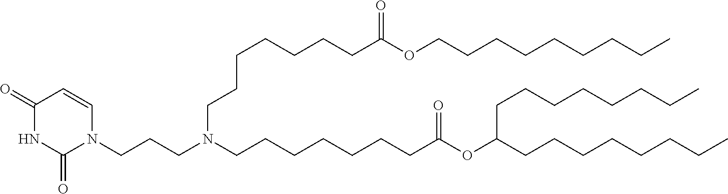 [SMILES: CCCCCCCCCOC(=O)CCCCCCCN(CCCCCCCC(=O)OC(CCCCCCCC)CCCCCCCC)CCCn1ccc(=O)[nH]c1=O] |
| I-115 | 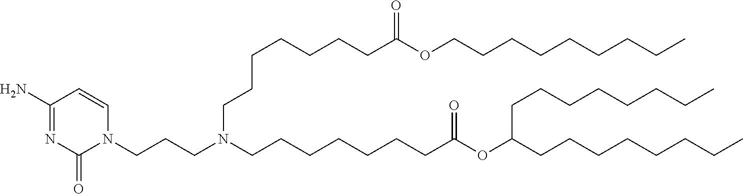 [SMILES: CCCCCCCCCOC(=O)CCCCCCCN(CCCCCCCC(=O)OC(CCCCCCCC)CCCCCCCC)CCCn1ccc(N)nc1=O] |
| I-116 | 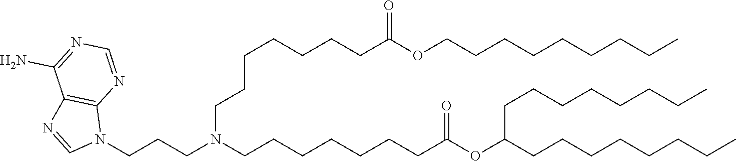 [SMILES: CCCCCCCCCOC(=O)CCCCCCCN(CCCCCCCC(=O)OC(CCCCCCCC)CCCCCCCC)CCCn1cnc2c(N)ncnc21] |
| I-117 | 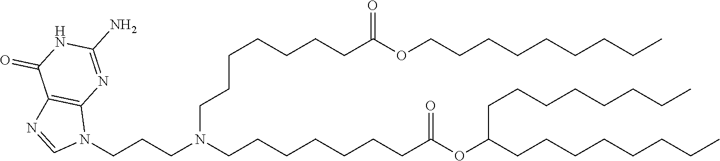 [SMILES: CCCCCCCCCOC(=O)CCCCCCCN(CCCCCCCC(=O)OC(CCCCCCCC)CCCCCCCC)CCCn1cnc2c(=O)[nH]c(N)nc21] |
| I-118 | 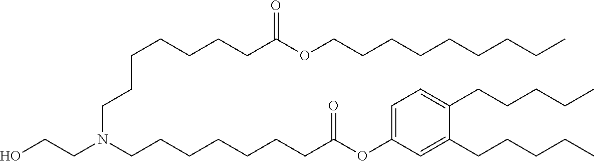 [SMILES: CCCCCCCCCOC(=O)CCCCCCCN(CCO)CCCCCCCC(=O)Oc1ccc(CCCCC)c(CCCCC)c1] |
| I-119 | 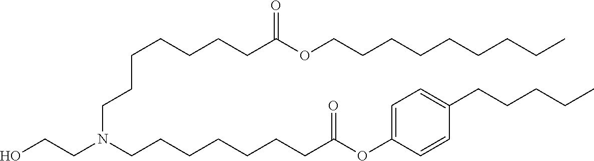 [SMILES: CCCCCCCCCOC(=O)CCCCCCCN(CCO)CCCCCCCC(=O)Oc1ccc(CCCCC)cc1] |
| I-120 | 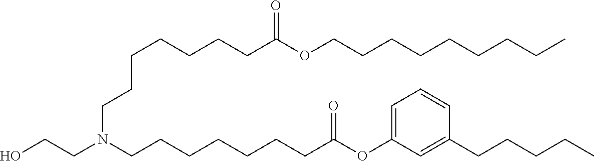 [SMILES: CCCCCCCCCOC(=O)CCCCCCCN(CCO)CCCCCCCC(=O)Oc1cccc(CCCCC)c1] |
| I-121 | 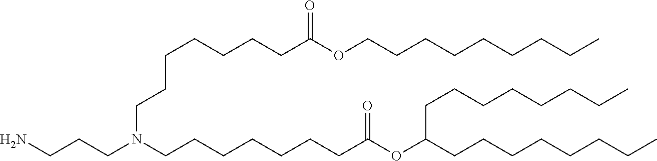 [SMILES: CCCCCCCCCOC(=O)CCCCCCCN(CCCN)CCCCCCCC(=O)OC(CCCCCCCC)CCCCCCCC] |
| I-122 | 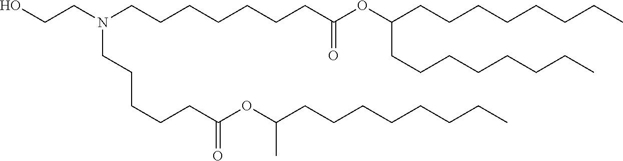 [SMILES: CCCCCCCCC(C)OC(=O)CCCCCN(CCO)CCCCCCCC(=O)OC(CCCCCCCC)CCCCCCCC] |
| I-123 | 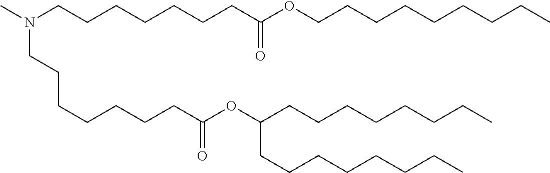 [SMILES: CCCCCCCCCOC(=O)CCCCCCCN(C)CCCCCCCC(=O)OC(CCCCCCCC)CCCCCCCC] |
| I-124 | 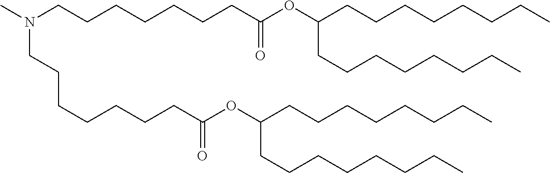 [SMILES: CCCCCCCCC(CCCCCCCC)OC(=O)CCCCCCCN(C)CCCCCCCC(=O)OC(CCCCCCCC)CCCCCCCC] |
| I-125 | 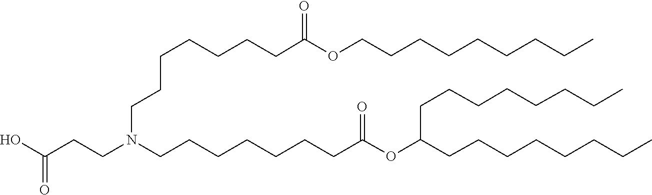 [SMILES: CCCCCCCCCOC(=O)CCCCCCCN(CCCCCCCC(=O)OC(CCCCCCCC)CCCCCCCC)CCC(=O)O] |
| I-126 | 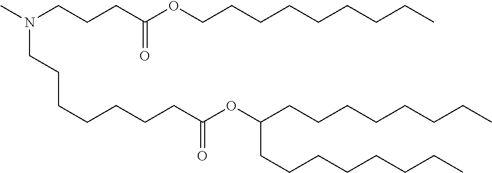 [SMILES: CCCCCCCCCOC(=O)CCCN(C)CCCCCCCC(=O)OC(CCCCCCCC)CCCCCCCC] |
| I-127 | 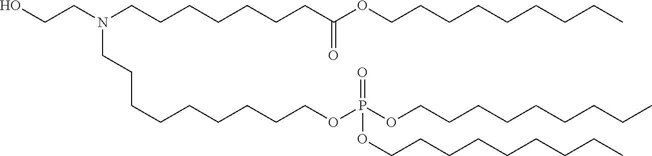 [SMILES: CCCCCCCCCOC(=O)CCCCCCCN(CCO)CCCCCCCCCOP(=O)(OCCCCCCCCC)OCCCCCCCCC] |
| I-128 | 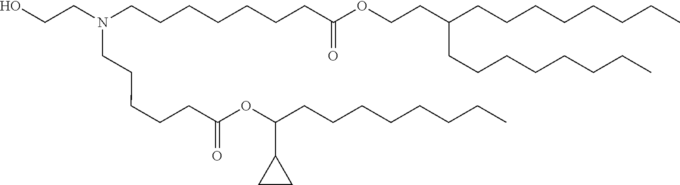 [SMILES: CCCCCCCCC(CCCCCCCC)CCOC(=O)CCCCCCCN(CCO)CCCCCC(=O)OC(CCCCCCCC)C1CC1] |
| I-129 | 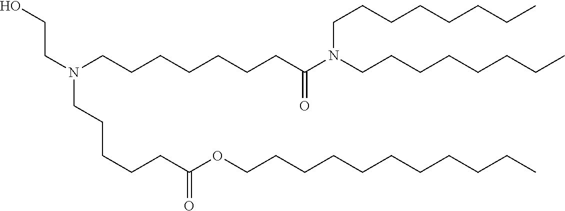 [SMILES: CCCCCCCCCCCOC(=O)CCCCCN(CCO)CCCCCCCC(=O)N(CCCCCCCC)CCCCCCCC] |
| I-130 | 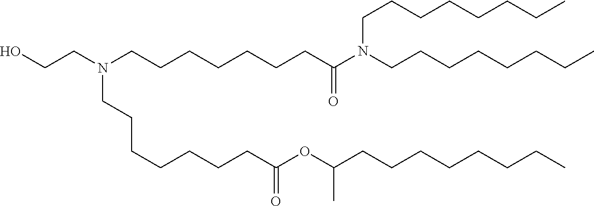 [SMILES: CCCCCCCCC(C)OC(=O)CCCCCCCN(CCO)CCCCCCCC(=O)N(CCCCCCCC)CCCCCCCC] |
| I-131 | 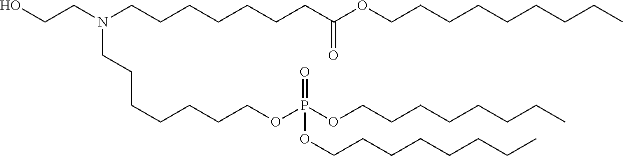 [SMILES: CCCCCCCCCOC(=O)CCCCCCCN(CCO)CCCCCCCOP(=O)(OCCCCCCCC)OCCCCCCCC] |
| I-132 | 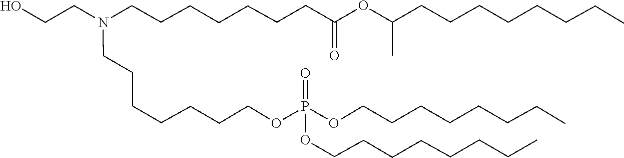 [SMILES: CCCCCCCCOP(=O)(OCCCCCCCC)OCCCCCCCN(CCO)CCCCCCCC(=O)OC(C)CCCCCCCC] |
| I-133 | 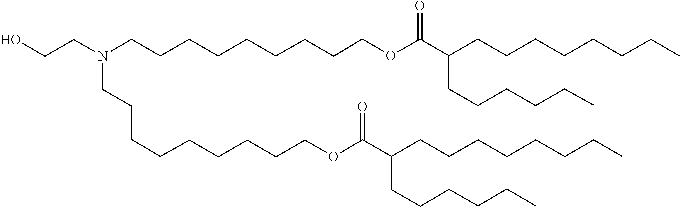 [SMILES: CCCCCCCCC(CCCCCC)C(=O)OCCCCCCCCCN(CCO)CCCCCCCCCOC(=O)C(CCCCCC)CCCCCCCC] |
| I-134 | 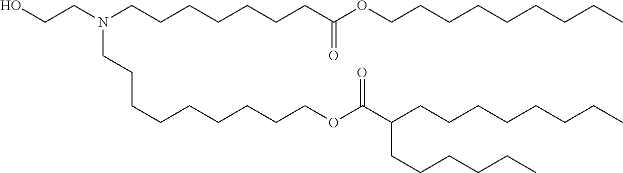 [SMILES: CCCCCCCCCOC(=O)CCCCCCCN(CCO)CCCCCCCCCOC(=O)C(CCCCCC)CCCCCCCC] |
| I-135 | 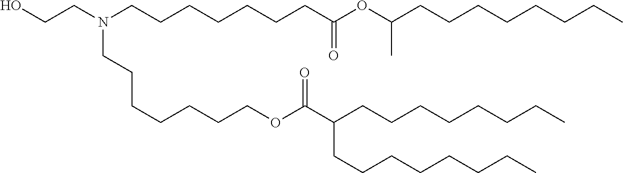 [SMILES: CCCCCCCCC(C)OC(=O)CCCCCCCN(CCO)CCCCCCCOC(=O)C(CCCCCCCC)CCCCCCCC] |
| I-136 | 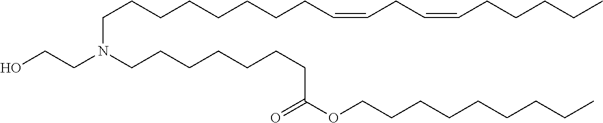 [SMILES: CCCCC/C=C\C/C=C\CCCCCCCCN(CCO)CCCCCCCC(=O)OCCCCCCCCC] |
| I-137 | 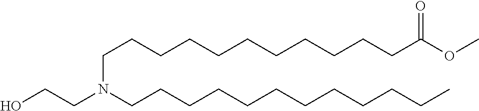 [SMILES: CCCCCCCCCCCCN(CCO)CCCCCCCCCCCC(=O)OC] |
| I-138 | 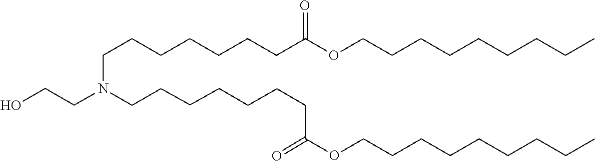 [SMILES: CCCCCCCCCOC(=O)CCCCCCCN(CCO)CCCCCCCC(=O)OCCCCCCCCC] |
| I-139 | 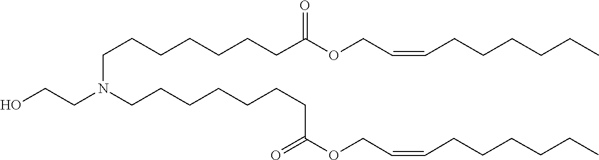 [SMILES: CCCCCC/C=C\COC(=O)CCCCCCCN(CCO)CCCCCCCC(=O)OC/C=C\CCCCCC] |
| I-140 | 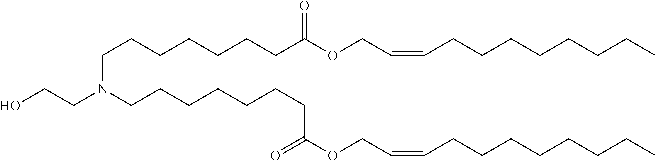 [SMILES: CCCCCCCC/C=C\COC(=O)CCCCCCCN(CCO)CCCCCCCC(=O)OC/C=C\CCCCCCCC] |
| I-141 | 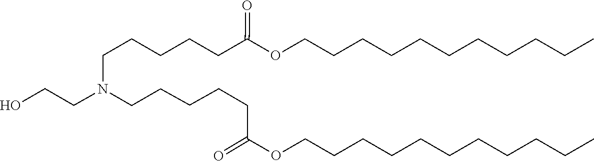 [SMILES: CCCCCCCCCCCOC(=O)CCCCCN(CCO)CCCCCC(=O)OCCCCCCCCCCC] |
| I-142 | 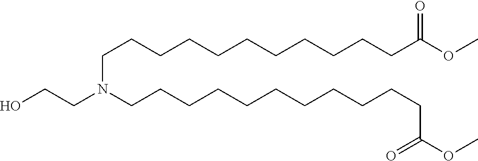 [SMILES: COC(=O)CCCCCCCCCCCN(CCO)CCCCCCCCCCCC(=O)OC] |
| I-143 | 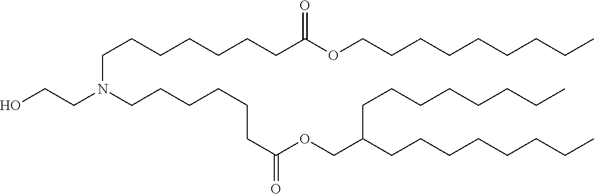 [SMILES: CCCCCCCCCOC(=O)CCCCCCCN(CCO)CCCCCCC(=O)OCC(CCCCCCCC)CCCCCCCC] |
| I-144 | 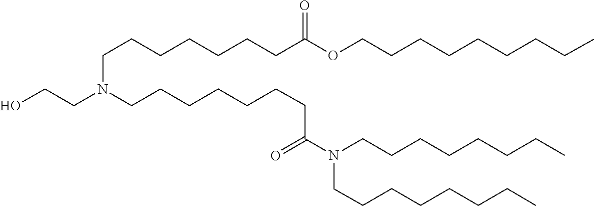 [SMILES: CCCCCCCCCOC(=O)CCCCCCCN(CCO)CCCCCCCC(=O)N(CCCCCCCC)CCCCCCCC] |
| I-145 | 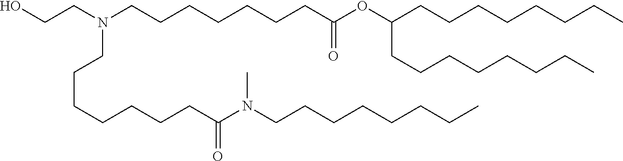 [SMILES: CCCCCCCCC(CCCCCCCC)OC(=O)CCCCCCCN(CCO)CCCCCCCC(=O)N(C)CCCCCCCC] |
| I-146 | 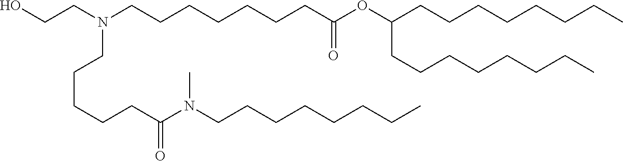 [SMILES: CCCCCCCCC(CCCCCCCC)OC(=O)CCCCCCCN(CCO)CCCCCC(=O)N(C)CCCCCCCC] |
| I-147 | 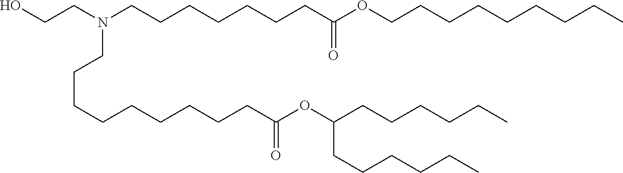 [SMILES: CCCCCCCCCOC(=O)CCCCCCCN(CCO)CCCCCCCCCC(=O)OC(CCCCCC)CCCCCC] |
| I-148 | 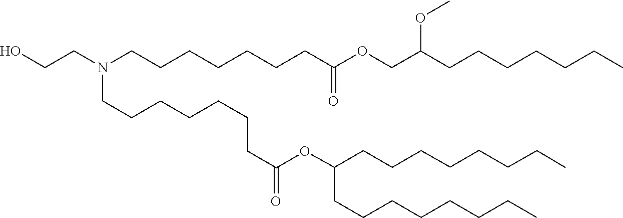 [SMILES: CCCCCCCCC(CCCCCCCC)OC(=O)CCCCCCCN(CCO)CCCCCCCC(=O)OCC(CCCCCCC)OC] |
| I-149 | 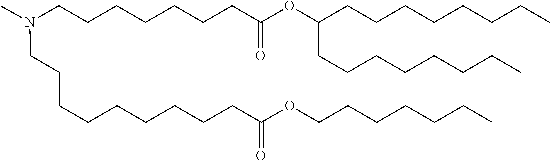 [SMILES: CCCCCCCCC(CCCCCCCC)OC(=O)CCCCCCCN(C)CCCCCCCCCC(=O)OCCCCCCC] |
| I-150 | 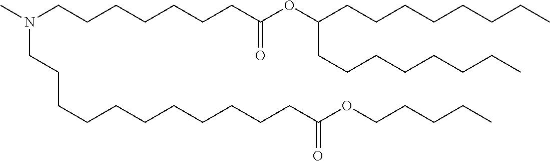 [SMILES: CCCCCCCCC(CCCCCCCC)OC(=O)CCCCCCCN(C)CCCCCCCCCCCC(=O)OCCCCC] |
| I-151 | 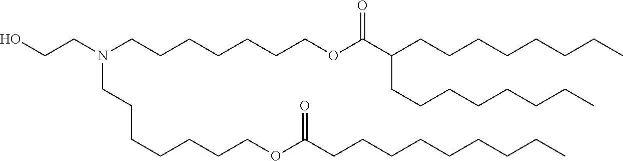 [SMILES: CCCCCCCCCC(=O)OCCCCCCCN(CCO)CCCCCCCOC(=O)C(CCCCCCCC)CCCCCCCC] |
| I-152 | 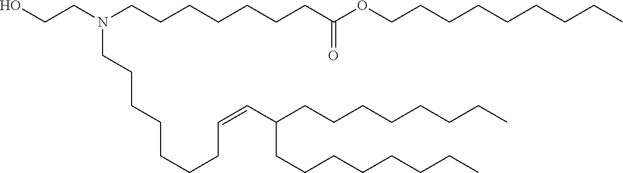 [SMILES: CCCCCCCCCOC(=O)CCCCCCCN(CCO)CCCCCCC/C=C\C(CCCCCCCC)CCCCCCCC] |
| I-153 | 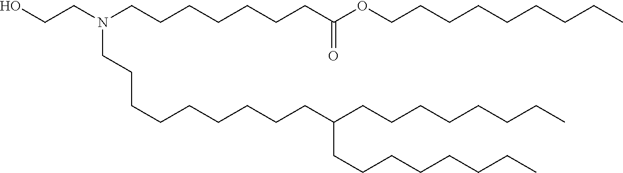 [SMILES: CCCCCCCCCOC(=O)CCCCCCCN(CCO)CCCCCCCCCC(CCCCCCCC)CCCCCCCC] |
| I-154 | 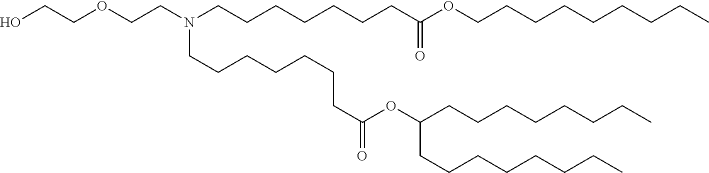 [SMILES: CCCCCCCCCOC(=O)CCCCCCCN(CCCCCCCC(=O)OC(CCCCCCCC)CCCCCCCC)CCOCCO] |
| I-155 | 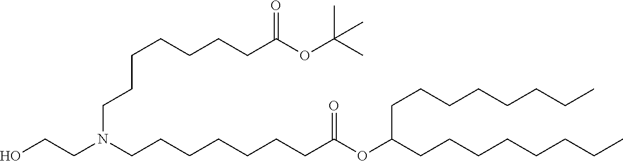 [SMILES: CCCCCCCCC(CCCCCCCC)OC(=O)CCCCCCCN(CCO)CCCCCCCC(=O)OC(C)(C)C] |
| I-156 | 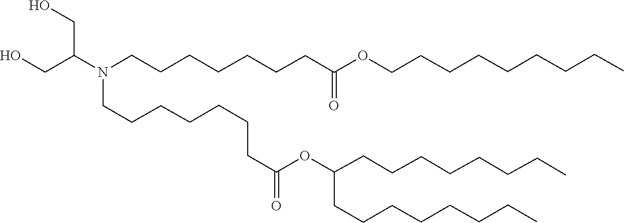 [SMILES: CCCCCCCCCOC(=O)CCCCCCCN(CCCCCCCC(=O)OC(CCCCCCCC)CCCCCCCC)C(CO)CO] |
| I-157 | 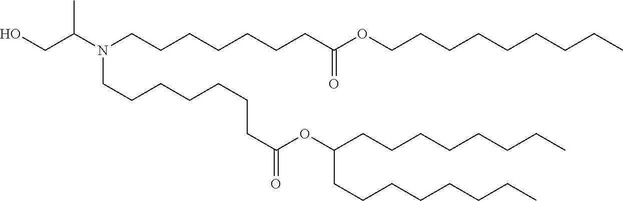 [SMILES: CCCCCCCCCOC(=O)CCCCCCCN(CCCCCCCC(=O)OC(CCCCCCCC)CCCCCCCC)C(C)CO] |
| I-158 | 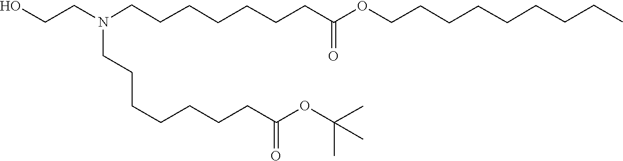 [SMILES: CCCCCCCCCOC(=O)CCCCCCCN(CCO)CCCCCCCC(=O)OC(C)(C)C] |
| I-159 | 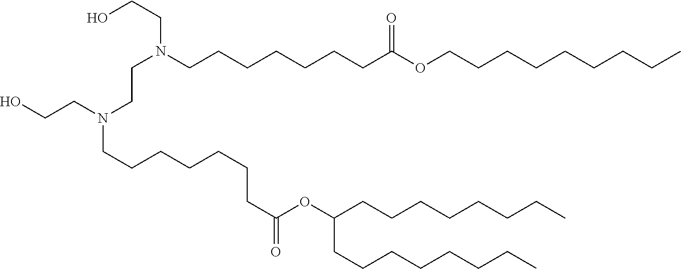 [SMILES: CCCCCCCCCOC(=O)CCCCCCCN(CCO)CCN(CCO)CCCCCCCC(=O)OC(CCCCCCCC)CCCCCCCC] |
| I-160 | 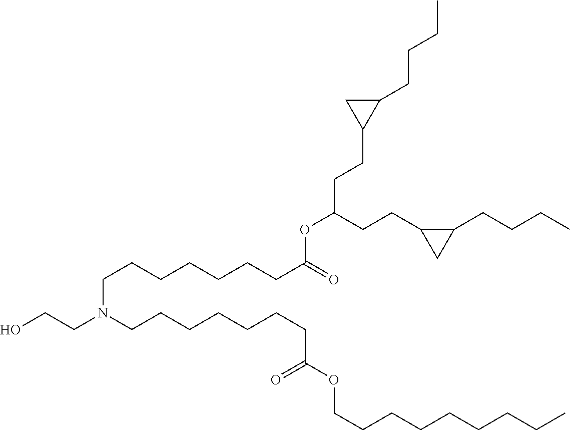 [SMILES: CCCCCCCCCOC(=O)CCCCCCCN(CCO)CCCCCCCC(=O)OC(CCC1CC1CCCC)CCC1CC1CCCC] |
| I-161 | 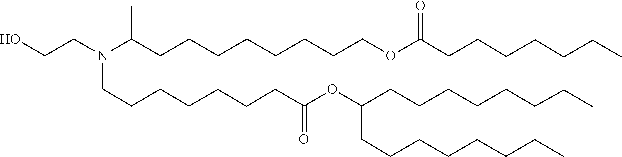 [SMILES: CCCCCCCCC(CCCCCCCC)OC(=O)CCCCCCCN(CCO)C(C)CCCCCCCCOC(=O)CCCCCCC] |
| I-162 | 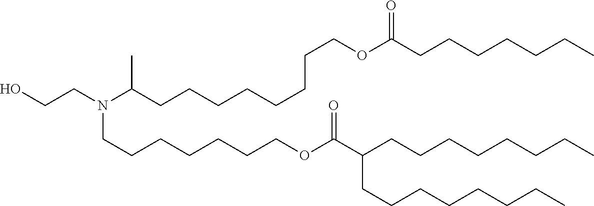 [SMILES: CCCCCCCCC(CCCCCCCC)C(=O)OCCCCCCCN(CCO)C(C)CCCCCCCCOC(=O)CCCCCCC] |
| I-163 | 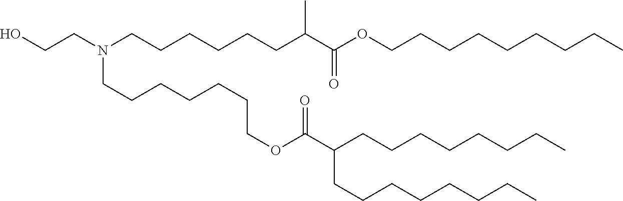 [SMILES: CCCCCCCCCOC(=O)C(C)CCCCCCN(CCO)CCCCCCCOC(=O)C(CCCCCCCC)CCCCCCCC] |
| I-164 | 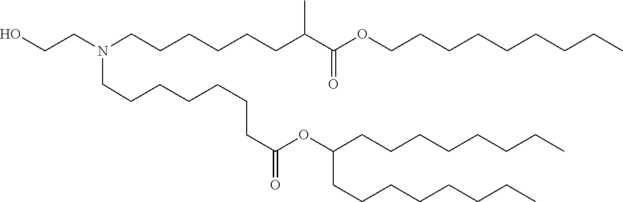 [SMILES: CCCCCCCCCOC(=O)C(C)CCCCCCN(CCO)CCCCCCCC(=O)OC(CCCCCCCC)CCCCCCCC] |
| I-165 | 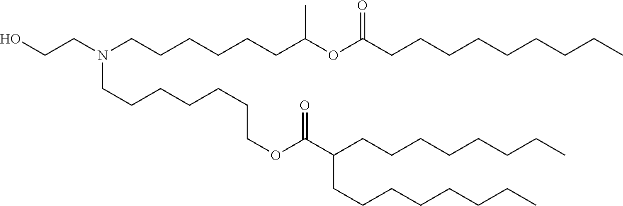 [SMILES: CCCCCCCCCC(=O)OC(C)CCCCCCN(CCO)CCCCCCCOC(=O)C(CCCCCCCC)CCCCCCCC] |
| I-166 | 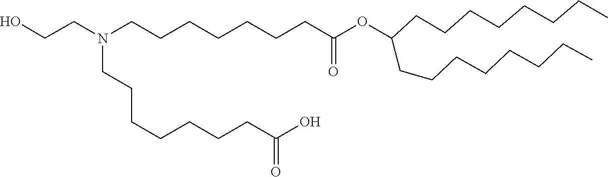 [SMILES: CCCCCCCCC(CCCCCCCC)OC(=O)CCCCCCCN(CCO)CCCCCCCC(=O)O] |
| I-167 | 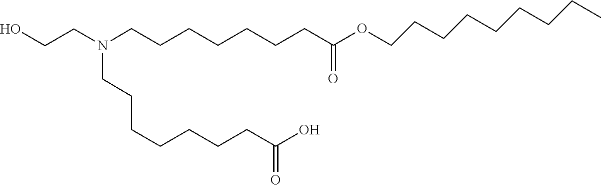 [SMILES: CCCCCCCCCOC(=O)CCCCCCCN(CCO)CCCCCCCC(=O)O] |
| I-168 | 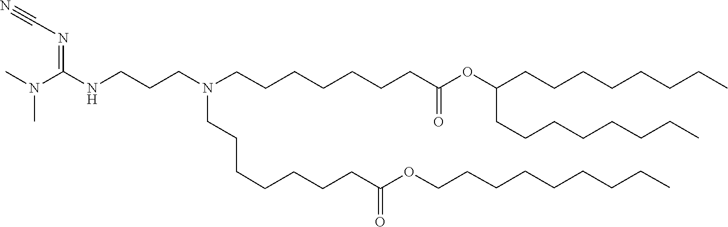 [SMILES: CCCCCCCCCOC(=O)CCCCCCCN(CCCCCCCC(=O)OC(CCCCCCCC)CCCCCCCC)CCCN/C(=N/C#N)N(C)C] |
| I-169 | 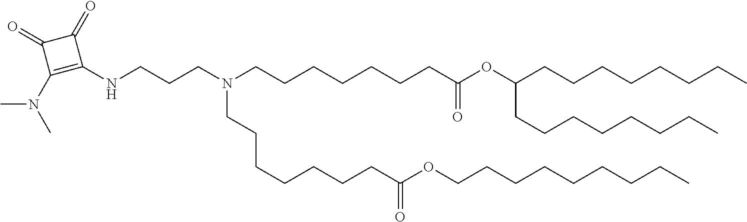 [SMILES: CCCCCCCCCOC(=O)CCCCCCCN(CCCCCCCC(=O)OC(CCCCCCCC)CCCCCCCC)CCCNc1c(N(C)C)c(=O)c1=O] |
| I-170 | 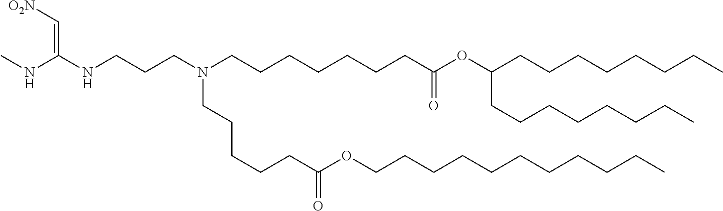 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I-171 | 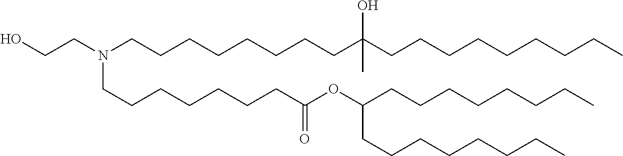 [SMILES: CCCCCCCCCC(C)(O)CCCCCCCCN(CCO)CCCCCCCC(=O)OC(CCCCCCCC)CCCCCCCC] |
| I-172 | 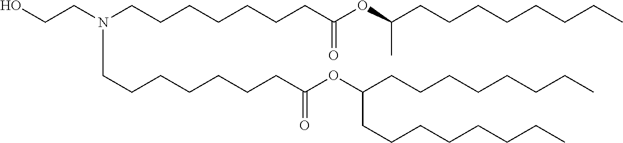 [SMILES: CCCCCCCCC(CCCCCCCC)OC(=O)CCCCCCCN(CCO)CCCCCCCC(=O)O[C@H](C)CCCCCCCC] |
| I-173 | 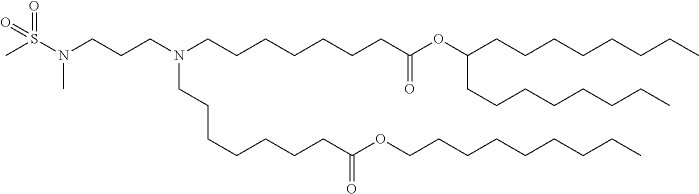 [SMILES: CCCCCCCCCOC(=O)CCCCCCCN(CCCCCCCC(=O)OC(CCCCCCCC)CCCCCCCC)CCCN(C)S(C)(=O)=O] |
| I-174 | 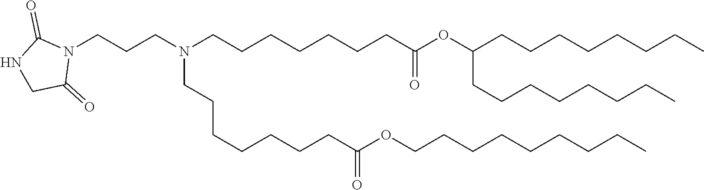 [SMILES: CCCCCCCCCOC(=O)CCCCCCCN(CCCCCCCC(=O)OC(CCCCCCCC)CCCCCCCC)CCCN1C(=O)CNC1=O] |
| I-175 | 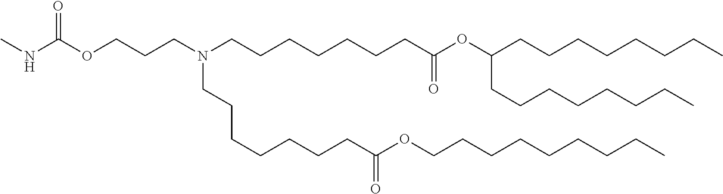 [SMILES: CCCCCCCCCOC(=O)CCCCCCCN(CCCCCCCC(=O)OC(CCCCCCCC)CCCCCCCC)CCCOC(=O)NC] |
| I-176 | 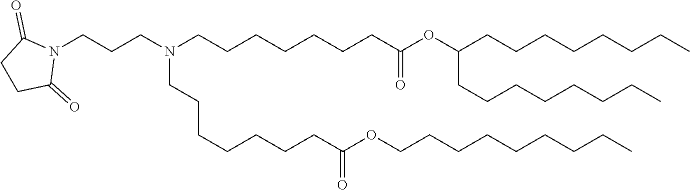 [SMILES: CCCCCCCCCOC(=O)CCCCCCCN(CCCCCCCC(=O)OC(CCCCCCCC)CCCCCCCC)CCCN1C(=O)CCC1=O] |
| I-177 | 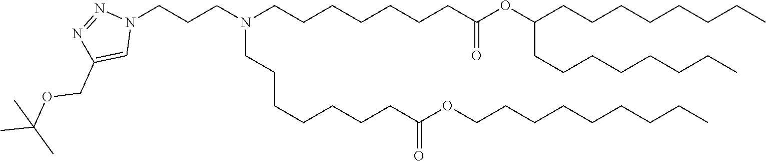 [SMILES: CCCCCCCCCOC(=O)CCCCCCCN(CCCCCCCC(=O)OC(CCCCCCCC)CCCCCCCC)CCCn1cc(COC(C)(C)C)nn1] |
| I-178 | 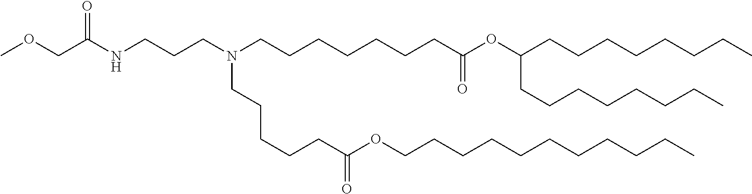 [SMILES: CCCCCCCCCCCOC(=O)CCCCCN(CCCCCCCC(=O)OC(CCCCCCCC)CCCCCCCC)CCCNC(=O)COC] |
| I-179 | 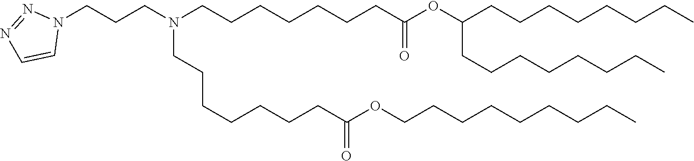 [SMILES: CCCCCCCCCOC(=O)CCCCCCCN(CCCCCCCC(=O)OC(CCCCCCCC)CCCCCCCC)CCCn1ccnn1] |
| I-180 | 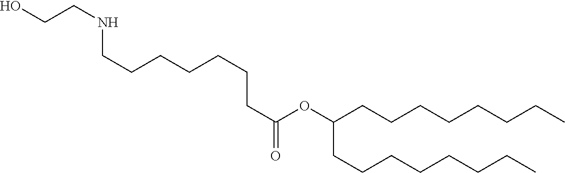 [SMILES: CCCCCCCCC(CCCCCCCC)OC(=O)CCCCCCCNCCO] |
| I-181 | 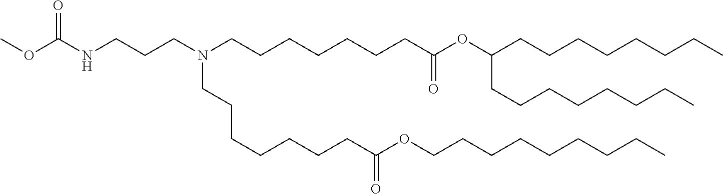 [SMILES: CCCCCCCCCOC(=O)CCCCCCCN(CCCCCCCC(=O)OC(CCCCCCCC)CCCCCCCC)CCCNC(=O)OC] |
| I-182 | 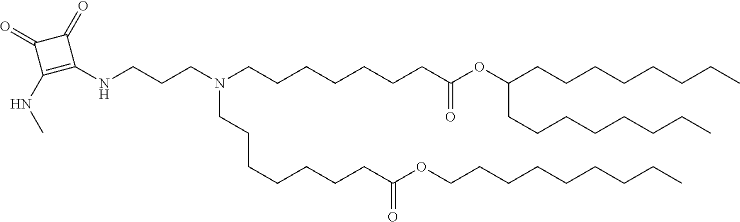 [SMILES: CCCCCCCCCOC(=O)CCCCCCCN(CCCCCCCC(=O)OC(CCCCCCCC)CCCCCCCC)CCCNc1c(NC)c(=O)c1=O] |
| I-183 | 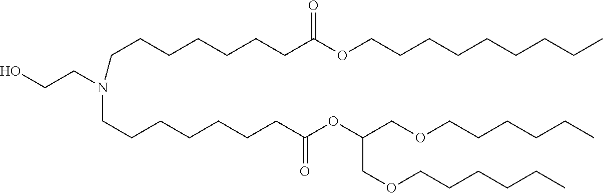 [SMILES: CCCCCCCCCOC(=O)CCCCCCCN(CCO)CCCCCCCC(=O)OC(COCCCCCC)COCCCCCC] |
| I-184 | 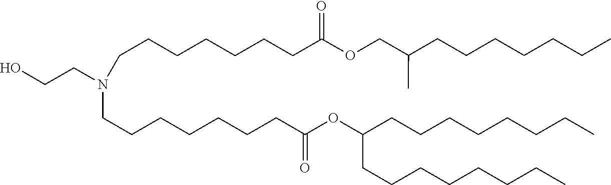 [SMILES: CCCCCCCCC(CCCCCCCC)OC(=O)CCCCCCCN(CCO)CCCCCCCC(=O)OCC(C)CCCCCCC] |
| I-185 | 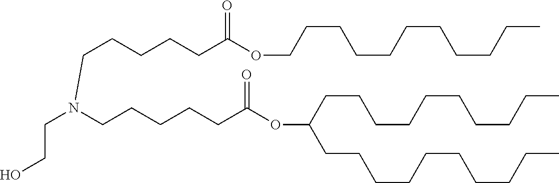 [SMILES: CCCCCCCCCCCOC(=O)CCCCCN(CCO)CCCCCC(=O)OC(CCCCCCCCCC)CCCCCCCCCC] |
| I-186 | 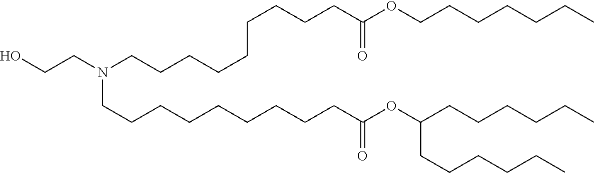 [SMILES: CCCCCCCOC(=O)CCCCCCCCCN(CCO)CCCCCCCCCC(=O)OC(CCCCCC)CCCCCC] |
| I-187 | 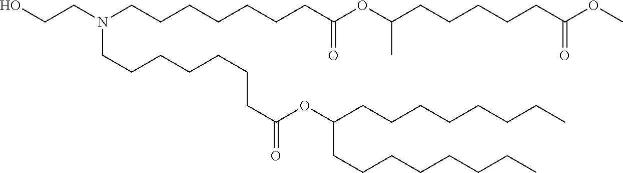 [SMILES: CCCCCCCCC(CCCCCCCC)OC(=O)CCCCCCCN(CCO)CCCCCCCC(=O)OC(C)CCCCCC(=O)OC] |
| I-188 | 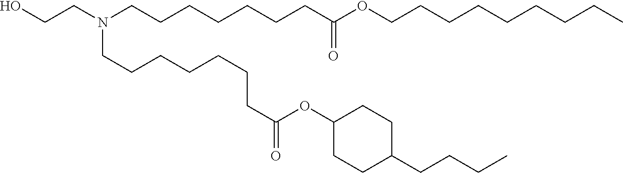 [SMILES: CCCCCCCCCOC(=O)CCCCCCCN(CCO)CCCCCCCC(=O)OC1CCC(CCCC)CC1] |
| I-189 | 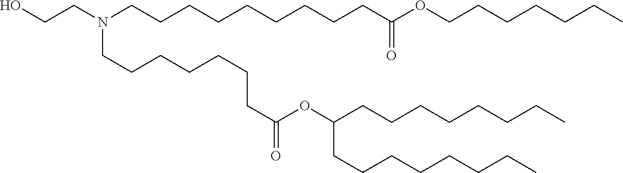 [SMILES: CCCCCCCCC(CCCCCCCC)OC(=O)CCCCCCCN(CCO)CCCCCCCCCC(=O)OCCCCCCC] |
| I-190 | 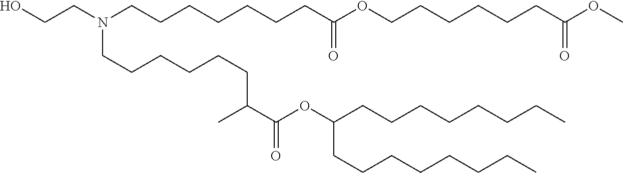 [SMILES: CCCCCCCCC(CCCCCCCC)OC(=O)C(C)CCCCCCN(CCO)CCCCCCCC(=O)OCCCCCCC(=O)OC] |
| I-191 | 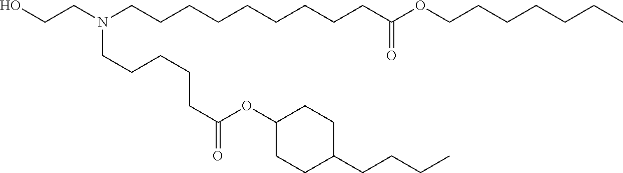 [SMILES: CCCCCCCOC(=O)CCCCCCCCCN(CCO)CCCCCC(=O)OC1CCC(CCCC)CC1] |
| I-192 | 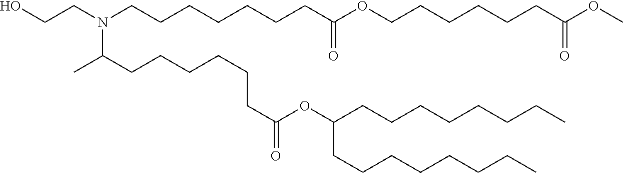 [SMILES: CCCCCCCCC(CCCCCCCC)OC(=O)CCCCCCC(C)N(CCO)CCCCCCCC(=O)OCCCCCCC(=O)OC] |
| I-193 | 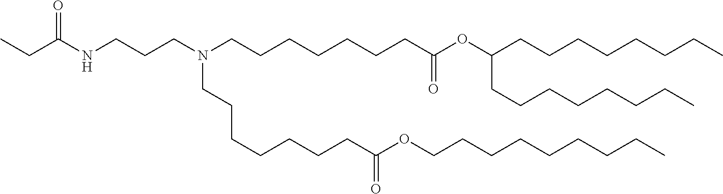 [SMILES: CCCCCCCCCOC(=O)CCCCCCCN(CCCCCCCC(=O)OC(CCCCCCCC)CCCCCCCC)CCCNC(=O)CC] |
| I-194 | 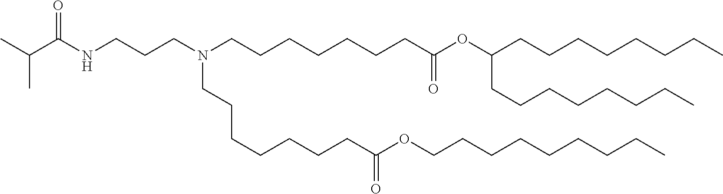 [SMILES: CCCCCCCCCOC(=O)CCCCCCCN(CCCCCCCC(=O)OC(CCCCCCCC)CCCCCCCC)CCCNC(=O)C(C)C] |
| I-195 | 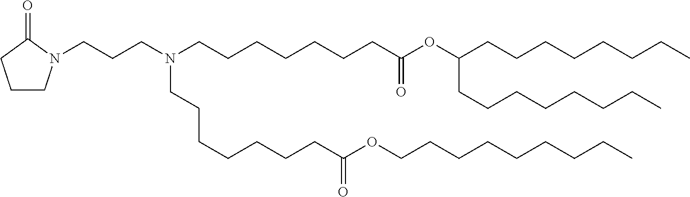 [SMILES: CCCCCCCCCOC(=O)CCCCCCCN(CCCCCCCC(=O)OC(CCCCCCCC)CCCCCCCC)CCCN1CCCC1=O] |
| I-196 | 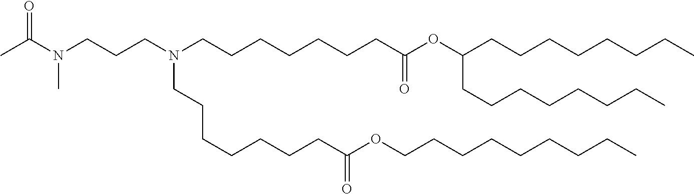 [SMILES: CCCCCCCCCOC(=O)CCCCCCCN(CCCCCCCC(=O)OC(CCCCCCCC)CCCCCCCC)CCCN(C)C(C)=O] |
| I-197 | 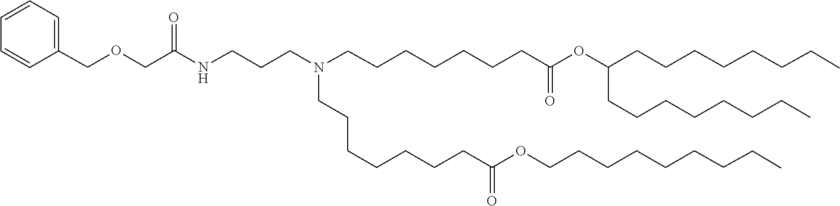 [SMILES: CCCCCCCCCOC(=O)CCCCCCCN(CCCCCCCC(=O)OC(CCCCCCCC)CCCCCCCC)CCCNC(=O)COCc1ccccc1] |
| I-198 | 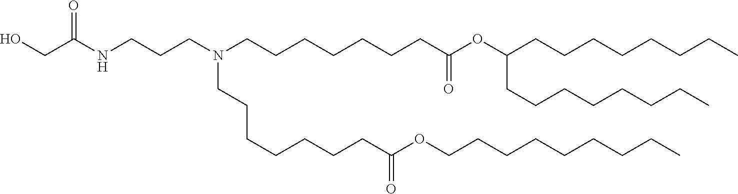 [SMILES: CCCCCCCCCOC(=O)CCCCCCCN(CCCCCCCC(=O)OC(CCCCCCCC)CCCCCCCC)CCCNC(=O)CO] |
| I-199 | 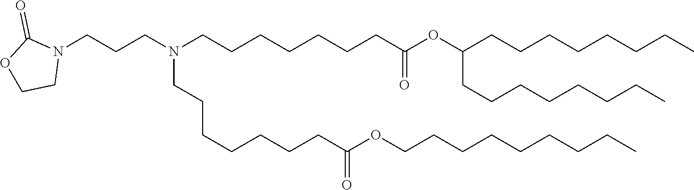 [SMILES: CCCCCCCCCOC(=O)CCCCCCCN(CCCCCCCC(=O)OC(CCCCCCCC)CCCCCCCC)CCCN1CCOC1=O] |
| I-200 | 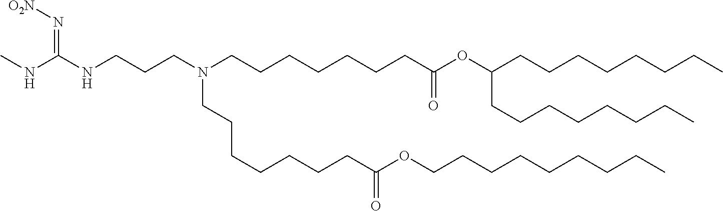 [SMILES: CCCCCCCCCOC(=O)CCCCCCCN(CCCCCCCC(=O)OC(CCCCCCCC)CCCCCCCC)CCCN/C(=N/[N+](=O)[O-])NC] |
| I-201 | 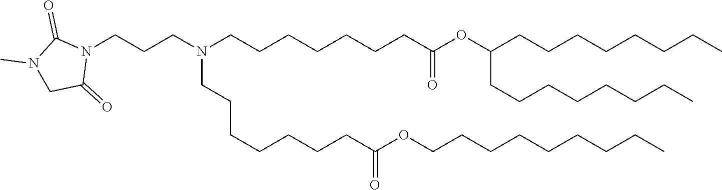 [SMILES: CCCCCCCCCOC(=O)CCCCCCCN(CCCCCCCC(=O)OC(CCCCCCCC)CCCCCCCC)CCCN1C(=O)CN(C)C1=O] |
| I-202 | 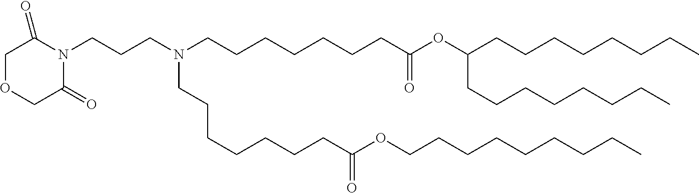 [SMILES: CCCCCCCCCOC(=O)CCCCCCCN(CCCCCCCC(=O)OC(CCCCCCCC)CCCCCCCC)CCCN1C(=O)COCC1=O] |
| I-203 | 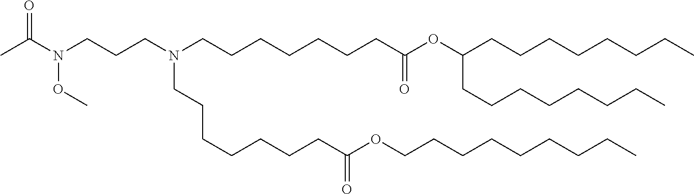 [SMILES: CCCCCCCCCOC(=O)CCCCCCCN(CCCCCCCC(=O)OC(CCCCCCCC)CCCCCCCC)CCCN(OC)C(C)=O] |
| I-204 | 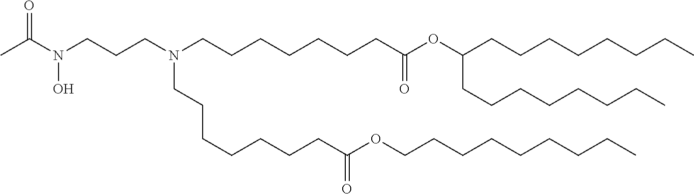 [SMILES: CCCCCCCCCOC(=O)CCCCCCCN(CCCCCCCC(=O)OC(CCCCCCCC)CCCCCCCC)CCCN(O)C(C)=O] |
| I-205 | 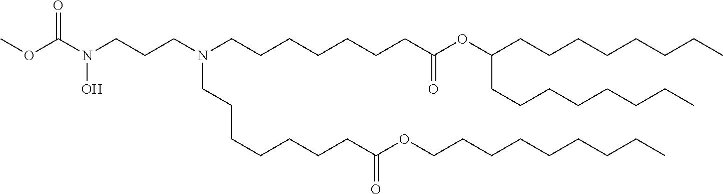 [SMILES: CCCCCCCCCOC(=O)CCCCCCCN(CCCCCCCC(=O)OC(CCCCCCCC)CCCCCCCC)CCCN(O)C(=O)OC] |
| I-206 | 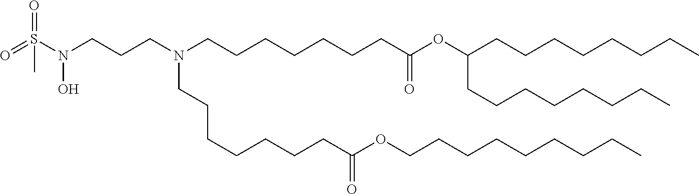 [SMILES: CCCCCCCCCOC(=O)CCCCCCCN(CCCCCCCC(=O)OC(CCCCCCCC)CCCCCCCC)CCCN(O)S(C)(=O)=O] |
| I-207 | 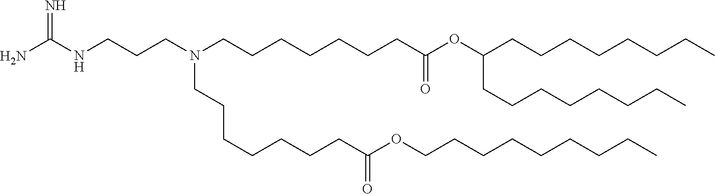 [SMILES: CCCCCCCCCOC(=O)CCCCCCCN(CCCCCCCC(=O)OC(CCCCCCCC)CCCCCCCC)CCCNC(=N)N] |
| I-208 | 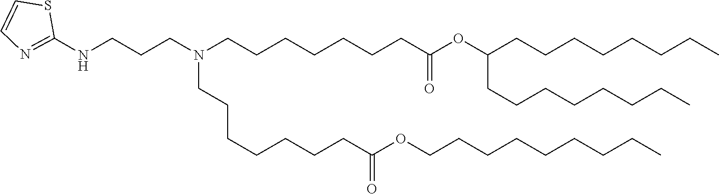 [SMILES: CCCCCCCCCOC(=O)CCCCCCCN(CCCCCCCC(=O)OC(CCCCCCCC)CCCCCCCC)CCCNc1nccs1] |
| I-209 | 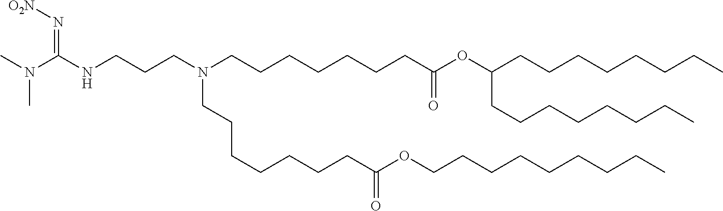 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I-210 | 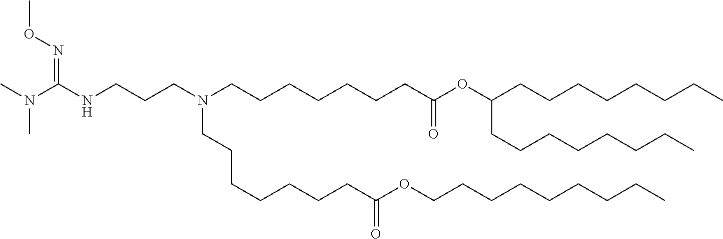 [SMILES: CCCCCCCCCOC(=O)CCCCCCCN(CCCCCCCC(=O)OC(CCCCCCCC)CCCCCCCC)CCCN/C(=N/OC)N(C)C] |
| I-211 | 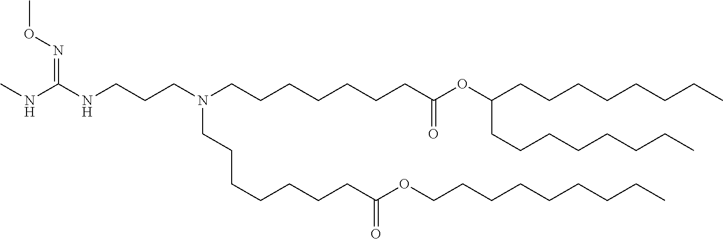 [SMILES: CCCCCCCCCOC(=O)CCCCCCCN(CCCCCCCC(=O)OC(CCCCCCCC)CCCCCCCC)CCCN/C(=N/OC)NC] |
| I-212 | 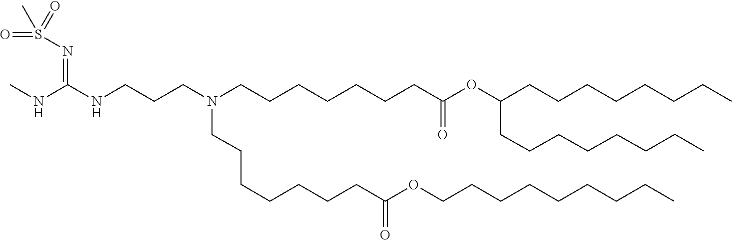 [SMILES: CCCCCCCCCOC(=O)CCCCCCCN(CCCCCCCC(=O)OC(CCCCCCCC)CCCCCCCC)CCCN/C(=N/S(C)(=O)=O)NC] |
| I-213 | 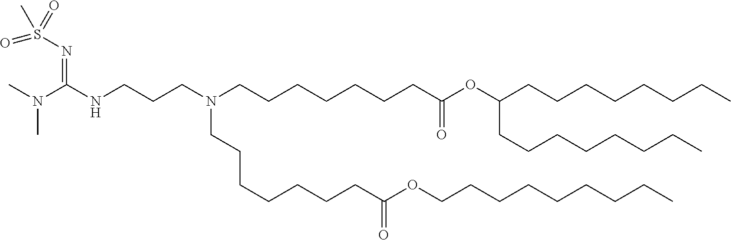 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I-214 | 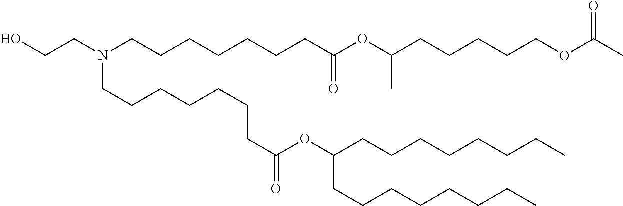 [SMILES: CCCCCCCCC(CCCCCCCC)OC(=O)CCCCCCCN(CCO)CCCCCCCC(=O)OC(C)CCCCCOC(C)=O] |
| I-215 | 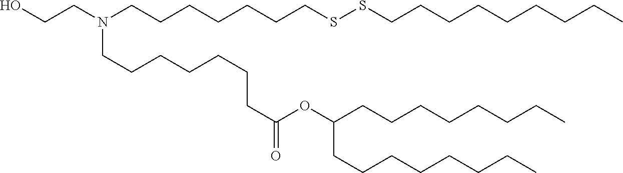 [SMILES: CCCCCCCCCSSCCCCCCCN(CCO)CCCCCCCC(=O)OC(CCCCCCCC)CCCCCCCC] |
| I-216 | 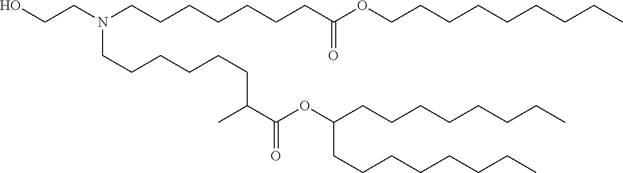 [SMILES: CCCCCCCCCOC(=O)CCCCCCCN(CCO)CCCCCCC(C)C(=O)OC(CCCCCCCC)CCCCCCCC] |
| I-217 | 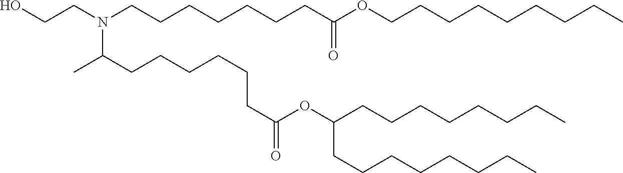 [SMILES: CCCCCCCCCOC(=O)CCCCCCCN(CCO)C(C)CCCCCCC(=O)OC(CCCCCCCC)CCCCCCCC] |
| I-218 | 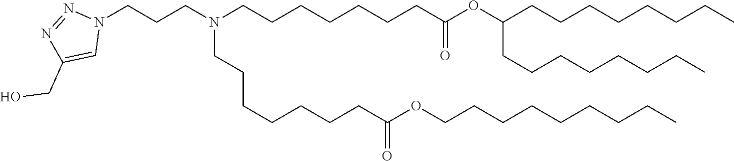 [SMILES: CCCCCCCCCOC(=O)CCCCCCCN(CCCCCCCC(=O)OC(CCCCCCCC)CCCCCCCC)CCCn1cc(CO)nn1] |
| I-219 | 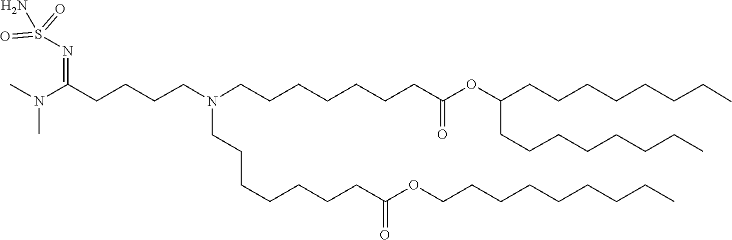 [SMILES: CCCCCCCCCOC(=O)CCCCCCCN(CCCCCCCC(=O)OC(CCCCCCCC)CCCCCCCC)CCCC/C(=N/S(N)(=O)=O)N(C)C] |
| I-220 | 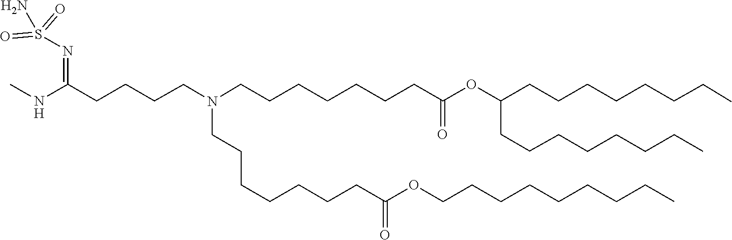 [SMILES: CCCCCCCCCOC(=O)CCCCCCCN(CCCCCCCC(=O)OC(CCCCCCCC)CCCCCCCC)CCCC/C(=N/S(N)(=O)=O)NC] |
| I-221 | 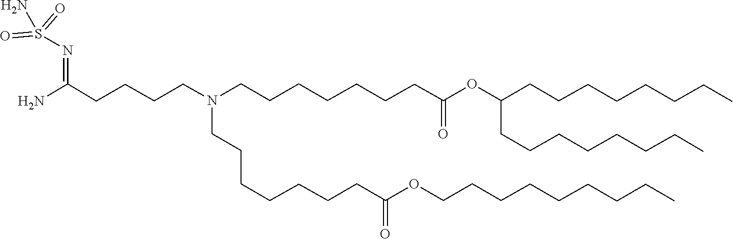 [SMILES: CCCCCCCCCOC(=O)CCCCCCCN(CCCCCCCC(=O)OC(CCCCCCCC)CCCCCCCC)CCCC/C(N)=N/S(N)(=O)=O] |
| I-222 | 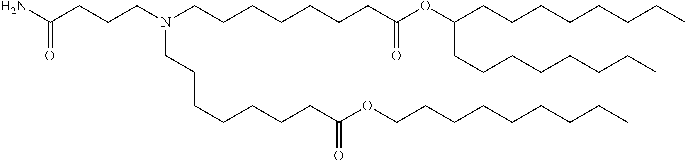 [SMILES: CCCCCCCCCOC(=O)CCCCCCCN(CCCCCCCC(=O)OC(CCCCCCCC)CCCCCCCC)CCCC(N)=O] |
| I-223 | 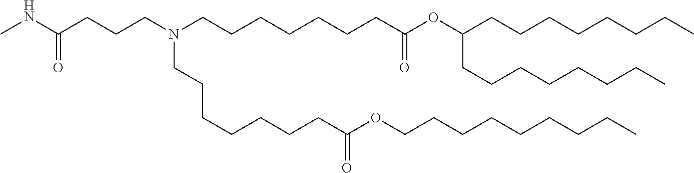 [SMILES: CCCCCCCCCOC(=O)CCCCCCCN(CCCCCCCC(=O)OC(CCCCCCCC)CCCCCCCC)CCCC(=O)NC] |
| I-224 | 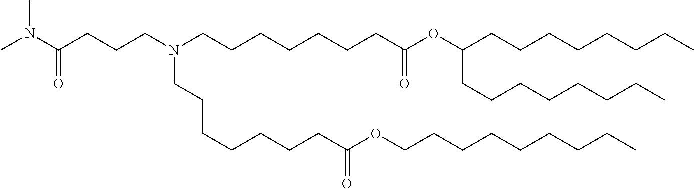 [SMILES: CCCCCCCCCOC(=O)CCCCCCCN(CCCCCCCC(=O)OC(CCCCCCCC)CCCCCCCC)CCCC(=O)N(C)C] |
| I-225 | 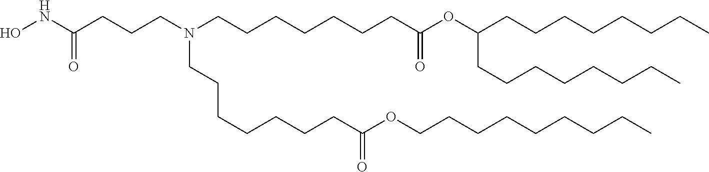 [SMILES: CCCCCCCCCOC(=O)CCCCCCCN(CCCCCCCC(=O)OC(CCCCCCCC)CCCCCCCC)CCCC(=O)NO] |
| I-226 | 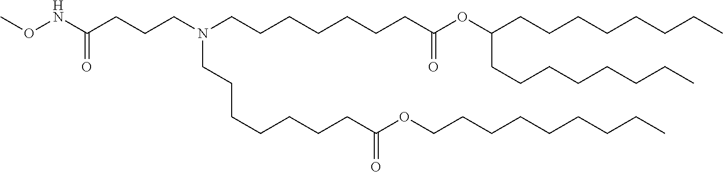 [SMILES: CCCCCCCCCOC(=O)CCCCCCCN(CCCCCCCC(=O)OC(CCCCCCCC)CCCCCCCC)CCCC(=O)NOC] |
| I-227 | 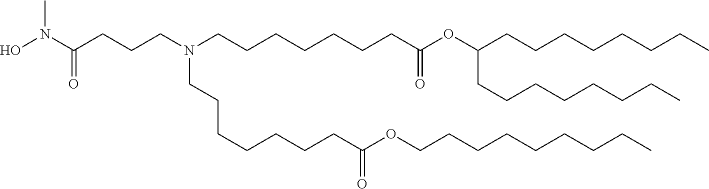 [SMILES: CCCCCCCCCOC(=O)CCCCCCCN(CCCCCCCC(=O)OC(CCCCCCCC)CCCCCCCC)CCCC(=O)N(C)O] |
| I-228 | 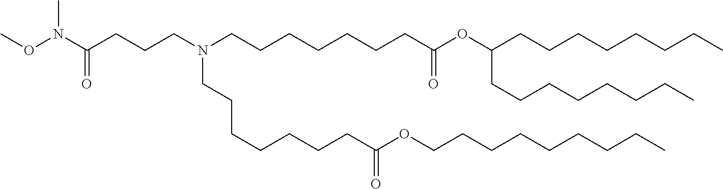 [SMILES: CCCCCCCCCOC(=O)CCCCCCCN(CCCCCCCC(=O)OC(CCCCCCCC)CCCCCCCC)CCCC(=O)N(C)OC] |
| I-229 | 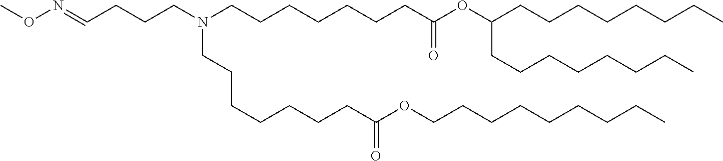 [SMILES: CCCCCCCCCOC(=O)CCCCCCCN(CCC/C=N/OC)CCCCCCCC(=O)OC(CCCCCCCC)CCCCCCCC] |
| I-230 | 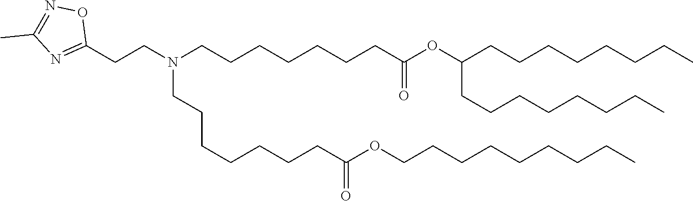 [SMILES: CCCCCCCCCOC(=O)CCCCCCCN(CCCCCCCC(=O)OC(CCCCCCCC)CCCCCCCC)CCc1nc(C)no1] |
| I-231 | 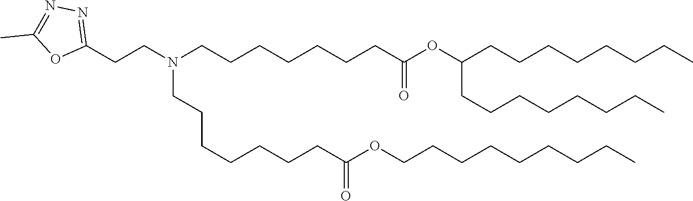 [SMILES: CCCCCCCCCOC(=O)CCCCCCCN(CCCCCCCC(=O)OC(CCCCCCCC)CCCCCCCC)CCc1nnc(C)o1] |
| I-232 | 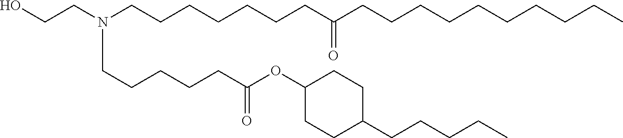 [SMILES: CCCCCCCCCCC(=O)CCCCCCCN(CCO)CCCCCC(=O)OC1CCC(CCCCC)CC1] |
| I-233 | 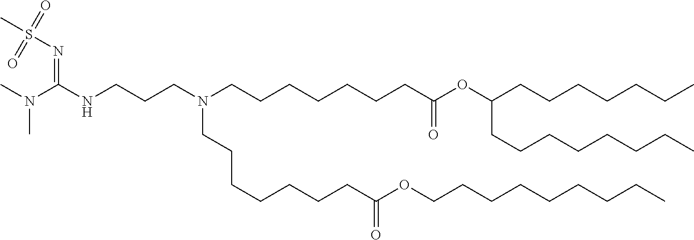 [SMILES: CCCCCCCCCOC(=O)CCCCCCCN(CCCCCCCC(=O)OC(CCCCCCC)CCCCCCCC)CCCN/C(=N/S(C)(=O)=O)N(C)C] |
| I-234 | 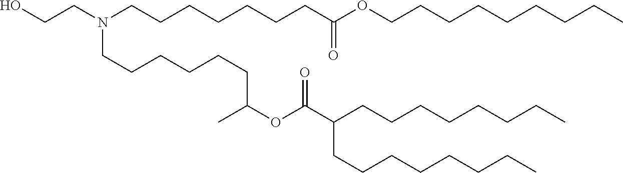 [SMILES: CCCCCCCCCOC(=O)CCCCCCCN(CCO)CCCCCCC(C)OC(=O)C(CCCCCCCC)CCCCCCCC] |
| I-235 | 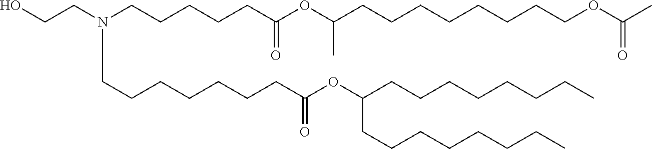 [SMILES: CCCCCCCCC(CCCCCCCC)OC(=O)CCCCCCCN(CCO)CCCCCC(=O)OC(C)CCCCCCCCOC(C)=O] |
| I-236 | 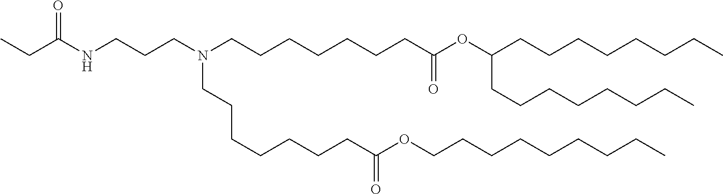 [SMILES: CCCCCCCCCOC(=O)CCCCCCCN(CCCCCCCC(=O)OC(CCCCCCCC)CCCCCCCC)CCCNC(=O)CC] |
| I-237 | 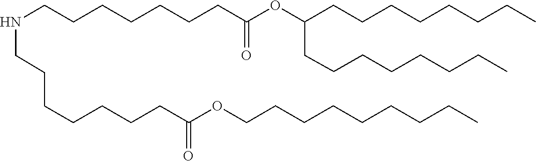 [SMILES: CCCCCCCCCOC(=O)CCCCCCCNCCCCCCCC(=O)OC(CCCCCCCC)CCCCCCCC] |
| I-238 | 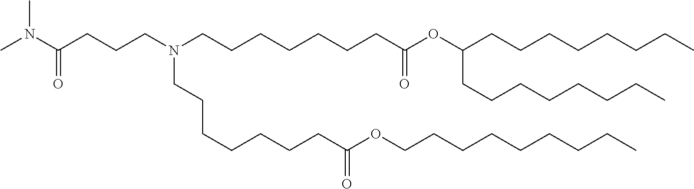 [SMILES: CCCCCCCCCOC(=O)CCCCCCCN(CCCCCCCC(=O)OC(CCCCCCCC)CCCCCCCC)CCCC(=O)N(C)C] |
| I-239 | 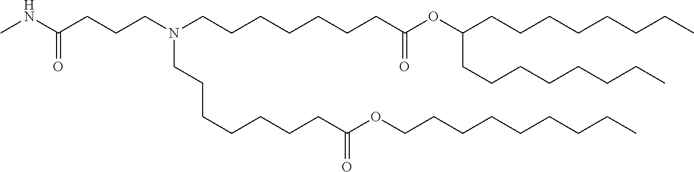 [SMILES: CCCCCCCCCOC(=O)CCCCCCCN(CCCCCCCC(=O)OC(CCCCCCCC)CCCCCCCC)CCCC(=O)NC] |
| I-240 | 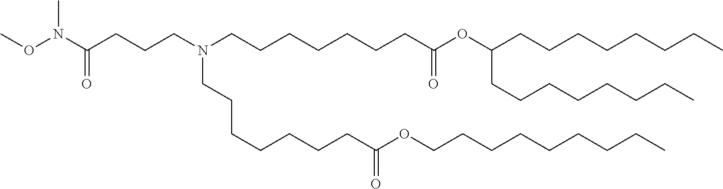 [SMILES: CCCCCCCCCOC(=O)CCCCCCCN(CCCCCCCC(=O)OC(CCCCCCCC)CCCCCCCC)CCCC(=O)N(C)OC] |
| I-241 | 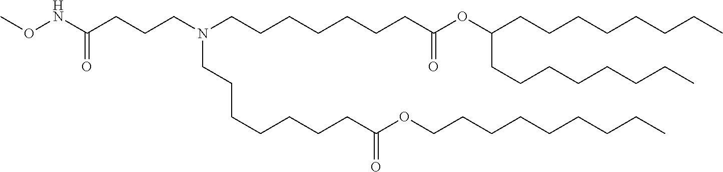 [SMILES: CCCCCCCCCOC(=O)CCCCCCCN(CCCCCCCC(=O)OC(CCCCCCCC)CCCCCCCC)CCCC(=O)NOC] |
| I-242 | 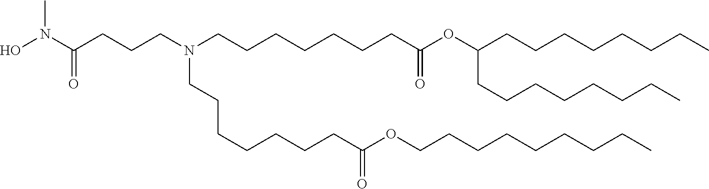 [SMILES: CCCCCCCCCOC(=O)CCCCCCCN(CCCCCCCC(=O)OC(CCCCCCCC)CCCCCCCC)CCCC(=O)N(C)O] |
| I-243 | 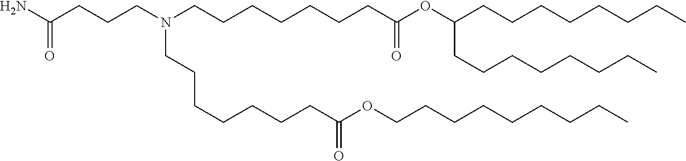 [SMILES: CCCCCCCCCOC(=O)CCCCCCCN(CCCCCCCC(=O)OC(CCCCCCCC)CCCCCCCC)CCCC(N)=O] |
| I-244 | 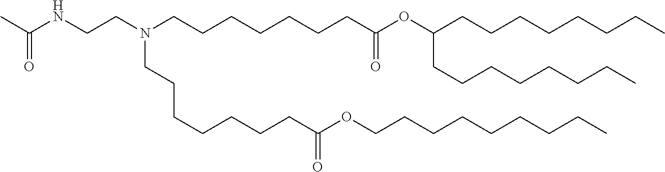 [SMILES: CCCCCCCCCOC(=O)CCCCCCCN(CCCCCCCC(=O)OC(CCCCCCCC)CCCCCCCC)CCNC(C)=O] |
| I-245 | 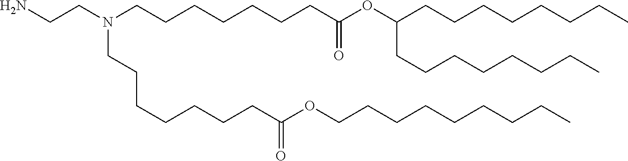 [SMILES: CCCCCCCCCOC(=O)CCCCCCCN(CCN)CCCCCCCC(=O)OC(CCCCCCCC)CCCCCCCC] |
| I-246 | 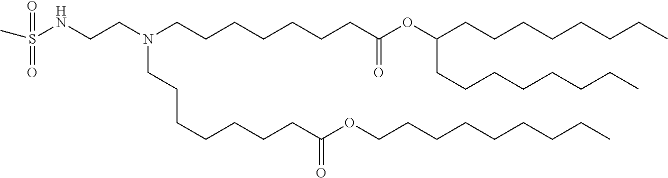 [SMILES: CCCCCCCCCOC(=O)CCCCCCCN(CCCCCCCC(=O)OC(CCCCCCCC)CCCCCCCC)CCNS(C)(=O)=O] |
| I-247 | 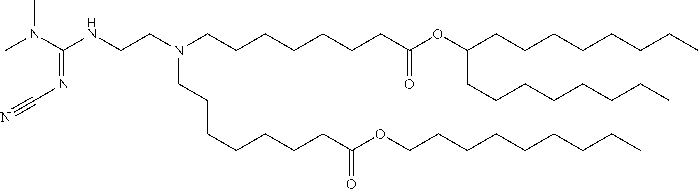 [SMILES: CCCCCCCCCOC(=O)CCCCCCCN(CCCCCCCC(=O)OC(CCCCCCCC)CCCCCCCC)CCN/C(=N/C#N)N(C)C] |
| I-248 | 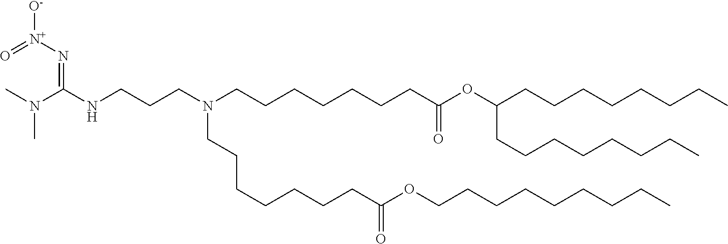 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I-249 | 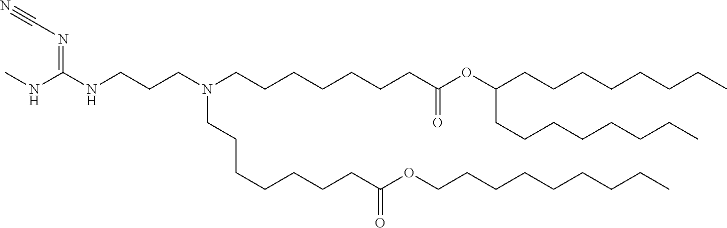 [SMILES: CCCCCCCCCOC(=O)CCCCCCCN(CCCCCCCC(=O)OC(CCCCCCCC)CCCCCCCC)CCCN/C(=N/C#N)NC] |
| I-250 | 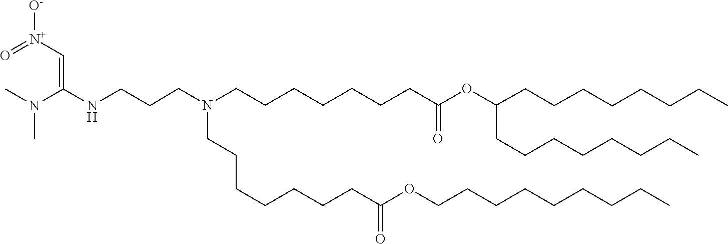 [SMILES: CCCCCCCCCOC(=O)CCCCCCCN(CCCCCCCC(=O)OC(CCCCCCCC)CCCCCCCC)CCCN/C(=C/[N+](=O)[O-])N(C)C] |
| I-251 | 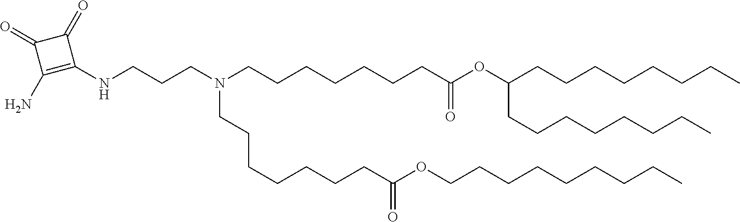 [SMILES: CCCCCCCCCOC(=O)CCCCCCCN(CCCCCCCC(=O)OC(CCCCCCCC)CCCCCCCC)CCCNc1c(N)c(=O)c1=O] |
| I-252 | 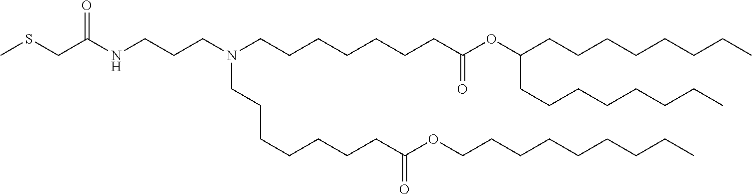 [SMILES: CCCCCCCCCOC(=O)CCCCCCCN(CCCCCCCC(=O)OC(CCCCCCCC)CCCCCCCC)CCCNC(=O)CSC] |
| I-253 | 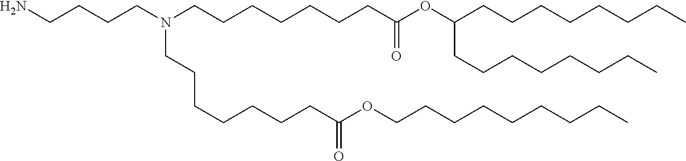 [SMILES: CCCCCCCCCOC(=O)CCCCCCCN(CCCCN)CCCCCCCC(=O)OC(CCCCCCCC)CCCCCCCC] |
| I-254 | 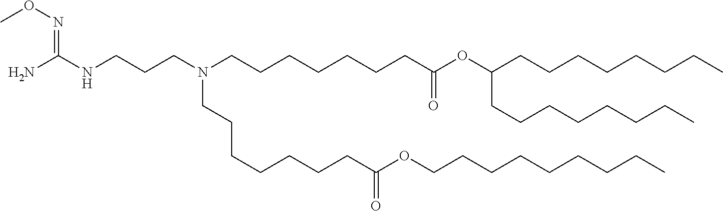 [SMILES: CCCCCCCCCOC(=O)CCCCCCCN(CCCCCCCC(=O)OC(CCCCCCCC)CCCCCCCC)CCCN/C(N)=N/OC] |
| I-255 | 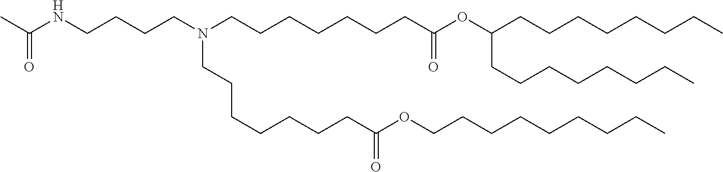 [SMILES: CCCCCCCCCOC(=O)CCCCCCCN(CCCCCCCC(=O)OC(CCCCCCCC)CCCCCCCC)CCCCNC(C)=O] |
| I-256 | 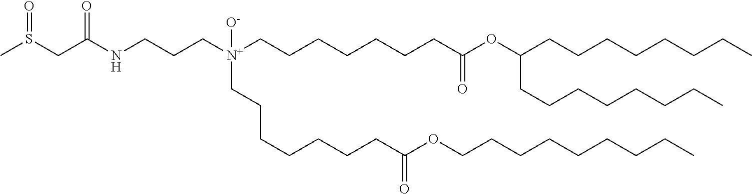 [SMILES: CCCCCCCCCOC(=O)CCCCCCC[N+]([O-])(CCCCCCCC(=O)OC(CCCCCCCC)CCCCCCCC)CCCNC(=O)CS(C)=O] |
| I-257 | 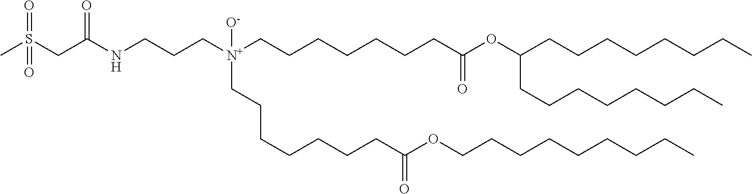 [SMILES: CCCCCCCCCOC(=O)CCCCCCC[N+]([O-])(CCCCCCCC(=O)OC(CCCCCCCC)CCCCCCCC)CCCNC(=O)CS(C)(=O)=O] |
| I-258 | 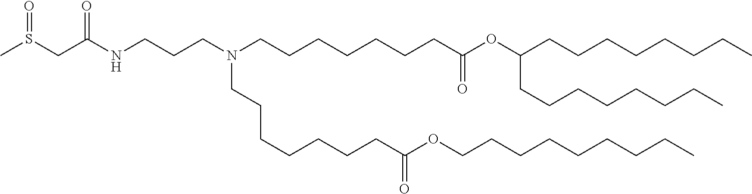 [SMILES: CCCCCCCCCOC(=O)CCCCCCCN(CCCCCCCC(=O)OC(CCCCCCCC)CCCCCCCC)CCCNC(=O)CS(C)=O] |
| I-259 | 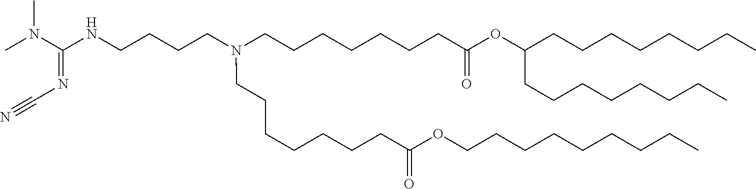 [SMILES: CCCCCCCCCOC(=O)CCCCCCCN(CCCCCCCC(=O)OC(CCCCCCCC)CCCCCCCC)CCCCN/C(=N/C#N)N(C)C] |
| I-260 | 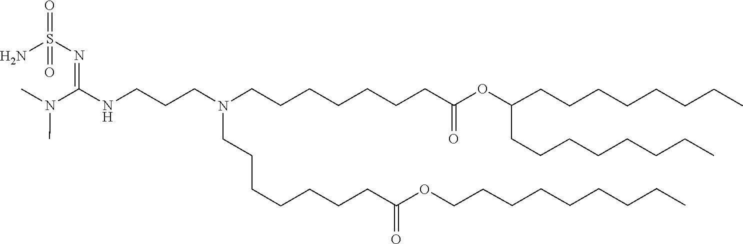 [SMILES: CCCCCCCCCOC(=O)CCCCCCCN(CCCCCCCC(=O)OC(CCCCCCCC)CCCCCCCC)CCCN/C(=N/S(N)(=O)=O)N(C)C] |
| I-261 | 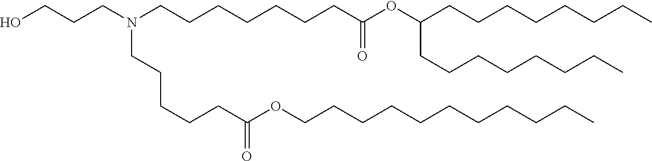 [SMILES: CCCCCCCCCCCOC(=O)CCCCCN(CCCO)CCCCCCCC(=O)OC(CCCCCCCC)CCCCCCCC] |
| I-262 | 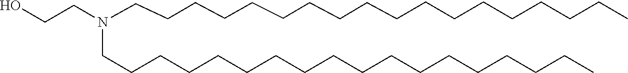 [SMILES: CCCCCCCCCCCCCCCCCCN(CCO)CCCCCCCCCCCCCCCCCC] |
| I-263 | 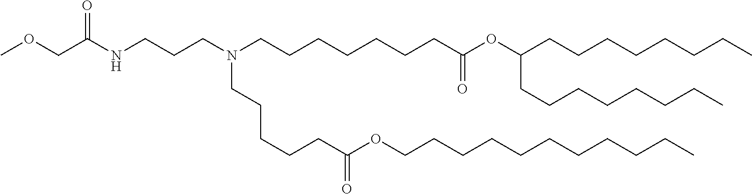 [SMILES: CCCCCCCCCCCOC(=O)CCCCCN(CCCCCCCC(=O)OC(CCCCCCCC)CCCCCCCC)CCCNC(=O)COC] |
| I-264 | 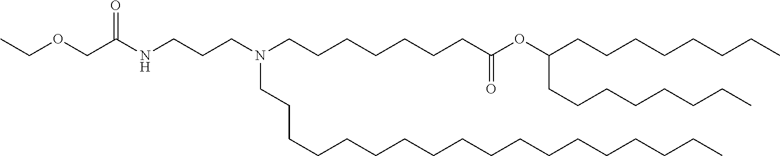 [SMILES: CCCCCCCCCCCCCCCCCCN(CCCCCCCC(=O)OC(CCCCCCCC)CCCCCCCC)CCCNC(=O)COCC] |
| I-265 | 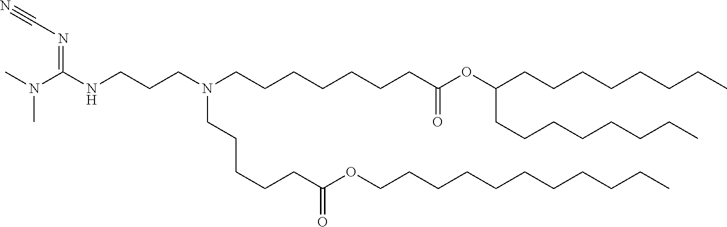 [SMILES: CCCCCCCCCCCOC(=O)CCCCCN(CCCCCCCC(=O)OC(CCCCCCCC)CCCCCCCC)CCCN/C(=N/C#N)N(C)C] |
| I-266 | 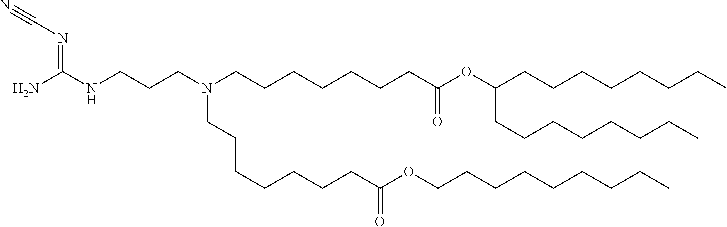 [SMILES: CCCCCCCCCOC(=O)CCCCCCCN(CCCCCCCC(=O)OC(CCCCCCCC)CCCCCCCC)CCCN/C(N)=N/C#N] |
| I-267 | 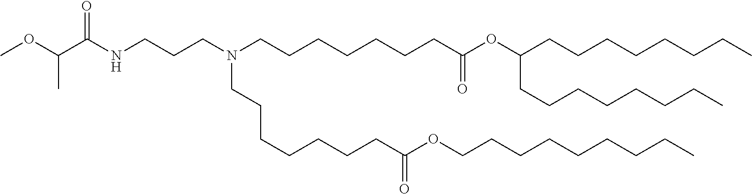 [SMILES: CCCCCCCCCOC(=O)CCCCCCCN(CCCCCCCC(=O)OC(CCCCCCCC)CCCCCCCC)CCCNC(=O)C(C)OC] |
| I-268 | 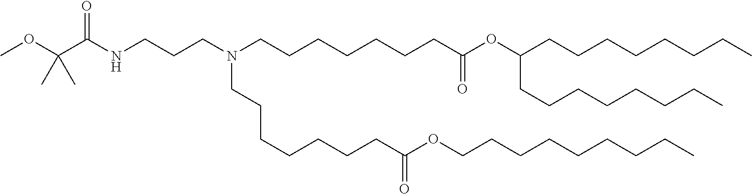 [SMILES: CCCCCCCCCOC(=O)CCCCCCCN(CCCCCCCC(=O)OC(CCCCCCCC)CCCCCCCC)CCCNC(=O)C(C)(C)OC] |
| I-269 | 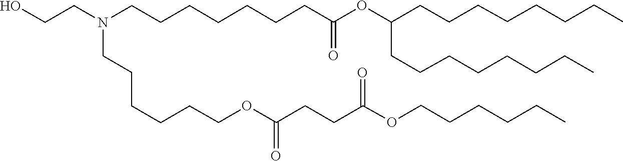 [SMILES: CCCCCCCCC(CCCCCCCC)OC(=O)CCCCCCCN(CCO)CCCCCCOC(=O)CCC(=O)OCCCCCC] |
| I-270 | 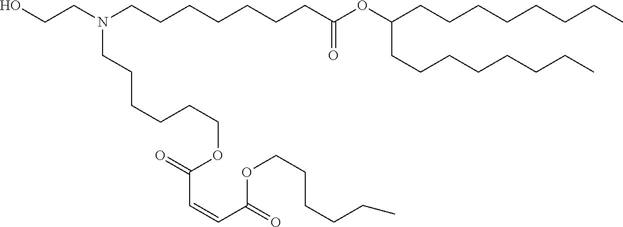 [SMILES: CCCCCCCCC(CCCCCCCC)OC(=O)CCCCCCCN(CCO)CCCCCCOC(=O)/C=C\C(=O)OCCCCCC] |
| I-271 | 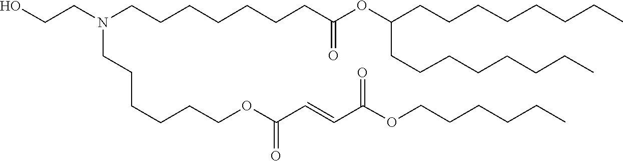 [SMILES: CCCCCCCCC(CCCCCCCC)OC(=O)CCCCCCCN(CCO)CCCCCCOC(=O)/C=C/C(=O)OCCCCCC] |
| I-272 | 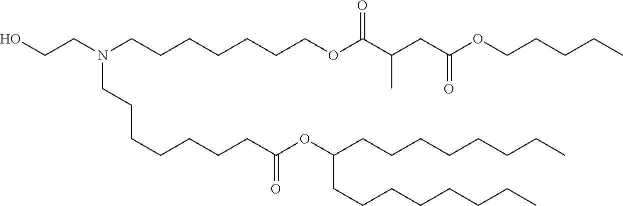 [SMILES: CCCCCCCCC(CCCCCCCC)OC(=O)CCCCCCCN(CCO)CCCCCCCOC(=O)C(C)CC(=O)OCCCCC] |
| I-273 | 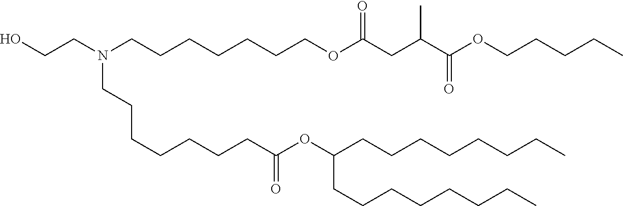 [SMILES: CCCCCCCCC(CCCCCCCC)OC(=O)CCCCCCCN(CCO)CCCCCCCOC(=O)CC(C)C(=O)OCCCCC] |
| I-274 | 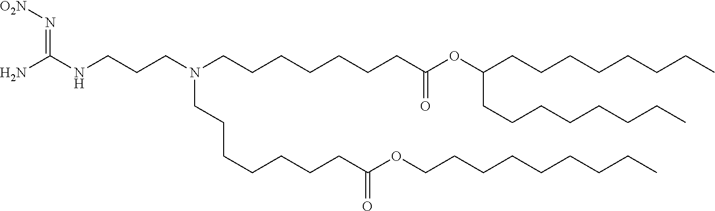 [SMILES: CCCCCCCCCOC(=O)CCCCCCCN(CCCCCCCC(=O)OC(CCCCCCCC)CCCCCCCC)CCCN/C(N)=N/[N+](=O)[O-]] |
| I-275 | 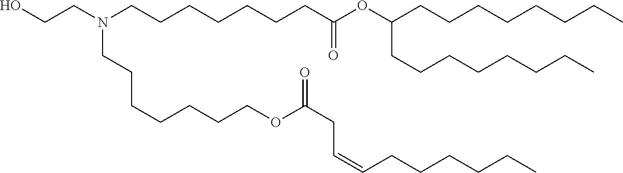 [SMILES: CCCCCC/C=C\CC(=O)OCCCCCCCN(CCO)CCCCCCCC(=O)OC(CCCCCCCC)CCCCCCCC] |
| I-276 | 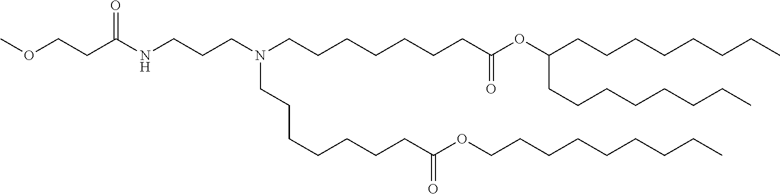 [SMILES: CCCCCCCCCOC(=O)CCCCCCCN(CCCCCCCC(=O)OC(CCCCCCCC)CCCCCCCC)CCCNC(=O)CCOC] |
| I-277 | 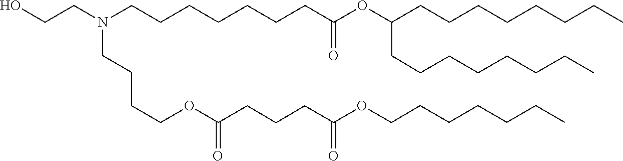 [SMILES: CCCCCCCCC(CCCCCCCC)OC(=O)CCCCCCCN(CCO)CCCCOC(=O)CCCC(=O)OCCCCCCC] |
| I-278 | 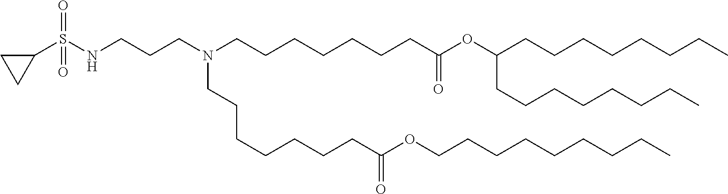 [SMILES: CCCCCCCCCOC(=O)CCCCCCCN(CCCCCCCC(=O)OC(CCCCCCCC)CCCCCCCC)CCCNS(=O)(=O)C1CC1] |
| I-279 | 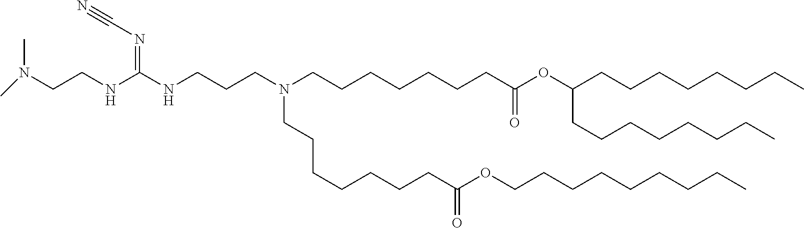 [SMILES: CCCCCCCCCOC(=O)CCCCCCCN(CCCCCCCC(=O)OC(CCCCCCCC)CCCCCCCC)CCCN/C(=N/C#N)NCCN(C)C] |
| I-280 | 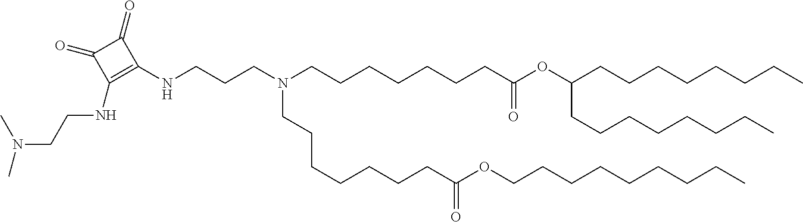 [SMILES: CCCCCCCCCOC(=O)CCCCCCCN(CCCCCCCC(=O)OC(CCCCCCCC)CCCCCCCC)CCCNc1c(NCCN(C)C)c(=O)c1=O] |
| I-281 | 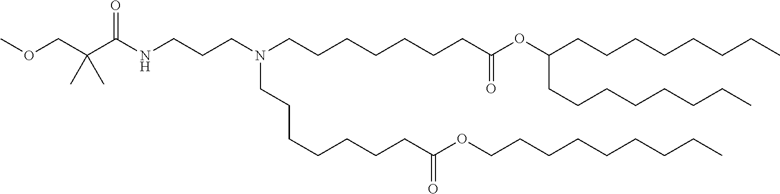 [SMILES: CCCCCCCCCOC(=O)CCCCCCCN(CCCCCCCC(=O)OC(CCCCCCCC)CCCCCCCC)CCCNC(=O)C(C)(C)COC] |
| I-282 | 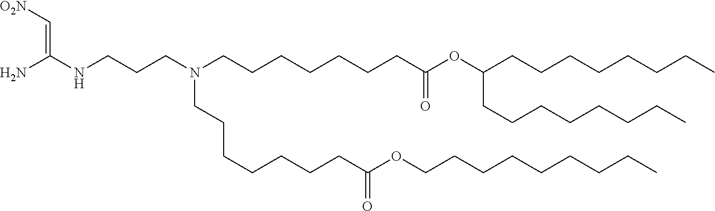 [SMILES: CCCCCCCCCOC(=O)CCCCCCCN(CCCCCCCC(=O)OC(CCCCCCCC)CCCCCCCC)CCCN/C(N)=C/[N+](=O)[O-]] |
| I-283 | 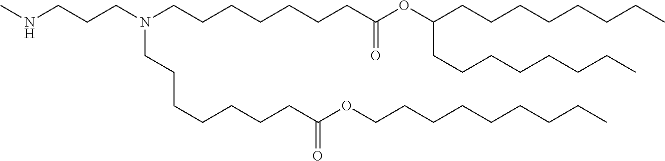 [SMILES: CCCCCCCCCOC(=O)CCCCCCCN(CCCCCCCC(=O)OC(CCCCCCCC)CCCCCCCC)CCCNC] |
| I-284 | 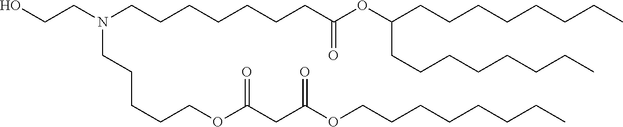 [SMILES: CCCCCCCCOC(=O)CC(=O)OCCCCCN(CCO)CCCCCCCC(=O)OC(CCCCCCCC)CCCCCCCC] |
| I-285 | 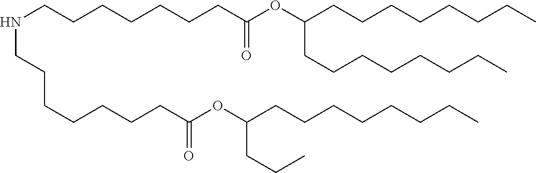 [SMILES: CCCCCCCCC(CCC)OC(=O)CCCCCCCNCCCCCCCC(=O)OC(CCCCCCCC)CCCCCCCC] |
| I-286 | 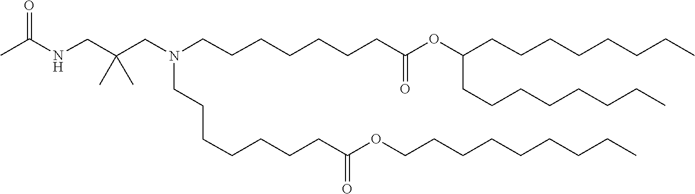 [SMILES: CCCCCCCCCOC(=O)CCCCCCCN(CCCCCCCC(=O)OC(CCCCCCCC)CCCCCCCC)CC(C)(C)CNC(C)=O] |
| I-287 | 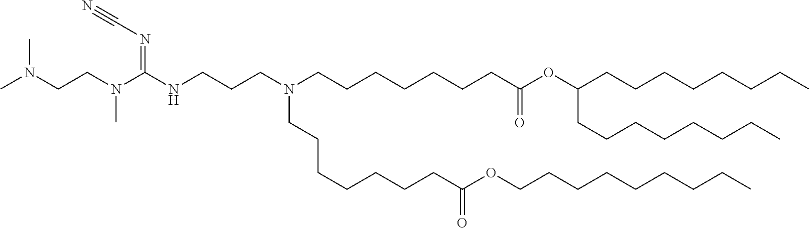 [SMILES: CCCCCCCCCOC(=O)CCCCCCCN(CCCCCCCC(=O)OC(CCCCCCCC)CCCCCCCC)CCCN/C(=N/C#N)N(C)CCN(C)C] |
| I-288 | 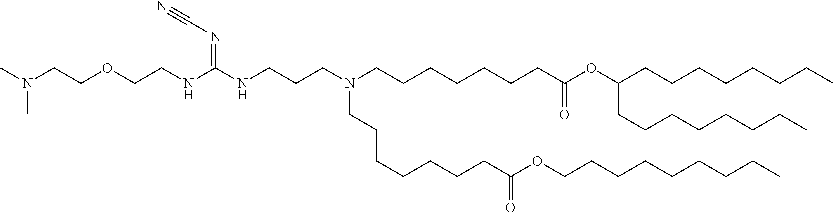 [SMILES: CCCCCCCCCOC(=O)CCCCCCCN(CCCCCCCC(=O)OC(CCCCCCCC)CCCCCCCC)CCCN/C(=N/C#N)NCCOCCN(C)C] |
| I-289 | 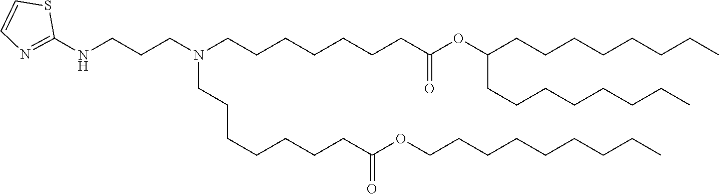 [SMILES: CCCCCCCCCOC(=O)CCCCCCCN(CCCCCCCC(=O)OC(CCCCCCCC)CCCCCCCC)CCCNc1nccs1] |
| I-290 | 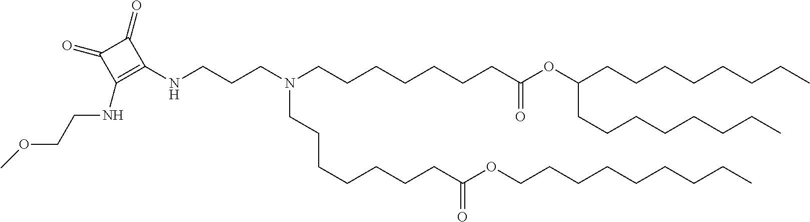 [SMILES: CCCCCCCCCOC(=O)CCCCCCCN(CCCCCCCC(=O)OC(CCCCCCCC)CCCCCCCC)CCCNc1c(NCCOC)c(=O)c1=O] |
| I-291 | 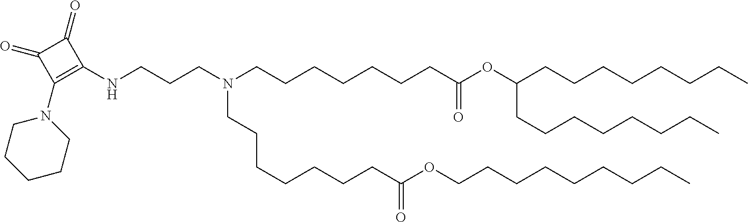 [SMILES: CCCCCCCCCOC(=O)CCCCCCCN(CCCCCCCC(=O)OC(CCCCCCCC)CCCCCCCC)CCCNc1c(N2CCCCC2)c(=O)c1=O] |
| I-292 | 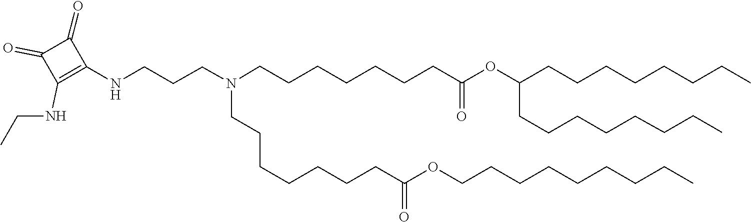 [SMILES: CCCCCCCCCOC(=O)CCCCCCCN(CCCCCCCC(=O)OC(CCCCCCCC)CCCCCCCC)CCCNc1c(NCC)c(=O)c1=O] |
| I-293 | 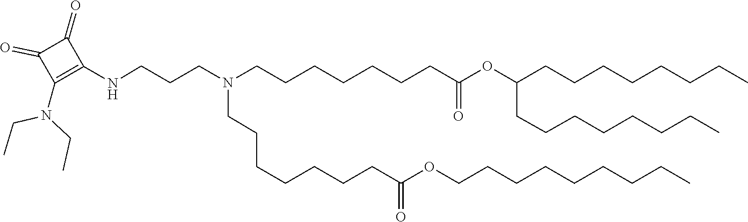 [SMILES: CCCCCCCCCOC(=O)CCCCCCCN(CCCCCCCC(=O)OC(CCCCCCCC)CCCCCCCC)CCCNc1c(N(CC)CC)c(=O)c1=O] |
| I-294 | 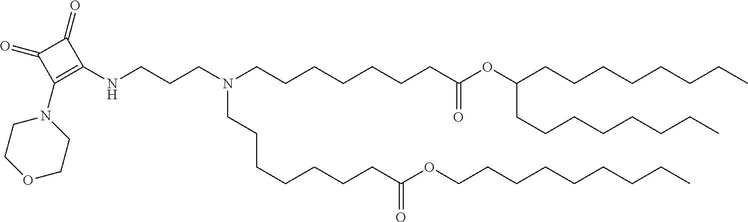 [SMILES: CCCCCCCCCOC(=O)CCCCCCCN(CCCCCCCC(=O)OC(CCCCCCCC)CCCCCCCC)CCCNc1c(N2CCOCC2)c(=O)c1=O] |
| I-295 | 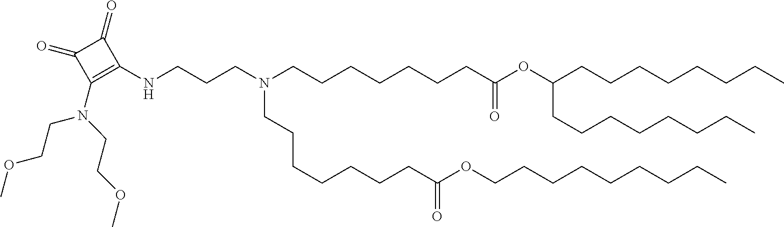 [SMILES: CCCCCCCCCOC(=O)CCCCCCCN(CCCCCCCC(=O)OC(CCCCCCCC)CCCCCCCC)CCCNc1c(N(CCOC)CCOC)c(=O)c1=O] |
| I-296 | 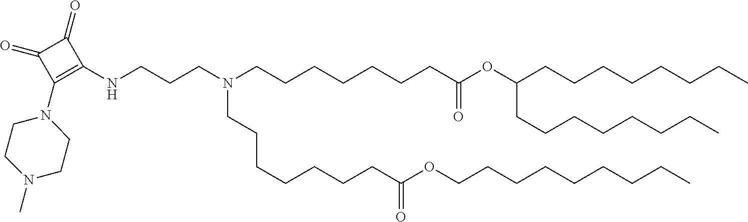 [SMILES: CCCCCCCCCOC(=O)CCCCCCCN(CCCCCCCC(=O)OC(CCCCCCCC)CCCCCCCC)CCCNc1c(N2CCN(C)CC2)c(=O)c1=O] |
| I-297 | 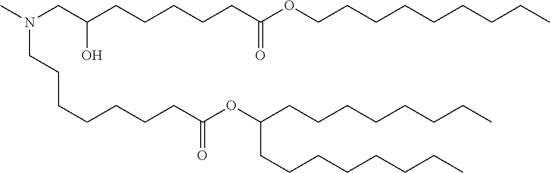 [SMILES: CCCCCCCCCOC(=O)CCCCCC(O)CN(C)CCCCCCCC(=O)OC(CCCCCCCC)CCCCCCCC] |
| I-298 | 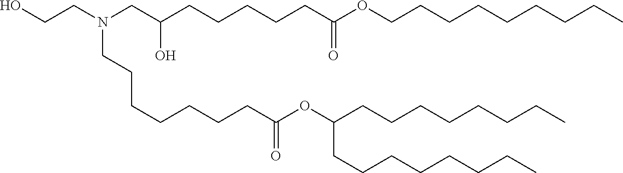 [SMILES: CCCCCCCCCOC(=O)CCCCCC(O)CN(CCO)CCCCCCCC(=O)OC(CCCCCCCC)CCCCCCCC] |
| I-299 | 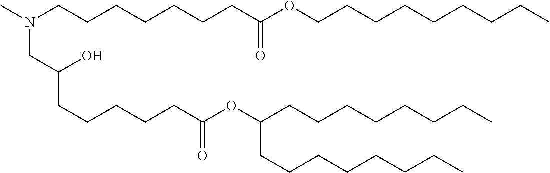 [SMILES: CCCCCCCCCOC(=O)CCCCCCCN(C)CC(O)CCCCCC(=O)OC(CCCCCCCC)CCCCCCCC] |
| I-300 | 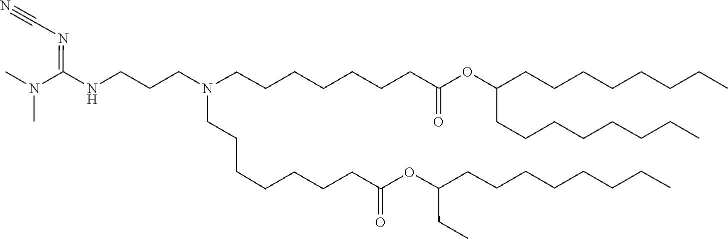 [SMILES: CCCCCCCCC(CC)OC(=O)CCCCCCCN(CCCCCCCC(=O)OC(CCCCCCCC)CCCCCCCC)CCCN/C(=N/C#N)N(C)C] |
| I-301 | 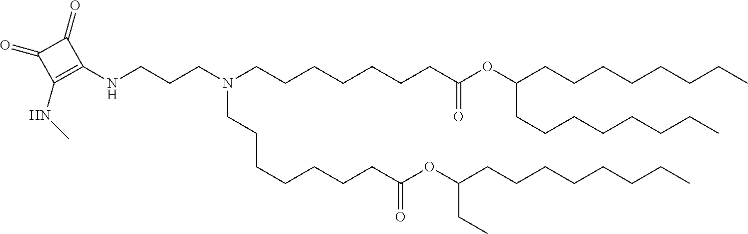 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I-302 | 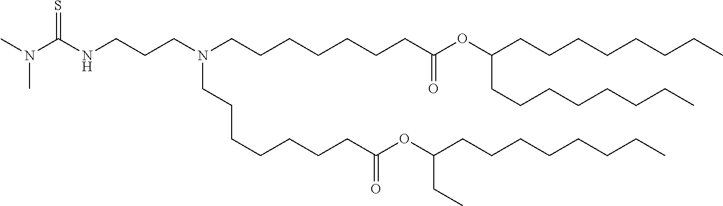 [SMILES: CCCCCCCCC(CC)OC(=O)CCCCCCCN(CCCCCCCC(=O)OC(CCCCCCCC)CCCCCCCC)CCCNC(=S)N(C)C] |
| I-303 | 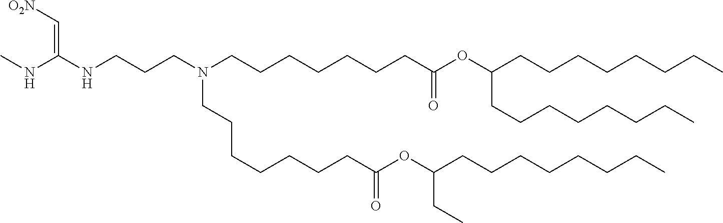 [SMILES: CCCCCCCCC(CC)OC(=O)CCCCCCCN(CCCCCCCC(=O)OC(CCCCCCCC)CCCCCCCC)CCCN/C(=C/[N+](=O)[O-])NC] |
| I-304 | 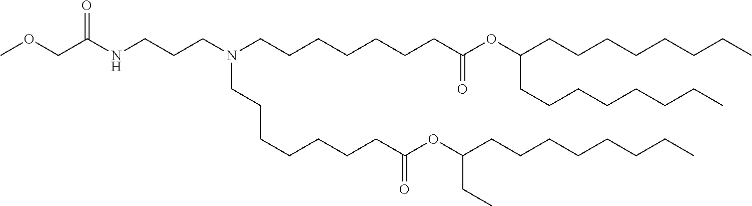 [SMILES: CCCCCCCCC(CC)OC(=O)CCCCCCCN(CCCCCCCC(=O)OC(CCCCCCCC)CCCCCCCC)CCCNC(=O)COC] |
| I-305 | 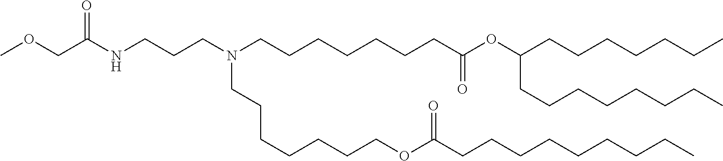 [SMILES: CCCCCCCCCC(=O)OCCCCCCCN(CCCCCCCC(=O)OC(CCCCCCC)CCCCCCCC)CCCNC(=O)COC] |
| I-306 | 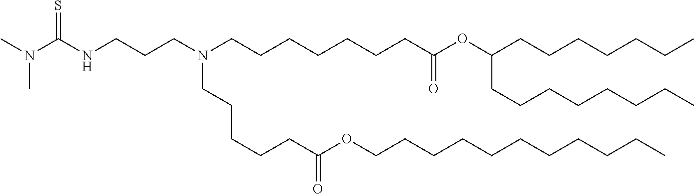 [SMILES: CCCCCCCCCCCOC(=O)CCCCCN(CCCCCCCC(=O)OC(CCCCCCC)CCCCCCCC)CCCNC(=S)N(C)C] |
| I-307 | 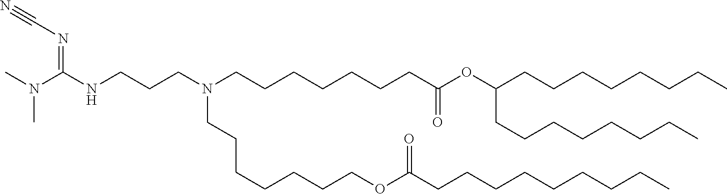 [SMILES: CCCCCCCCCC(=O)OCCCCCCCN(CCCCCCCC(=O)OC(CCCCCCCC)CCCCCCCC)CCCN/C(=N/C#N)N(C)C] |
| I-308 | 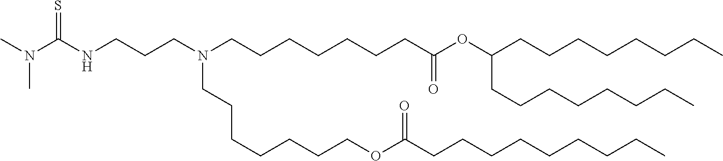 [SMILES: CCCCCCCCCC(=O)OCCCCCCCN(CCCCCCCC(=O)OC(CCCCCCCC)CCCCCCCC)CCCNC(=S)N(C)C] |
| I-309 | 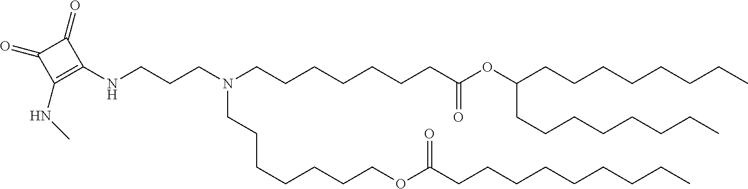 [SMILES: CCCCCCCCCC(=O)OCCCCCCCN(CCCCCCCC(=O)OC(CCCCCCCC)CCCCCCCC)CCCNc1c(NC)c(=O)c1=O] |
| I-310 | 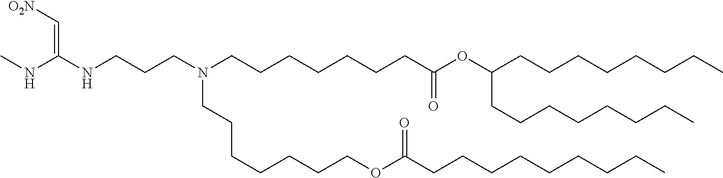 [SMILES: CCCCCCCCCC(=O)OCCCCCCCN(CCCCCCCC(=O)OC(CCCCCCCC)CCCCCCCC)CCCN/C(=C/[N+](=O)[O-])NC] |
| I-311 | 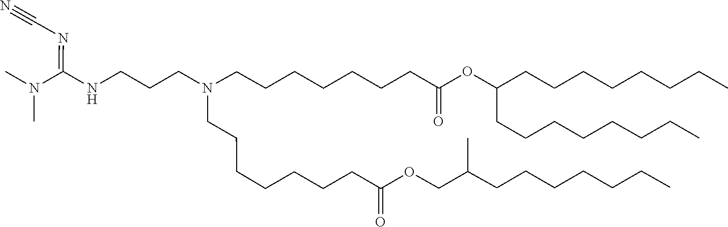 [SMILES: CCCCCCCCC(CCCCCCCC)OC(=O)CCCCCCCN(CCCCCCCC(=O)OCC(C)CCCCCCC)CCCN/C(=N/C#N)N(C)C] |
| I-312 | 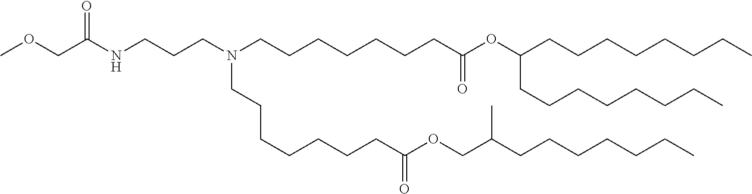 [SMILES: CCCCCCCCC(CCCCCCCC)OC(=O)CCCCCCCN(CCCCCCCC(=O)OCC(C)CCCCCCC)CCCNC(=O)COC] |
| I-313 | 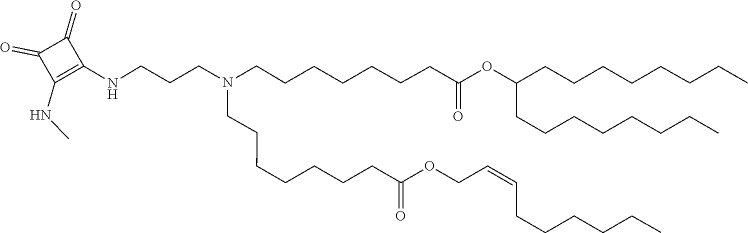 [SMILES: CCCCCC/C=C\COC(=O)CCCCCCCN(CCCCCCCC(=O)OC(CCCCCCCC)CCCCCCCC)CCCNc1c(NC)c(=O)c1=O] |
| I-314 | 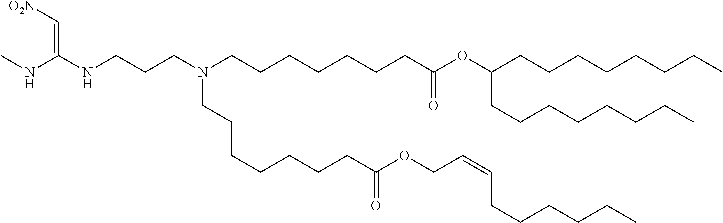 [SMILES: CCCCCC/C=C\COC(=O)CCCCCCCN(CCCCCCCC(=O)OC(CCCCCCCC)CCCCCCCC)CCCN/C(=C/[N+](=O)[O-])NC] |
| I-315 | 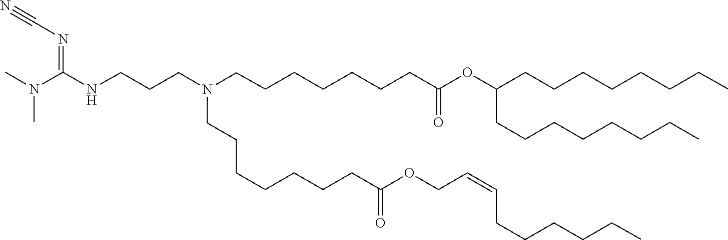 [SMILES: CCCCCC/C=C\COC(=O)CCCCCCCN(CCCCCCCC(=O)OC(CCCCCCCC)CCCCCCCC)CCCN/C(=N/C#N)N(C)C] |
| I-316 | 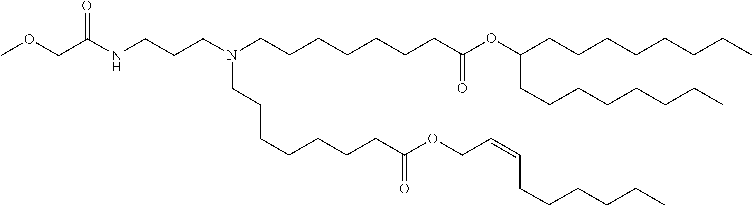 [SMILES: CCCCCC/C=C\COC(=O)CCCCCCCN(CCCCCCCC(=O)OC(CCCCCCCC)CCCCCCCC)CCCNC(=O)COC] |
| I-317 | 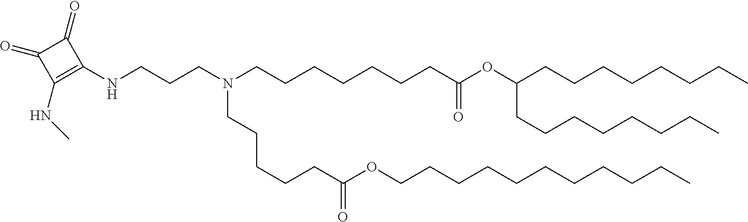 [SMILES: CCCCCCCCCCCOC(=O)CCCCCN(CCCCCCCC(=O)OC(CCCCCCCC)CCCCCCCC)CCCNc1c(NC)c(=O)c1=O] |
| I-318 | 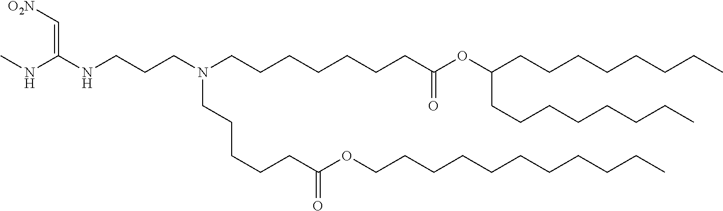 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I-319 | 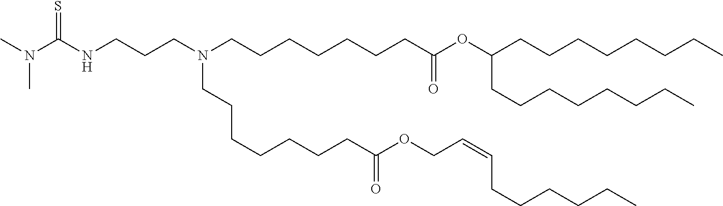 [SMILES: CCCCCC/C=C\COC(=O)CCCCCCCN(CCCCCCCC(=O)OC(CCCCCCCC)CCCCCCCC)CCCNC(=S)N(C)C] |
| I-320 | 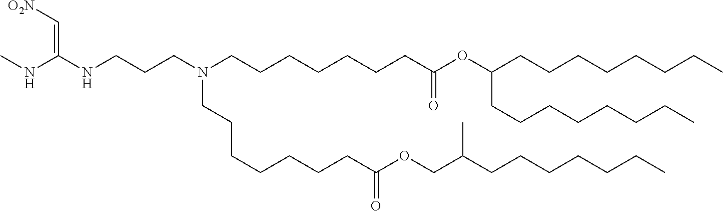 [SMILES: CCCCCCCCC(CCCCCCCC)OC(=O)CCCCCCCN(CCCCCCCC(=O)OCC(C)CCCCCCC)CCCN/C(=C/[N+](=O)[O-])NC] |
| I-321 | 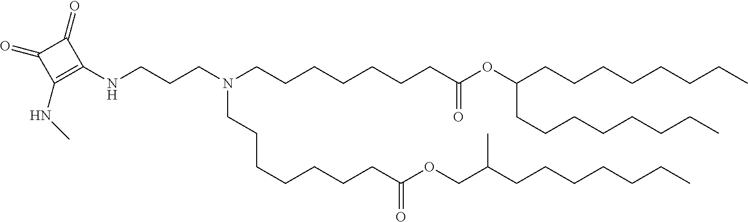 [SMILES: CCCCCCCCC(CCCCCCCC)OC(=O)CCCCCCCN(CCCCCCCC(=O)OCC(C)CCCCCCC)CCCNc1c(NC)c(=O)c1=O] |
| I-322 | 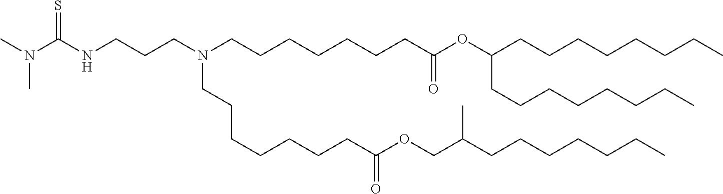 [SMILES: CCCCCCCCC(CCCCCCCC)OC(=O)CCCCCCCN(CCCCCCCC(=O)OCC(C)CCCCCCC)CCCNC(=S)N(C)C] |
| I-323 | 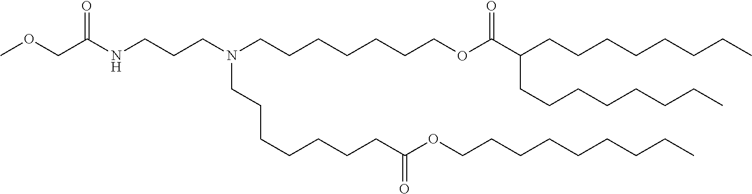 [SMILES: CCCCCCCCCOC(=O)CCCCCCCN(CCCCCCCOC(=O)C(CCCCCCCC)CCCCCCCC)CCCNC(=O)COC] |
| I-324 | 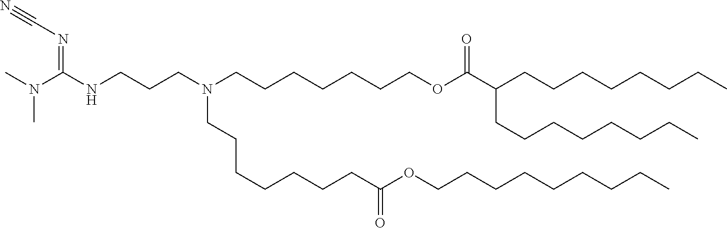 [SMILES: CCCCCCCCCOC(=O)CCCCCCCN(CCCCCCCOC(=O)C(CCCCCCCC)CCCCCCCC)CCCN/C(=N/C#N)N(C)C] |
| I-325 | 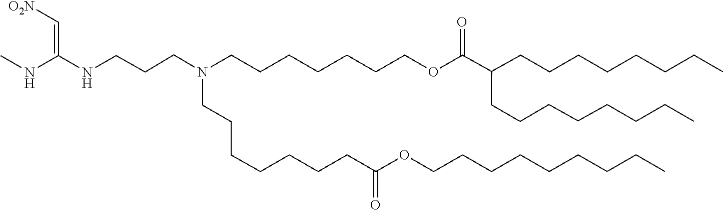 [SMILES: CCCCCCCCCOC(=O)CCCCCCCN(CCCCCCCOC(=O)C(CCCCCCCC)CCCCCCCC)CCCN/C(=C/[N+](=O)[O-])NC] |
| I-326 | 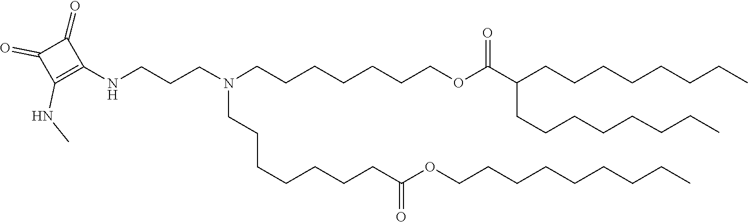 [SMILES: CCCCCCCCCOC(=O)CCCCCCCN(CCCCCCCOC(=O)C(CCCCCCCC)CCCCCCCC)CCCNc1c(NC)c(=O)c1=O] |
| I-327 | 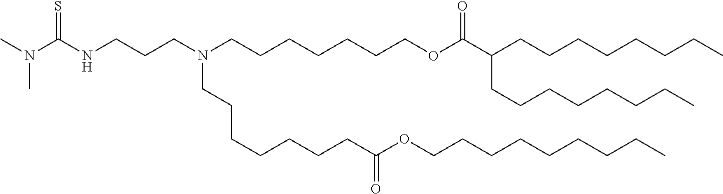 [SMILES: CCCCCCCCCOC(=O)CCCCCCCN(CCCCCCCOC(=O)C(CCCCCCCC)CCCCCCCC)CCCNC(=S)N(C)C] |
| I-328 | 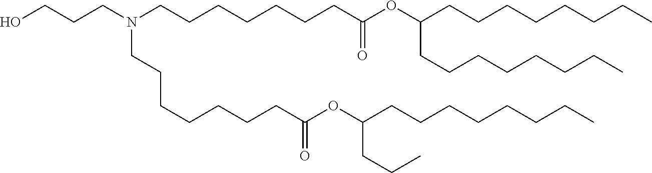 [SMILES: CCCCCCCCC(CCC)OC(=O)CCCCCCCN(CCCO)CCCCCCCC(=O)OC(CCCCCCCC)CCCCCCCC] |
| I-329 | 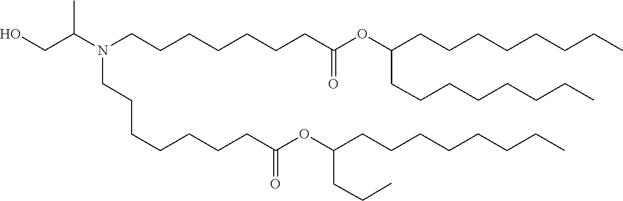 [SMILES: CCCCCCCCC(CCC)OC(=O)CCCCCCCN(CCCCCCCC(=O)OC(CCCCCCCC)CCCCCCCC)C(C)CO] |
| I-330 | 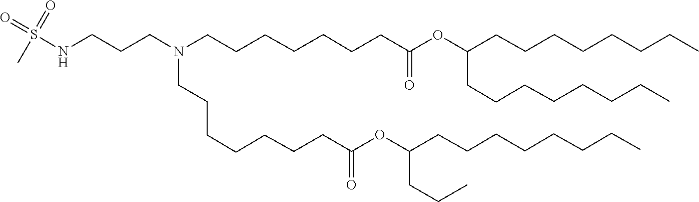 [SMILES: CCCCCCCCC(CCC)OC(=O)CCCCCCCN(CCCCCCCC(=O)OC(CCCCCCCC)CCCCCCCC)CCCNS(C)(=O)=O] |
| I-331 | 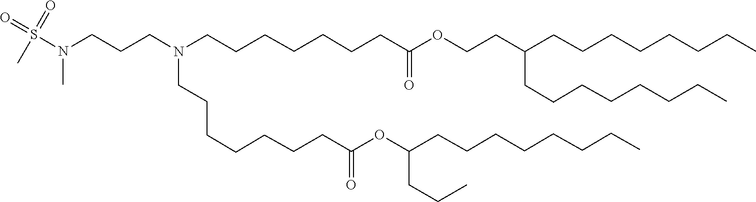 [SMILES: CCCCCCCCC(CCCCCCCC)CCOC(=O)CCCCCCCN(CCCCCCCC(=O)OC(CCC)CCCCCCCC)CCCN(C)S(C)(=O)=O] |
| I-332 | 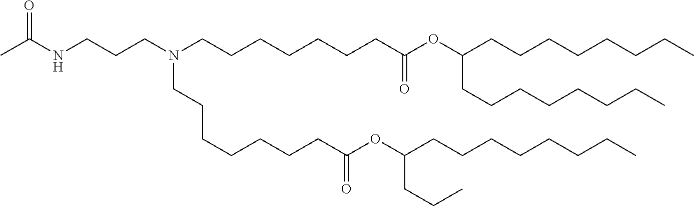 [SMILES: CCCCCCCCC(CCC)OC(=O)CCCCCCCN(CCCCCCCC(=O)OC(CCCCCCCC)CCCCCCCC)CCCNC(C)=O] |
| I-333 | 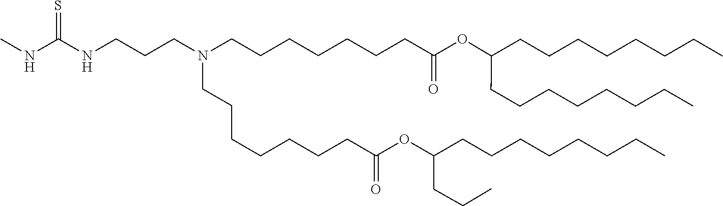 [SMILES: CCCCCCCCC(CCC)OC(=O)CCCCCCCN(CCCCCCCC(=O)OC(CCCCCCCC)CCCCCCCC)CCCNC(=S)NC] |
| I-334 | 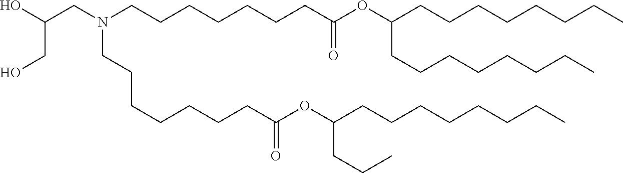 [SMILES: CCCCCCCCC(CCC)OC(=O)CCCCCCCN(CCCCCCCC(=O)OC(CCCCCCCC)CCCCCCCC)CC(O)CO] |
| I-335 | 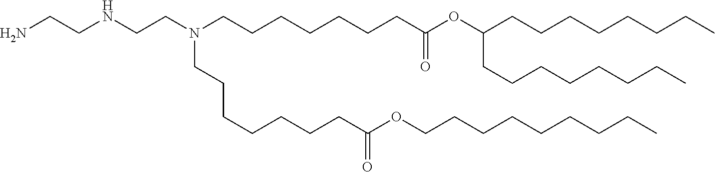 [SMILES: CCCCCCCCCOC(=O)CCCCCCCN(CCCCCCCC(=O)OC(CCCCCCCC)CCCCCCCC)CCNCCN] |
| I-336 | 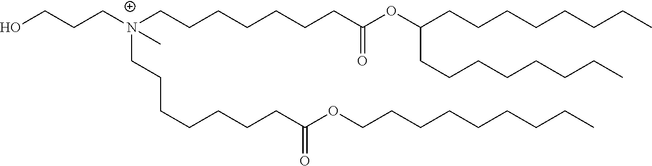 [SMILES: CCCCCCCCCOC(=O)CCCCCCC[N+](C)(CCCO)CCCCCCCC(=O)OC(CCCCCCCC)CCCCCCCC] |
| I-337 | 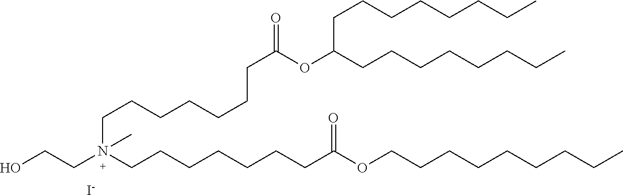 |
| I-338 | 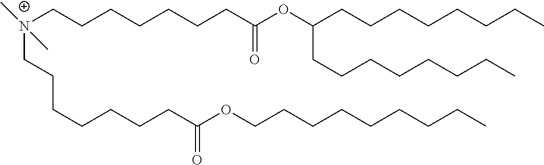 [SMILES: CCCCCCCCCOC(=O)CCCCCCC[N+](C)(C)CCCCCCCC(=O)OC(CCCCCCCC)CCCCCCCC] |
| I-339 | 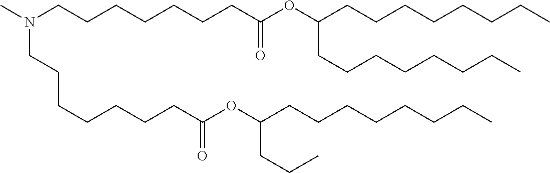 [SMILES: CCCCCCCCC(CCC)OC(=O)CCCCCCCN(C)CCCCCCCC(=O)OC(CCCCCCCC)CCCCCCCC] |
| I-340 | 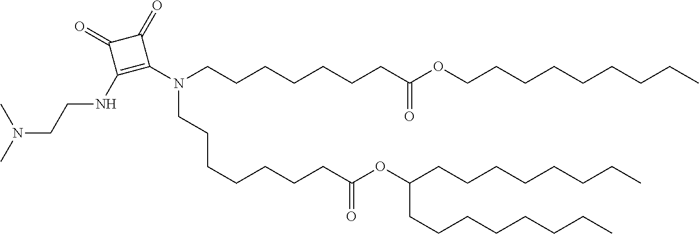 [SMILES: CCCCCCCCCOC(=O)CCCCCCCN(CCCCCCCC(=O)OC(CCCCCCCC)CCCCCCCC)c1c(NCCN(C)C)c(=O)c1=O] |
| I-341 | 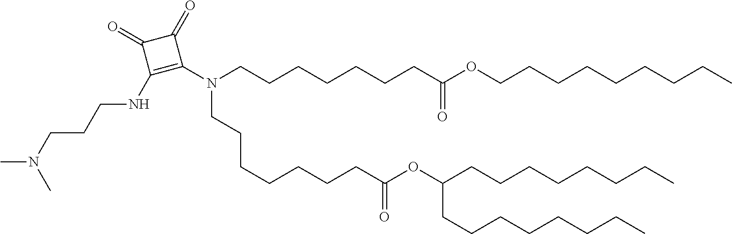 [SMILES: CCCCCCCCCOC(=O)CCCCCCCN(CCCCCCCC(=O)OC(CCCCCCCC)CCCCCCCC)c1c(NCCCN(C)C)c(=O)c1=O] |
| I-342 | 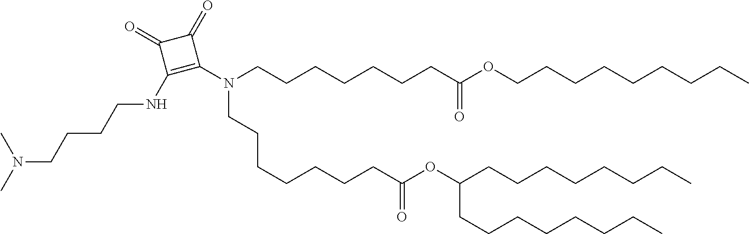 [SMILES: CCCCCCCCCOC(=O)CCCCCCCN(CCCCCCCC(=O)OC(CCCCCCCC)CCCCCCCC)c1c(NCCCCN(C)C)c(=O)c1=O] |
| I-343 | 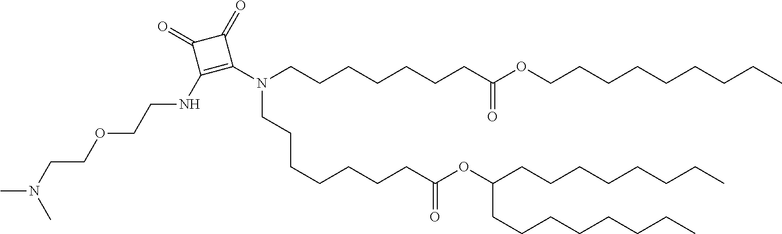 [SMILES: CCCCCCCCCOC(=O)CCCCCCCN(CCCCCCCC(=O)OC(CCCCCCCC)CCCCCCCC)c1c(NCCOCCN(C)C)c(=O)c1=O] |
| I-344 | 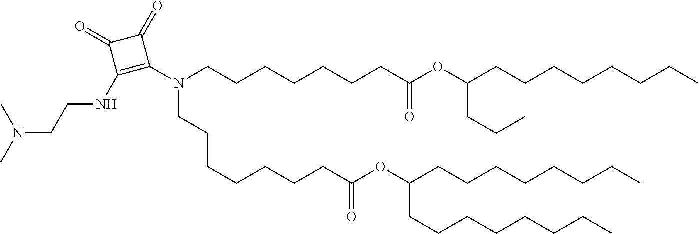 [SMILES: CCCCCCCCC(CCC)OC(=O)CCCCCCCN(CCCCCCCC(=O)OC(CCCCCCCC)CCCCCCCC)c1c(NCCN(C)C)c(=O)c1=O] |
| I-345 | 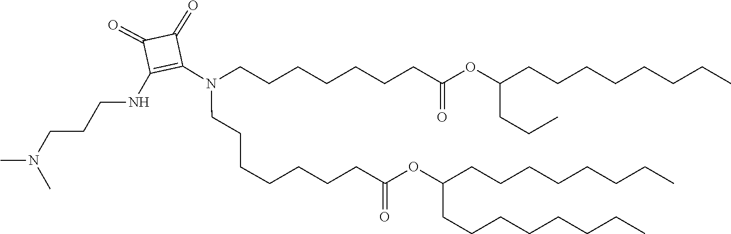 [SMILES: CCCCCCCCC(CCC)OC(=O)CCCCCCCN(CCCCCCCC(=O)OC(CCCCCCCC)CCCCCCCC)c1c(NCCCN(C)C)c(=O)c1=O] |
| I-346 | 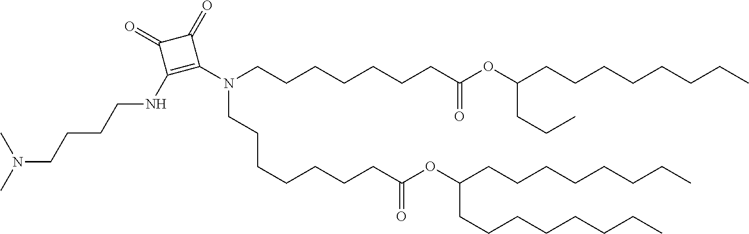 [SMILES: CCCCCCCCC(CCC)OC(=O)CCCCCCCN(CCCCCCCC(=O)OC(CCCCCCCC)CCCCCCCC)c1c(NCCCCN(C)C)c(=O)c1=O] |
| I-347 | 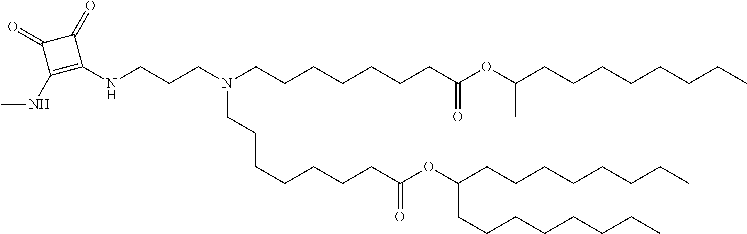 [SMILES: CCCCCCCCC(C)OC(=O)CCCCCCCN(CCCCCCCC(=O)OC(CCCCCCCC)CCCCCCCC)CCCNc1c(NC)c(=O)c1=O] |
| I-348 | 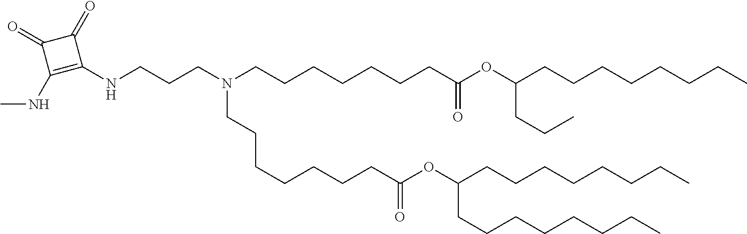 [SMILES: CCCCCCCCC(CCC)OC(=O)CCCCCCCN(CCCCCCCC(=O)OC(CCCCCCCC)CCCCCCCC)CCCNc1c(NC)c(=O)c1=O] |
| I-349 | 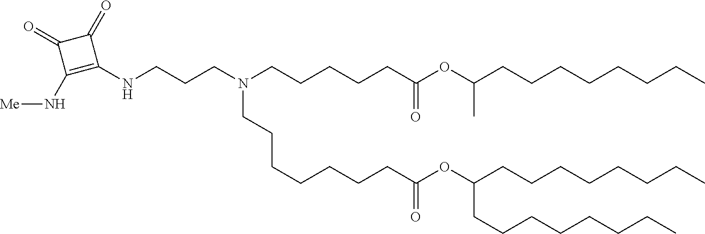 [SMILES: CCCCCCCCC(C)OC(=O)CCCCCN(CCCCCCCC(=O)OC(CCCCCCCC)CCCCCCCC)CCCNc1c(NC)c(=O)c1=O] |
| I-350 | 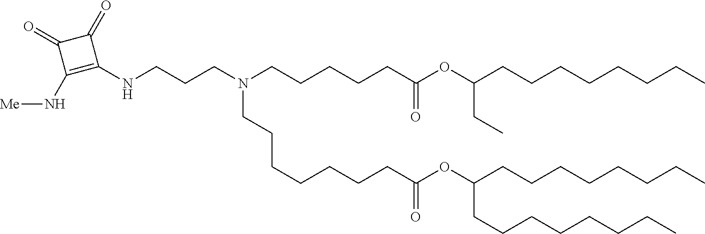 [SMILES: CCCCCCCCC(CC)OC(=O)CCCCCN(CCCCCCCC(=O)OC(CCCCCCCC)CCCCCCCC)CCCNc1c(NC)c(=O)c1=O] |
| I-351 | 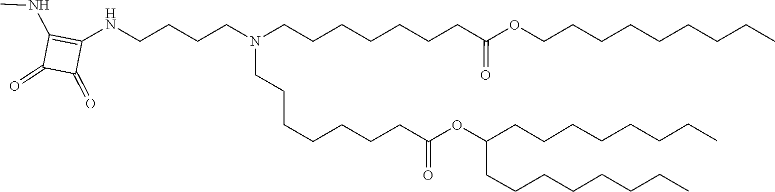 [SMILES: CCCCCCCCCOC(=O)CCCCCCCN(CCCCCCCC(=O)OC(CCCCCCCC)CCCCCCCC)CCCCNc1c(NC)c(=O)c1=O] |
| I-352 | 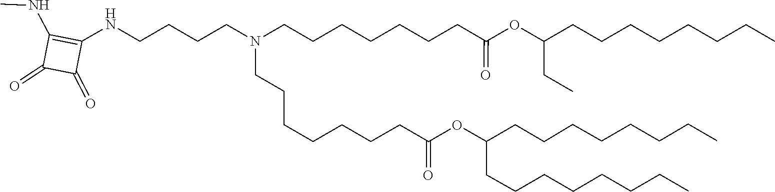 [SMILES: CCCCCCCCC(CC)OC(=O)CCCCCCCN(CCCCCCCC(=O)OC(CCCCCCCC)CCCCCCCC)CCCCNc1c(NC)c(=O)c1=O] |
| I-353 | 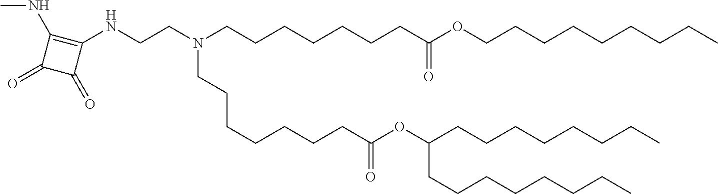 [SMILES: CCCCCCCCCOC(=O)CCCCCCCN(CCCCCCCC(=O)OC(CCCCCCCC)CCCCCCCC)CCNc1c(NC)c(=O)c1=O] |
| I-354 | 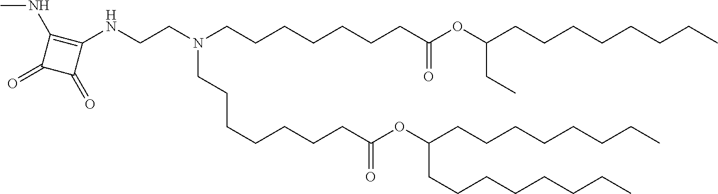 [SMILES: CCCCCCCCC(CC)OC(=O)CCCCCCCN(CCCCCCCC(=O)OC(CCCCCCCC)CCCCCCCC)CCNc1c(NC)c(=O)c1=O] |
| I-355 | 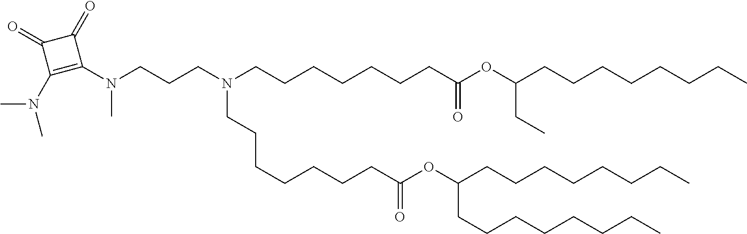 [SMILES: CCCCCCCCC(CC)OC(=O)CCCCCCCN(CCCCCCCC(=O)OC(CCCCCCCC)CCCCCCCC)CCCN(C)c1c(N(C)C)c(=O)c1=O] |
| I-356 | 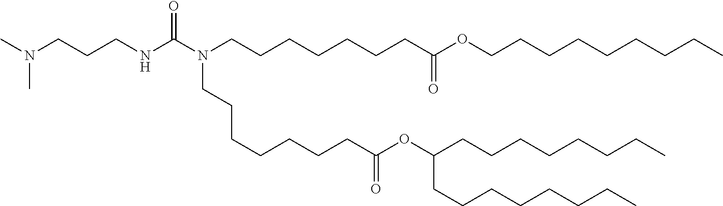 [SMILES: CCCCCCCCCOC(=O)CCCCCCCN(CCCCCCCC(=O)OC(CCCCCCCC)CCCCCCCC)C(=O)NCCCN(C)C] |
| I-357 | 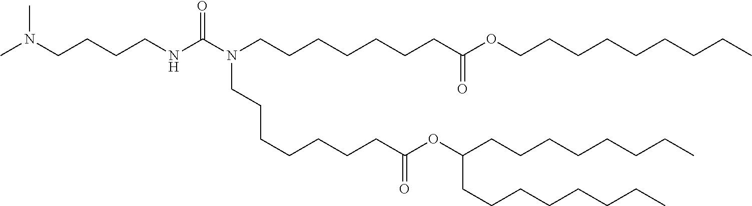 [SMILES: CCCCCCCCCOC(=O)CCCCCCCN(CCCCCCCC(=O)OC(CCCCCCCC)CCCCCCCC)C(=O)NCCCCN(C)C] |
| I-358 | 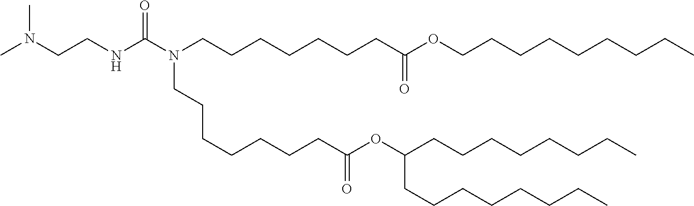 [SMILES: CCCCCCCCCOC(=O)CCCCCCCN(CCCCCCCC(=O)OC(CCCCCCCC)CCCCCCCC)C(=O)NCCN(C)C] |
| I-359 | 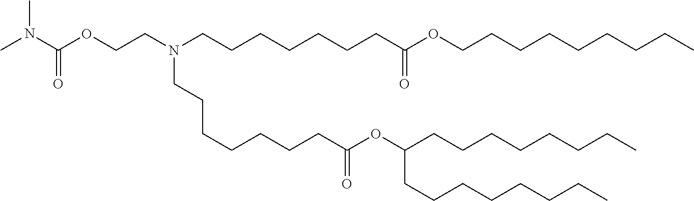 [SMILES: CCCCCCCCCOC(=O)CCCCCCCN(CCCCCCCC(=O)OC(CCCCCCCC)CCCCCCCC)CCOC(=O)N(C)C] |
| I-360 | 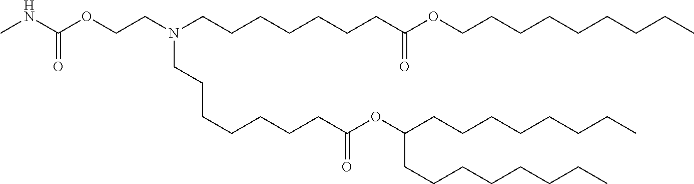 [SMILES: CCCCCCCCCOC(=O)CCCCCCCN(CCCCCCCC(=O)OC(CCCCCCCC)CCCCCCCC)CCOC(=O)NC] |
| I-361 | 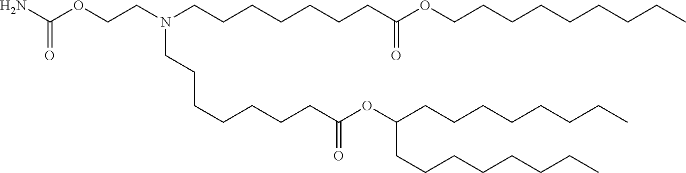 [SMILES: CCCCCCCCCOC(=O)CCCCCCCN(CCCCCCCC(=O)OC(CCCCCCCC)CCCCCCCC)CCOC(N)=O] |
| I-362 | 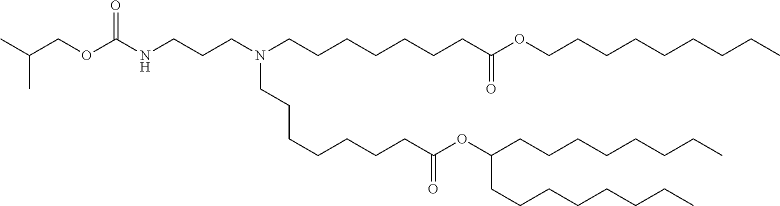 [SMILES: CCCCCCCCCOC(=O)CCCCCCCN(CCCCCCCC(=O)OC(CCCCCCCC)CCCCCCCC)CCCNC(=O)OCC(C)C] |
| I-363 | 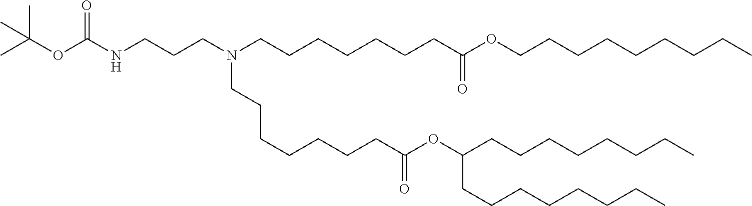 [SMILES: CCCCCCCCCOC(=O)CCCCCCCN(CCCCCCCC(=O)OC(CCCCCCCC)CCCCCCCC)CCCNC(=O)OC(C)(C)C] |
| I-364 | 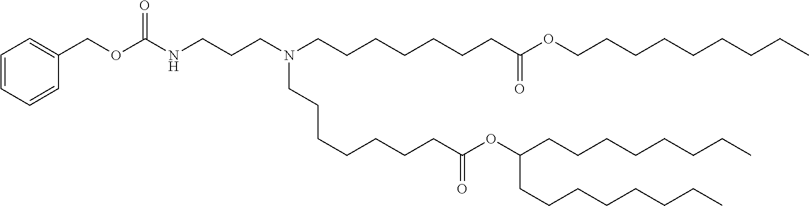 [SMILES: CCCCCCCCCOC(=O)CCCCCCCN(CCCCCCCC(=O)OC(CCCCCCCC)CCCCCCCC)CCCNC(=O)OCc1ccccc1] |
| I-365 | 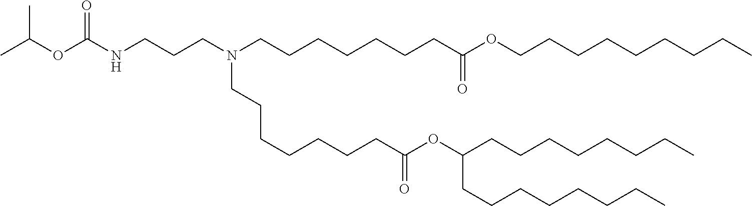 [SMILES: CCCCCCCCCOC(=O)CCCCCCCN(CCCCCCCC(=O)OC(CCCCCCCC)CCCCCCCC)CCCNC(=O)OC(C)C] |
| I-366 | 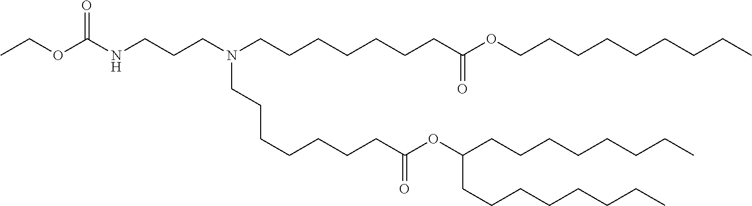 [SMILES: CCCCCCCCCOC(=O)CCCCCCCN(CCCCCCCC(=O)OC(CCCCCCCC)CCCCCCCC)CCCNC(=O)OCC] |
| I-367 | 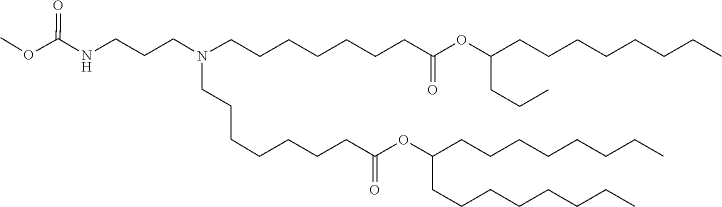 [SMILES: CCCCCCCCC(CCC)OC(=O)CCCCCCCN(CCCCCCCC(=O)OC(CCCCCCCC)CCCCCCCC)CCCNC(=O)OC] |
| I-368 | 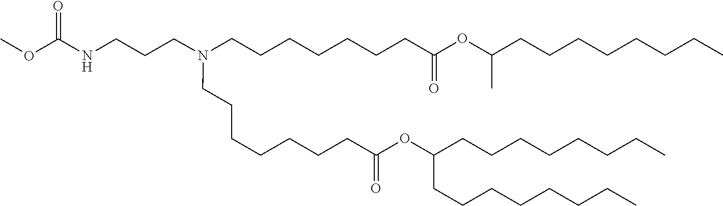 [SMILES: CCCCCCCCC(C)OC(=O)CCCCCCCN(CCCCCCCC(=O)OC(CCCCCCCC)CCCCCCCC)CCCNC(=O)OC] |
| I-369 | 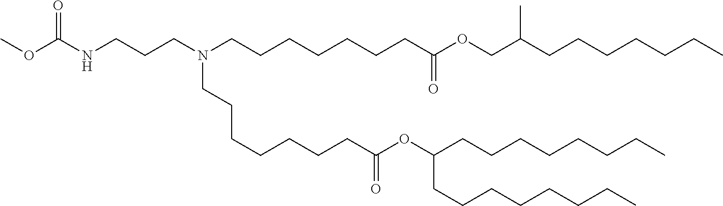 [SMILES: CCCCCCCCC(CCCCCCCC)OC(=O)CCCCCCCN(CCCCCCCC(=O)OCC(C)CCCCCCC)CCCNC(=O)OC] |
| I-370 | 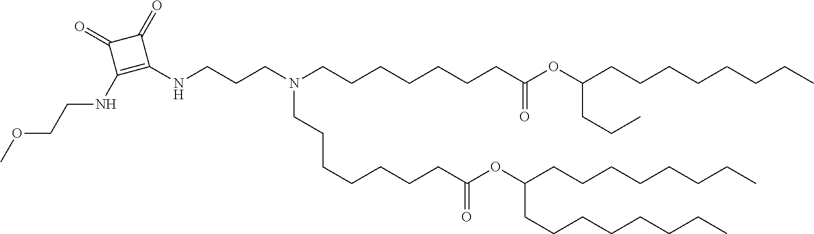 [SMILES: CCCCCCCCC(CCC)OC(=O)CCCCCCCN(CCCCCCCC(=O)OC(CCCCCCCC)CCCCCCCC)CCCNc1c(NCCOC)c(=O)c1=O] |
| I-371 | 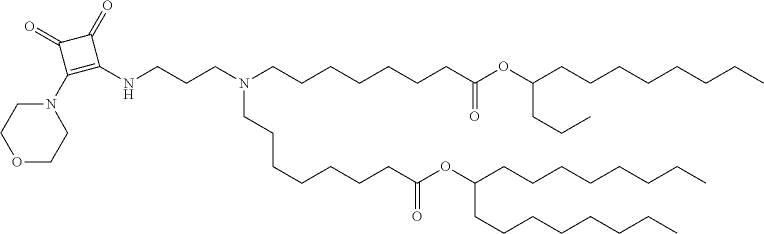 [SMILES: CCCCCCCCC(CCC)OC(=O)CCCCCCCN(CCCCCCCC(=O)OC(CCCCCCCC)CCCCCCCC)CCCNc1c(N2CCOCC2)c(=O)c1=O] |
| I-372 | 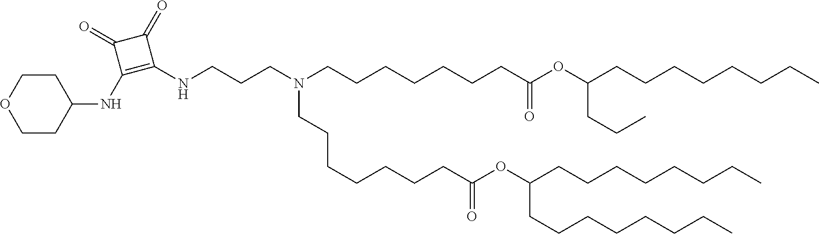 [SMILES: CCCCCCCCC(CCC)OC(=O)CCCCCCCN(CCCCCCCC(=O)OC(CCCCCCCC)CCCCCCCC)CCCNc1c(NC2CCOCC2)c(=O)c1=O] |
| I-373 | 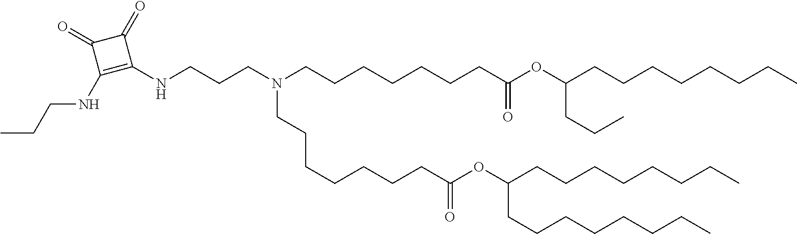 [SMILES: CCCCCCCCC(CCC)OC(=O)CCCCCCCN(CCCCCCCC(=O)OC(CCCCCCCC)CCCCCCCC)CCCNc1c(NCCC)c(=O)c1=O] |
| I-374 | 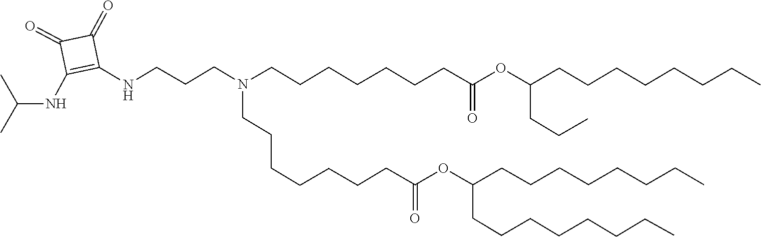 [SMILES: CCCCCCCCC(CCC)OC(=O)CCCCCCCN(CCCCCCCC(=O)OC(CCCCCCCC)CCCCCCCC)CCCNc1c(NC(C)C)c(=O)c1=O] |
| I-375 | 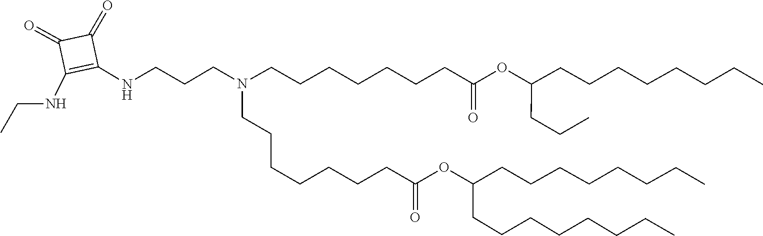 [SMILES: CCCCCCCCC(CCC)OC(=O)CCCCCCCN(CCCCCCCC(=O)OC(CCCCCCCC)CCCCCCCC)CCCNc1c(NCC)c(=O)c1=O] |
| I-376 | 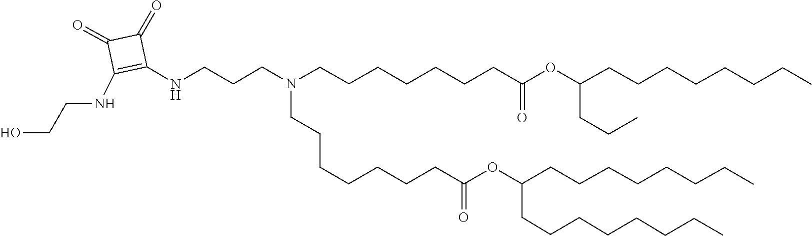 [SMILES: CCCCCCCCC(CCC)OC(=O)CCCCCCCN(CCCCCCCC(=O)OC(CCCCCCCC)CCCCCCCC)CCCNc1c(NCCO)c(=O)c1=O] |
| I-377 | 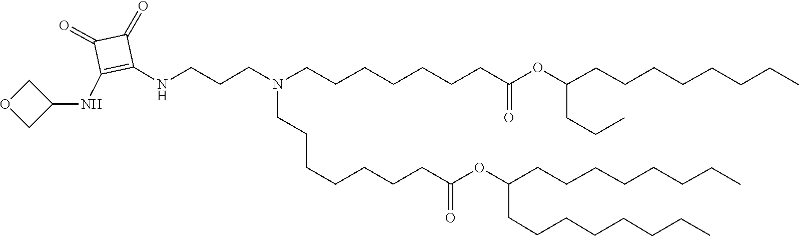 [SMILES: CCCCCCCCC(CCC)OC(=O)CCCCCCCN(CCCCCCCC(=O)OC(CCCCCCCC)CCCCCCCC)CCCNc1c(NC2COC2)c(=O)c1=O] |
| I-378 | 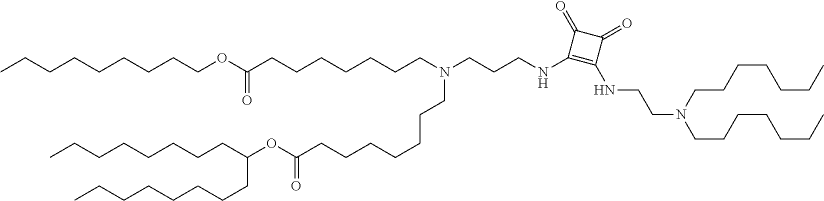 [SMILES: CCCCCCCCCOC(=O)CCCCCCCN(CCCCCCCC(=O)OC(CCCCCCCC)CCCCCCCC)CCCNc1c(NCCN(CCCCCCC)CCCCCCC)c(=O)c1=O] |
| I-379 | 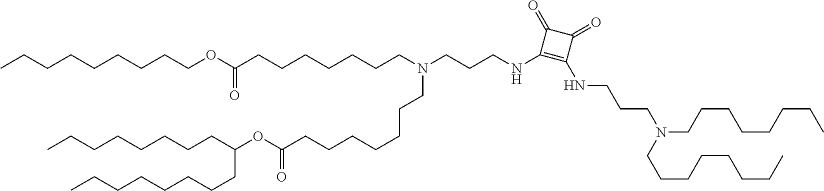 [SMILES: CCCCCCCCCOC(=O)CCCCCCCN(CCCCCCCC(=O)OC(CCCCCCCC)CCCCCCCC)CCCNc1c(NCCCN(CCCCCCCC)CCCCCCCC)c(=O)c1=O] |
| I-380 | 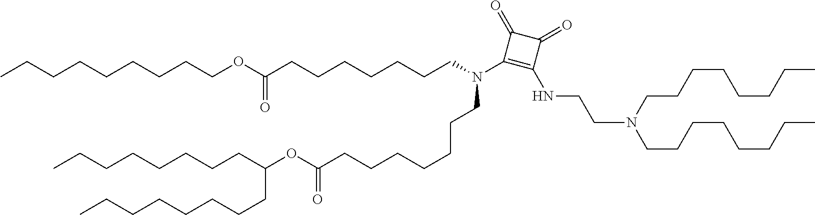 |
| I-381 | 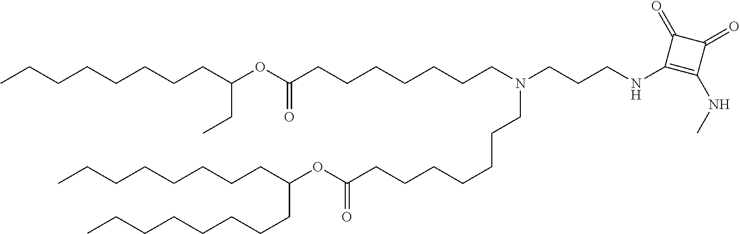 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I-382 | 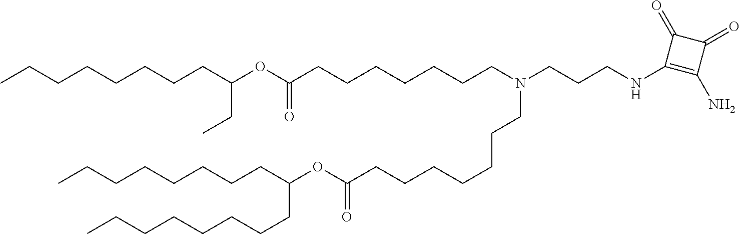 [SMILES: CCCCCCCCC(CC)OC(=O)CCCCCCCN(CCCCCCCC(=O)OC(CCCCCCCC)CCCCCCCC)CCCNc1c(N)c(=O)c1=O] |
| I-383 | 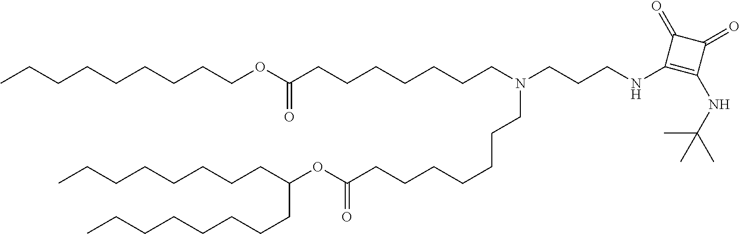 [SMILES: CCCCCCCCCOC(=O)CCCCCCCN(CCCCCCCC(=O)OC(CCCCCCCC)CCCCCCCC)CCCNc1c(NC(C)(C)C)c(=O)c1=O] |
| I-384 | 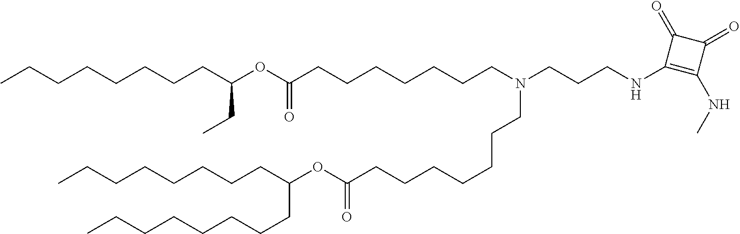 [SMILES: CCCCCCCCC(CCCCCCCC)OC(=O)CCCCCCCN(CCCCCCCC(=O)O[C@@H](CC)CCCCCCCC)CCCNc1c(NC)c(=O)c1=O] |
| I-385 | 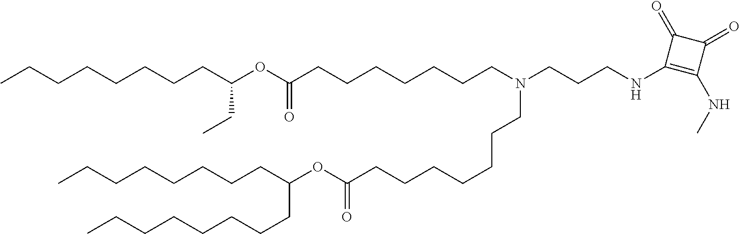 [SMILES: CCCCCCCCC(CCCCCCCC)OC(=O)CCCCCCCN(CCCCCCCC(=O)O[C@H](CC)CCCCCCCC)CCCNc1c(NC)c(=O)c1=O] |
| I-386 | 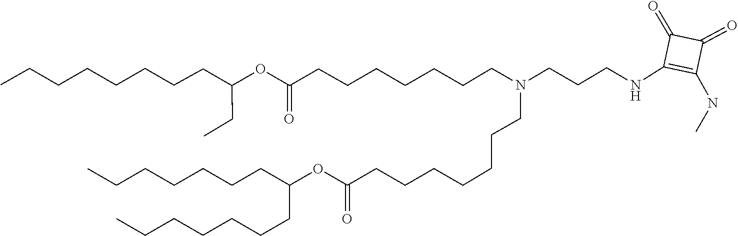 [SMILES: CCCCCCCCC(CC)OC(=O)CCCCCCCN(CCCCCCCC(=O)OC(CCCCCCC)CCCCCCC)CCCNc1c(NC)c(=O)c1=O] |
| I-387 | 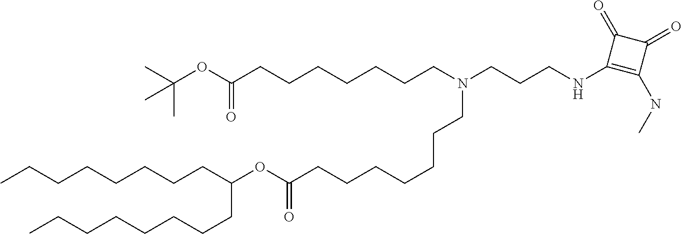 [SMILES: CCCCCCCCC(CCCCCCCC)OC(=O)CCCCCCCN(CCCCCCCC(=O)OC(C)(C)C)CCCNc1c(NC)c(=O)c1=O] |
| I-388 | 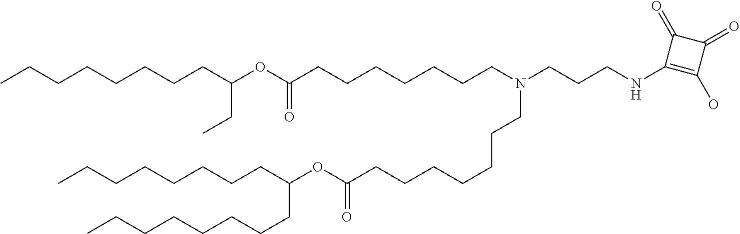 [SMILES: CCCCCCCCC(CC)OC(=O)CCCCCCCN(CCCCCCCC(=O)OC(CCCCCCCC)CCCCCCCC)CCCNc1c(O)c(=O)c1=O] |
| I-389 | 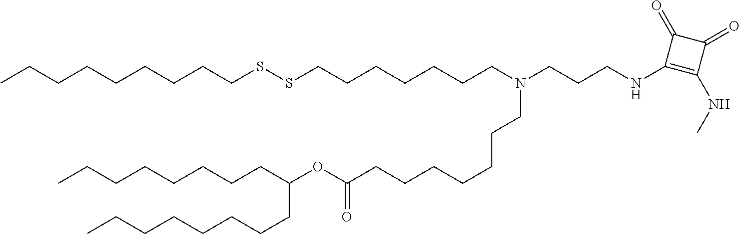 [SMILES: CCCCCCCCCSSCCCCCCCN(CCCCCCCC(=O)OC(CCCCCCCC)CCCCCCCC)CCCNc1c(NC)c(=O)c1=O] |
| I-390 | 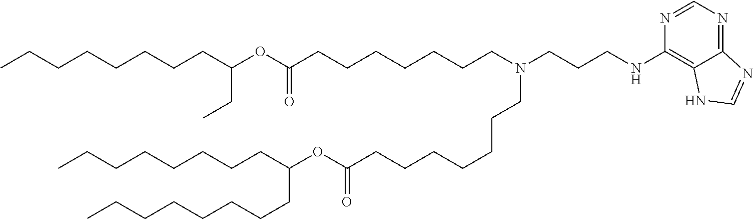 [SMILES: CCCCCCCCC(CC)OC(=O)CCCCCCCN(CCCCCCCC(=O)OC(CCCCCCCC)CCCCCCCC)CCCNc1ncnc2nc[nH]c12] |
| I-391 | 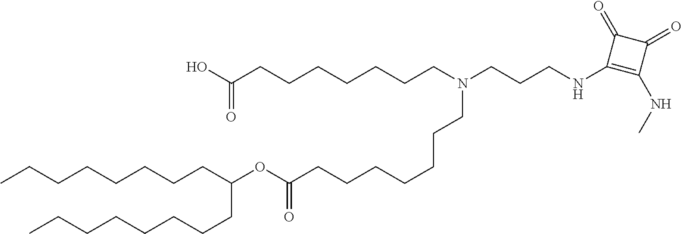 [SMILES: CCCCCCCCC(CCCCCCCC)OC(=O)CCCCCCCN(CCCCCCCC(=O)O)CCCNc1c(NC)c(=O)c1=O] |
| I-392 | 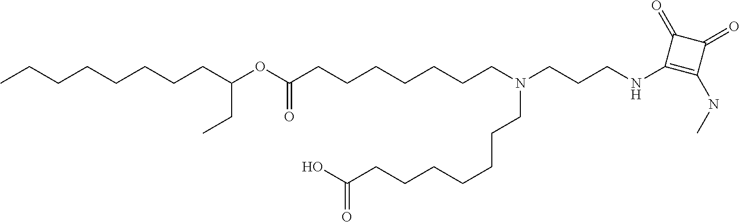 [SMILES: CCCCCCCCC(CC)OC(=O)CCCCCCCN(CCCCCCCC(=O)O)CCCNc1c(NC)c(=O)c1=O] |

In some embodiments, the ionizable lipid is a compound selected from Table IL-2.

TABLE IL-2

| Cpd | Structure |
|---|---|
| II-1 | 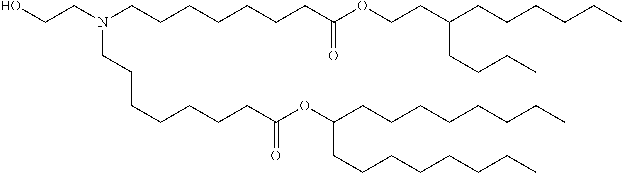 [SMILES: CCCCCCCCC(CCCCCCCC)OC(=O)CCCCCCCN(CCO)CCCCCCCC(=O)OCCC(CCCC)CCCCCC] |
| II-2 | 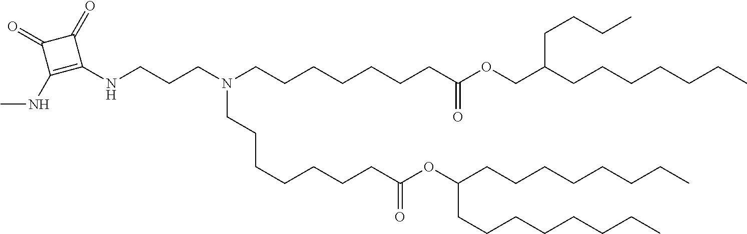 [SMILES: CCCCCCCCC(CCCCCCCC)OC(=O)CCCCCCCN(CCCCCCCC(=O)OCC(CCCC)CCCCCCC)CCCNc1c(NC)c(=O)c1=O] |
| II-3 | 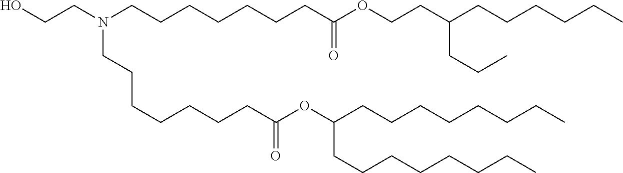 [SMILES: CCCCCCCCC(CCCCCCCC)OC(=O)CCCCCCCN(CCO)CCCCCCCC(=O)OCCC(CCC)CCCCCC] |
| II-4 | 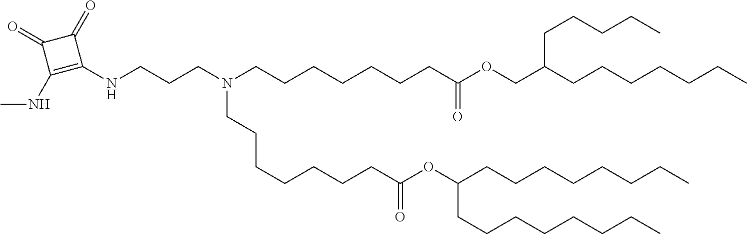 [SMILES: CCCCCCCCC(CCCCCCCC)OC(=O)CCCCCCCN(CCCCCCCC(=O)OCC(CCCCC)CCCCCCC)CCCNc1c(NC)c(=O)c1=O] |
| II-5 | 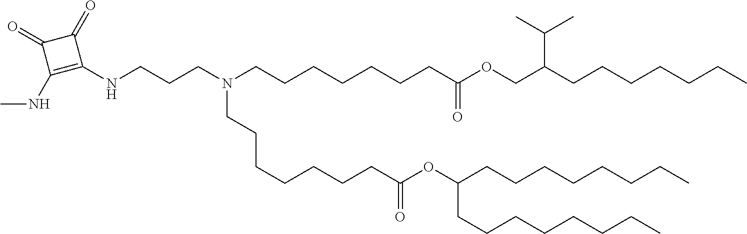 [SMILES: CCCCCCCCC(CCCCCCCC)OC(=O)CCCCCCCN(CCCCCCCC(=O)OCC(CCCCCCC)C(C)C)CCCNc1c(NC)c(=O)c1=O] |
| II-6 | 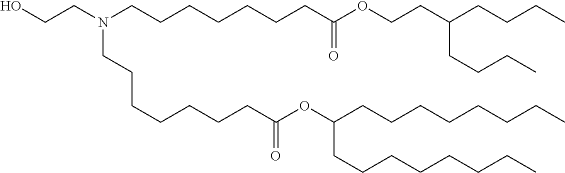 [SMILES: CCCCCCCCC(CCCCCCCC)OC(=O)CCCCCCCN(CCO)CCCCCCCC(=O)OCCC(CCCC)CCCC] |
| II-7 | 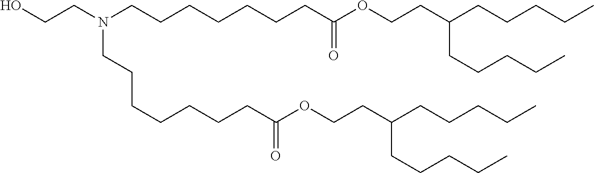 [SMILES: CCCCCC(CCCCC)CCOC(=O)CCCCCCCN(CCO)CCCCCCCC(=O)OCCC(CCCCC)CCCCC] |
| II-8 | 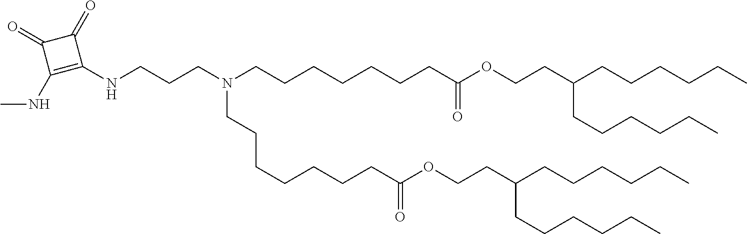 [SMILES: CCCCCCC(CCCCCC)CCOC(=O)CCCCCCCN(CCCCCCCC(=O)OCCC(CCCCCC)CCCCCC)CCCNc1c(NC)c(=O)c1=O] |
| II-9 | 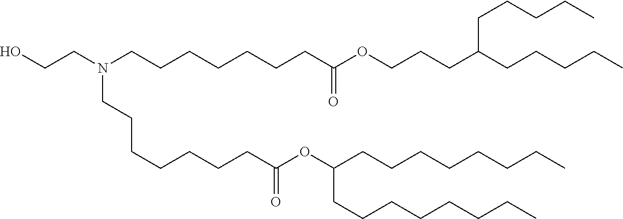 [SMILES: CCCCCCCCC(CCCCCCCC)OC(=O)CCCCCCCN(CCO)CCCCCCCC(=O)OCCCC(CCCCC)CCCCC] |
| II-10 | 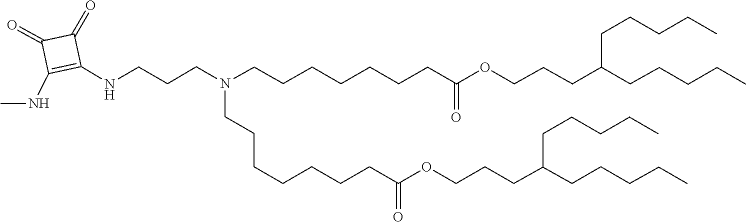 [SMILES: CCCCCC(CCCCC)CCCOC(=O)CCCCCCCN(CCCCCCCC(=O)OCCCC(CCCCC)CCCCC)CCCNc1c(NC)c(=O)c1=O] |
| II-11 | 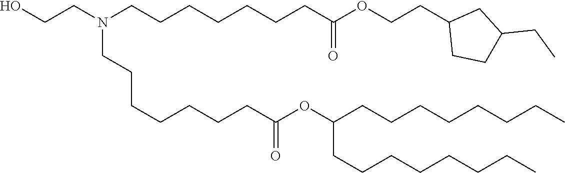 [SMILES: CCCCCCCCC(CCCCCCCC)OC(=O)CCCCCCCN(CCO)CCCCCCCC(=O)OCCC1CCC(CC)C1] |
| II-12 | 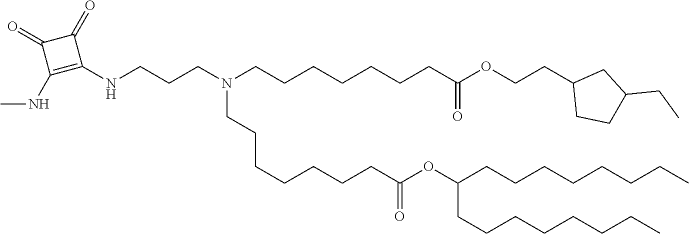 [SMILES: CCCCCCCCC(CCCCCCCC)OC(=O)CCCCCCCN(CCCCCCCC(=O)OCCC1CCC(CC)C1)CCCNc1c(NC)c(=O)c1=O] |
| II-13 | 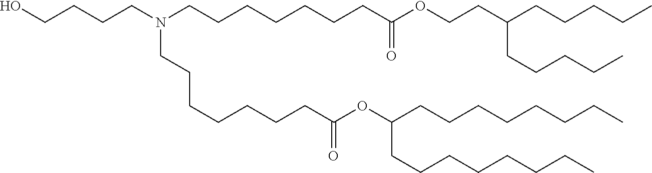 [SMILES: CCCCCCCCC(CCCCCCCC)OC(=O)CCCCCCCN(CCCCO)CCCCCCCC(=O)OCCC(CCCCC)CCCCC] |
| II-14 | 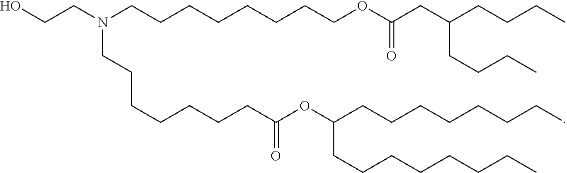 |
| II-15 | 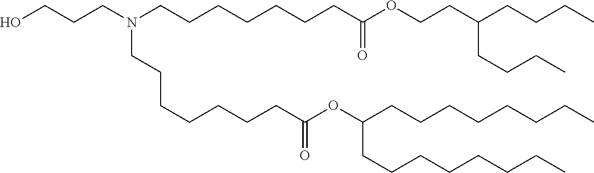 [SMILES: CCCCCCCCC(CCCCCCCC)OC(=O)CCCCCCCN(CCCO)CCCCCCCC(=O)OCCC(CCCC)CCCC] |
| II-16 | 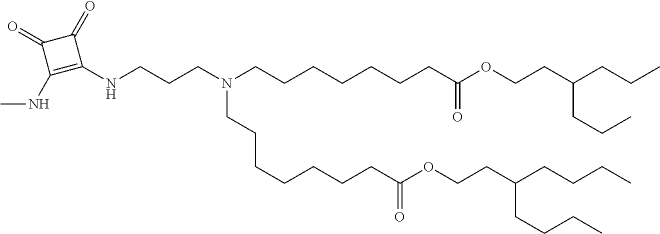 [SMILES: CCCCC(CCCC)CCOC(=O)CCCCCCCN(CCCCCCCC(=O)OCCC(CCC)CCC)CCCNc1c(NC)c(=O)c1=O] |
| II-17 | 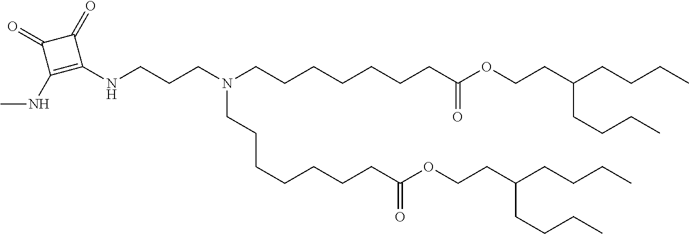 [SMILES: CCCCC(CCCC)CCOC(=O)CCCCCCCN(CCCCCCCC(=O)OCCC(CCCC)CCCC)CCCNc1c(NC)c(=O)c1=O] |
| II-18 | 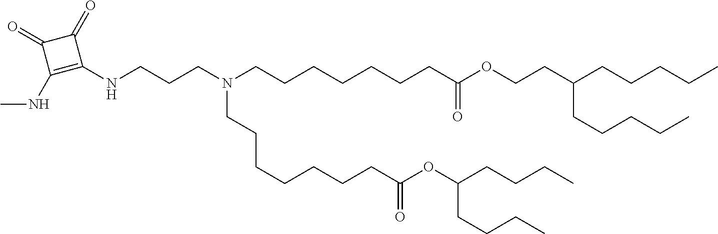 [SMILES: CCCCCC(CCCCC)CCOC(=O)CCCCCCCN(CCCCCCCC(=O)OC(CCCC)CCCC)CCCNc1c(NC)c(=O)c1=O] |
| II-19 | 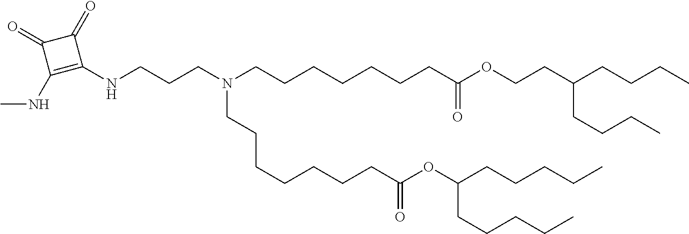 [SMILES: CCCCCC(CCCCC)OC(=O)CCCCCCCN(CCCCCCCC(=O)OCCC(CCCC)CCCC)CCCNc1c(NC)c(=O)c1=O] |
| II-20 | 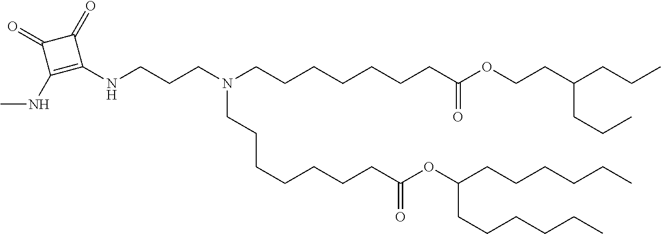 [SMILES: CCCCCCC(CCCCCC)OC(=O)CCCCCCCN(CCCCCCCC(=O)OCCC(CCC)CCC)CCCNc1c(NC)c(=O)c1=O] |
| II-21 | 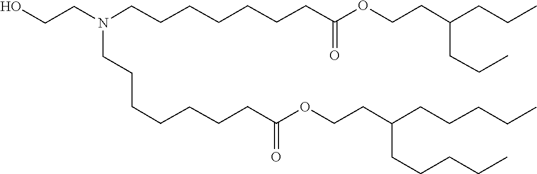 [SMILES: CCCCCC(CCCCC)CCOC(=O)CCCCCCCN(CCO)CCCCCCCC(=O)OCCC(CCC)CCC] |
| II-22 | 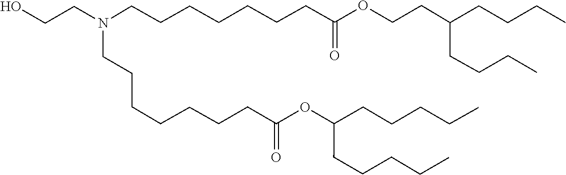 [SMILES: CCCCCC(CCCCC)OC(=O)CCCCCCCN(CCO)CCCCCCCC(=O)OCCC(CCCC)CCCC] |
| II-23 | 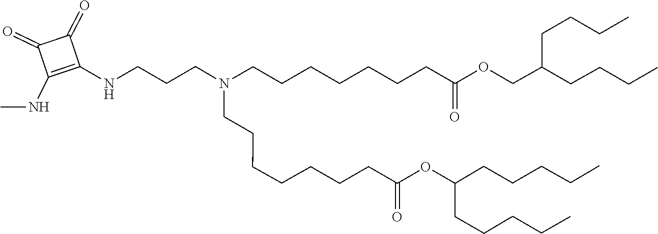 [SMILES: CCCCCC(CCCCC)OC(=O)CCCCCCCN(CCCCCCCC(=O)OCC(CCCC)CCCC)CCCNc1c(NC)c(=O)c1=O] |
| II-24 | 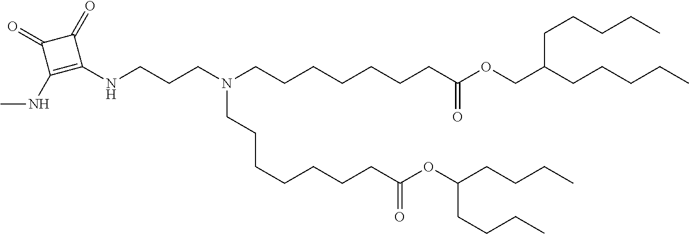 [SMILES: CCCCCC(CCCCC)COC(=O)CCCCCCCN(CCCCCCCC(=O)OC(CCCC)CCCC)CCCNc1c(NC)c(=O)c1=O] |
| II-25 | 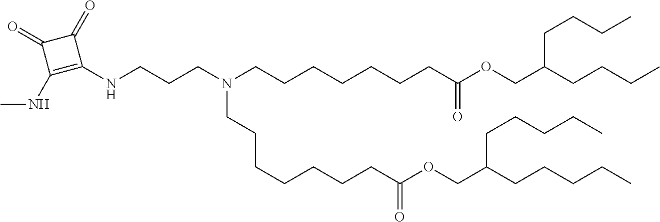 [SMILES: CCCCCC(CCCCC)COC(=O)CCCCCCCN(CCCCCCCC(=O)OCC(CCCC)CCCC)CCCNc1c(NC)c(=O)c1=O] |
| II-26 | 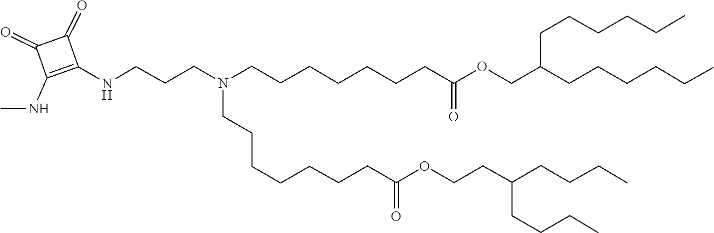 [SMILES: CCCCCCC(CCCCCC)COC(=O)CCCCCCCN(CCCCCCCC(=O)OCCC(CCCC)CCCC)CCCNc1c(NC)c(=O)c1=O] |
| II-27 | 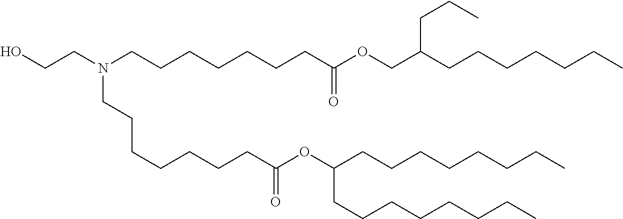 [SMILES: CCCCCCCCC(CCCCCCCC)OC(=O)CCCCCCCN(CCO)CCCCCCCC(=O)OCC(CCC)CCCCCCC] |
| II-28 | 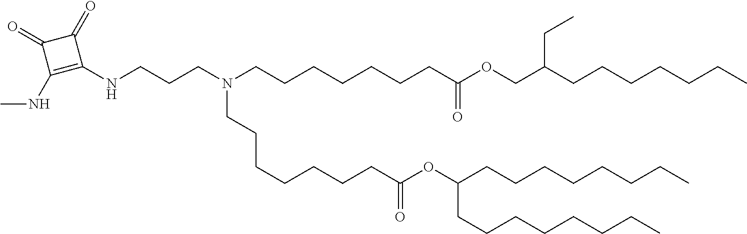 [SMILES: CCCCCCCCC(CCCCCCCC)OC(=O)CCCCCCCN(CCCCCCCC(=O)OCC(CC)CCCCCCC)CCCNc1c(NC)c(=O)c1=O] |
| II-29 | 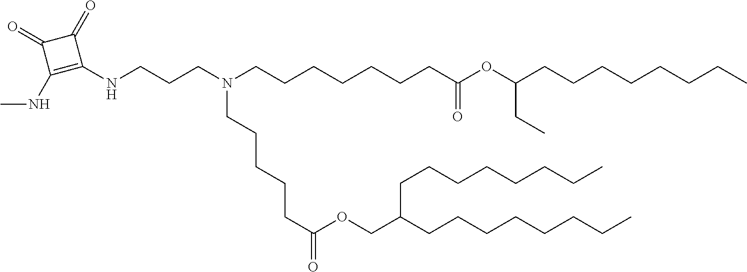 [SMILES: CCCCCCCCC(CCCCCCCC)COC(=O)CCCCCN(CCCCCCCC(=O)OC(CC)CCCCCCCC)CCCNc1c(NC)c(=O)c1=O] |
| II-30 | 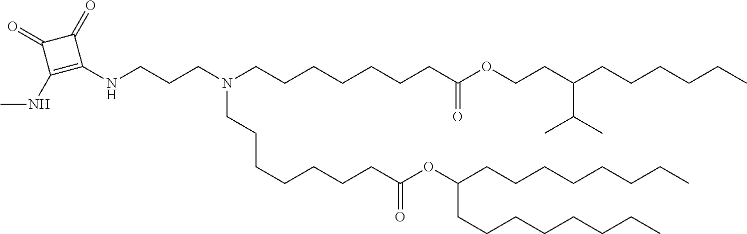 [SMILES: CCCCCCCCC(CCCCCCCC)OC(=O)CCCCCCCN(CCCCCCCC(=O)OCCC(CCCCCC)C(C)C)CCCNc1c(NC)c(=O)c1=O] |
| II-31 | 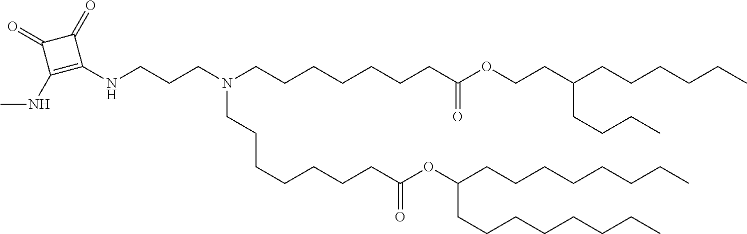 [SMILES: CCCCCCCCC(CCCCCCCC)OC(=O)CCCCCCCN(CCCCCCCC(=O)OCCC(CCCC)CCCCCC)CCCNc1c(NC)c(=O)c1=O] |
| II-32 | 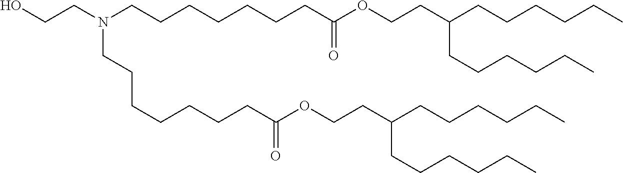 [SMILES: CCCCCCC(CCCCCC)CCOC(=O)CCCCCCCN(CCO)CCCCCCCC(=O)OCCC(CCCCCC)CCCCCC] |
| II-33 | 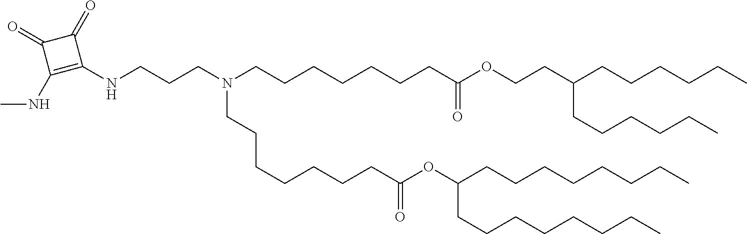 [SMILES: CCCCCCCCC(CCCCCCCC)OC(=O)CCCCCCCN(CCCCCCCC(=O)OCCC(CCCCCC)CCCCCC)CCCNc1c(NC)c(=O)c1=O] |
| II-34 | 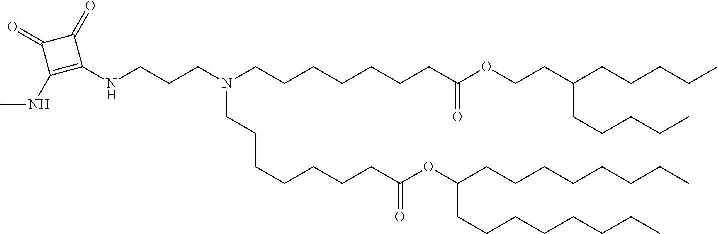 [SMILES: CCCCCCCCC(CCCCCCCC)OC(=O)CCCCCCCN(CCCCCCCC(=O)OCCC(CCCCC)CCCCC)CCCNc1c(NC)c(=O)c1=O] |
| II-35 | 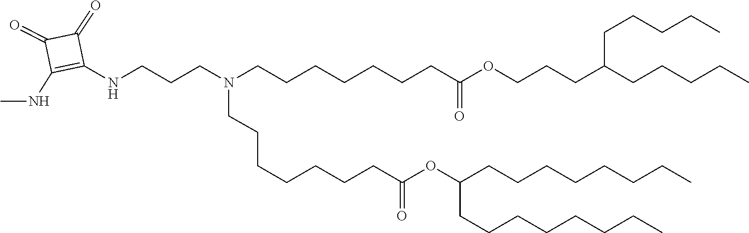 [SMILES: CCCCCCCCC(CCCCCCCC)OC(=O)CCCCCCCN(CCCCCCCC(=O)OCCCC(CCCCC)CCCCC)CCCNc1c(NC)c(=O)c1=O] |
| II-36 | 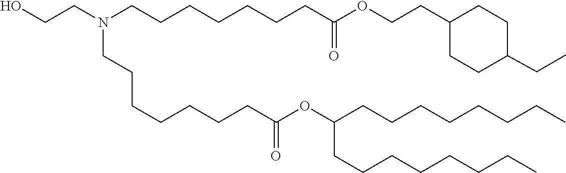 [SMILES: CCCCCCCCC(CCCCCCCC)OC(=O)CCCCCCCN(CCO)CCCCCCCC(=O)OCCC1CCC(CC)CC1] |
| II-37 | 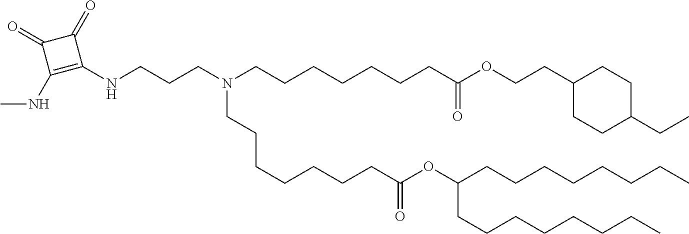 [SMILES: CCCCCCCCC(CCCCCCCC)OC(=O)CCCCCCCN(CCCCCCCC(=O)OCCC1CCC(CC)CC1)CCCNc1c(NC)c(=O)c1=O] |
| II-38 | 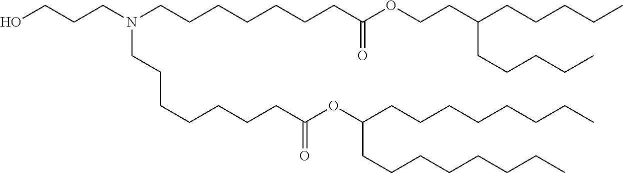 [SMILES: CCCCCCCCC(CCCCCCCC)OC(=O)CCCCCCCN(CCCO)CCCCCCCC(=O)OCCC(CCCCC)CCCCC] |
| II-39 | 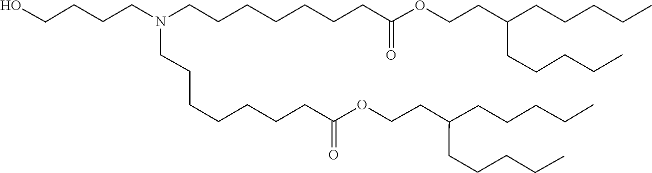 [SMILES: CCCCCC(CCCCC)CCOC(=O)CCCCCCCN(CCCCO)CCCCCCCC(=O)OCCC(CCCCC)CCCCC] |
| II-40 | 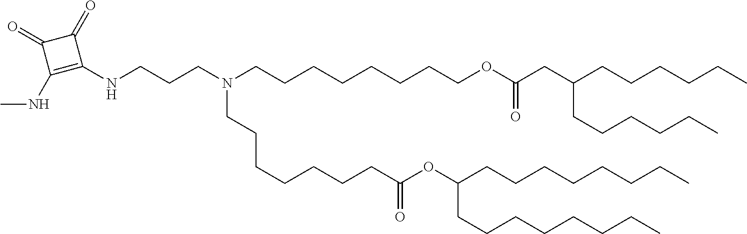 [SMILES: CCCCCCCCC(CCCCCCCC)OC(=O)CCCCCCCN(CCCCCCCCOC(=O)CC(CCCCCC)CCCCCC)CCCNc1c(NC)c(=O)c1=O] |
| II-41 | 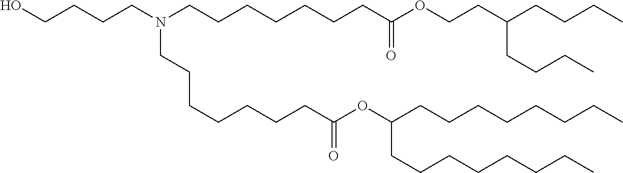 [SMILES: CCCCCCCCC(CCCCCCCC)OC(=O)CCCCCCCN(CCCCO)CCCCCCCC(=O)OCCC(CCCC)CCCC] |
| II-42 | 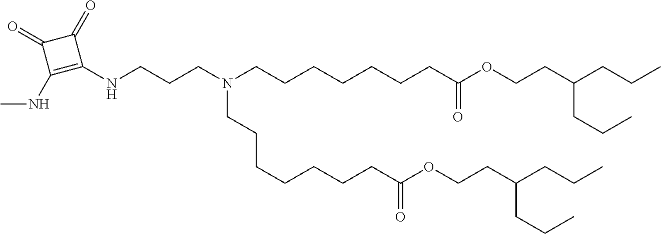 [SMILES: CCCC(CCC)CCOC(=O)CCCCCCCN(CCCCCCCC(=O)OCCC(CCC)CCC)CCCNc1c(NC)c(=O)c1=O] |
| II-43 | 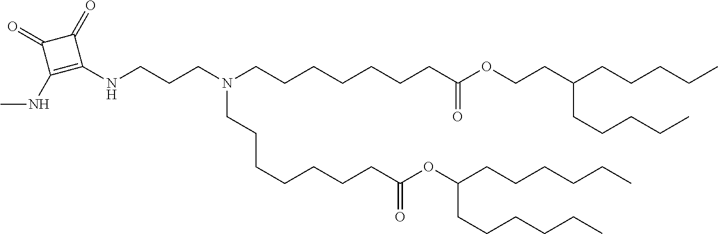 [SMILES: CCCCCCC(CCCCCC)OC(=O)CCCCCCCN(CCCCCCCC(=O)OCCC(CCCCC)CCCCC)CCCNc1c(NC)c(=O)c1=O] |
| II-44 | 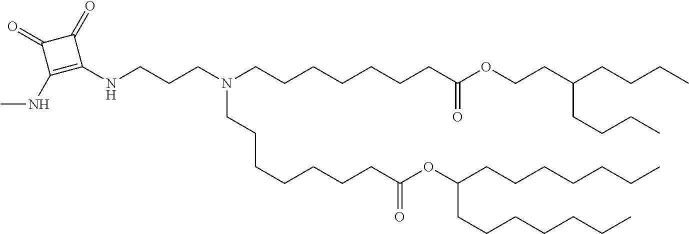 [SMILES: CCCCCCCC(CCCCCCC)OC(=O)CCCCCCCN(CCCCCCCC(=O)OCCC(CCCC)CCCC)CCCNc1c(NC)c(=O)c1=O] |
| II-45 | 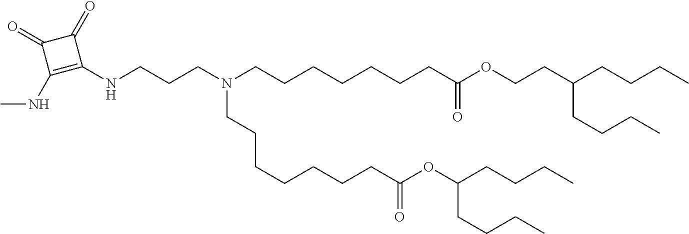 [SMILES: CCCCC(CCCC)CCOC(=O)CCCCCCCN(CCCCCCCC(=O)OC(CCCC)CCCC)CCCNc1c(NC)c(=O)c1=O] |
| II-46 | 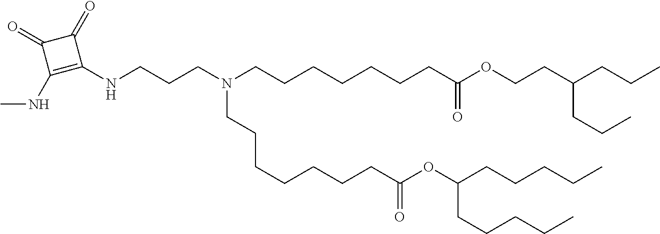 [SMILES: CCCCCC(CCCCC)OC(=O)CCCCCCCN(CCCCCCCC(=O)OCCC(CCC)CCC)CCCNc1c(NC)c(=O)c1=O] |
| II-47 | 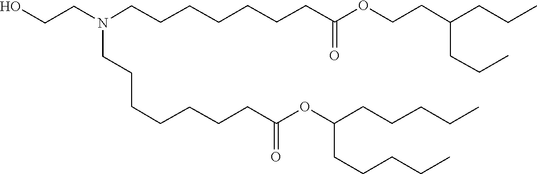 [SMILES: CCCCCC(CCCCC)OC(=O)CCCCCCCN(CCO)CCCCCCCC(=O)OCCC(CCC)CCC] |
| II-48 | 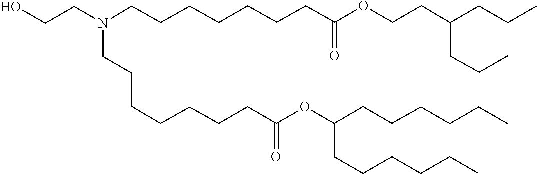 [SMILES: CCCCCCC(CCCCCC)OC(=O)CCCCCCCN(CCO)CCCCCCCC(=O)OCCC(CCC)CCC] |
| II-49 | 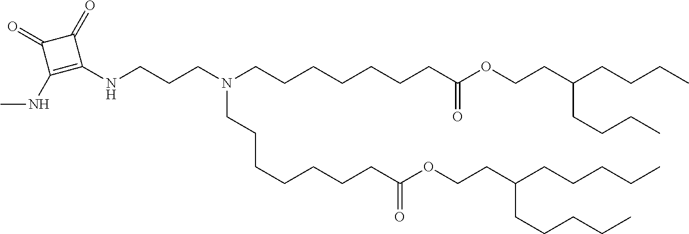 [SMILES: CCCCCC(CCCCC)CCOC(=O)CCCCCCCN(CCCCCCCC(=O)OCCC(CCCC)CCCC)CCCNc1c(NC)c(=O)c1=O] |
| II-50 | 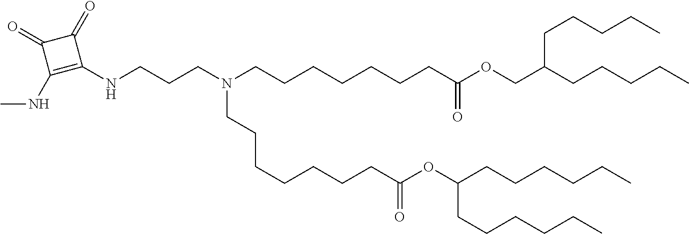 [SMILES: CCCCCCC(CCCCCC)OC(=O)CCCCCCCN(CCCCCCCC(=O)OCC(CCCCC)CCCCC)CCCNc1c(NC)c(=O)c1=O] |
| II-51 | 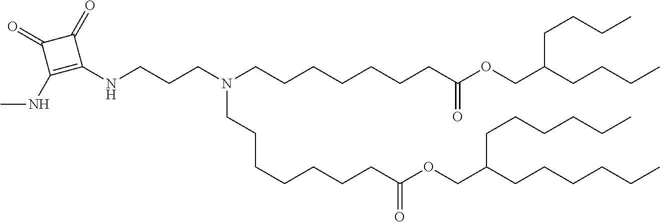 [SMILES: CCCCCCC(CCCCCC)COC(=O)CCCCCCCN(CCCCCCCC(=O)OCC(CCCC)CCCC)CCCNc1c(NC)c(=O)c1=O] |
| II-52 | 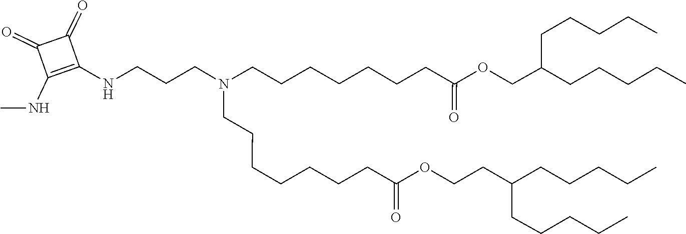 [SMILES: CCCCCC(CCCCC)CCOC(=O)CCCCCCCN(CCCCCCCC(=O)OCC(CCCCC)CCCCC)CCCNc1c(NC)c(=O)c1=O] |
| II-53 | 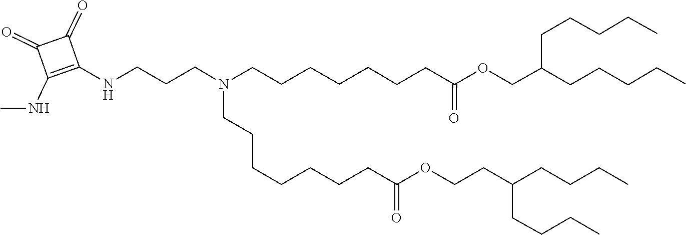 [SMILES: CCCCCC(CCCCC)COC(=O)CCCCCCCN(CCCCCCCC(=O)OCCC(CCCC)CCCC)CCCNc1c(NC)c(=O)c1=O] |
| II-54 | 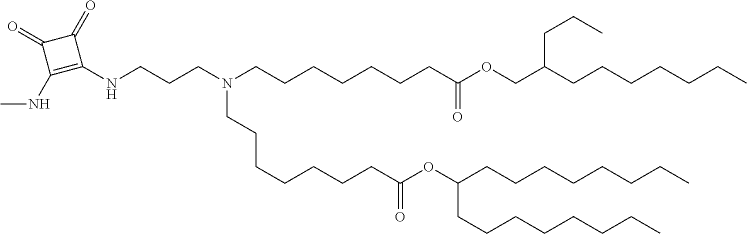 [SMILES: CCCCCCCCC(CCCCCCCC)OC(=O)CCCCCCCN(CCCCCCCC(=O)OCC(CCC)CCCCCCC)CCCNc1c(NC)c(=O)c1=O] |
| II-55 | 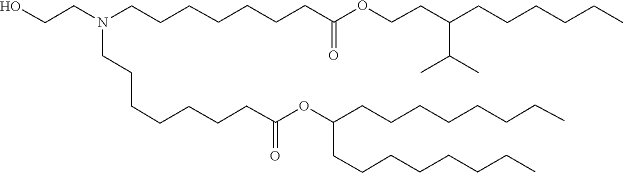 [SMILES: CCCCCCCCC(CCCCCCCC)OC(=O)CCCCCCCN(CCO)CCCCCCCC(=O)OCCC(CCCCCC)C(C)C] |
| II-56 | 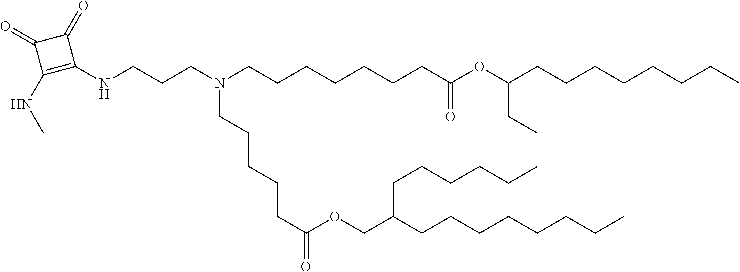 [SMILES: CCCCCCCCC(CCCCCC)COC(=O)CCCCCN(CCCCCCCC(=O)OC(CC)CCCCCCCC)CCCNc1c(NC)c(=O)c1=O] |
| II-57 | 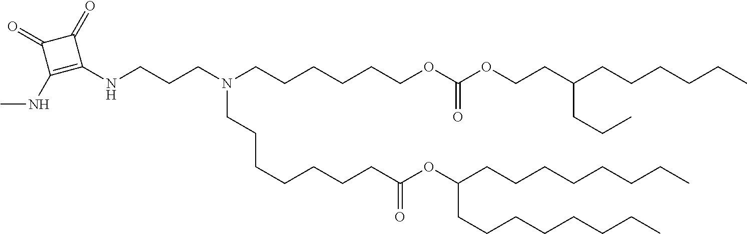 [SMILES: CCCCCCCCC(CCCCCCCC)OC(=O)CCCCCCCN(CCCCCCOC(=O)OCCC(CCC)CCCCCC)CCCNc1c(NC)c(=O)c1=O] |
| II-58 | 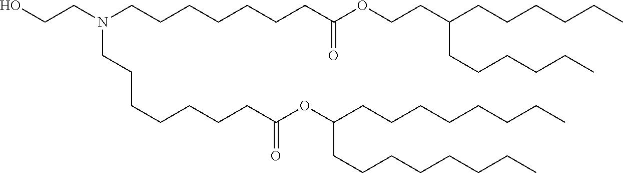 [SMILES: CCCCCCCCC(CCCCCCCC)OC(=O)CCCCCCCN(CCO)CCCCCCCC(=O)OCCC(CCCCCC)CCCCCC] |
| II-59 | 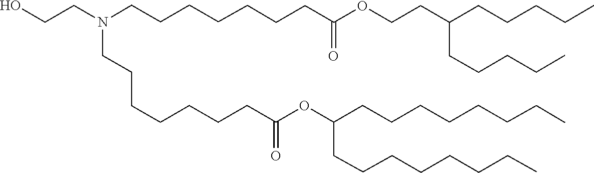 [SMILES: CCCCCCCCC(CCCCCCCC)OC(=O)CCCCCCCN(CCO)CCCCCCCC(=O)OCCC(CCCCC)CCCCC] |
| II-60 | 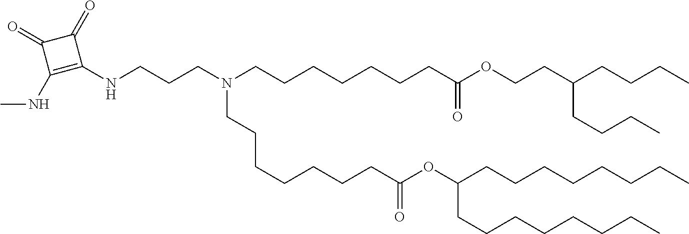 [SMILES: CCCCCCCCC(CCCCCCCC)OC(=O)CCCCCCCN(CCCCCCCC(=O)OCCC(CCCC)CCCC)CCCNc1c(NC)c(=O)c1=O] |
| II-61 | 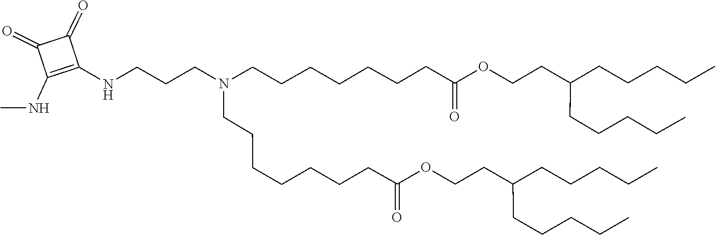 [SMILES: CCCCCC(CCCCC)CCOC(=O)CCCCCCCN(CCCCCCCC(=O)OCCC(CCCCC)CCCCC)CCCNc1c(NC)c(=O)c1=O] |
| II-62 | 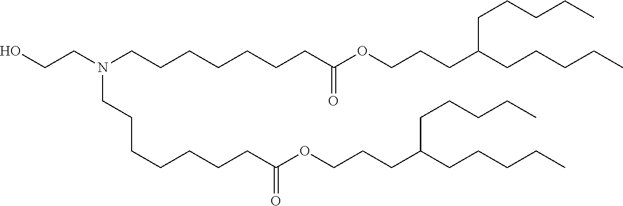 [SMILES: CCCCCC(CCCCC)CCCOC(=O)CCCCCCCN(CCO)CCCCCCCC(=O)OCCCC(CCCCC)CCCCC] |
| II-63 | 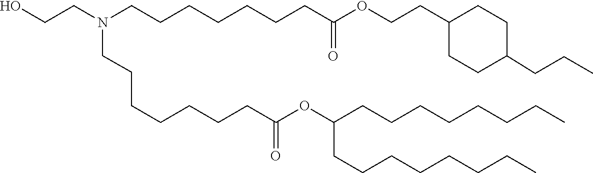 [SMILES: CCCCCCCCC(CCCCCCCC)OC(=O)CCCCCCCN(CCO)CCCCCCCC(=O)OCCC1CCC(CCC)CC1] |
| II-64 | 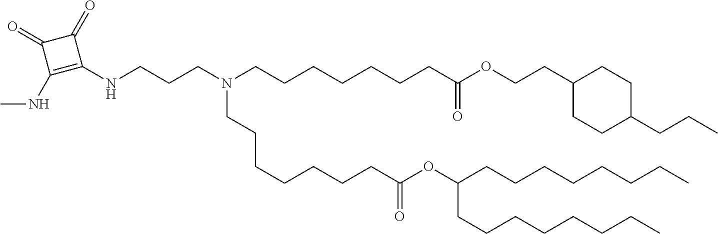 [SMILES: CCCCCCCCC(CCCCCCCC)OC(=O)CCCCCCCN(CCCCCCCC(=O)OCCC1CCC(CCC)CC1)CCCNc1c(NC)c(=O)c1=O] |
| II-65 | 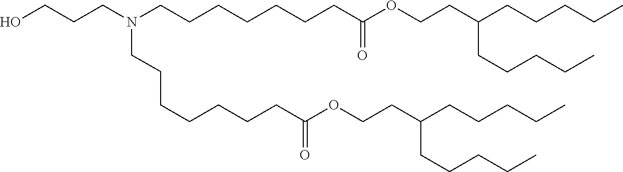 [SMILES: CCCCCC(CCCCC)CCOC(=O)CCCCCCCN(CCCO)CCCCCCCC(=O)OCCC(CCCCC)CCCCC] |
| II-66 | 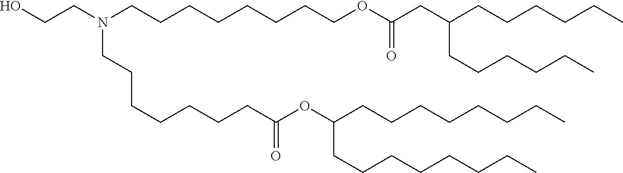 [SMILES: CCCCCCCCC(CCCCCCCC)OC(=O)CCCCCCCN(CCO)CCCCCCCCOC(=O)CC(CCCCCC)CCCCCC] |
| II-67 | 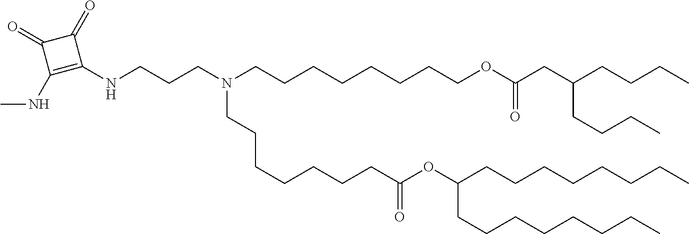 [SMILES: CCCCCCCCC(CCCCCCCC)OC(=O)CCCCCCCN(CCCCCCCCOC(=O)CC(CCCC)CCCC)CCCNc1c(NC)c(=O)c1=O] |
| II-68 | 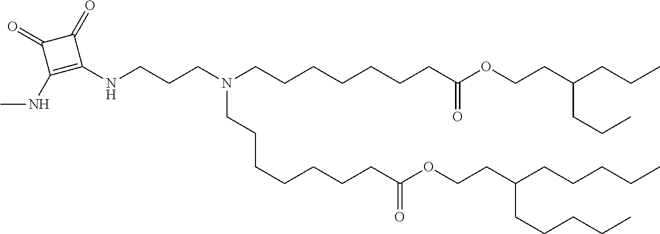 [SMILES: CCCCCC(CCCCC)CCOC(=O)CCCCCCCN(CCCCCCCC(=O)OCCC(CCC)CCC)CCCNc1c(NC)c(=O)c1=O] |
| II-69 | 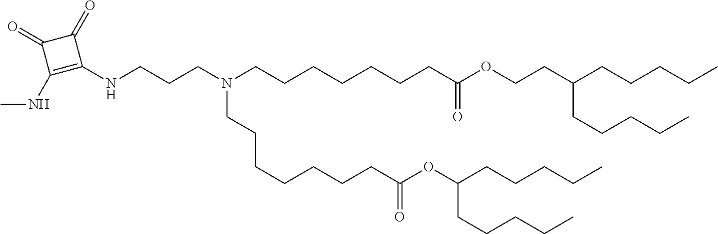 [SMILES: CCCCCC(CCCCC)CCOC(=O)CCCCCCCN(CCCCCCCC(=O)OC(CCCCC)CCCCC)CCCNc1c(NC)c(=O)c1=O] |
| II-70 | 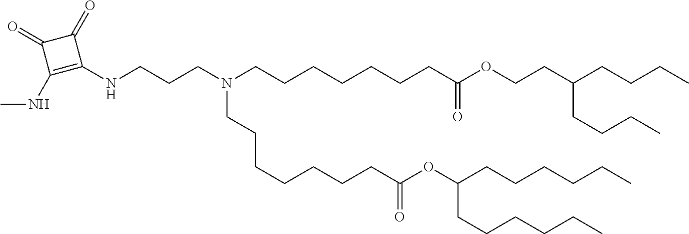 [SMILES: CCCCCCC(CCCCCC)OC(=O)CCCCCCCN(CCCCCCCC(=O)OCCC(CCCC)CCCC)CCCNc1c(NC)c(=O)c1=O] |
| II-71 | 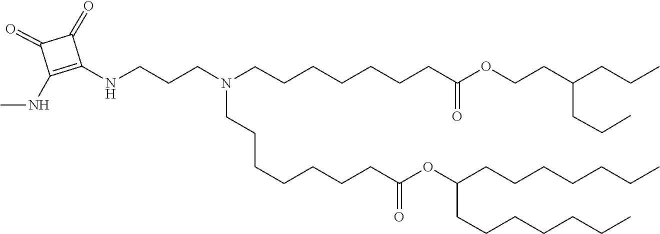 [SMILES: CCCCCCCC(CCCCCCC)OC(=O)CCCCCCCN(CCCCCCCC(=O)OCCC(CCC)CCC)CCCNc1c(NC)c(=O)c1=O] |
| II-72 | 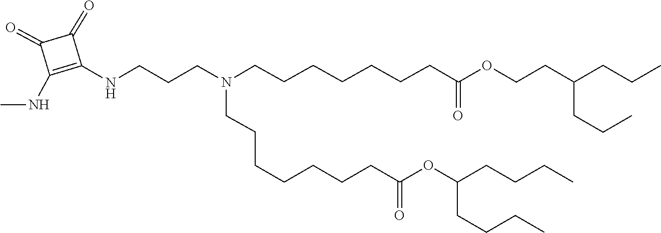 [SMILES: CCCCC(CCCC)OC(=O)CCCCCCCN(CCCCCCCC(=O)OCCC(CCC)CCC)CCCNc1c(NC)c(=O)c1=O] |
| II-73 | 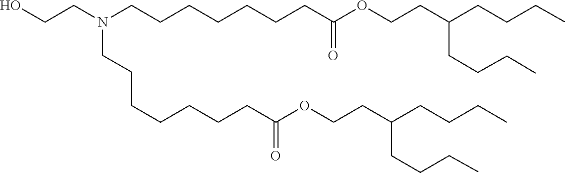 [SMILES: CCCCC(CCCC)CCOC(=O)CCCCCCCN(CCO)CCCCCCCC(=O)OCCC(CCCC)CCCC] |
| II-74 | 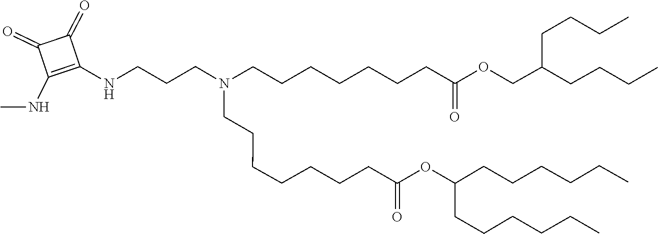 [SMILES: CCCCCCC(CCCCCC)OC(=O)CCCCCCCN(CCCCCCCC(=O)OCC(CCCC)CCCC)CCCNc1c(NC)c(=O)c1=O] |
| II-75 | 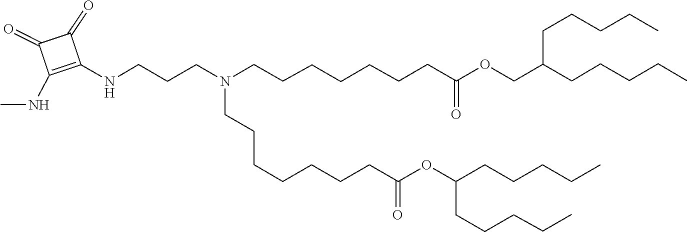 [SMILES: CCCCCC(CCCCC)COC(=O)CCCCCCCN(CCCCCCCC(=O)OC(CCCCC)CCCCC)CCCNc1c(NC)c(=O)c1=O] |
| II-76 | 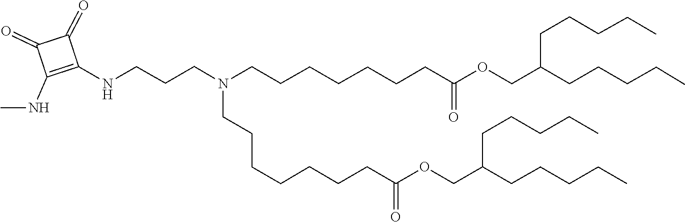 [SMILES: CCCCCC(CCCCC)COC(=O)CCCCCCCN(CCCCCCCC(=O)OCC(CCCCC)CCCCC)CCCNc1c(NC)c(=O)c1=O] |
| II-77 | 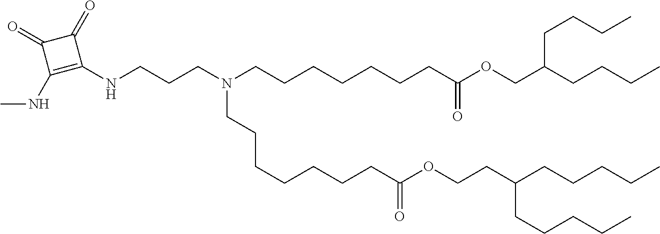 [SMILES: CCCCCC(CCCCC)CCOC(=O)CCCCCCCN(CCCCCCCC(=O)OCC(CCCC)CCCC)CCCNc1c(NC)c(=O)c1=O] |
| II-78 | 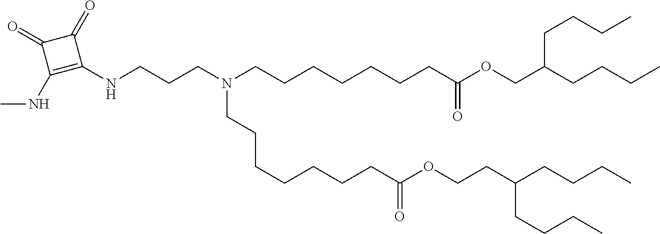 [SMILES: CCCCC(CCCC)CCOC(=O)CCCCCCCN(CCCCCCCC(=O)OCC(CCCC)CCCC)CCCNc1c(NC)c(=O)c1=O] |

In some embodiments, the ionizable lipid is a compound of Formula (IL-IIA):

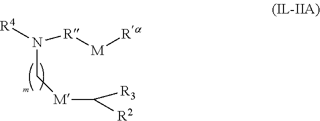

### or its N-oxide,

or a salt or isomer thereof, wherein:
m is selected from 5, 6, 7, 8, and 9;
R² and R³ are each independently selected from the group consisting of H, C₁₋₁₄ alkyl and C₂₋₁₄ alkenyl;
R⁴ is selected from —(CH₂)ₙOH, wherein n is selected from 1, 2, 3, 4, and 5, and

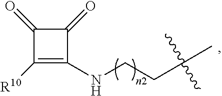

wherein n2 is selected from 1, 2, 3, 4, 5, 6, 7, 8, 9, and 10; and
R¹⁰ is —N(R)₂, wherein each R is independently selected from the group consisting of C₁₋₆ alkyl, C₂₋₃ alkenyl, and H;
M is selected from —OC(O)O—, —C(O)O—, —O-M″-O—, and —N(R^{M})C(O)—, in which M″ is
—(CH₂)_{z}C(O)—, wherein z is 1, 2, 3, or 4;
M′ is selected from —OC(O)O—, —C(O)O—, —O-M″-O—, —N(R^{M})C(O)O—, and —O—N═C(R^{M})—, wherein:
   M″ is —(CH₂)_{z}C(O)—, C₁₋₁₃ alkyl, —B(R**)—, or —Si(R**)₂—;
   z is 1, 2, 3, or 4;
   each R^{M} is independently selected from H and C₁₋₆ alkyl;
   each R** is independently selected from H and C₁₋₁₂ alkyl;
R′^{a} is C₁₋₁₈ alkyl, C₂₋₁₈ alkenyl, or —R*YR*″, wherein:
   each R*″ is independently C₁₋₁₅ alkyl;
   each R* is independently C₁₋₁₂ alkyl;
   each Y is independently a C₃₋₆ carbocycle; and
R″ is a C₃-C₁₃ alkyl, optionally substituted with OH.

In some embodiments, the ionizable lipid is a compound of Formula (IL-IIAX):

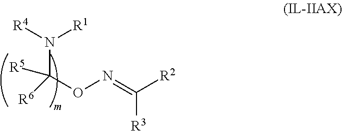

### or its N-oxide,

or a salt or isomer thereof, wherein:
   R¹ is —R″M′R′, wherein:
      each R′ is independently C₁₋₁₈ alkyl;
      M′ is selected from —C(O)O— and —O—N—C(R^{M})—, wherein each R^{M} is independently selected from H and C₁₋₆ alkyl;
      each R″ is independently C₃₋₁₅ alkyl;
R² and R³ are each independently selected from the group consisting of H, C₁₋₁₄ alkyl, and C₂₋₁₄ alkenyl;
R⁴ is selected from —(CH₂)ₙOH, wherein n is selected from 1, 2, 3, 4, 5, and

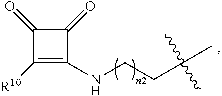

wherein n2 is selected from 1, 2, 3, 4, 5, 6, 7, 8, 9, and 10; and
R¹⁰ is —N(R)₂, wherein each R is independently selected from the group consisting of C₁₋₆ alkyl, C₂₋₃ alkenyl, and H;
each R⁵ is H;
each R⁶ is H; and
m is selected from 5, 6, 7, 8, 9, 10, 11, 12, and 13.

In some embodiments, the ionizable lipid is a compound selected from Table IL-3.

TABLE IL-3

| Cpd | Structure |
|---|---|
| III-1 | 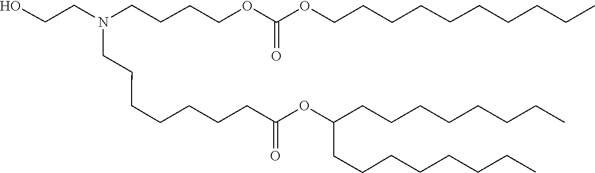 [SMILES: CCCCCCCCCCOC(=O)OCCCCN(CCO)CCCCCCCC(=O)OC(CCCCCCCC)CCCCCCCC] |
| III-2 | 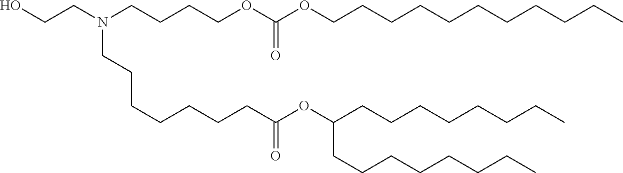 [SMILES: CCCCCCCCCCCOC(=O)OCCCCN(CCO)CCCCCCCC(=O)OC(CCCCCCCC)CCCCCCCC] |
| III-3 | 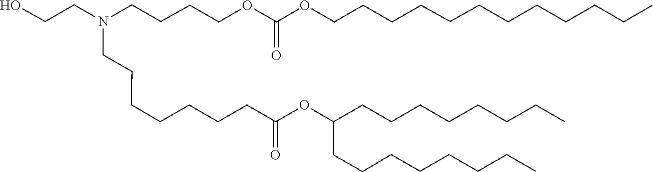 [SMILES: CCCCCCCCCCCCOC(=O)OCCCCN(CCO)CCCCCCCC(=O)OC(CCCCCCCC)CCCCCCCC] |
| III-4 | 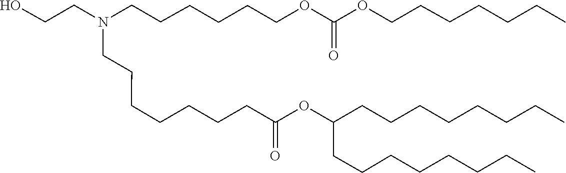 [SMILES: CCCCCCCCC(CCCCCCCC)OC(=O)CCCCCCCN(CCO)CCCCCCOC(=O)OCCCCCCC] |
| III-5 | 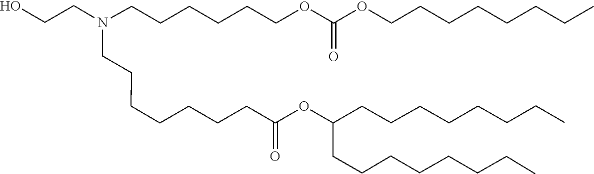 [SMILES: CCCCCCCCOC(=O)OCCCCCCN(CCO)CCCCCCCC(=O)OC(CCCCCCCC)CCCCCCCC] |
| III-6 | 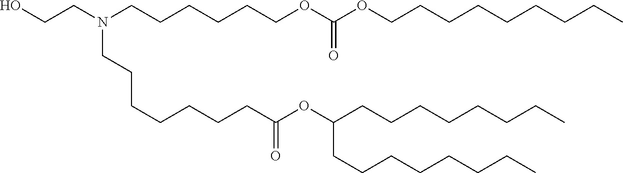 [SMILES: CCCCCCCCCOC(=O)OCCCCCCN(CCO)CCCCCCCC(=O)OC(CCCCCCCC)CCCCCCCC] |
| III-7 | 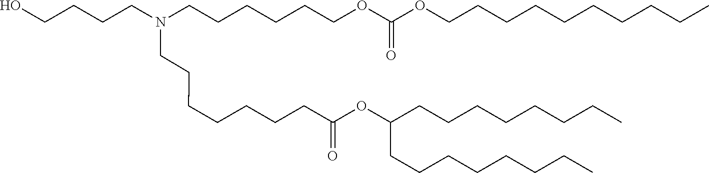 [SMILES: CCCCCCCCCCOC(=O)OCCCCCCN(CCCCO)CCCCCCCC(=O)OC(CCCCCCCC)CCCCCCCC] |
| III-8 | 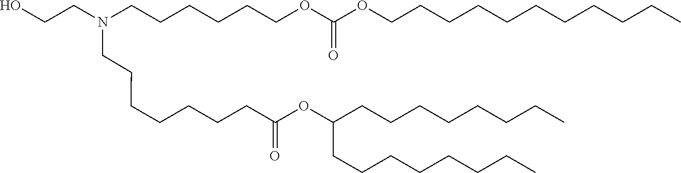 [SMILES: CCCCCCCCCCCOC(=O)OCCCCCCN(CCO)CCCCCCCC(=O)OC(CCCCCCCC)CCCCCCCC] |
| III-9 | 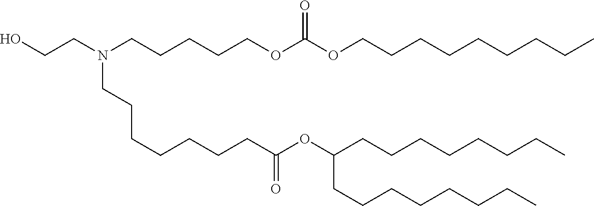 [SMILES: CCCCCCCCCOC(=O)OCCCCCN(CCO)CCCCCCCC(=O)OC(CCCCCCCC)CCCCCCCC] |
| III-10 | 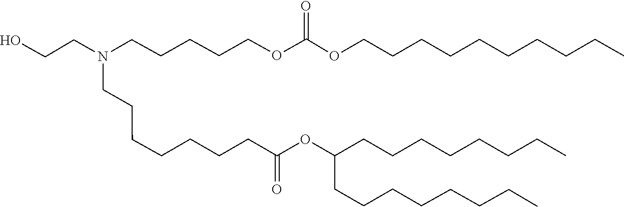 [SMILES: CCCCCCCCCCOC(=O)OCCCCCN(CCO)CCCCCCCC(=O)OC(CCCCCCCC)CCCCCCCC] |
| III-11 | 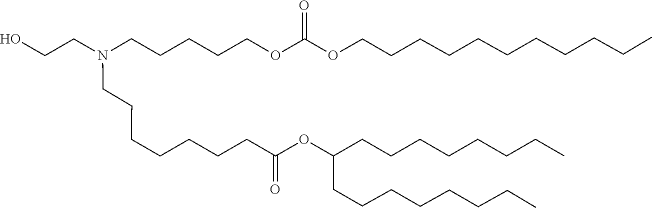 |
| III-12 | 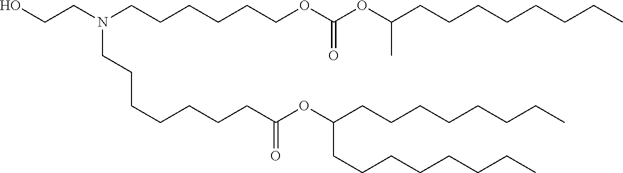 [SMILES: CCCCCCCCC(C)OC(=O)OCCCCCCN(CCO)CCCCCCCC(=O)OC(CCCCCCCC)CCCCCCCC] |
| III-13 | 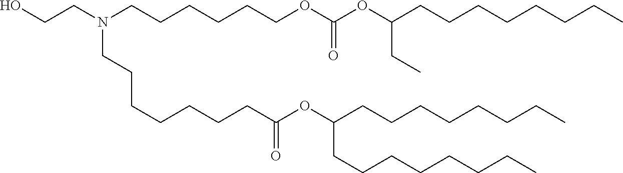 [SMILES: CCCCCCCCC(CCCCCCCC)OC(=O)CCCCCCCN(CCO)CCCCCCOC(=O)OC(CC)CCCCCCCC] |
| III-14 | 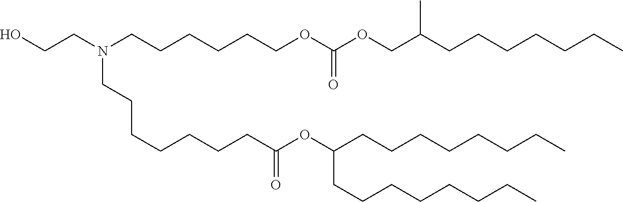 [SMILES: CCCCCCCCC(CCCCCCCC)OC(=O)CCCCCCCN(CCO)CCCCCCOC(=O)OCC(C)CCCCCCC] |
| III-15 | 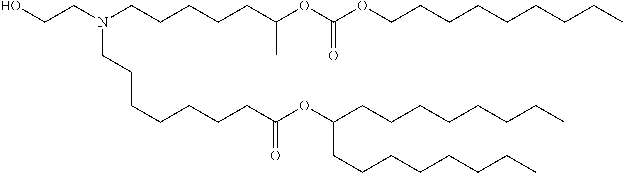 [SMILES: CCCCCCCCCOC(=O)OC(C)CCCCCN(CCO)CCCCCCCC(=O)OC(CCCCCCCC)CCCCCCCC] |
| III-16 | 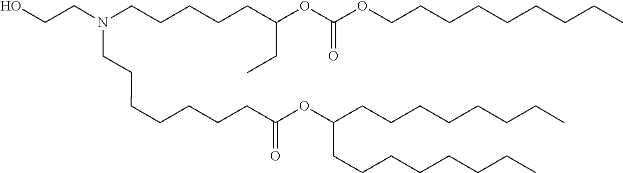 [SMILES: CCCCCCCCCOC(=O)OC(CC)CCCCCN(CCO)CCCCCCCC(=O)OC(CCCCCCCC)CCCCCCCC] |
| III-17 | 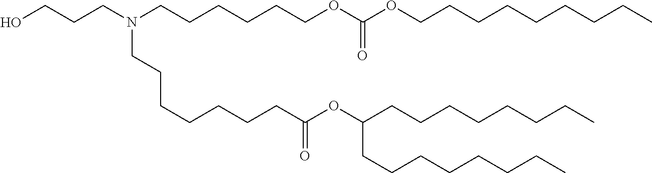 [SMILES: CCCCCCCCCOC(=O)OCCCCCCN(CCCO)CCCCCCCC(=O)OC(CCCCCCCC)CCCCCCCC] |
| III-18 | 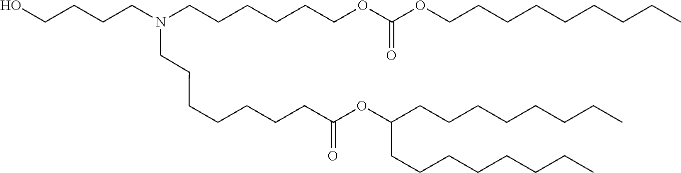 [SMILES: CCCCCCCCCOC(=O)OCCCCCCN(CCCCO)CCCCCCCC(=O)OC(CCCCCCCC)CCCCCCCC] |
| III-19 | 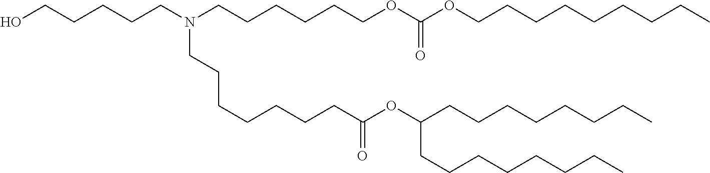 [SMILES: CCCCCCCCCOC(=O)OCCCCCCN(CCCCCO)CCCCCCCC(=O)OC(CCCCCCCC)CCCCCCCC] |
| III-20 | 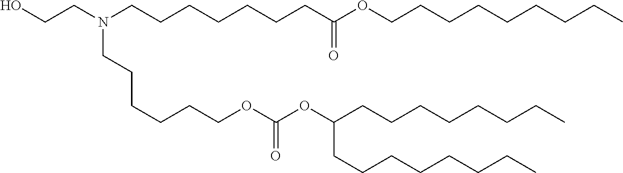 [SMILES: CCCCCCCCCOC(=O)CCCCCCCN(CCO)CCCCCCOC(=O)OC(CCCCCCCC)CCCCCCCC] |
| III-21 | 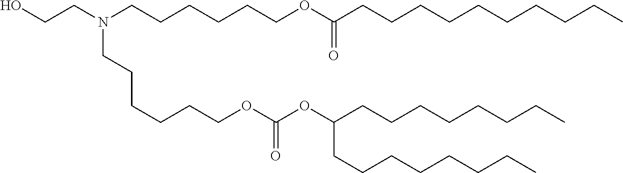 [SMILES: CCCCCCCCCCC(=O)OCCCCCCN(CCO)CCCCCCOC(=O)OC(CCCCCCCC)CCCCCCCC] |
| III-22 | 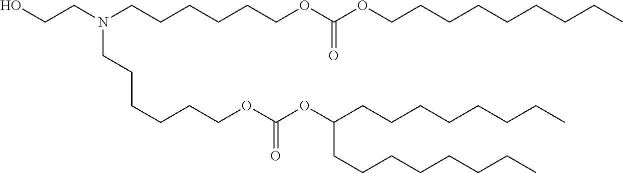 [SMILES: CCCCCCCCCOC(=O)OCCCCCCN(CCO)CCCCCCOC(=O)OC(CCCCCCCC)CCCCCCCC] |
| III-23 | 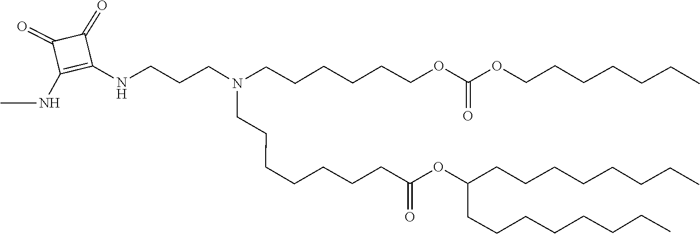 [SMILES: CCCCCCCCC(CCCCCCCC)OC(=O)CCCCCCCN(CCCCCCOC(=O)OCCCCCCC)CCCNc1c(NC)c(=O)c1=O] |
| III-24 | 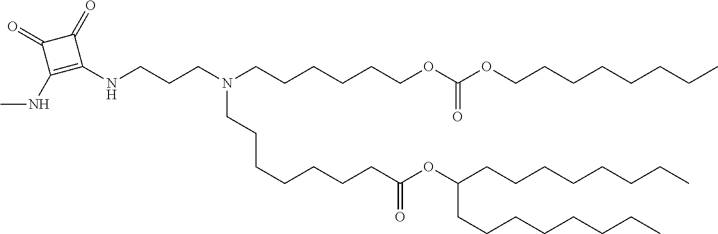 [SMILES: CCCCCCCCOC(=O)OCCCCCCN(CCCCCCCC(=O)OC(CCCCCCCC)CCCCCCCC)CCCNc1c(NC)c(=O)c1=O] |
| III-25 | 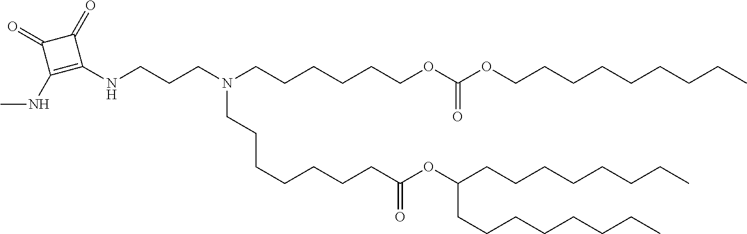 [SMILES: CCCCCCCCCOC(=O)OCCCCCCN(CCCCCCCC(=O)OC(CCCCCCCC)CCCCCCCC)CCCNc1c(NC)c(=O)c1=O] |
| III-26 | 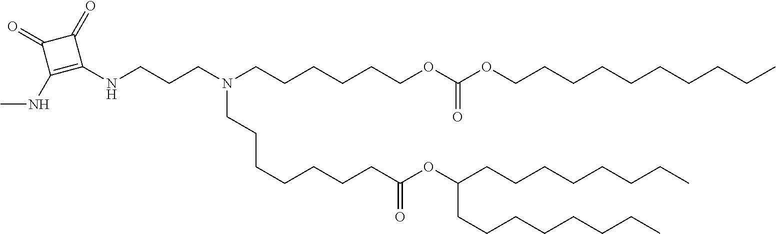 [SMILES: CCCCCCCCCCOC(=O)OCCCCCCN(CCCCCCCC(=O)OC(CCCCCCCC)CCCCCCCC)CCCNc1c(NC)c(=O)c1=O] |
| III-27 | 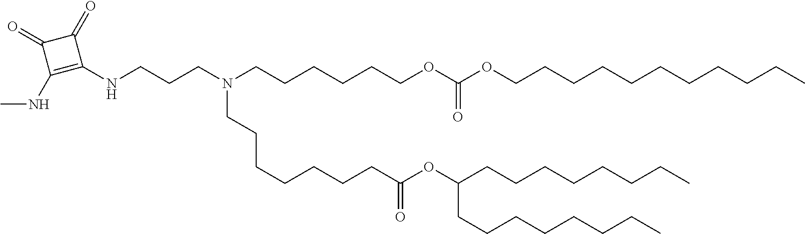 [SMILES: CCCCCCCCCCCOC(=O)OCCCCCCN(CCCCCCCC(=O)OC(CCCCCCCC)CCCCCCCC)CCCNc1c(NC)c(=O)c1=O] |
| III-29 | 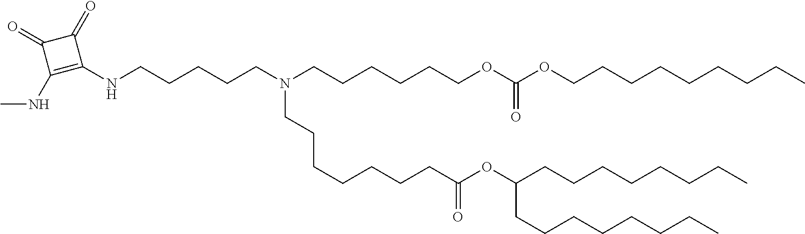 [SMILES: CCCCCCCCCOC(=O)OCCCCCCN(CCCCCCCC(=O)OC(CCCCCCCC)CCCCCCCC)CCCCCNc1c(NC)c(=O)c1=O] |
| III-30 | 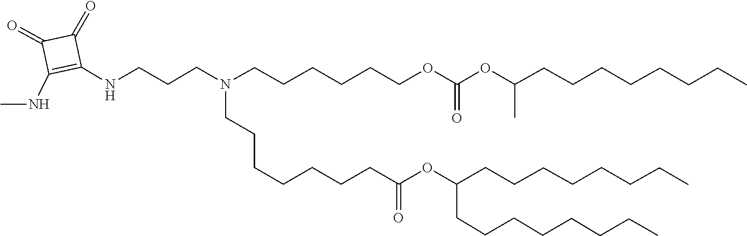 [SMILES: CCCCCCCCC(C)OC(=O)OCCCCCCN(CCCCCCCC(=O)OC(CCCCCCCC)CCCCCCCC)CCCNc1c(NC)c(=O)c1=O] |
| III-31 | 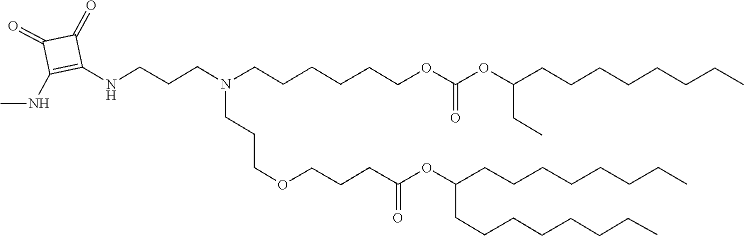 [SMILES: CCCCCCCCC(CCCCCCCC)OC(=O)CCCOCCCN(CCCCCCOC(=O)OC(CC)CCCCCCCC)CCCNc1c(NC)c(=O)c1=O] |
| III-32 | 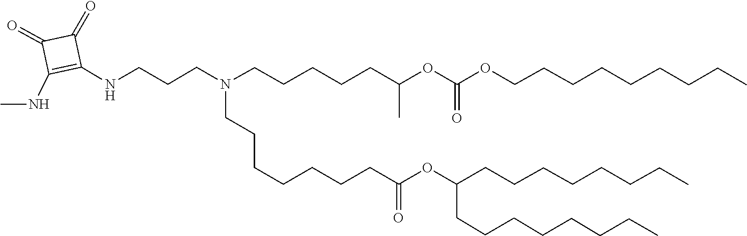 [SMILES: CCCCCCCCCOC(=O)OC(C)CCCCCN(CCCCCCCC(=O)OC(CCCCCCCC)CCCCCCCC)CCCNc1c(NC)c(=O)c1=O] |
| III-33 | 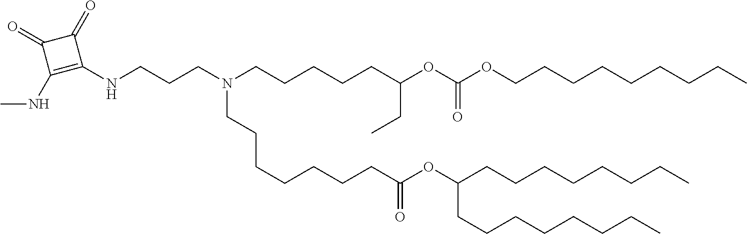 [SMILES: CCCCCCCCCOC(=O)OC(CC)CCCCCN(CCCCCCCC(=O)OC(CCCCCCCC)CCCCCCCC)CCCNc1c(NC)c(=O)c1=O] |
| III-34 | 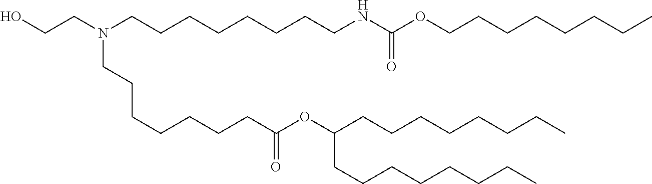 [SMILES: CCCCCCCCOC(=O)NCCCCCCCCN(CCO)CCCCCCCC(=O)OC(CCCCCCCC)CCCCCCCC] |
| III-35 | 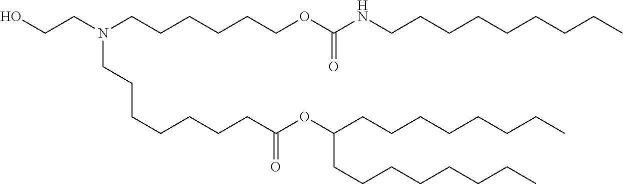 [SMILES: CCCCCCCCCNC(=O)OCCCCCCN(CCO)CCCCCCCC(=O)OC(CCCCCCCC)CCCCCCCC] |
| III-36 | 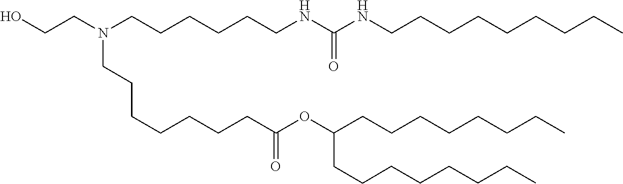 [SMILES: CCCCCCCCCNC(=O)NCCCCCCN(CCO)CCCCCCCC(=O)OC(CCCCCCCC)CCCCCCCC] |
| III-37 | 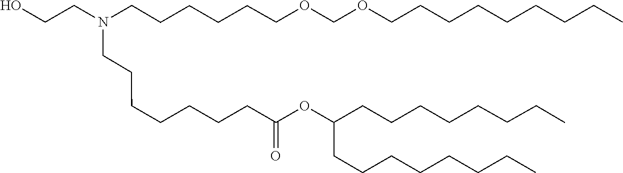 [SMILES: CCCCCCCCCOCOCCCCCCN(CCO)CCCCCCCC(=O)OC(CCCCCCCC)CCCCCCCC] |
| III-38 | 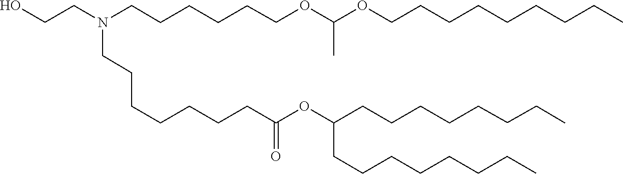 [SMILES: CCCCCCCCCOC(C)OCCCCCCN(CCO)CCCCCCCC(=O)OC(CCCCCCCC)CCCCCCCC] |
| III-39 | 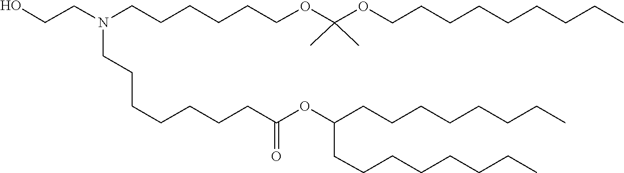 [SMILES: CCCCCCCCCOC(C)(C)OCCCCCCN(CCO)CCCCCCCC(=O)OC(CCCCCCCC)CCCCCCCC] |
| III-40 | 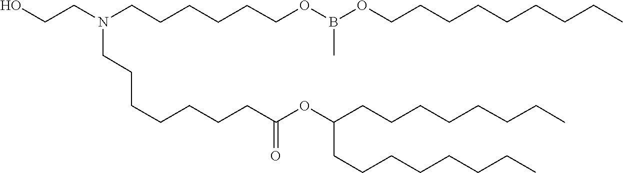 [SMILES: CCCCCCCCCOB(C)OCCCCCCN(CCO)CCCCCCCC(=O)OC(CCCCCCCC)CCCCCCCC] |
| III-41 | 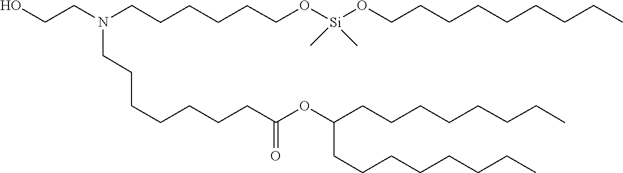 [SMILES: CCCCCCCCCO[Si](C)(C)OCCCCCCN(CCO)CCCCCCCC(=O)OC(CCCCCCCC)CCCCCCCC] |
| III-42 | 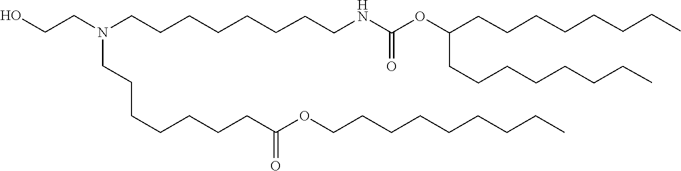 [SMILES: CCCCCCCCCOC(=O)CCCCCCCN(CCO)CCCCCCCCNC(=O)OC(CCCCCCCC)CCCCCCCC] |
| III-43 | 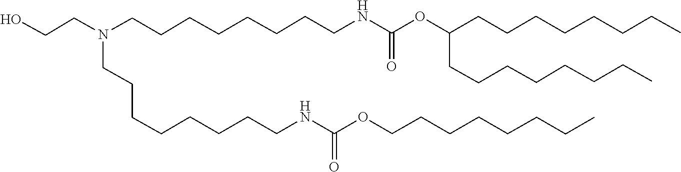 [SMILES: CCCCCCCCOC(=O)NCCCCCCCCN(CCO)CCCCCCCCNC(=O)OC(CCCCCCCC)CCCCCCCC] |
| III-44 | 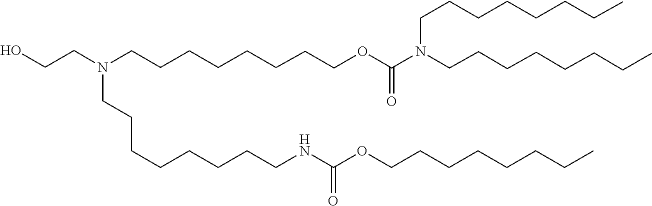 [SMILES: CCCCCCCCOC(=O)NCCCCCCCCN(CCO)CCCCCCCCOC(=O)N(CCCCCCCC)CCCCCCCC] |
| III-45 | 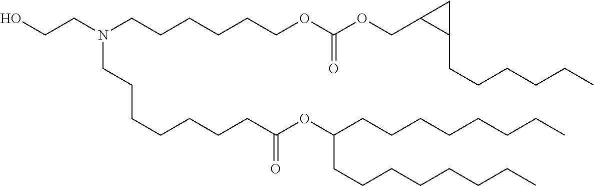 [SMILES: CCCCCCCCC(CCCCCCCC)OC(=O)CCCCCCCN(CCO)CCCCCCOC(=O)OCC1CC1CCCCCC] |
| III-46 | 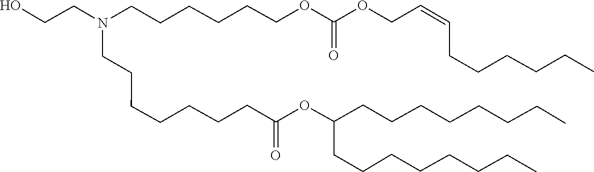 [SMILES: CCCCCC/C=C\COC(=O)OCCCCCCN(CCO)CCCCCCCC(=O)OC(CCCCCCCC)CCCCCCCC] |
| III-47 | 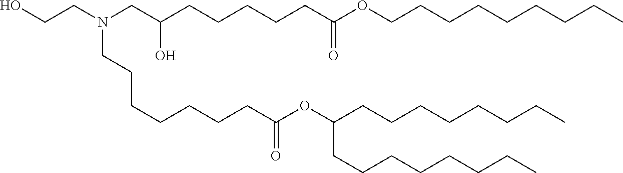 [SMILES: CCCCCCCCCOC(=O)CCCCCC(O)CN(CCO)CCCCCCCC(=O)OC(CCCCCCCC)CCCCCCCC] |
| III-48 | 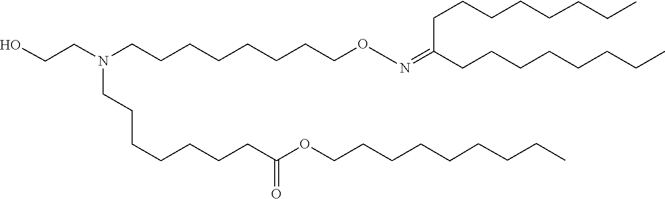 [SMILES: CCCCCCCCCOC(=O)CCCCCCCN(CCO)CCCCCCCCON=C(CCCCCCCC)CCCCCCCC] |
| III-49 | 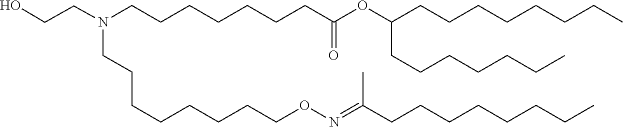 [SMILES: CCCCCCCC/C(C)=N/OCCCCCCCCN(CCO)CCCCCCCC(=O)OC(CCCCCCC)CCCCCCCC] |
| III-50 | 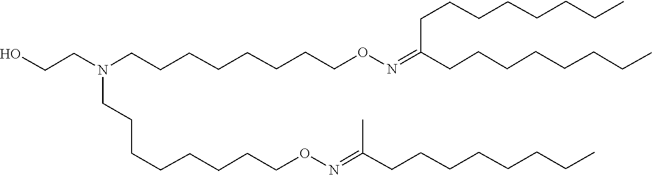 [SMILES: CCCCCCCCC(CCCCCCCC)=NOCCCCCCCCN(CCO)CCCCCCCCO/N=C(\C)CCCCCCCC] |
| III-51 | 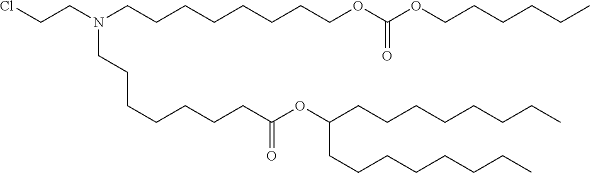 [SMILES: CCCCCCCCC(CCCCCCCC)OC(=O)CCCCCCCN(CCCl)CCCCCCCCOC(=O)OCCCCCC] |
| III-52 | 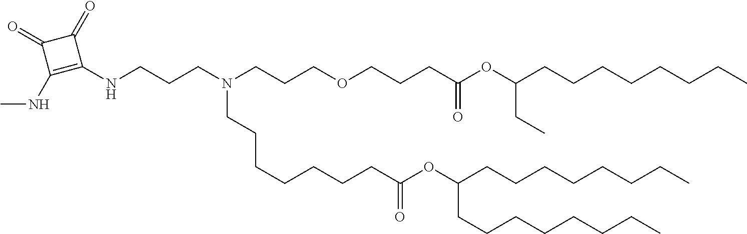 [SMILES: CCCCCCCCC(CC)OC(=O)CCCOCCCN(CCCCCCCC(=O)OC(CCCCCCCC)CCCCCCCC)CCCNc1c(NC)c(=O)c1=O] |
| III-53 | 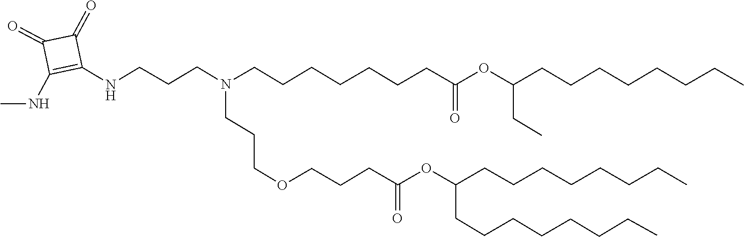 [SMILES: CCCCCCCCC(CC)OC(=O)CCCCCCCN(CCCNc1c(NC)c(=O)c1=O)CCCOCCCC(=O)OC(CCCCCCCC)CCCCCCCC] |
| III-54 | 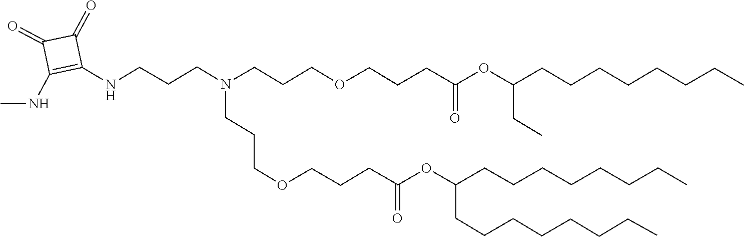 [SMILES: CCCCCCCCC(CC)OC(=O)CCCOCCCN(CCCNc1c(NC)c(=O)c1=O)CCCOCCCC(=O)OC(CCCCCCCC)CCCCCCCC] |
| III-55 | 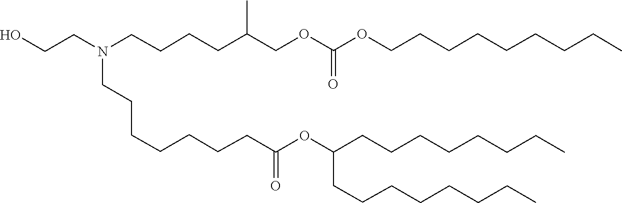 |
| III-56 | 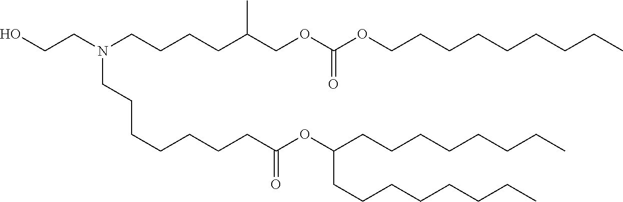 [SMILES: CCCCCCCCCOC(=O)OCC(C)CCCCN(CCO)CCCCCCCC(=O)OC(CCCCCCCC)CCCCCCCC] |
| III-57 | 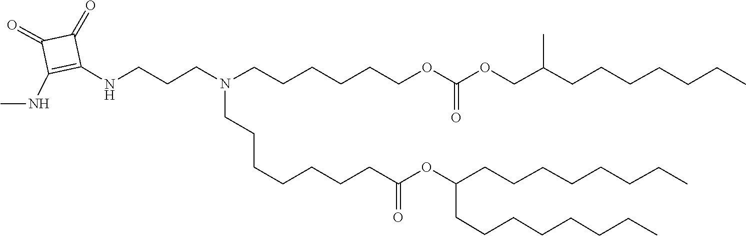 [SMILES: CCCCCCCCC(CCCCCCCC)OC(=O)CCCCCCCN(CCCCCCOC(=O)OCC(C)CCCCCCC)CCCNc1c(NC)c(=O)c1=O] |
| III-58 | 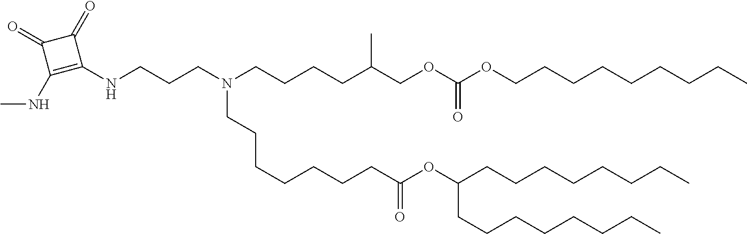 [SMILES: CCCCCCCCCOC(=O)OCC(C)CCCCN(CCCCCCCC(=O)OC(CCCCCCCC)CCCCCCCC)CCCNc1c(NC)c(=O)c1=O] |
| III-59 | 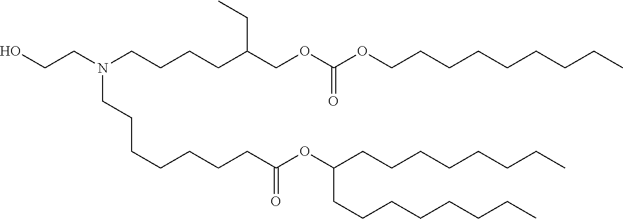 [SMILES: CCCCCCCCCOC(=O)OCC(CC)CCCCN(CCO)CCCCCCCC(=O)OC(CCCCCCCC)CCCCCCCC] |
| III-60 | 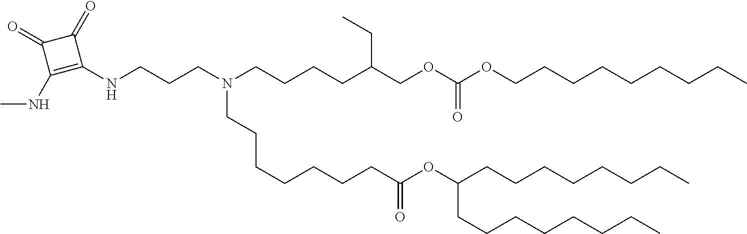 [SMILES: CCCCCCCCCOC(=O)OCC(CC)CCCCN(CCCCCCCC(=O)OC(CCCCCCCC)CCCCCCCC)CCCNc1c(NC)c(=O)c1=O] |
| III-61 | 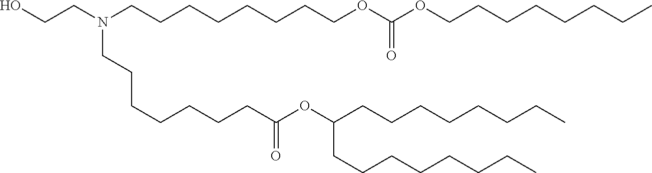 [SMILES: CCCCCCCCOC(=O)OCCCCCCCCN(CCO)CCCCCCCC(=O)OC(CCCCCCCC)CCCCCCCC] |
| III-62 | 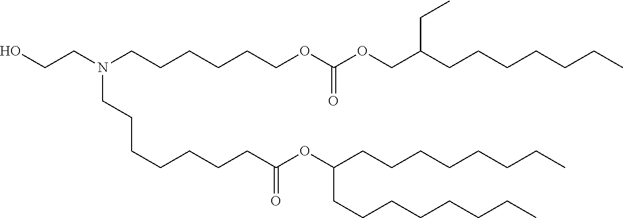 [SMILES: CCCCCCCCC(CCCCCCCC)OC(=O)CCCCCCCN(CCO)CCCCCCOC(=O)OCC(CC)CCCCCCC] |
| III-63 | 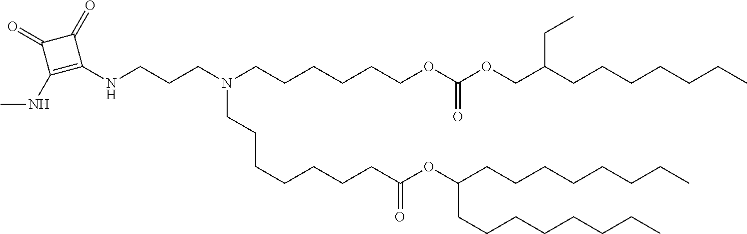 [SMILES: CCCCCCCCC(CCCCCCCC)OC(=O)CCCCCCCN(CCCCCCOC(=O)OCC(CC)CCCCCCC)CCCNc1c(NC)c(=O)c1=O] |
| III-64 | 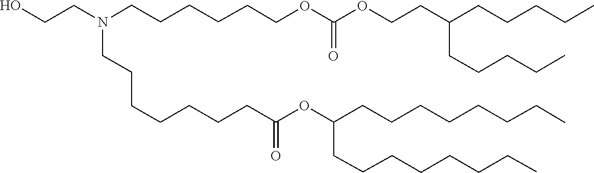 [SMILES: CCCCCCCCC(CCCCCCCC)OC(=O)CCCCCCCN(CCO)CCCCCCOC(=O)OCCC(CCCCC)CCCCC] |
| III-65 | 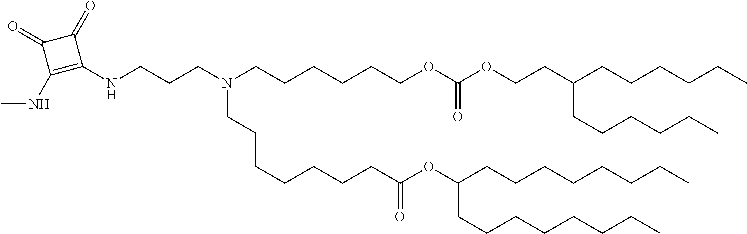 [SMILES: CCCCCCCCC(CCCCCCCC)OC(=O)CCCCCCCN(CCCCCCOC(=O)OCCC(CCCCCC)CCCCCC)CCCNc1c(NC)c(=O)c1=O] |
| III-66 | 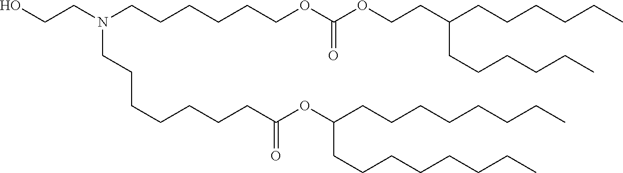 [SMILES: CCCCCCCCC(CCCCCCCC)OC(=O)CCCCCCCN(CCO)CCCCCCOC(=O)OCCC(CCCCCC)CCCCCC] |
| III-67 | 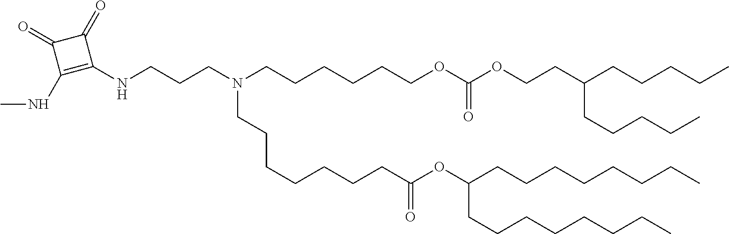 [SMILES: CCCCCCCCC(CCCCCCCC)OC(=O)CCCCCCCN(CCCCCCOC(=O)OCCC(CCCCC)CCCCC)CCCNc1c(NC)c(=O)c1=O] |

In some embodiments, the ionizable lipid is a compound of Formula (IL-IIB):

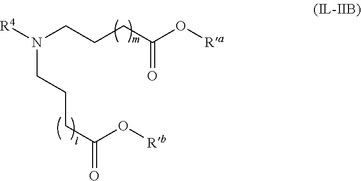

or its N-oxide, or a salt or isomer thereof,
wherein R′^{a} is:

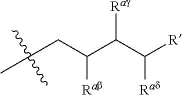

### and R′^{b} is:

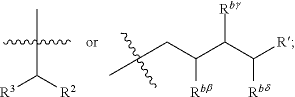

wherein

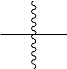

denotes a point of attachment;
R^{aβ}, R^{aγ}, and R^{aδ} are each independently selected from the group consisting of H, C₁₋₁₂ alkyl, and C₂₋₁₂ alkenyl;
R^{bβ}, R^{bγ}, and R^{bδ} are each independently selected from the group consisting of H, C₁₋₁₂ alkyl, and C₂₋₁₂ alkenyl, wherein at least one of R^{bβ}, R^{bγ}, and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from —(CH₂)ₙNRTQ, —(CH₂)ₙNRS(O)₂TQ, —(CH₂)ₙNRC(O)H, and —(CH₂)ₙNRC(O)TQ, wherein n is selected from 1, 2, 3, 4, and 5;
T is a bond or a C₁₋₃ alkyl linker, C₂₋₃ alkenyl linker, or C₂₋₃ alkynyl linker;
Q is selected from 3-14 membered heterocycle containing 1-5 heteroatoms selected from N, O, and S, C₃₋₁₀ carbocycle, C₁₋₆ alkyl, and C₂₋₆ alkenyl, wherein the alkyl, alkenyl, heterocycle, and carbocycle are each optionally substituted with one or more R^{Q};
each R^{Q} independently is selected from the group consisting of oxo, hydroxyl, cyano, amino, C₁₋₆ alkylamino, di-C₁₋₆ alkylamino, C₁₋₆ alkyl, C₁₋₆ alkoxy, C₂₋₆ alkenyl, C₁₋₆ alkanolyl, —C(O)C₁₋₆ alkyl, and —NRC(O)C₁₋₆ alkyl;
each R is independently selected from H, C₁₋₆ alkyl, and C₂₋₆ alkenyl;
each R′ is independently selected from C₁₋₁₂ alkyl and C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound selected from Table IL-4.

TABLE IL-4

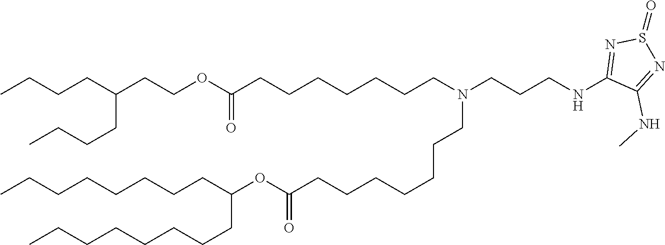 [SMILES: CCCCCCCCC(CCCCCCCC)OC(=O)CCCCCCCN(CCCCCCCC(=O)OCCC(CCCC)CCCC)CCCNC1=NS(=O)N=C1NC]

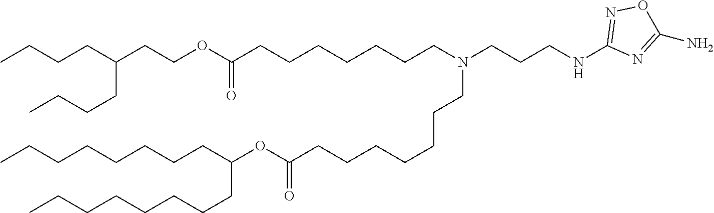 [SMILES: CCCCCCCCC(CCCCCCCC)OC(=O)CCCCCCCN(CCCCCCCC(=O)OCCC(CCCC)CCCC)CCCNc1noc(N)n1]

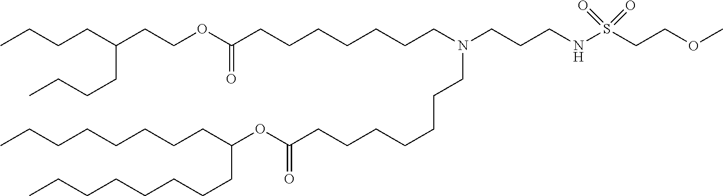 [SMILES: CCCCCCCCC(CCCCCCCC)OC(=O)CCCCCCCN(CCCCCCCC(=O)OCCC(CCCC)CCCC)CCCNS(=O)(=O)CCOC]

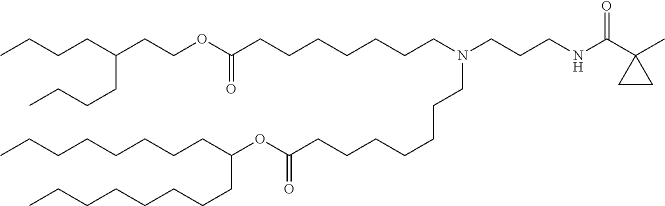 [SMILES: CCCCCCCCC(CCCCCCCC)OC(=O)CCCCCCCN(CCCCCCCC(=O)OCCC(CCCC)CCCC)CCCNC(=O)C1(C)CC1]

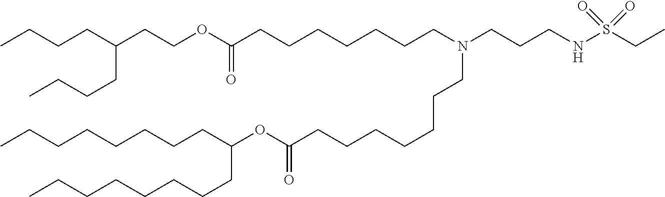 [SMILES: CCCCCCCCC(CCCCCCCC)OC(=O)CCCCCCCN(CCCCCCCC(=O)OCCC(CCCC)CCCC)CCCNS(=O)(=O)CC]

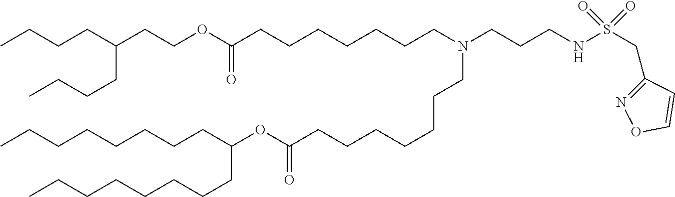 [SMILES: CCCCCCCCC(CCCCCCCC)OC(=O)CCCCCCCN(CCCCCCCC(=O)OCCC(CCCC)CCCC)CCCNS(=O)(=O)Cc1ccon1]

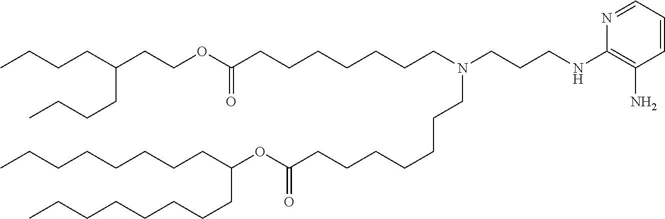 [SMILES: CCCCCCCCC(CCCCCCCC)OC(=O)CCCCCCCN(CCCCCCCC(=O)OCCC(CCCC)CCCC)CCCNc1ncccc1N]

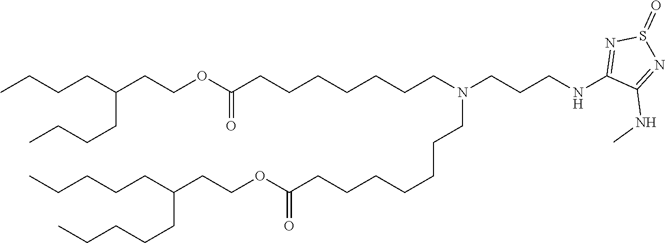 [SMILES: CCCCCC(CCCCC)CCOC(=O)CCCCCCCN(CCCCCCCC(=O)OCCC(CCCC)CCCC)CCCNC1=NS(=O)N=C1NC]

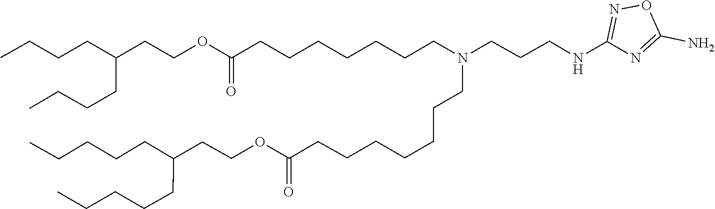 [SMILES: CCCCCC(CCCCC)CCOC(=O)CCCCCCCN(CCCCCCCC(=O)OCCC(CCCC)CCCC)CCCNc1noc(N)n1]

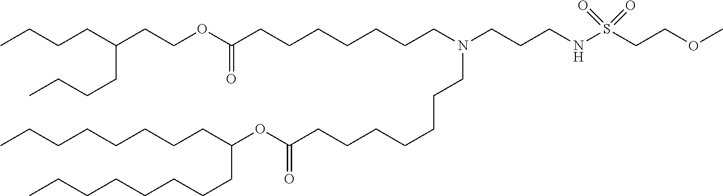 [SMILES: CCCCCCCCC(CCCCCCCC)OC(=O)CCCCCCCN(CCCCCCCC(=O)OCCC(CCCC)CCCC)CCCNS(=O)(=O)CCOC]

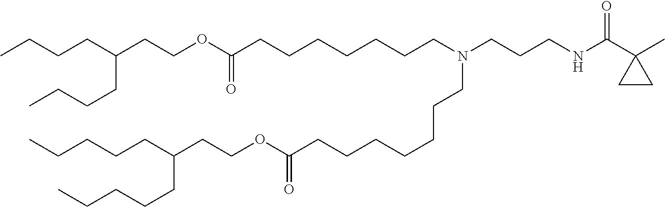

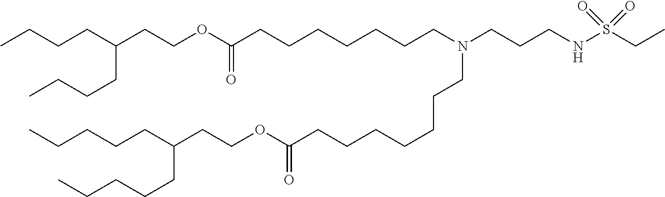 [SMILES: CCCCCC(CCCCC)CCOC(=O)CCCCCCCN(CCCCCCCC(=O)OCCC(CCCC)CCCC)CCCNS(=O)(=O)CC]

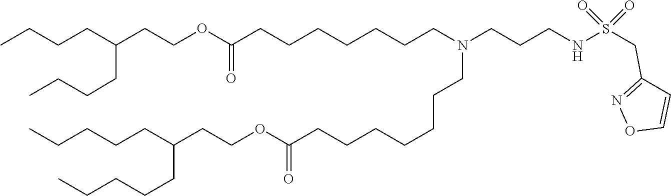 [SMILES: CCCCCC(CCCCC)CCOC(=O)CCCCCCCN(CCCCCCCC(=O)OCCC(CCCC)CCCC)CCCNS(=O)(=O)Cc1ccon1]

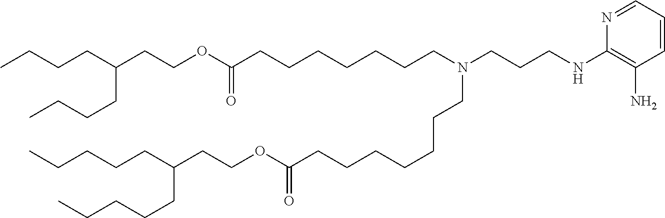 [SMILES: CCCCCC(CCCCC)CCOC(=O)CCCCCCCN(CCCCCCCC(=O)OCCC(CCCC)CCCC)CCCNc1ncccc1N]

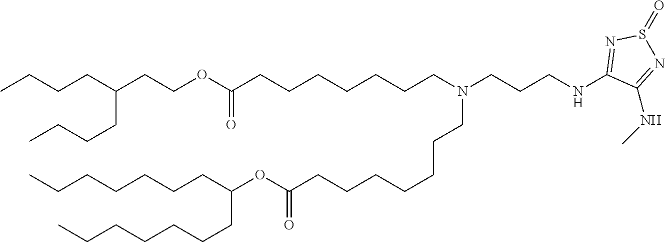 [SMILES: CCCCCCCC(CCCCCCC)OC(=O)CCCCCCCN(CCCCCCCC(=O)OCCC(CCCC)CCCC)CCCNC1=NS(=O)N=C1NC]

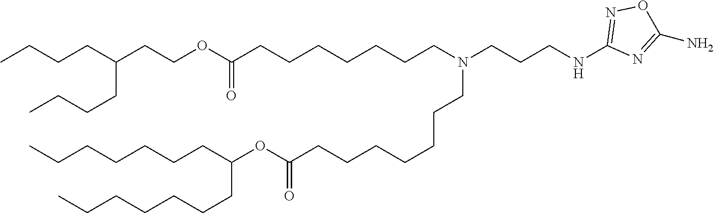 [SMILES: CCCCCCCC(CCCCCCC)OC(=O)CCCCCCCN(CCCCCCCC(=O)OCCC(CCCC)CCCC)CCCNc1noc(N)n1]

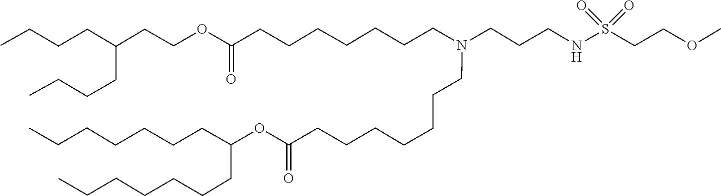 [SMILES: CCCCCCCC(CCCCCCC)OC(=O)CCCCCCCN(CCCCCCCC(=O)OCCC(CCCC)CCCC)CCCNS(=O)(=O)CCOC]

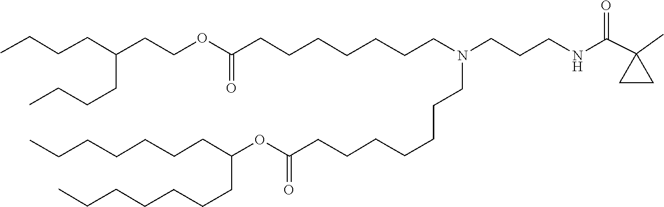 [SMILES: CCCCCCCC(CCCCCCC)OC(=O)CCCCCCCN(CCCCCCCC(=O)OCCC(CCCC)CCCC)CCCNC(=O)C1(C)CC1]

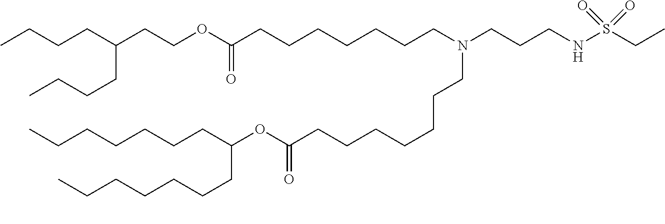

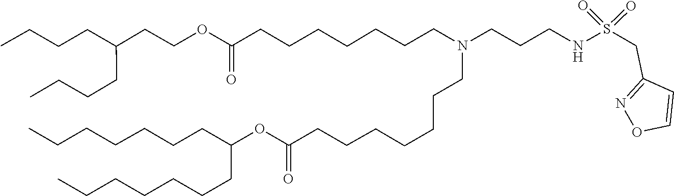 [SMILES: CCCCCCCC(CCCCCCC)OC(=O)CCCCCCCN(CCCCCCCC(=O)OCCC(CCCC)CCCC)CCCNS(=O)(=O)Cc1ccon1]

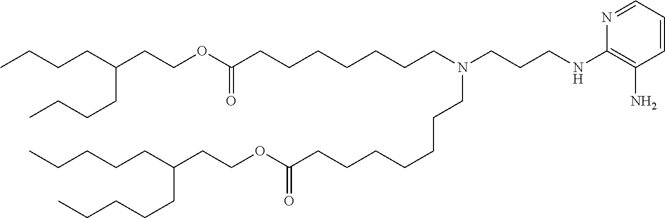 [SMILES: CCCCCC(CCCCC)CCOC(=O)CCCCCCCN(CCCCCCCC(=O)OCCC(CCCC)CCCC)CCCNc1ncccc1N]

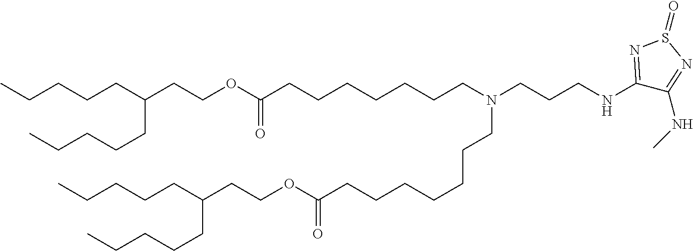

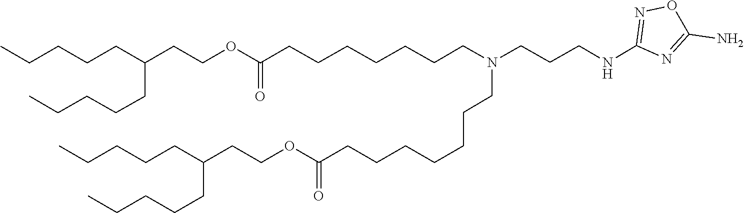 [SMILES: CCCCCC(CCCCC)CCOC(=O)CCCCCCCN(CCCCCCCC(=O)OCCC(CCCCC)CCCCC)CCCNc1noc(N)n1]

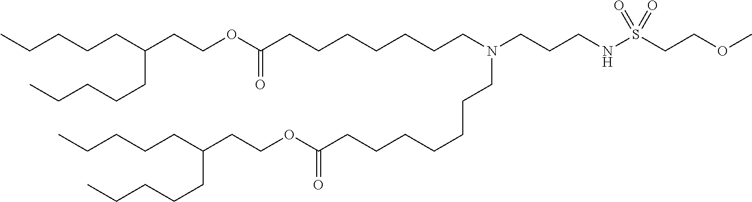 [SMILES: CCCCCC(CCCCC)CCOC(=O)CCCCCCCN(CCCCCCCC(=O)OCCC(CCCCC)CCCCC)CCCNS(=O)(=O)CCOC]

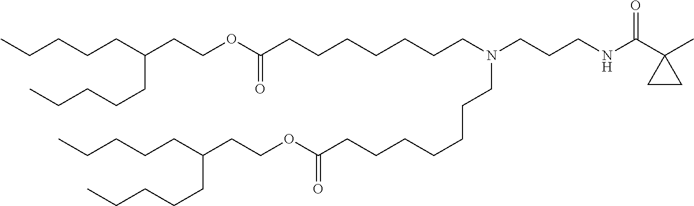 [SMILES: CCCCCC(CCCCC)CCOC(=O)CCCCCCCN(CCCCCCCC(=O)OCCC(CCCCC)CCCCC)CCCNC(=O)C1(C)CC1]

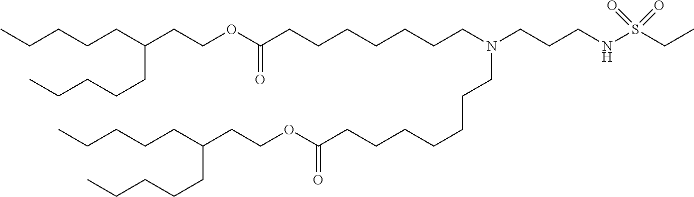 [SMILES: CCCCCC(CCCCC)CCOC(=O)CCCCCCCN(CCCCCCCC(=O)OCCC(CCCCC)CCCCC)CCCNS(=O)(=O)CC]

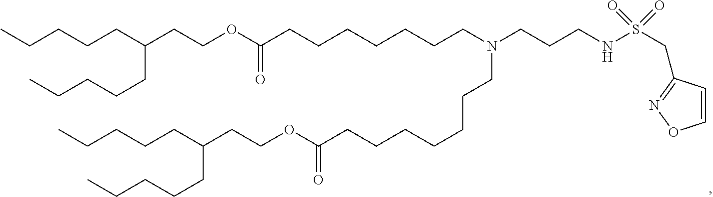 [SMILES: CCCCCC(CCCCC)CCOC(=O)CCCCCCCN(CCCCCCCC(=O)OCCC(CCCCC)CCCCC)CCCNS(=O)(=O)Cc1ccon1]

and

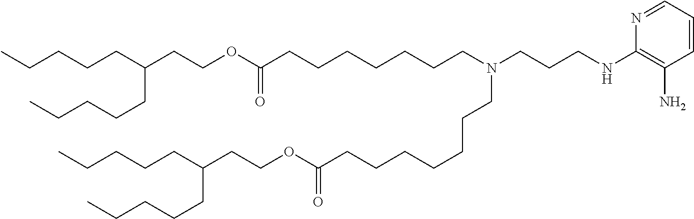 [SMILES: CCCCCC(CCCCC)CCOC(=O)CCCCCCCN(CCCCCCCC(=O)OCCC(CCCCC)CCCCC)CCCNc1ncccc1N]

In some embodiments, the ionizable lipid is a compound selected from Table IL-5.

TABLE IL-5

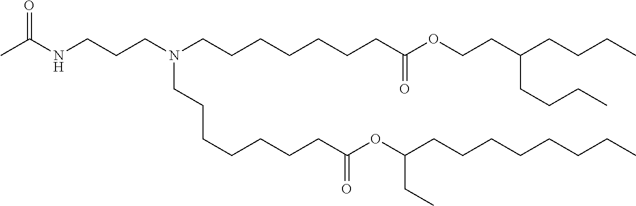 [SMILES: CCCCCCCCC(CC)OC(=O)CCCCCCCN(CCCCCCCC(=O)OCCC(CCCC)CCCC)CCCNC(C)=O]

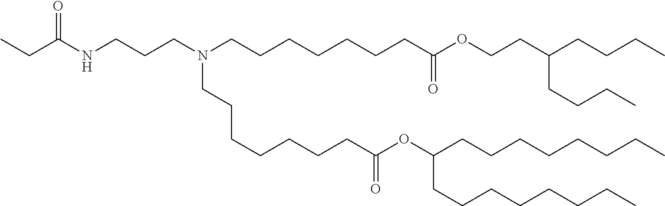 [SMILES: CCCCCCCCC(CCCCCCCC)OC(=O)CCCCCCCN(CCCCCCCC(=O)OCCC(CCCC)CCCC)CCCNC(=O)CC]

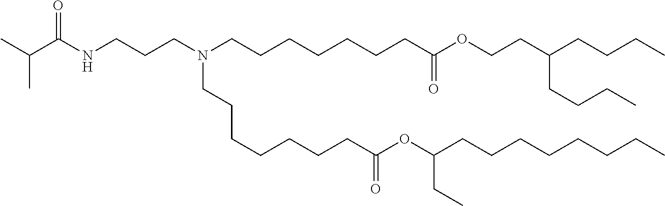 [SMILES: CCCCCCCCC(CC)OC(=O)CCCCCCCN(CCCCCCCC(=O)OCCC(CCCC)CCCC)CCCNC(=O)C(C)C]

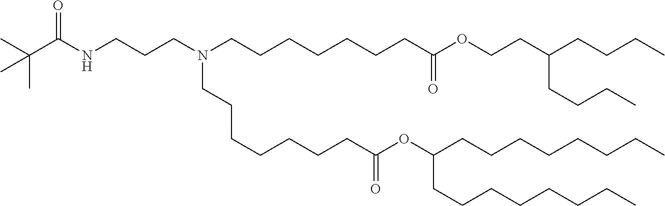 [SMILES: CCCCCCCCC(CCCCCCCC)OC(=O)CCCCCCCN(CCCCCCCC(=O)OCCC(CCCC)CCCC)CCCNC(=O)C(C)(C)C]

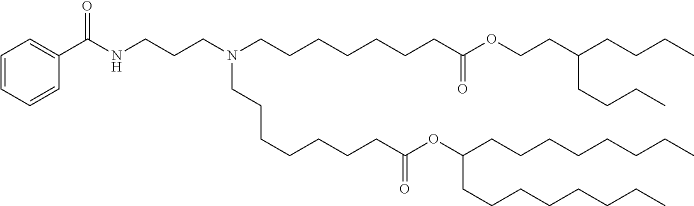 [SMILES: CCCCCCCCC(CCCCCCCC)OC(=O)CCCCCCCN(CCCCCCCC(=O)OCCC(CCCC)CCCC)CCCNC(=O)c1ccccc1]

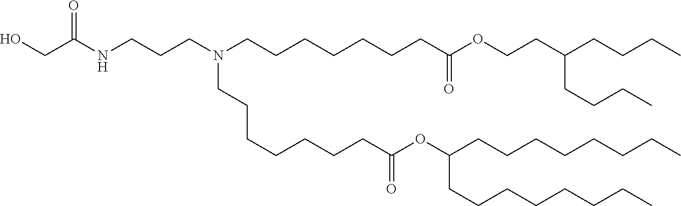 [SMILES: CCCCCCCCC(CCCCCCCC)OC(=O)CCCCCCCN(CCCCCCCC(=O)OCCC(CCCC)CCCC)CCCNC(=O)CO]

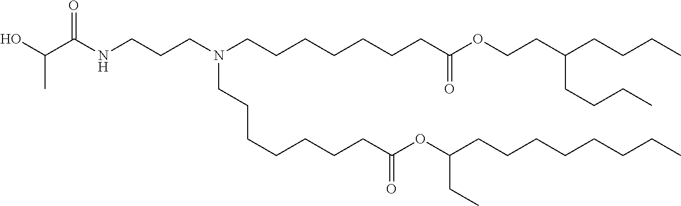 [SMILES: CCCCCCCCC(CC)OC(=O)CCCCCCCN(CCCCCCCC(=O)OCCC(CCCC)CCCC)CCCNC(=O)C(C)O]

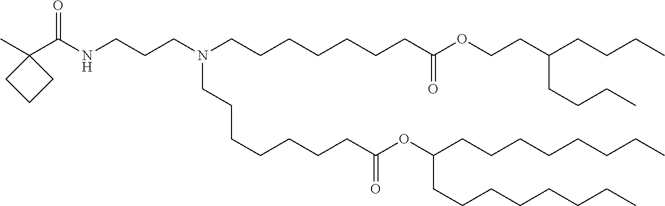 [SMILES: CCCCCCCCC(CCCCCCCC)OC(=O)CCCCCCCN(CCCCCCCC(=O)OCCC(CCCC)CCCC)CCCNC(=O)C1(C)CCC1]

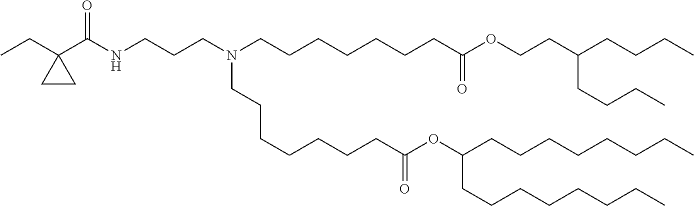 [SMILES: CCCCCCCCC(CCCCCCCC)OC(=O)CCCCCCCN(CCCCCCCC(=O)OCCC(CCCC)CCCC)CCCNC(=O)C1(CC)CC1]

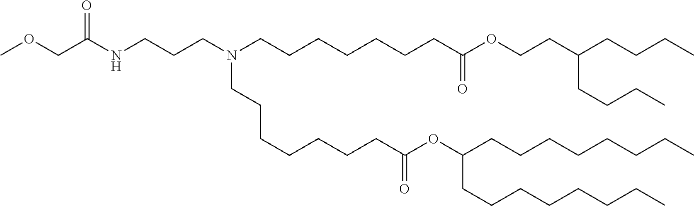 [SMILES: CCCCCCCCC(CCCCCCCC)OC(=O)CCCCCCCN(CCCCCCCC(=O)OCCC(CCCC)CCCC)CCCNC(=O)COC]

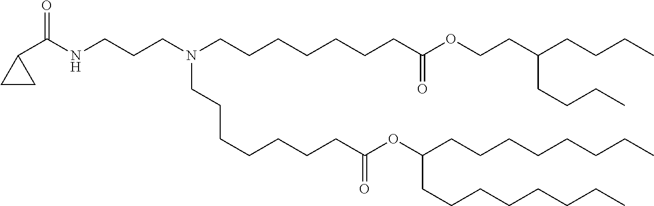 [SMILES: CCCCCCCCC(CCCCCCCC)OC(=O)CCCCCCCN(CCCCCCCC(=O)OCCC(CCCC)CCCC)CCCNC(=O)C1CC1]

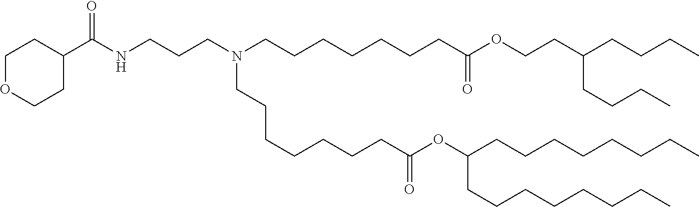 [SMILES: CCCCCCCCC(CCCCCCCC)OC(=O)CCCCCCCN(CCCCCCCC(=O)OCCC(CCCC)CCCC)CCCNC(=O)C1CCOCC1]

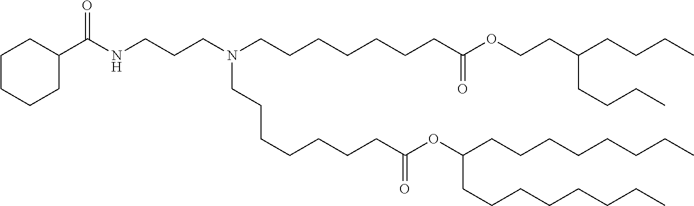 [SMILES: CCCCCCCCC(CCCCCCCC)OC(=O)CCCCCCCN(CCCCCCCC(=O)OCCC(CCCC)CCCC)CCCNC(=O)C1CCCCC1]

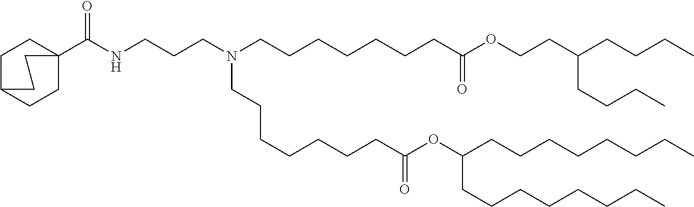 [SMILES: CCCCCCCCC(CCCCCCCC)OC(=O)CCCCCCCN(CCCCCCCC(=O)OCCC(CCCC)CCCC)CCCNC(=O)C12CCC(CC1)CC2]

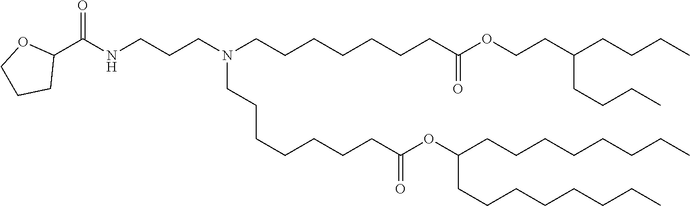 [SMILES: CCCCCCCCC(CCCCCCCC)OC(=O)CCCCCCCN(CCCCCCCC(=O)OCCC(CCCC)CCCC)CCCNC(=O)C1CCCO1]

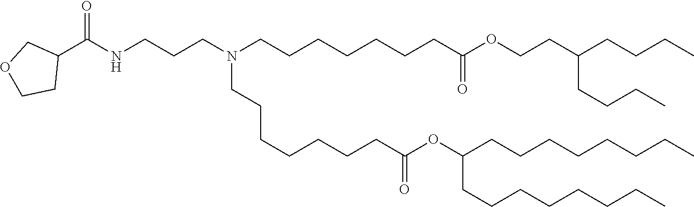 [SMILES: CCCCCCCCC(CCCCCCCC)OC(=O)CCCCCCCN(CCCCCCCC(=O)OCCC(CCCC)CCCC)CCCNC(=O)C1CCOC1]

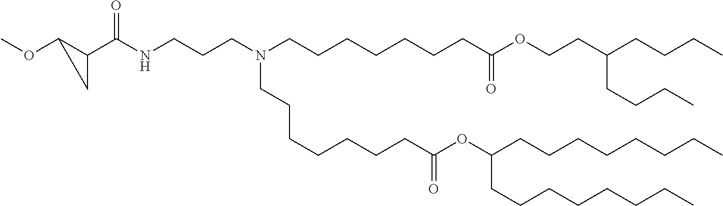 [SMILES: CCCCCCCCC(CCCCCCCC)OC(=O)CCCCCCCN(CCCCCCCC(=O)OCCC(CCCC)CCCC)CCCNC(=O)C1CC1OC]

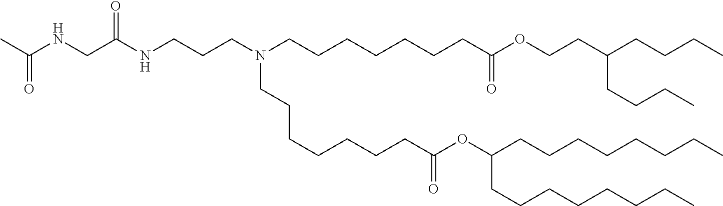 [SMILES: CCCCCCCCC(CCCCCCCC)OC(=O)CCCCCCCN(CCCCCCCC(=O)OCCC(CCCC)CCCC)CCCNC(=O)CNC(C)=O]

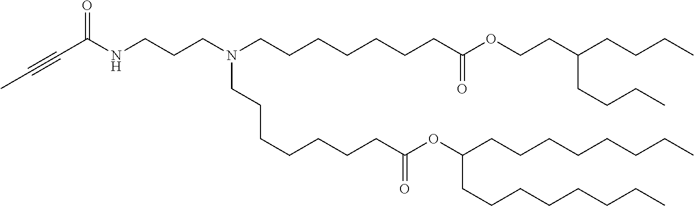 [SMILES: CC#CC(=O)NCCCN(CCCCCCCC(=O)OCCC(CCCC)CCCC)CCCCCCCC(=O)OC(CCCCCCCC)CCCCCCCC]

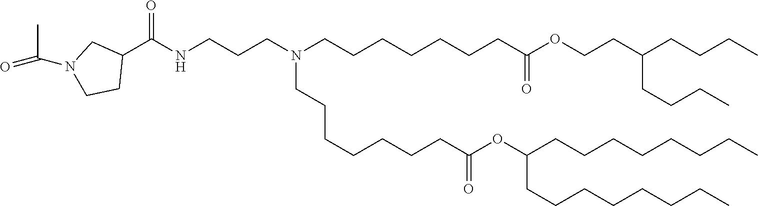 [SMILES: CCCCCCCCC(CCCCCCCC)OC(=O)CCCCCCCN(CCCCCCCC(=O)OCCC(CCCC)CCCC)CCCNC(=O)C1CCN(C(C)=O)C1]

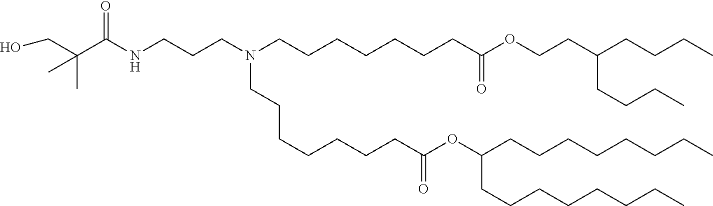 [SMILES: CCCCCCCCC(CCCCCCCC)OC(=O)CCCCCCCN(CCCCCCCC(=O)OCCC(CCCC)CCCC)CCCNC(=O)C(C)(C)CO]

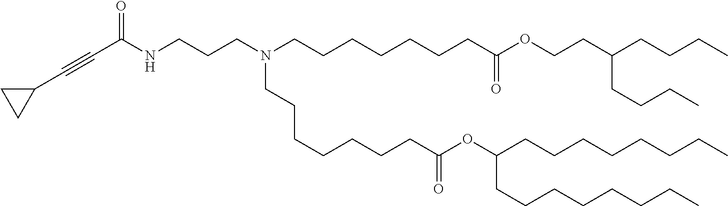 [SMILES: CCCCCCCCC(CCCCCCCC)OC(=O)CCCCCCCN(CCCCCCCC(=O)OCCC(CCCC)CCCC)CCCNC(=O)C#CC1CC1]

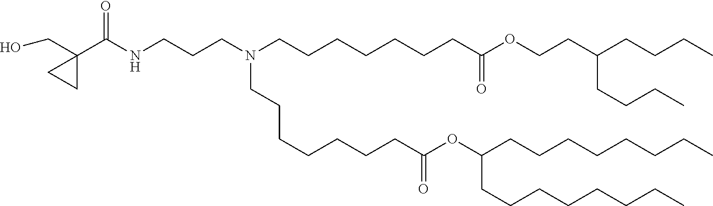 [SMILES: CCCCCCCCC(CCCCCCCC)OC(=O)CCCCCCCN(CCCCCCCC(=O)OCCC(CCCC)CCCC)CCCNC(=O)C1(CO)CC1]

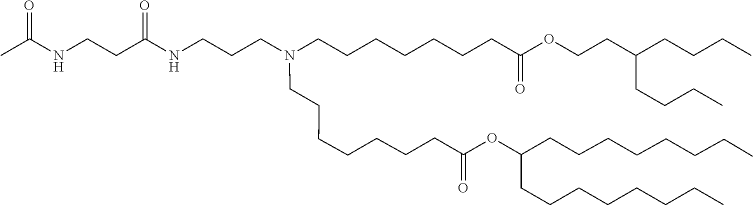 [SMILES: CCCCCCCCC(CCCCCCCC)OC(=O)CCCCCCCN(CCCCCCCC(=O)OCCC(CCCC)CCCC)CCCNC(=O)CCNC(C)=O]

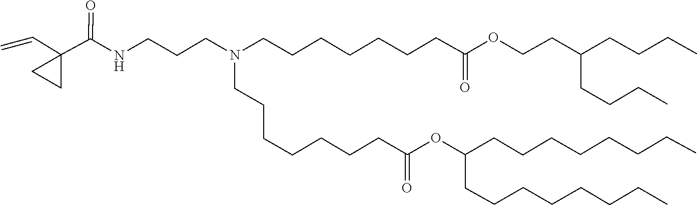 [SMILES: C=CC1(C(=O)NCCCN(CCCCCCCC(=O)OCCC(CCCC)CCCC)CCCCCCCC(=O)OC(CCCCCCCC)CCCCCCCC)CC1]

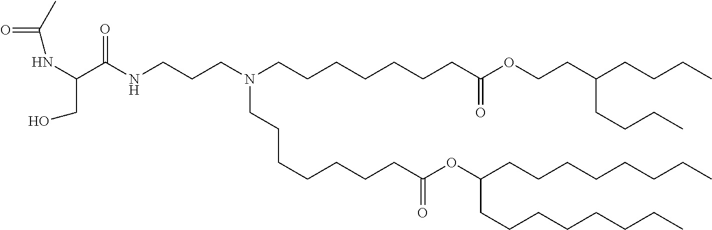 [SMILES: CCCCCCCCC(CCCCCCCC)OC(=O)CCCCCCCN(CCCCCCCC(=O)OCCC(CCCC)CCCC)CCCNC(=O)C(CO)NC(C)=O]

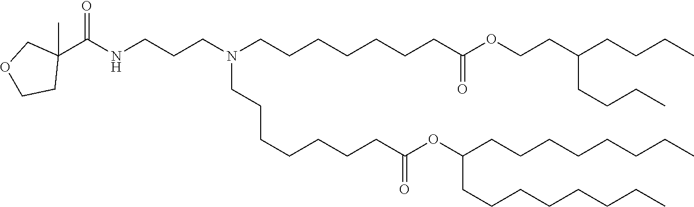 [SMILES: CCCCCCCCC(CCCCCCCC)OC(=O)CCCCCCCN(CCCCCCCC(=O)OCCC(CCCC)CCCC)CCCNC(=O)C1(C)CCOC1]

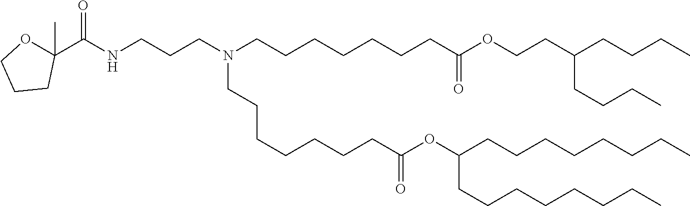 [SMILES: CCCCCCCCC(CCCCCCCC)OC(=O)CCCCCCCN(CCCCCCCC(=O)OCCC(CCCC)CCCC)CCCNC(=O)C1(C)CCCO1]

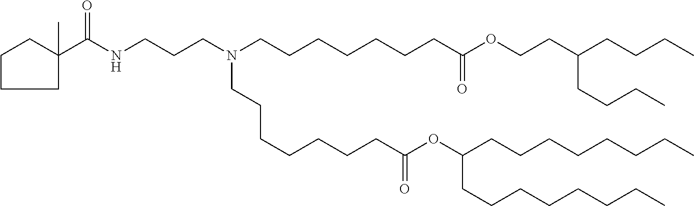 [SMILES: CCCCCCCCC(CCCCCCCC)OC(=O)CCCCCCCN(CCCCCCCC(=O)OCCC(CCCC)CCCC)CCCNC(=O)C1(C)CCCC1]

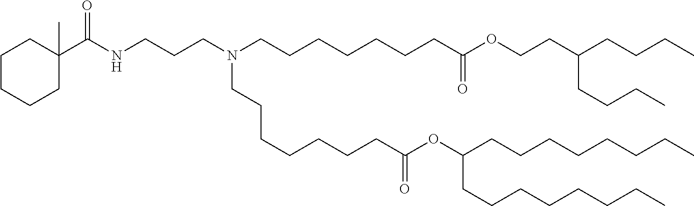 [SMILES: CCCCCCCCC(CCCCCCCC)OC(=O)CCCCCCCN(CCCCCCCC(=O)OCCC(CCCC)CCCC)CCCNC(=O)C1(C)CCCCC1]

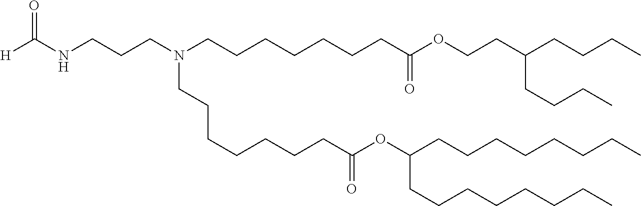 [SMILES: CCCCCCCCC(CCCCCCCC)OC(=O)CCCCCCCN(CCCCCCCC(=O)OCCC(CCCC)CCCC)CCCNC=O]

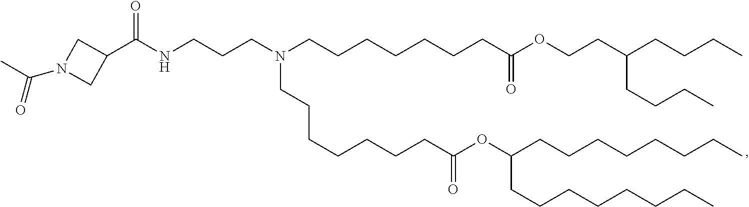 [SMILES: CCCCCCCCC(CCCCCCCC)OC(=O)CCCCCCCN(CCCCCCCC(=O)OCCC(CCCC)CCCC)CCCNC(=O)C1CN(C(C)=O)C1]

and

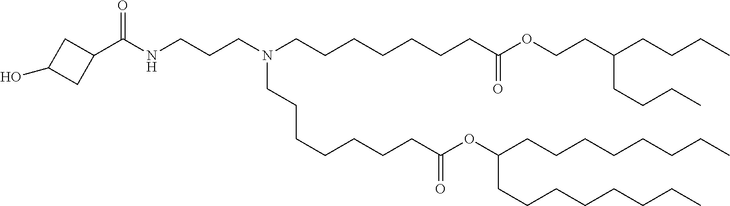 [SMILES: CCCCCCCCC(CCCCCCCC)OC(=O)CCCCCCCN(CCCCCCCC(=O)OCCC(CCCC)CCCC)CCCNC(=O)C1CC(O)C1]

In some embodiments, the ionizable lipid is a compound of Formula (IL-IIC):

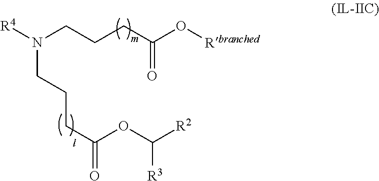

or its N-oxide, or a salt or isomer thereof, wherein:
R′^{branched} is

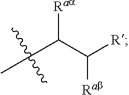

wherein

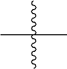

denotes a point of attachment;
wherein R^{aα} and R^{aβ} are each independently selected from the group consisting of H and C₁₋₂ alkyl, wherein at least one of R^{aα} and R^{aβ} is a C₁ or C₂ alkyl;
R′ is selected from the group consisting of C₁₋₁₈ alkyl and C₂₋₁₈ alkenyl;
R² and R³ are each independently selected from the group consisting of C₁₋₁₄ alkyl and C₂₋₁₄ alkenyl;
R⁴ is —(CH₂)ₙQ, wherein n is independently selected from 1, 2, 3, 4, and 5, where Q is selected from NRS(O)₂R^{SX} and

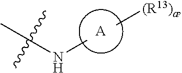

wherein A is a 3-14 membered heterocycle containing one or more heteroatoms selected from N, O and S; and a is 1, 2, 3, or 4; wherein

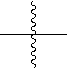

denotes a point of attachment;
R is selected from H and C₁₋₃ alkyl;
R^{SX} is selected from a C₃₋₈ carbocycle, a 3-14 membered heterocycle containing one or more heteroatoms selected from N, O and S, C₁₋₆ alkyl, C₂₋₆ alkenyl, (C₁₋₃ alkoxy)C₁₋₃ alkyl, (CH₂)ₚ₁O(CH₂)ₚ₂R^{SX1}, and (CH₂)ₚ₁R^{SX1}, wherein the carbocycle and heterocycle are optionally substituted with one or more groups selected from oxo, C₁₋₆ alkyl, and (C₁₋₃ alkoxy)C₁₋₃ alkyl;
R^{SX1} is selected from C(O)NR¹⁴R¹⁴′, a C₃₋₈ carbocycle, and a 3-14 membered heterocycle containing one or more heteroatoms selected from N, O and S, wherein the carbocycle and heterocycle are each optionally substituted with one or more groups selected from oxo, halo, C₁₋₃ alkyl, (C₁₋₃ alkoxy)C₁₋₃ alkyl, C₁₋₆ alkylamino, di-(C₁₋₆ alkyl)amino, and NH₂;
each R¹³ is selected from the group consisting of OH, oxo, halo, C₁₋₆ alkyl, C₁₋₆ alkoxy, C₂₋₆ alkenyl, C₁₋₆ alkylamino, di-(C₁₋₆ alkyl)amino, NH₂, C(O)NH₂, CN, and NO₂;
R¹⁴ and R^{14′} are each independently selected from the group consisting of H and C₁₋₆ alkyl;
m is selected from 1, 2, 3, 4, 5, 6, 7, 8, and 9;
l is selected from 1, 2, 3, 4, 5, 6, 7, 8, and 9;
p₁ is selected from 1, 2, 3, 4, and 5; and
p₂ is selected from 1, 2, 3, 4, and 5.

In some embodiments, the ionizable lipid is a compound selected from Table IL-6.

TABLE IL-6

| Cpd | Structure |
|---|---|
| V-1 | 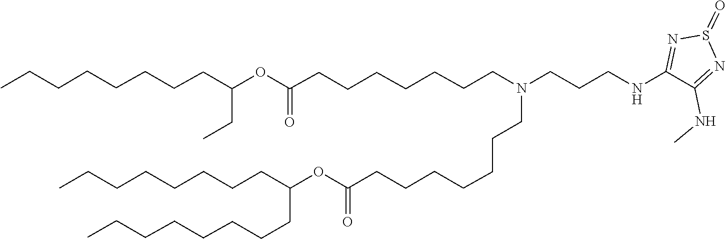 [SMILES: CCCCCCCCC(CC)OC(=O)CCCCCCCN(CCCCCCCC(=O)OC(CCCCCCCC)CCCCCCCC)CCCNC1=NS(=O)N=C1NC] |
| V-2 | 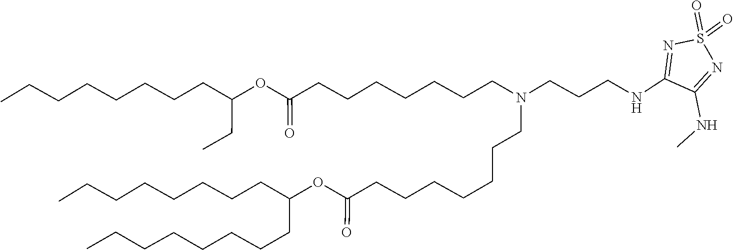 [SMILES: CCCCCCCCC(CC)OC(=O)CCCCCCCN(CCCCCCCC(=O)OC(CCCCCCCC)CCCCCCCC)CCCNC1=NS(=O)(=O)N=C1NC] |
| V-3 | 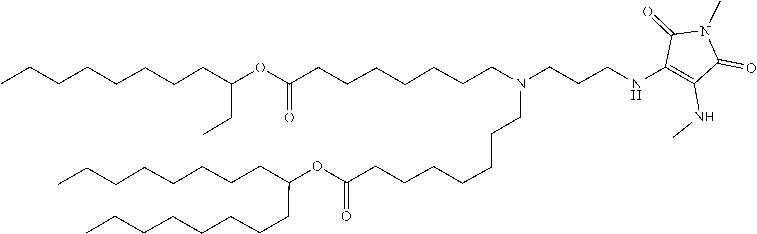 [SMILES: CCCCCCCCC(CC)OC(=O)CCCCCCCN(CCCCCCCC(=O)OC(CCCCCCCC)CCCCCCCC)CCCNC1=C(NC)C(=O)N(C)C1=O] |
| V-4 | 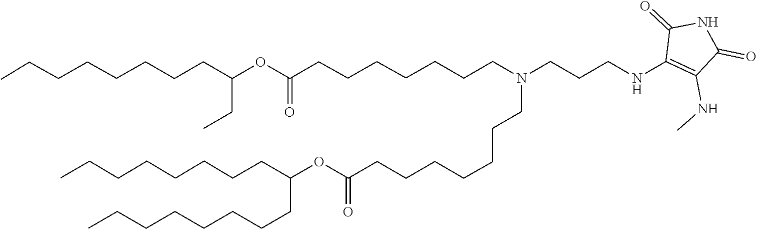 [SMILES: CCCCCCCCC(CC)OC(=O)CCCCCCCN(CCCCCCCC(=O)OC(CCCCCCCC)CCCCCCCC)CCCNC1=C(NC)C(=O)NC1=O] |
| V-5 | 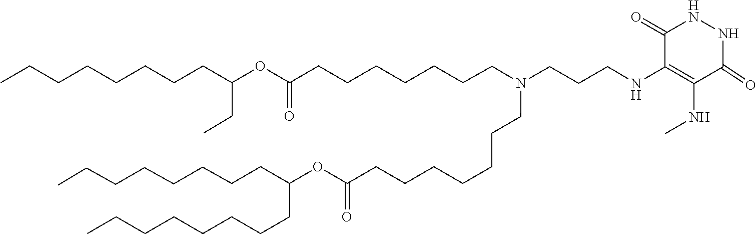 [SMILES: CCCCCCCCC(CC)OC(=O)CCCCCCCN(CCCCCCCC(=O)OC(CCCCCCCC)CCCCCCCC)CCCNc1c(NC)c(=O)[nH][nH]c1=O] |
| V-6 | 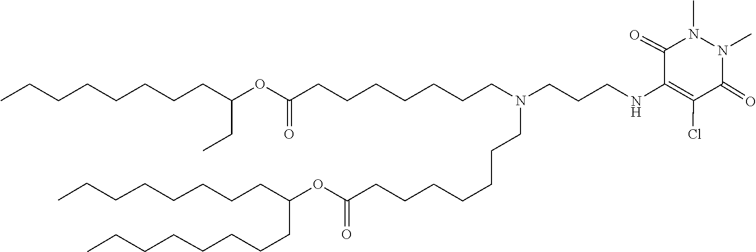 [SMILES: CCCCCCCCC(CC)OC(=O)CCCCCCCN(CCCCCCCC(=O)OC(CCCCCCCC)CCCCCCCC)CCCNc1c(Cl)c(=O)n(C)n(C)c1=O] |
| V-7 | 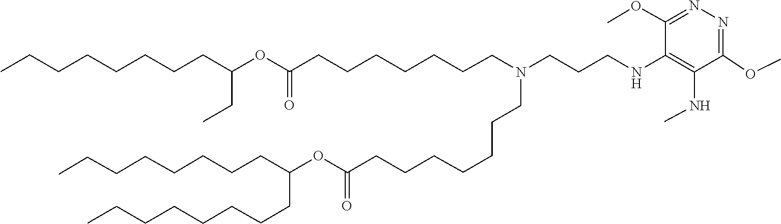 [SMILES: CCCCCCCCC(CC)OC(=O)CCCCCCCN(CCCCCCCC(=O)OC(CCCCCCCC)CCCCCCCC)CCCNc1c(OC)nnc(OC)c1NC] |
| V-8 | 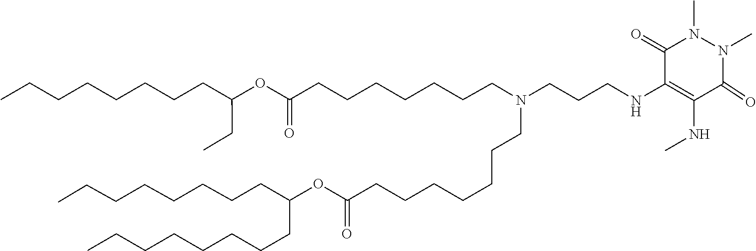 [SMILES: CCCCCCCCC(CC)OC(=O)CCCCCCCN(CCCCCCCC(=O)OC(CCCCCCCC)CCCCCCCC)CCCNc1c(NC)c(=O)n(C)n(C)c1=O] |
| V-9 | 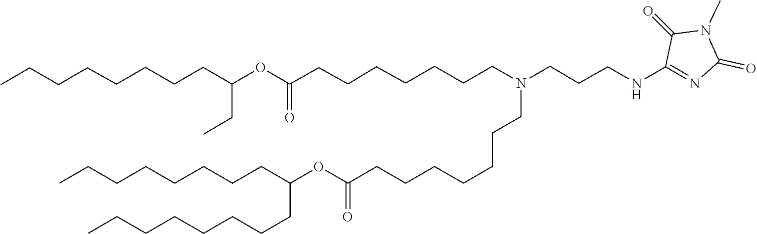 [SMILES: CCCCCCCCC(CC)OC(=O)CCCCCCCN(CCCCCCCC(=O)OC(CCCCCCCC)CCCCCCCC)CCCNC1=NC(=O)N(C)C1=O] |
| V-10 | 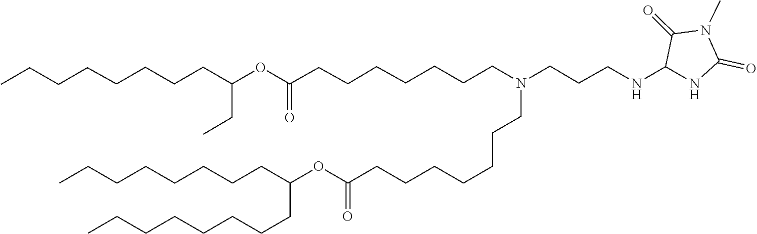 [SMILES: CCCCCCCCC(CC)OC(=O)CCCCCCCN(CCCCCCCC(=O)OC(CCCCCCCC)CCCCCCCC)CCCNC1NC(=O)N(C)C1=O] |
| V-11 | 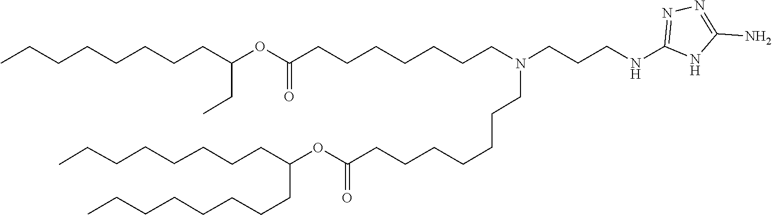 [SMILES: CCCCCCCCC(CC)OC(=O)CCCCCCCN(CCCCCCCC(=O)OC(CCCCCCCC)CCCCCCCC)CCCNc1nnc(N)[nH]1] |
| V-12 | 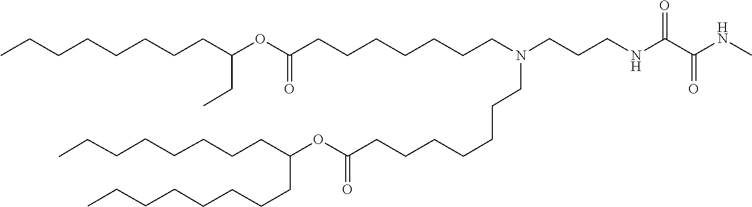 [SMILES: CCCCCCCCC(CC)OC(=O)CCCCCCCN(CCCCCCCC(=O)OC(CCCCCCCC)CCCCCCCC)CCCNC(=O)C(=O)NC] |
| V-13 | 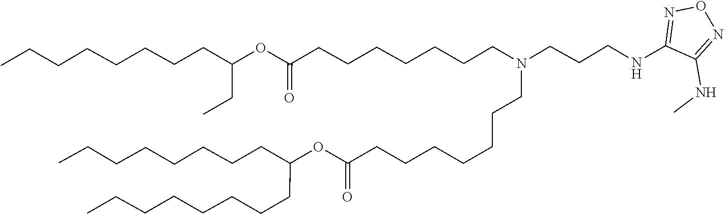 [SMILES: CCCCCCCCC(CC)OC(=O)CCCCCCCN(CCCCCCCC(=O)OC(CCCCCCCC)CCCCCCCC)CCCNc1nonc1NC] |
| V-14 | 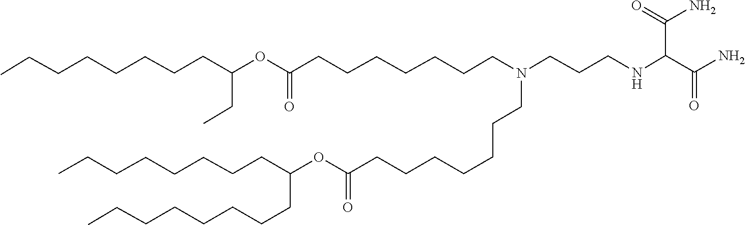 [SMILES: CCCCCCCCC(CC)OC(=O)CCCCCCCN(CCCCCCCC(=O)OC(CCCCCCCC)CCCCCCCC)CCCNC(C(N)=O)C(N)=O] |
| V-15 | 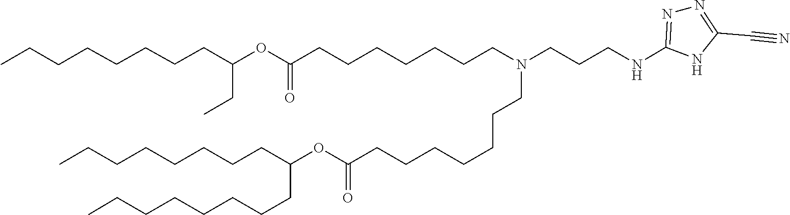 [SMILES: CCCCCCCCC(CC)OC(=O)CCCCCCCN(CCCCCCCC(=O)OC(CCCCCCCC)CCCCCCCC)CCCNc1nnc(C#N)[nH]1] |
| V-16 | 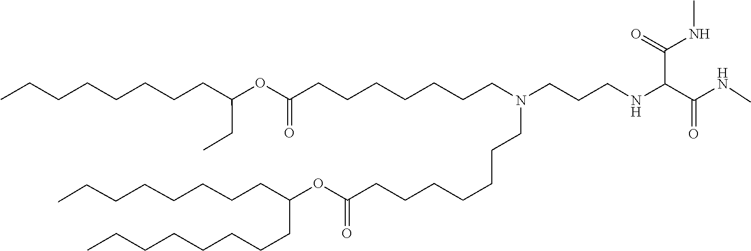 [SMILES: CCCCCCCCC(CC)OC(=O)CCCCCCCN(CCCCCCCC(=O)OC(CCCCCCCC)CCCCCCCC)CCCNC(C(=O)NC)C(=O)NC] |
| V-17 | 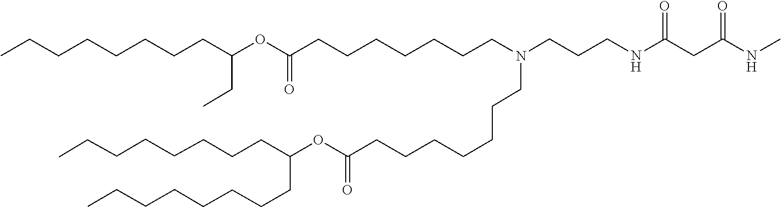 [SMILES: CCCCCCCCC(CC)OC(=O)CCCCCCCN(CCCCCCCC(=O)OC(CCCCCCCC)CCCCCCCC)CCCNC(=O)CC(=O)NC] |
| V-18 | 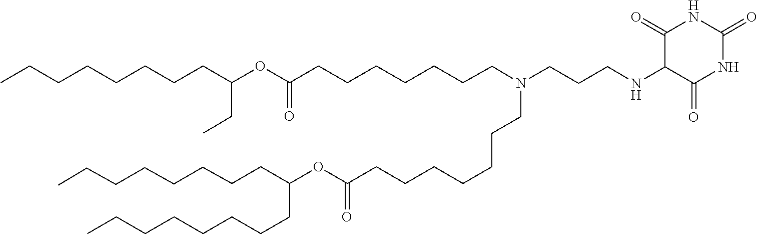 [SMILES: CCCCCCCCC(CC)OC(=O)CCCCCCCN(CCCCCCCC(=O)OC(CCCCCCCC)CCCCCCCC)CCCNC1C(=O)NC(=O)NC1=O] |
| V-19 | 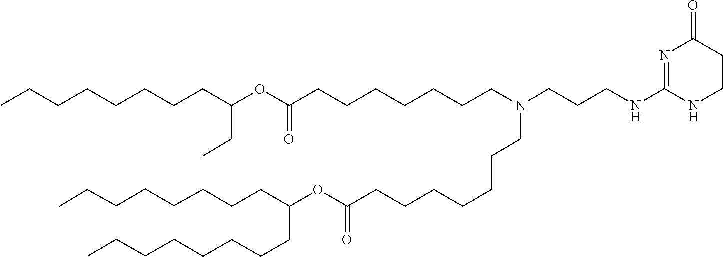 [SMILES: CCCCCCCCC(CC)OC(=O)CCCCCCCN(CCCCCCCC(=O)OC(CCCCCCCC)CCCCCCCC)CCCNC1=NC(=O)CCN1] |
| V-20 | 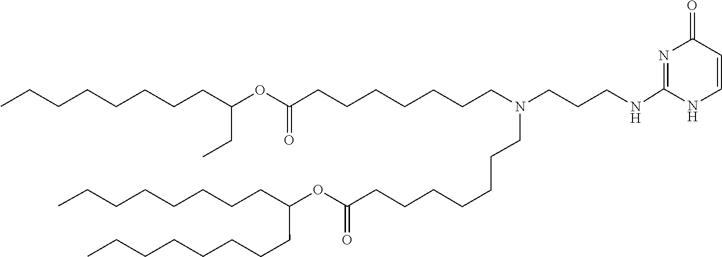 [SMILES: CCCCCCCCC(CC)OC(=O)CCCCCCCN(CCCCCCCC(=O)OC(CCCCCCCC)CCCCCCCC)CCCNc1nc(=O)cc[nH]1] |
| V-21 | 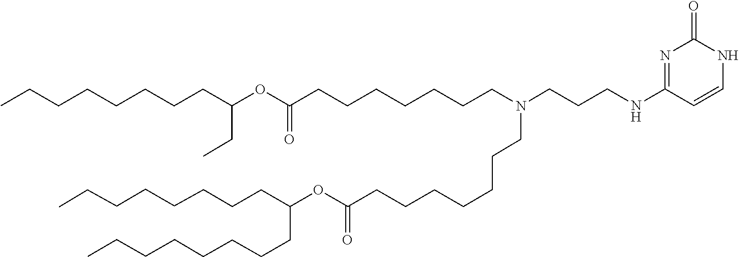 [SMILES: CCCCCCCCC(CC)OC(=O)CCCCCCCN(CCCCCCCC(=O)OC(CCCCCCCC)CCCCCCCC)CCCNc1cc[nH]c(=O)n1] |
| V-22 | 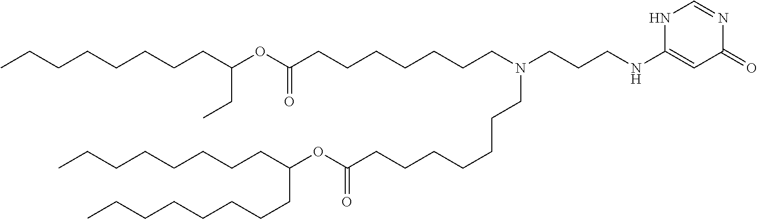 [SMILES: CCCCCCCCC(CC)OC(=O)CCCCCCCN(CCCCCCCC(=O)OC(CCCCCCCC)CCCCCCCC)CCCNc1cc(=O)nc[nH]1] |
| V-23 | 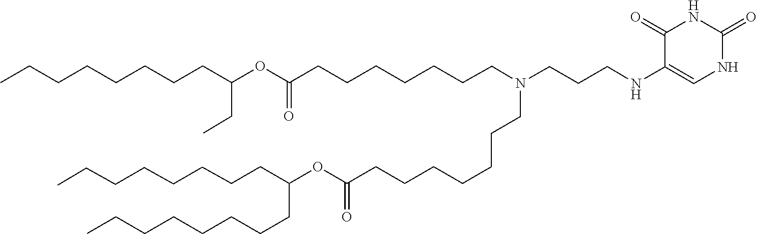 [SMILES: CCCCCCCCC(CC)OC(=O)CCCCCCCN(CCCCCCCC(=O)OC(CCCCCCCC)CCCCCCCC)CCCNc1c[nH]c(=O)[nH]c1=O] |
| V-24 | 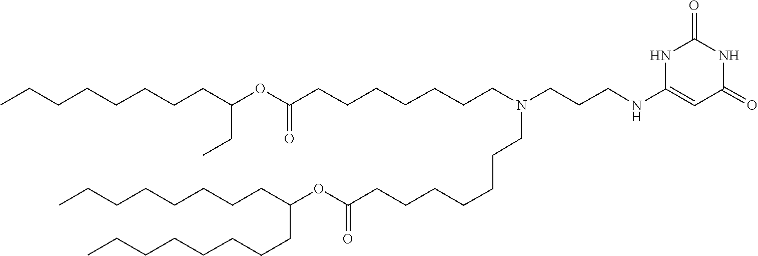 [SMILES: CCCCCCCCC(CC)OC(=O)CCCCCCCN(CCCCCCCC(=O)OC(CCCCCCCC)CCCCCCCC)CCCNc1cc(=O)[nH]c(=O)[nH]1] |
| V-25 | 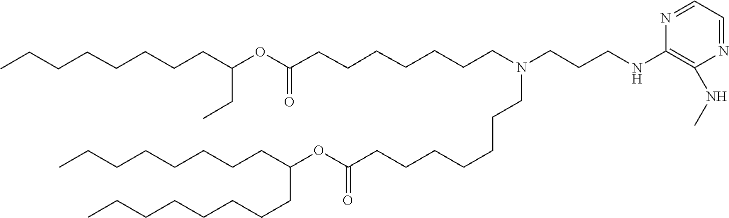 [SMILES: CCCCCCCCC(CC)OC(=O)CCCCCCCN(CCCCCCCC(=O)OC(CCCCCCCC)CCCCCCCC)CCCNc1nccnc1NC] |
| V-26 | 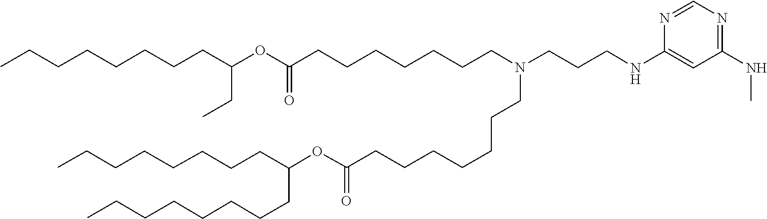 [SMILES: CCCCCCCCC(CC)OC(=O)CCCCCCCN(CCCCCCCC(=O)OC(CCCCCCCC)CCCCCCCC)CCCNc1cc(NC)ncn1] |
| V-27 | 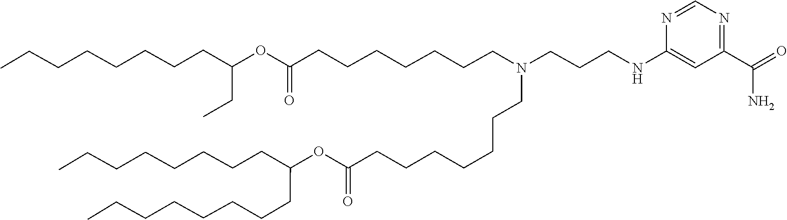 [SMILES: CCCCCCCCC(CC)OC(=O)CCCCCCCN(CCCCCCCC(=O)OC(CCCCCCCC)CCCCCCCC)CCCNc1cc(C(N)=O)ncn1] |
| V-28 | 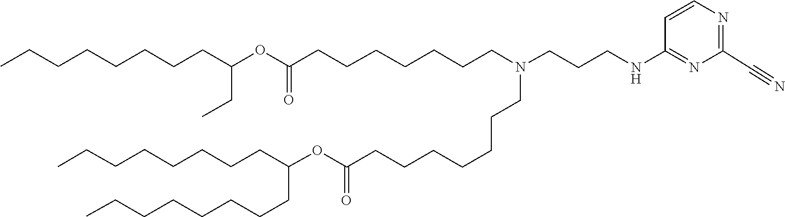 [SMILES: CCCCCCCCC(CC)OC(=O)CCCCCCCN(CCCCCCCC(=O)OC(CCCCCCCC)CCCCCCCC)CCCNc1ccnc(C#N)n1] |
| V-29 | 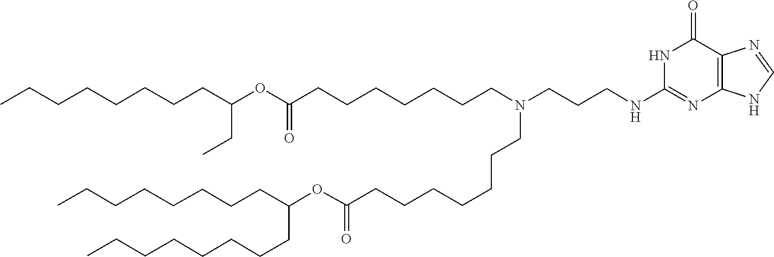 [SMILES: CCCCCCCCC(CC)OC(=O)CCCCCCCN(CCCCCCCC(=O)OC(CCCCCCCC)CCCCCCCC)CCCNc1nc2[nH]cnc2c(=O)[nH]1] |
| V-30 | 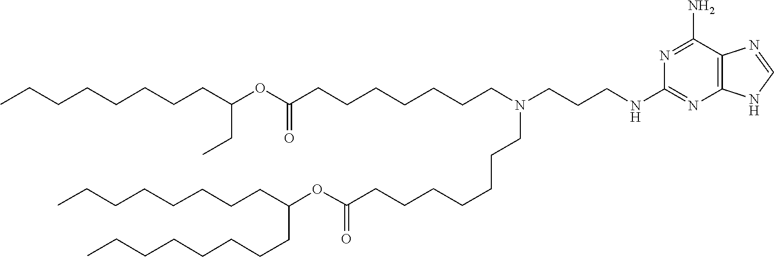 [SMILES: CCCCCCCCC(CC)OC(=O)CCCCCCCN(CCCCCCCC(=O)OC(CCCCCCCC)CCCCCCCC)CCCNc1nc(N)c2nc[nH]c2n1] |
| V-31 | 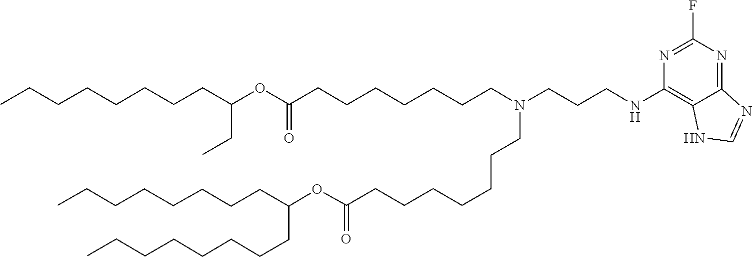 [SMILES: CCCCCCCCC(CC)OC(=O)CCCCCCCN(CCCCCCCC(=O)OC(CCCCCCCC)CCCCCCCC)CCCNc1nc(F)nc2nc[nH]c12] |
| V-32 | 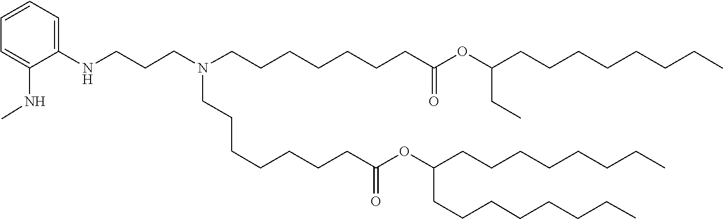 [SMILES: CCCCCCCCC(CC)OC(=O)CCCCCCCN(CCCCCCCC(=O)OC(CCCCCCCC)CCCCCCCC)CCCNc1ccccc1NC] |
| V-33 | 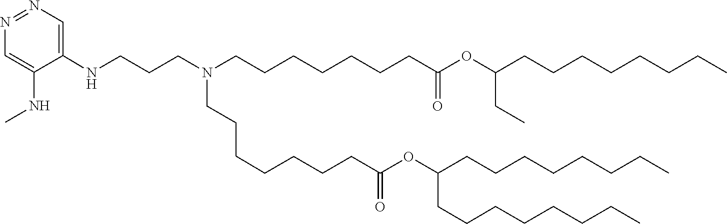 [SMILES: CCCCCCCCC(CC)OC(=O)CCCCCCCN(CCCCCCCC(=O)OC(CCCCCCCC)CCCCCCCC)CCCNc1cnncc1NC] |
| V-34 | 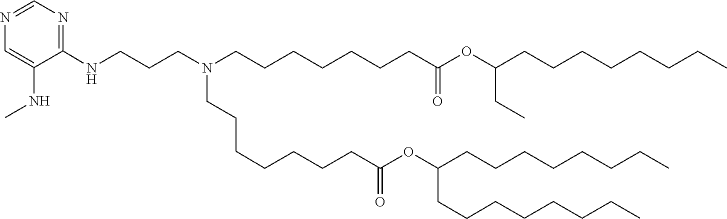 [SMILES: CCCCCCCCC(CC)OC(=O)CCCCCCCN(CCCCCCCC(=O)OC(CCCCCCCC)CCCCCCCC)CCCNc1ncncc1NC] |
| V-35 | 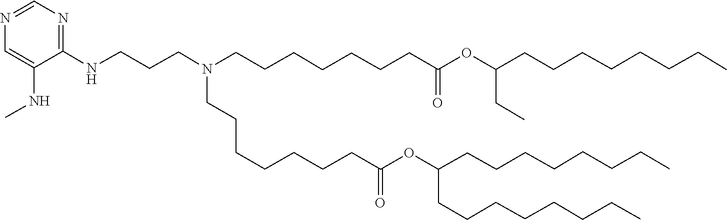 [SMILES: CCCCCCCCC(CC)OC(=O)CCCCCCCN(CCCCCCCC(=O)OC(CCCCCCCC)CCCCCCCC)CCCNc1ncncc1NC] |
| V-36 | 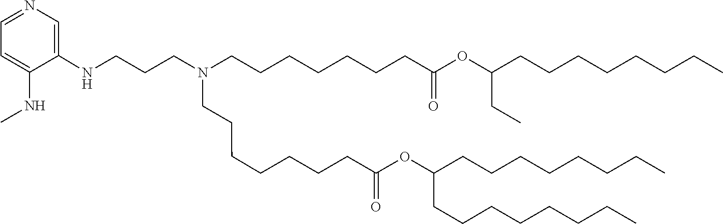 [SMILES: CCCCCCCCC(CC)OC(=O)CCCCCCCN(CCCCCCCC(=O)OC(CCCCCCCC)CCCCCCCC)CCCNc1cnccc1NC] |
| V-37 | 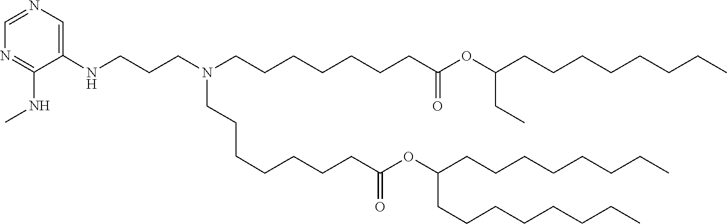 [SMILES: CCCCCCCCC(CC)OC(=O)CCCCCCCN(CCCCCCCC(=O)OC(CCCCCCCC)CCCCCCCC)CCCNc1cncnc1NC] |
| V-38 | 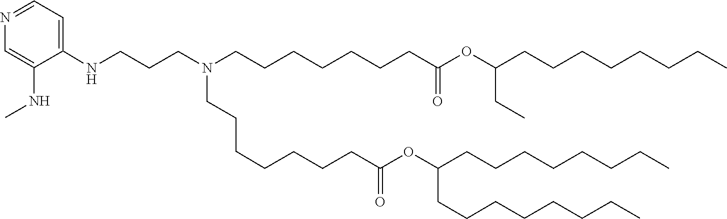 [SMILES: CCCCCCCCC(CC)OC(=O)CCCCCCCN(CCCCCCCC(=O)OC(CCCCCCCC)CCCCCCCC)CCCNc1ccncc1NC] |
| V-39 | 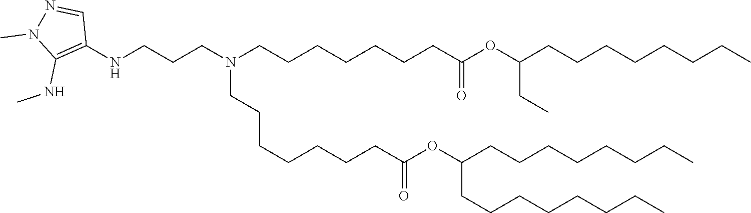 [SMILES: CCCCCCCCC(CC)OC(=O)CCCCCCCN(CCCCCCCC(=O)OC(CCCCCCCC)CCCCCCCC)CCCNc1cnn(C)c1NC] |
| V-40 | 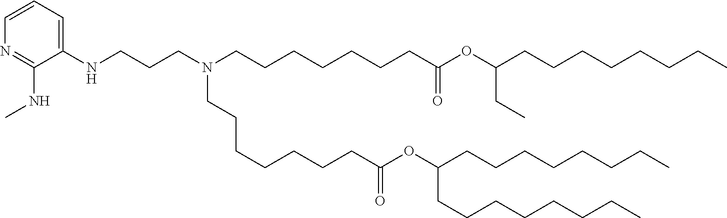 [SMILES: CCCCCCCCC(CC)OC(=O)CCCCCCCN(CCCCCCCC(=O)OC(CCCCCCCC)CCCCCCCC)CCCNc1cccnc1NC] |
| V-41 | 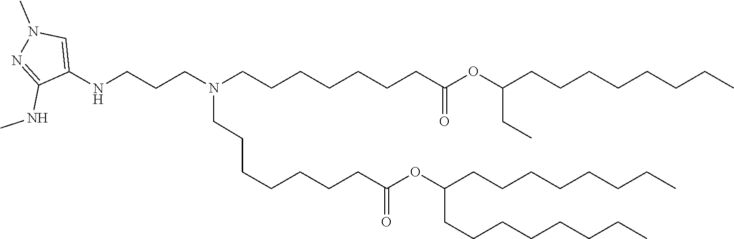 [SMILES: CCCCCCCCC(CC)OC(=O)CCCCCCCN(CCCCCCCC(=O)OC(CCCCCCCC)CCCCCCCC)CCCNc1cn(C)nc1NC] |
| V-42 | 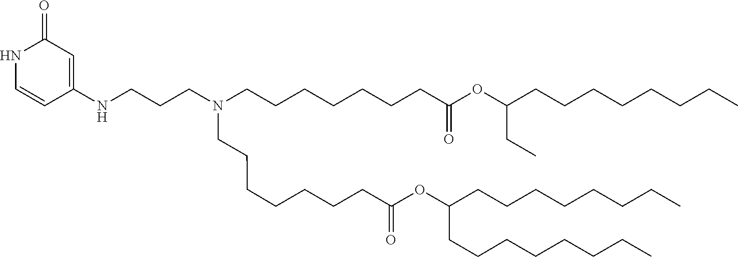 [SMILES: CCCCCCCCC(CC)OC(=O)CCCCCCCN(CCCCCCCC(=O)OC(CCCCCCCC)CCCCCCCC)CCCNc1cc[nH]c(=O)c1] |
| V-43 | 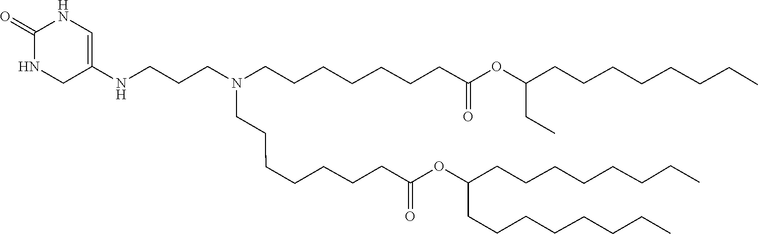 [SMILES: CCCCCCCCC(CC)OC(=O)CCCCCCCN(CCCCCCCC(=O)OC(CCCCCCCC)CCCCCCCC)CCCNC1=CNC(=O)NC1] |
| V-44 | 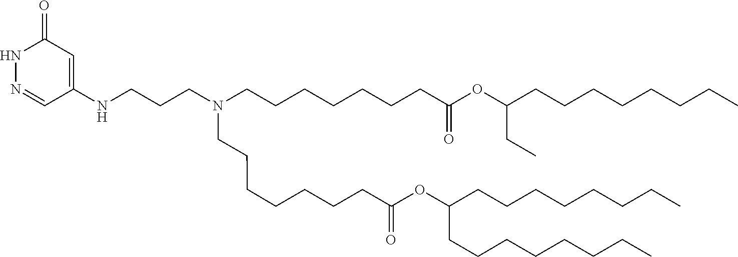 [SMILES: CCCCCCCCC(CC)OC(=O)CCCCCCCN(CCCCCCCC(=O)OC(CCCCCCCC)CCCCCCCC)CCCNc1cn[nH]c(=O)c1] |
| V-45 | 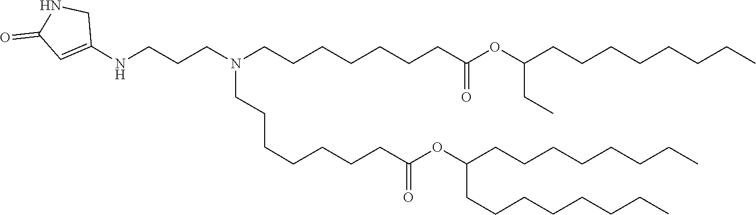 [SMILES: CCCCCCCCC(CC)OC(=O)CCCCCCCN(CCCCCCCC(=O)OC(CCCCCCCC)CCCCCCCC)CCCNC1=CC(=O)NC1] |
| V-46 | 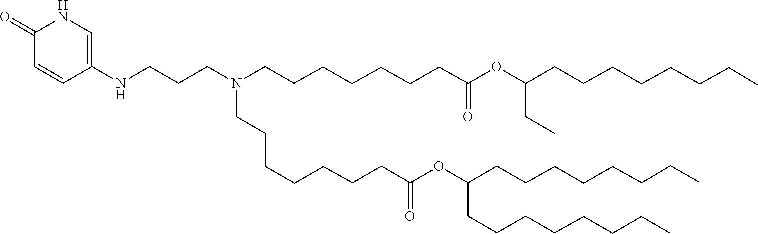 [SMILES: CCCCCCCCC(CC)OC(=O)CCCCCCCN(CCCCCCCC(=O)OC(CCCCCCCC)CCCCCCCC)CCCNc1ccc(=O)[nH]c1] |
| V-47 | 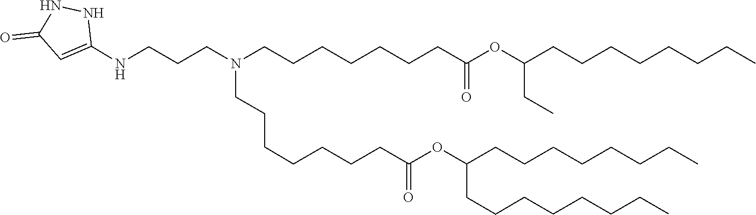 [SMILES: CCCCCCCCC(CC)OC(=O)CCCCCCCN(CCCCCCCC(=O)OC(CCCCCCCC)CCCCCCCC)CCCNc1cc(=O)[nH][nH]1] |
| V-48 | 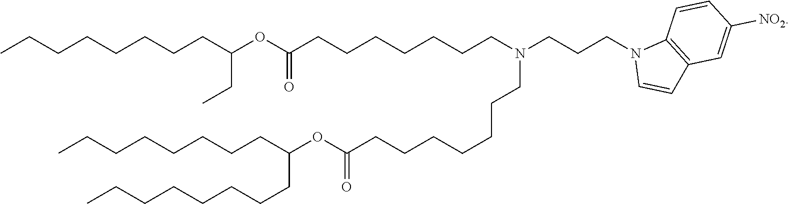 [SMILES: CCCCCCCCC(CC)OC(=O)CCCCCCCN(CCCCCCCC(=O)OC(CCCCCCCC)CCCCCCCC)CCCn1ccc2cc([N+](=O)[O-])ccc21] |
| V-49 | 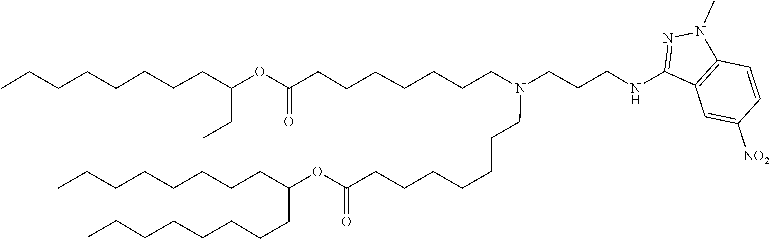 [SMILES: CCCCCCCCC(CC)OC(=O)CCCCCCCN(CCCCCCCC(=O)OC(CCCCCCCC)CCCCCCCC)CCCNc1nn(C)c2ccc([N+](=O)[O-])cc12] |
| V-50 | 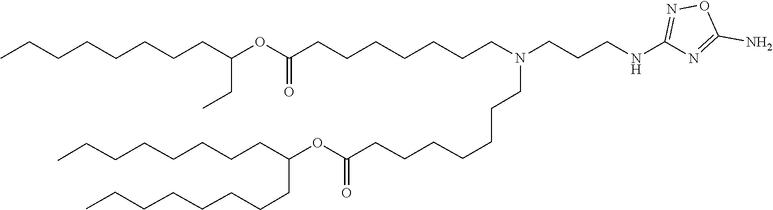 [SMILES: CCCCCCCCC(CC)OC(=O)CCCCCCCN(CCCCCCCC(=O)OC(CCCCCCCC)CCCCCCCC)CCCNc1noc(N)n1] |
| V-51 | 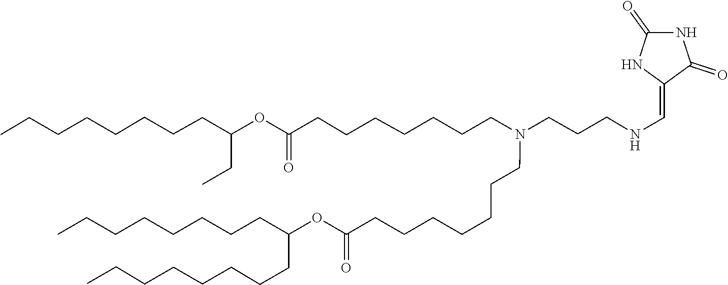 [SMILES: CCCCCCCCC(CC)OC(=O)CCCCCCCN(CCCCCCCC(=O)OC(CCCCCCCC)CCCCCCCC)CCCN/C=C1\NC(=O)NC1=O] |
| V-52 | 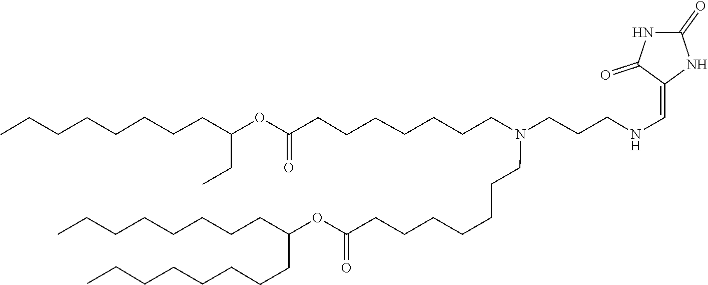 [SMILES: CCCCCCCCC(CC)OC(=O)CCCCCCCN(CCCCCCCC(=O)OC(CCCCCCCC)CCCCCCCC)CCCN/C=C1/NC(=O)NC1=O] |
| V-53 | 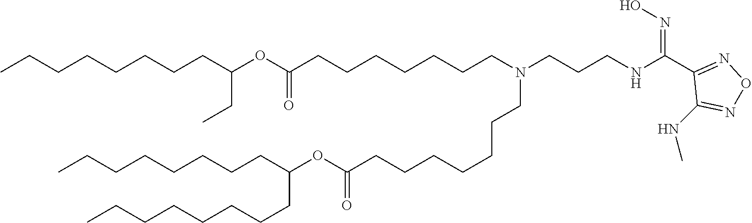 [SMILES: CCCCCCCCC(CC)OC(=O)CCCCCCCN(CCCCCCCC(=O)OC(CCCCCCCC)CCCCCCCC)CCCN/C(=N\O)c1nonc1NC] |
| V-54 | 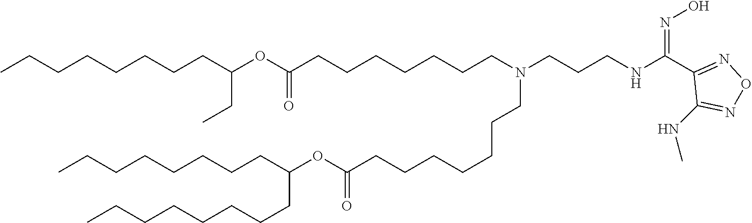 [SMILES: CCCCCCCCC(CC)OC(=O)CCCCCCCN(CCCCCCCC(=O)OC(CCCCCCCC)CCCCCCCC)CCCN/C(=N/O)c1nonc1NC] |
| V-55 | 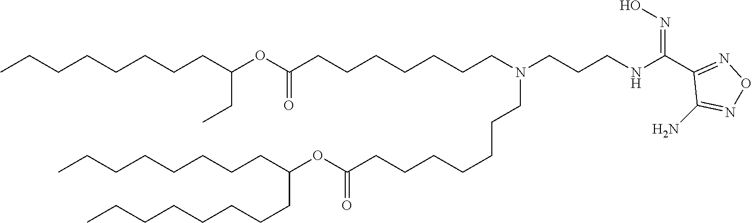 [SMILES: CCCCCCCCC(CC)OC(=O)CCCCCCCN(CCCCCCCC(=O)OC(CCCCCCCC)CCCCCCCC)CCCN/C(=N\O)c1nonc1N] |
| V-56 | 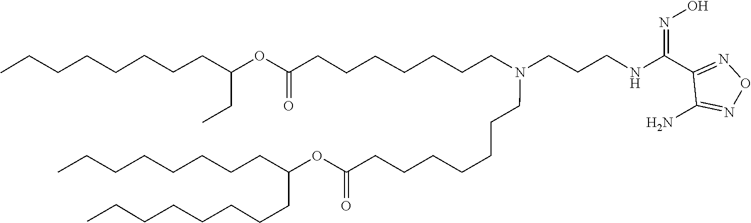 [SMILES: CCCCCCCCC(CC)OC(=O)CCCCCCCN(CCCCCCCC(=O)OC(CCCCCCCC)CCCCCCCC)CCCN/C(=N/O)c1nonc1N] |
| V-57 | 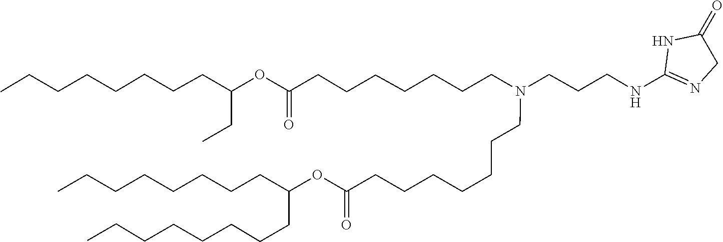 [SMILES: CCCCCCCCC(CC)OC(=O)CCCCCCCN(CCCCCCCC(=O)OC(CCCCCCCC)CCCCCCCC)CCCNC1=NCC(=O)N1] |

In some embodiments, the ionizable lipid is a compound of formula (IL-III):

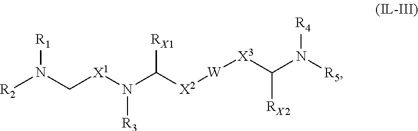

or salts or isomers thereof, wherein,
W is

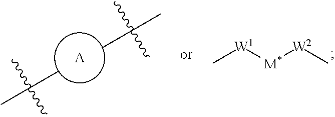

ring A is

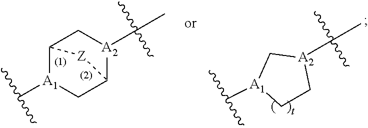

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6.

In some embodiments, the ionizable lipid is a compound of Formula (IL-IIIA):

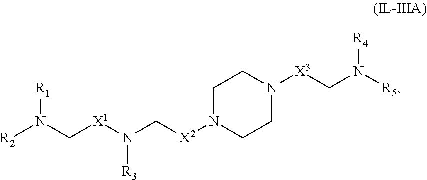

or a salt or isomer thereof, wherein
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
each M is independently selected from the group consisting

### of —C(O)O—, —OC(O)—, —OC(O)O—, —C(O)N(R′)—, —N(R′)C(O)—, —C(O)—, —C(S)—, —C(S)S—, —SC(S),

—CH(OH)—, —P(O)(OR′)O—, —S(O)₂—, an aryl group, and a heteroaryl group;
X¹, X², and X³ are independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl.

In some embodiments, the ionizable lipid is a compound selected from Table IL-7.

TABLE IL-7

| Cpd. # | Structure |
|---|---|
| VI-1 | 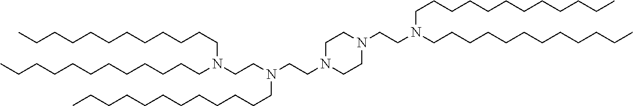 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1] |
| VI-2 | 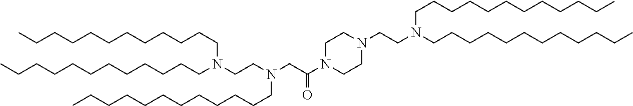 [SMILES: CCCCCCCCCCCCN(CCCCCCCCCCCC)CCN1CCN(C(=O)CN(CCCCCCCCCCCC)CCN(CCCCCCCCCCCC)CCCCCCCCCCCC)CC1] |
| VI-3 | 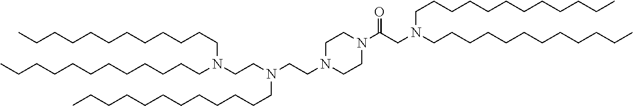 [SMILES: CCCCCCCCCCCCN(CCCCCCCCCCCC)CCN(CCCCCCCCCCCC)CCN1CCN(C(=O)CN(CCCCCCCCCCCC)CCCCCCCCCCCC)CC1] |
| VI-4 | 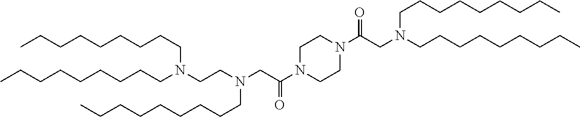 [SMILES: CCCCCCCCCN(CCCCCCCCC)CCN(CCCCCCCCC)CC(=O)N1CCN(C(=O)CN(CCCCCCCCC)CCCCCCCCC)CC1] |
| VI-5 | 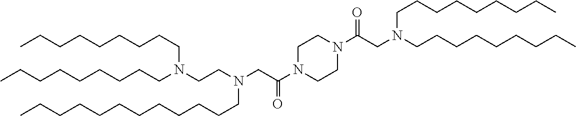 [SMILES: CCCCCCCCCCCCN(CCN(CCCCCCCCC)CCCCCCCCC)CC(=O)N1CCN(C(=O)CN(CCCCCCCCC)CCCCCCCCC)CC1] |
| VI-6 | 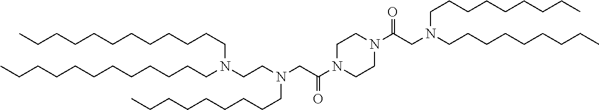 [SMILES: CCCCCCCCCCCCN(CCCCCCCCCCCC)CCN(CCCCCCCCC)CC(=O)N1CCN(C(=O)CN(CCCCCCCCC)CCCCCCCCC)CC1] |
| VI-7 | 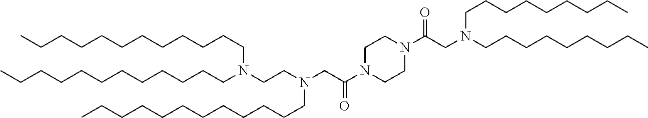 [SMILES: CCCCCCCCCCCCN(CCCCCCCCCCCC)CCN(CCCCCCCCCCCC)CC(=O)N1CCN(C(=O)CN(CCCCCCCCC)CCCCCCCCC)CC1] |
| VI-8 | 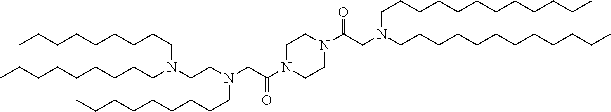 [SMILES: CCCCCCCCCCCCN(CCCCCCCCCCCC)CC(=O)N1CCN(C(=O)CN(CCCCCCCCC)CCN(CCCCCCCCC)CCCCCCCCC)CC1] |
| VI-9 | 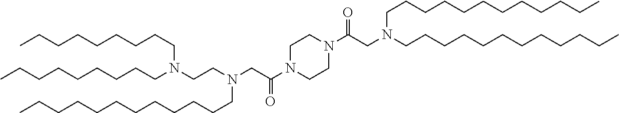 [SMILES: CCCCCCCCCCCCN(CCCCCCCCCCCC)CC(=O)N1CCN(C(=O)CN(CCCCCCCCCCCC)CCN(CCCCCCCCC)CCCCCCCCC)CC1] |
| VI-10 | 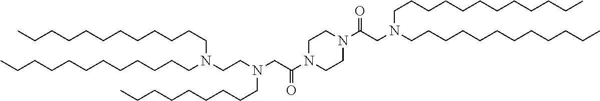 [SMILES: CCCCCCCCCCCCN(CCCCCCCCCCCC)CCN(CCCCCCCCC)CC(=O)N1CCN(C(=O)CN(CCCCCCCCCCCC)CCCCCCCCCCCC)CC1] |
| VI-11 | 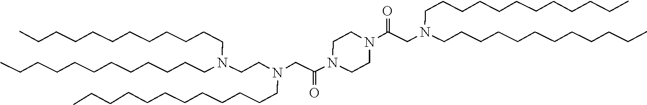 [SMILES: CCCCCCCCCCCCN(CCCCCCCCCCCC)CCN(CCCCCCCCCCCC)CC(=O)N1CCN(C(=O)CN(CCCCCCCCCCCC)CCCCCCCCCCCC)CC1] |
| VI-12 | 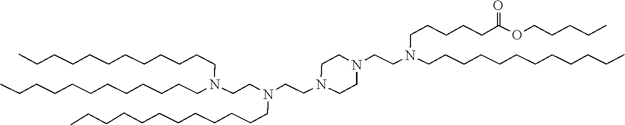 [SMILES: CCCCCCCCCCCCN(CCCCCCCCCCCC)CCN(CCCCCCCCCCCC)CCN1CCN(CCN(CCCCCCCCCCCC)CCCCCC(=O)OCCCCC)CC1] |
| VI-13 | 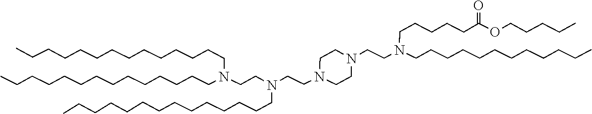 [SMILES: CCCCCCCCCCCCCCN(CCCCCCCCCCCCCC)CCN(CCCCCCCCCCCCCC)CCN1CCN(CCN(CCCCCCCCCCCC)CCCCCC(=O)OCCCCC)CC1] |
| VI-14 | 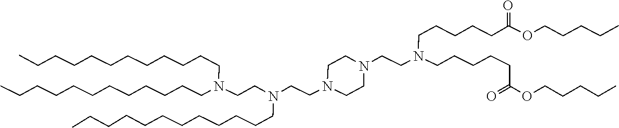 [SMILES: CCCCCCCCCCCCN(CCCCCCCCCCCC)CCN(CCCCCCCCCCCC)CCN1CCN(CCN(CCCCCC(=O)OCCCCC)CCCCCC(=O)OCCCCC)CC1] |
| VI-15 | 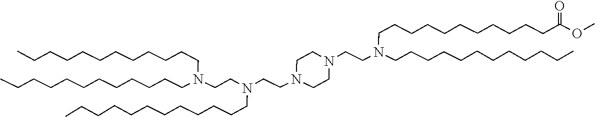 [SMILES: CCCCCCCCCCCCN(CCCCCCCCCCCC)CCN(CCCCCCCCCCCC)CCN1CCN(CCN(CCCCCCCCCCCC)CCCCCCCCCCCC(=O)OC)CC1] |
| VI-16 | 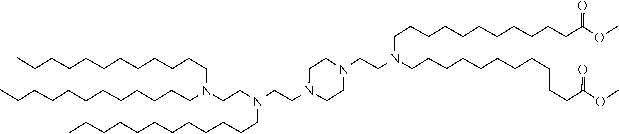 [SMILES: CCCCCCCCCCCCN(CCCCCCCCCCCC)CCN(CCCCCCCCCCCC)CCN1CCN(CCN(CCCCCCCCCCCC(=O)OC)CCCCCCCCCCCC(=O)OC)CC1] |
| VI-17 | 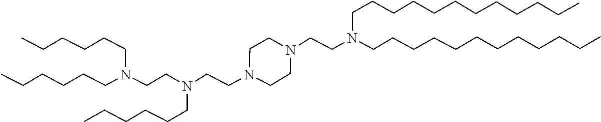 [SMILES: CCCCCCCCCCCCN(CCCCCCCCCCCC)CCN1CCN(CCN(CCCCCC)CCN(CCCCCC)CCCCCC)CC1] |
| VI-18 | 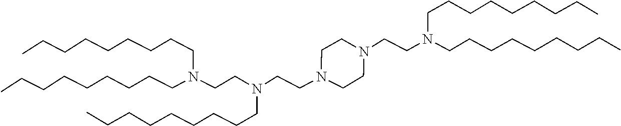 [SMILES: CCCCCCCCCN(CCCCCCCCC)CCN(CCCCCCCCC)CCN1CCN(CCN(CCCCCCCCC)CCCCCCCCC)CC1] |
| VI-19 | 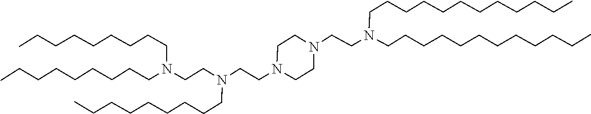 [SMILES: CCCCCCCCCCCCN(CCCCCCCCCCCC)CCN1CCN(CCN(CCCCCCCCC)CCN(CCCCCCCCC)CCCCCCCCC)CC1] |
| VI-20 | 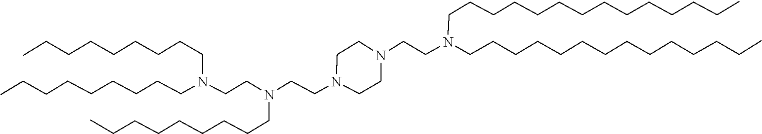 [SMILES: CCCCCCCCCCCCCCN(CCCCCCCCCCCCCC)CCN1CCN(CCN(CCCCCCCCC)CCN(CCCCCCCCC)CCCCCCCCC)CC1] |
| VI-21 | 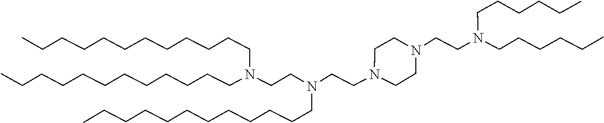 [SMILES: CCCCCCCCCCCCN(CCCCCCCCCCCC)CCN(CCCCCCCCCCCC)CCN1CCN(CCN(CCCCCC)CCCCCC)CC1] |
| VI-22 | 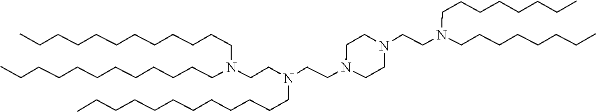 [SMILES: CCCCCCCCCCCCN(CCCCCCCCCCCC)CCN(CCCCCCCCCCCC)CCN1CCN(CCN(CCCCCCCC)CCCCCCCC)CC1] |
| VI-23 | 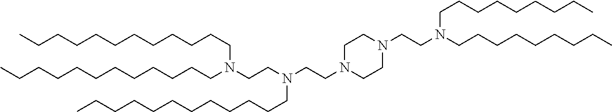 [SMILES: CCCCCCCCCCCCN(CCCCCCCCCCCC)CCN(CCCCCCCCCCCC)CCN1CCN(CCN(CCCCCCCCC)CCCCCCCCC)CC1] |
| VI-24 | 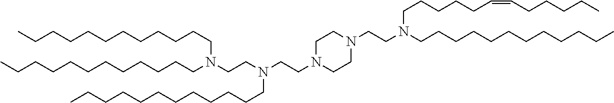 [SMILES: CCCCC/C=C\CCCCCN(CCCCCCCCCCCC)CCN1CCN(CCN(CCCCCCCCCCCC)CCN(CCCCCCCCCCCC)CCCCCCCCCCCC)CC1] |
| VI-25 | 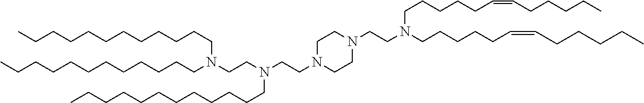 [SMILES: CCCCC/C=C\CCCCCN(CCCCC/C=C\CCCCC)CCN1CCN(CCN(CCCCCCCCCCCC)CCN(CCCCCCCCCCCC)CCCCCCCCCCCC)CC1] |
| VI-26 | 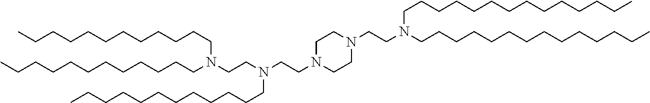 [SMILES: CCCCCCCCCCCCCCN(CCCCCCCCCCCCCC)CCN1CCN(CCN(CCCCCCCCCCCC)CCN(CCCCCCCCCCCC)CCCCCCCCCCCC)CC1] |
| VI-27 | 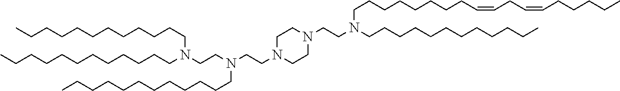 [SMILES: CCCCC/C=C\C/C=C\CCCCCCCCN(CCCCCCCCCCCC)CCN1CCN(CCN(CCCCCCCCCCCC)CCN(CCCCCCCCCCCC)CCCCCCCCCCCC)CC1] |
| VI-28 | 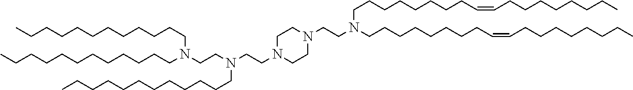 [SMILES: CCCCCCCC/C=C\CCCCCCCCN(CCCCCCCC/C=C\CCCCCCCC)CCN1CCN(CCN(CCCCCCCCCCCC)CCN(CCCCCCCCCCCC)CCCCCCCCCCCC)CC1] |
| VI-29 | 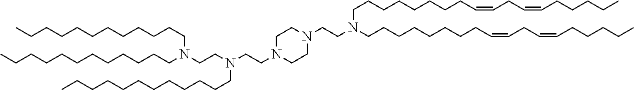 [SMILES: CCCCC/C=C\C/C=C\CCCCCCCCN(CCCCCCCC/C=C\C/C=C\CCCCC)CCN1CCN(CCN(CCCCCCCCCCCC)CCN(CCCCCCCCCCCC)CCCCCCCCCCCC)CC1] |
| VI-30 | 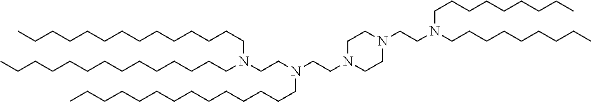 [SMILES: CCCCCCCCCCCCCCN(CCCCCCCCCCCCCC)CCN(CCCCCCCCCCCCCC)CCN1CCN(CCN(CCCCCCCCC)CCCCCCCCC)CC1] |
| VI-31 | 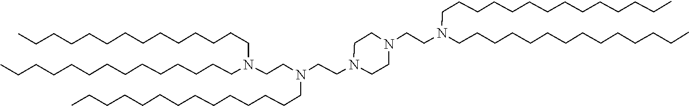 [SMILES: CCCCCCCCCCCCCCN(CCCCCCCCCCCCCC)CCN(CCCCCCCCCCCCCC)CCN1CCN(CCN(CCCCCCCCCCCCCC)CCCCCCCCCCCCCC)CC1] |
| VI-32 | 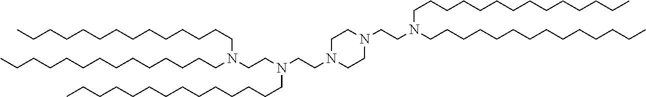 [SMILES: CCCCCCCCCCCCCCN(CCCCCCCCCCCCCC)CCN(CCCCCCCCCCCCCC)CCN1CCN(CCN(CCCCCCCCCCCCCC)CCCCCCCCCCCCCC)CC1] |
| VI-33 | 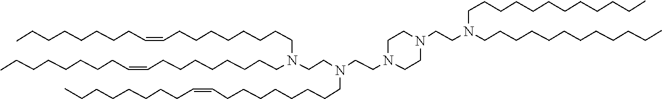 [SMILES: CCCCCCCC/C=C\CCCCCCCCN(CCCCCCCC/C=C\CCCCCCCC)CCN(CCCCCCCC/C=C\CCCCCCCC)CCN1CCN(CCN(CCCCCCCCCCCC)CCCCCCCCCCCC)CC1] |
| VI-34 | 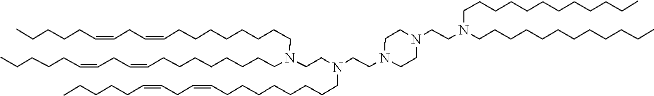 [SMILES: CCCCC/C=C\C/C=C\CCCCCCCCN(CCCCCCCC/C=C\C/C=C\CCCCC)CCN(CCCCCCCC/C=C\C/C=C\CCCCC)CCN1CCN(CCN(CCCCCCCCCCCC)CCCCCCCCCCCC)CC1] |
| VI-35 | 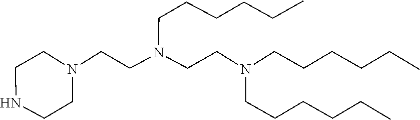 [SMILES: CCCCCCN(CCCCCC)CCN(CCCCCC)CCN1CCNCC1] |
| VI-36 | 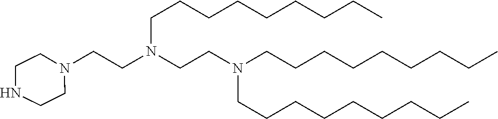 [SMILES: CCCCCCCCCN(CCCCCCCCC)CCN(CCCCCCCCC)CCN1CCNCC1] |
| VI-37 | 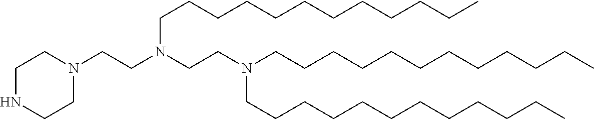 [SMILES: CCCCCCCCCCCCN(CCCCCCCCCCCC)CCN(CCCCCCCCCCCC)CCN1CCNCC1] |
| VI-38 | 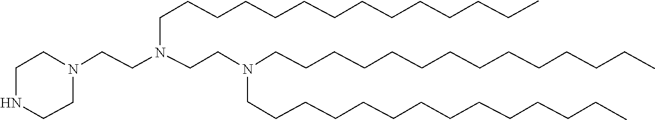 [SMILES: CCCCCCCCCCCCCCN(CCCCCCCCCCCCCC)CCN(CCCCCCCCCCCCCC)CCN1CCNCC1] |
| VI-39 | 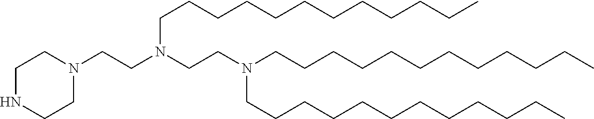 [SMILES: CCCCCCCCCCCCN(CCCCCCCCCCCC)CCN(CCCCCCCCCCCC)CCN1CCNCC1] |
| VI-40 | 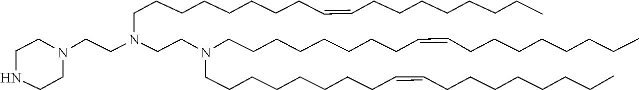 [SMILES: CCCCCCCC/C=C\CCCCCCCCN(CCCCCCCC/C=C\CCCCCCCC)CCN(CCCCCCCC/C=C\CCCCCCCC)CCN1CCNCC1] |
| VI-41 | 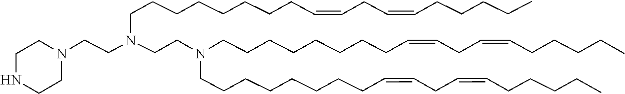 [SMILES: CCCCC/C=C\C/C=C\CCCCCCCCN(CCCCCCCC/C=C\C/C=C\CCCCC)CCN(CCCCCCCC/C=C\C/C=C\CCCCC)CCN1CCNCC1] |
| VI-IV | 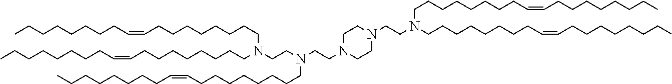 [SMILES: CCCCCCCC/C=C\CCCCCCCCN(CCCCCCCC/C=C\CCCCCCCC)CCN(CCCCCCCC/C=C\CCCCCCCC)CCN1CCN(CCN(CCCCCCCC/C=C\CCCCCCCC)CCCCCCCC/C=C\CCCCCCCC)CC1] |
| VI-42 | 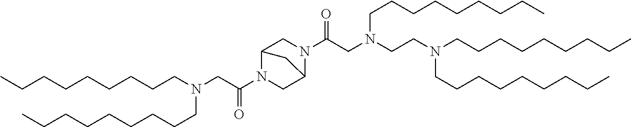 [SMILES: CCCCCCCCCN(CCCCCCCCC)CCN(CCCCCCCCC)CC(=O)N1CC2CC1CN2C(=O)CN(CCCCCCCCC)CCCCCCCCC] |
| VI-43 | 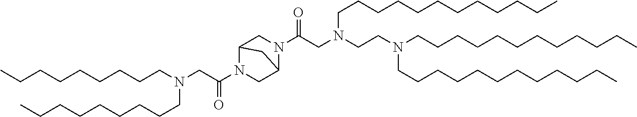 [SMILES: CCCCCCCCCCCCN(CCCCCCCCCCCC)CCN(CCCCCCCCCCCC)CC(=O)N1CC2CC1CN2C(=O)CN(CCCCCCCCC)CCCCCCCCC] |
| VI-44 | 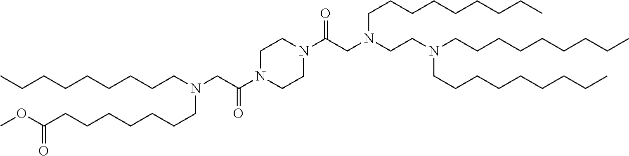 [SMILES: CCCCCCCCCN(CCCCCCCCC)CCN(CCCCCCCCC)CC(=O)N1CCN(C(=O)CN(CCCCCCCCC)CCCCCCCC(=O)OC)CC1] |
| VI-45 | 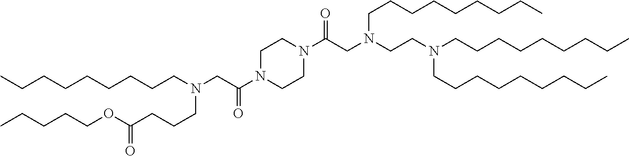 [SMILES: CCCCCCCCCN(CCCCCCCCC)CCN(CCCCCCCCC)CC(=O)N1CCN(C(=O)CN(CCCCCCCCC)CCCC(=O)OCCCCC)CC1] |
| VI-46 | 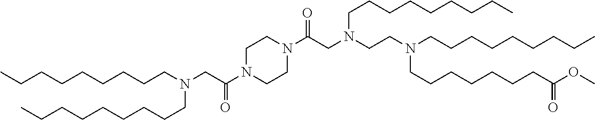 [SMILES: CCCCCCCCCN(CCCCCCCC(=O)OC)CCN(CCCCCCCCC)CC(=O)N1CCN(C(=O)CN(CCCCCCCCC)CCCCCCCCC)CC1] |
| VI-47 | 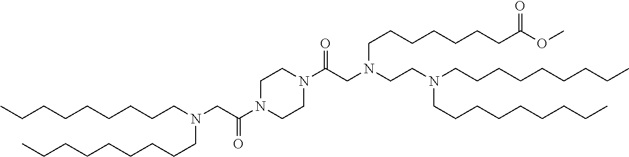 [SMILES: CCCCCCCCCN(CCCCCCCCC)CCN(CCCCCCCC(=O)OC)CC(=O)N1CCN(C(=O)CN(CCCCCCCCC)CCCCCCCCC)CC1] |
| VI-48 | 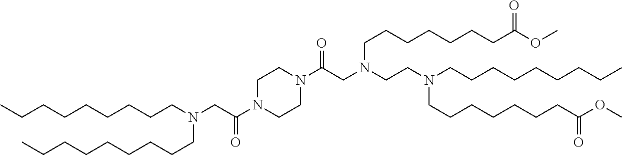 [SMILES: CCCCCCCCCN(CCCCCCCC(=O)OC)CCN(CCCCCCCC(=O)OC)CC(=O)N1CCN(C(=O)CN(CCCCCCCCC)CCCCCCCCC)CC1] |
| VI-49 | 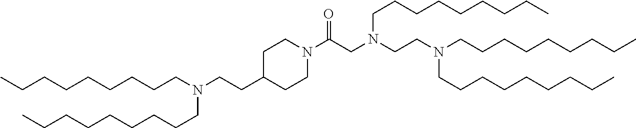 [SMILES: CCCCCCCCCN(CCCCCCCCC)CCC1CCN(C(=O)CN(CCCCCCCCC)CCN(CCCCCCCCC)CCCCCCCCC)CC1] |
| VI-50 | 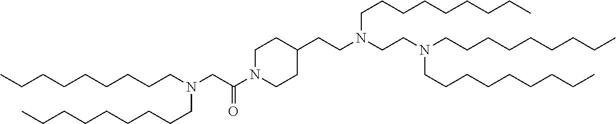 [SMILES: CCCCCCCCCN(CCCCCCCCC)CCN(CCCCCCCCC)CCC1CCN(C(=O)CN(CCCCCCCCC)CCCCCCCCC)CC1] |
| VI-51 | 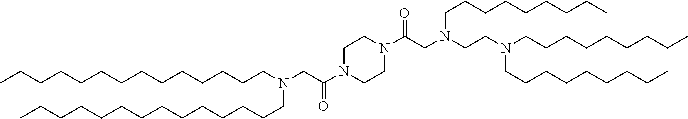 [SMILES: CCCCCCCCCCCCCCN(CCCCCCCCCCCCCC)CC(=O)N1CCN(C(=O)CN(CCCCCCCCC)CCN(CCCCCCCCC)CCCCCCCCC)CC1] |
| VI-52 | 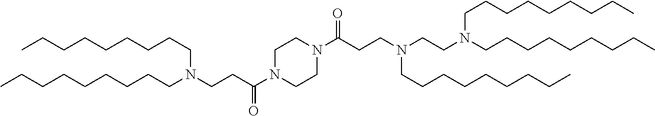 [SMILES: CCCCCCCCCN(CCCCCCCCC)CCC(=O)N1CCN(C(=O)CCN(CCCCCCCCC)CCN(CCCCCCCCC)CCCCCCCCC)CC1] |
| VI-53 | 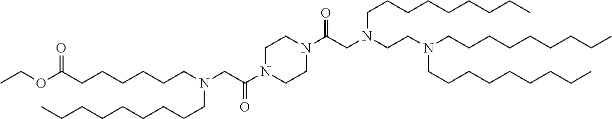 [SMILES: CCCCCCCCCN(CCCCCCCCC)CCN(CCCCCCCCC)CC(=O)N1CCN(C(=O)CN(CCCCCCCCC)CCCCCCC(=O)OCC)CC1] |
| VI-54 | 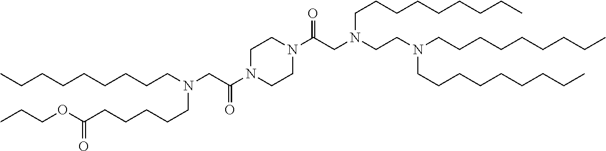 [SMILES: CCCCCCCCCN(CCCCCCCCC)CCN(CCCCCCCCC)CC(=O)N1CCN(C(=O)CN(CCCCCCCCC)CCCCCC(=O)OCCC)CC1] |
| VI-55 | 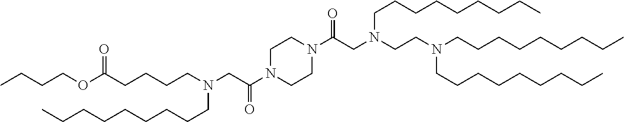 [SMILES: CCCCCCCCCN(CCCCCCCCC)CCN(CCCCCCCCC)CC(=O)N1CCN(C(=O)CN(CCCCCCCCC)CCCCC(=O)OCCCC)CC1] |
| VI-56 | 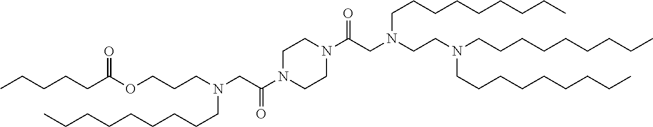 [SMILES: CCCCCCCCCN(CCCCCCCCC)CCN(CCCCCCCCC)CC(=O)N1CCN(C(=O)CN(CCCCCCCCC)CCCOC(=O)CCCCC)CC1] |
| VI-57 | 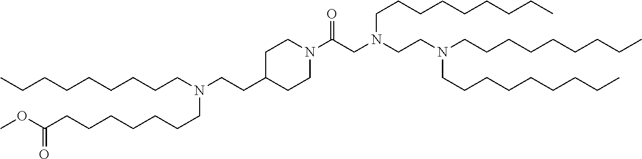 [SMILES: CCCCCCCCCN(CCCCCCCC(=O)OC)CCC1CCN(C(=O)CN(CCCCCCCCC)CCN(CCCCCCCCC)CCCCCCCCC)CC1] |
| VI-58 | 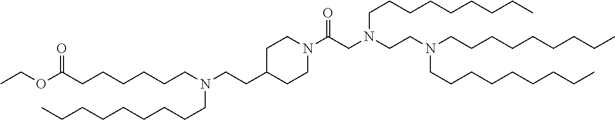 [SMILES: CCCCCCCCCN(CCCCCCC(=O)OCC)CCC1CCN(C(=O)CN(CCCCCCCCC)CCN(CCCCCCCCC)CCCCCCCCC)CC1] |
| VI-59 | 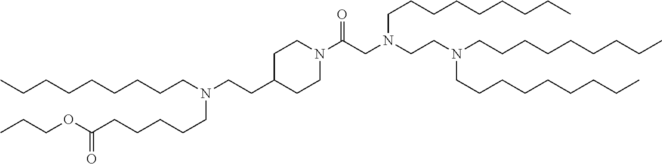 [SMILES: CCCCCCCCCN(CCCCCC(=O)OCCC)CCC1CCN(C(=O)CN(CCCCCCCCC)CCN(CCCCCCCCC)CCCCCCCCC)CC1] |
| VI-60 | 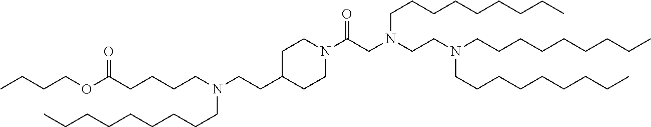 [SMILES: CCCCCCCCCN(CCCCC(=O)OCCCC)CCC1CCN(C(=O)CN(CCCCCCCCC)CCN(CCCCCCCCC)CCCCCCCCC)CC1] |
| VI-61 | 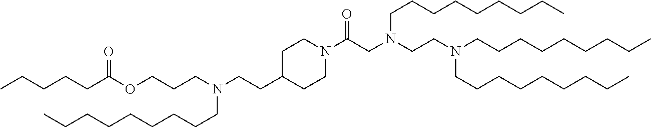 [SMILES: CCCCCCCCCN(CCCOC(=O)CCCCC)CCC1CCN(C(=O)CN(CCCCCCCCC)CCN(CCCCCCCCC)CCCCCCCCC)CC1] |
| VI-62 | 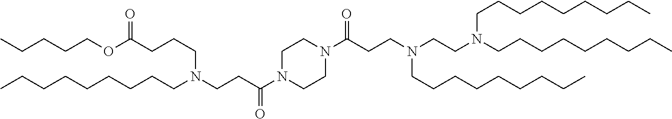 [SMILES: CCCCCCCCCN(CCCC(=O)OCCCCC)CCC(=O)N1CCN(C(=O)CCN(CCCCCCCCC)CCN(CCCCCCCCC)CCCCCCCCC)CC1] |
| VI-63 | 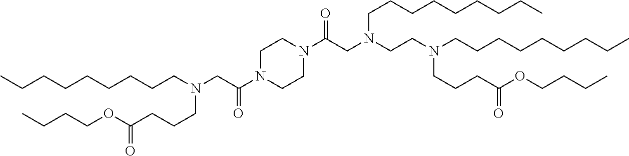 [SMILES: CCCCCCCCCN(CCCC(=O)OCCCC)CCN(CCCCCCCCC)CC(=O)N1CCN(C(=O)CN(CCCCCCCCC)CCCC(=O)OCCCC)CC1] |
| VI-64 | 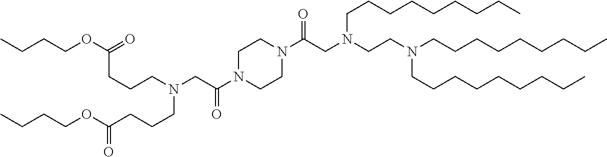 [SMILES: CCCCCCCCCN(CCCCCCCCC)CCN(CCCCCCCCC)CC(=O)N1CCN(C(=O)CN(CCCC(=O)OCCCC)CCCC(=O)OCCCC)CC1] |
| VI-65 | 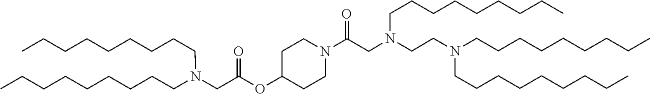 [SMILES: CCCCCCCCCN(CCCCCCCCC)CCN(CCCCCCCCC)CC(=O)N1CCC(OC(=O)CN(CCCCCCCCC)CCCCCCCCC)CC1] |
| VI-66 | 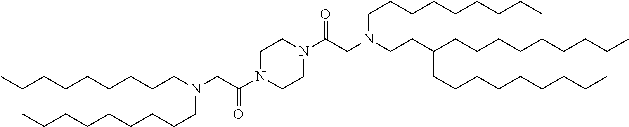 [SMILES: CCCCCCCCCC(CCCCCCCCC)CCN(CCCCCCCCC)CC(=O)N1CCN(C(=O)CN(CCCCCCCCC)CCCCCCCCC)CC1] |
| VI-67 | 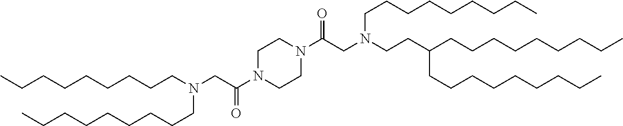 [SMILES: CCCCCCCCCC(CCCCCCCCC)CCN(CCCCCCCCC)CC(=O)N1CCN(C(=O)CN(CCCCCCCCC)CCCCCCCCC)CC1] |
| VI-68 | 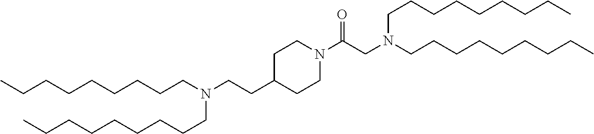 [SMILES: CCCCCCCCCN(CCCCCCCCC)CCC1CCN(C(=O)CN(CCCCCCCCC)CCCCCCCCC)CC1] |
| VI-69 | 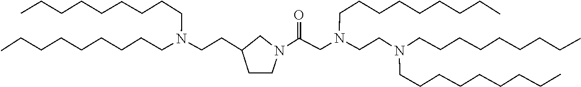 [SMILES: CCCCCCCCCN(CCCCCCCCC)CCC1CCN(C(=O)CN(CCCCCCCCC)CCN(CCCCCCCCC)CCCCCCCCC)C1] |
| VI-70 | 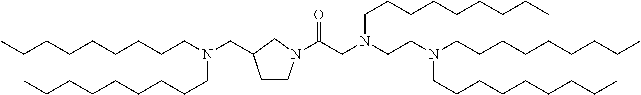 [SMILES: CCCCCCCCCN(CCCCCCCCC)CCN(CCCCCCCCC)CC(=O)N1CCC(CN(CCCCCCCCC)CCCCCCCCC)C1] |
| VI-71 | 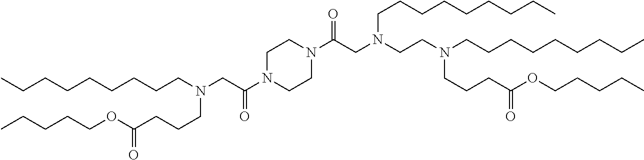 [SMILES: CCCCCCCCCN(CCCC(=O)OCCCCC)CCN(CCCCCCCCC)CC(=O)N1CCN(C(=O)CN(CCCCCCCCC)CCCC(=O)OCCCCC)CC1] |
| VI-72 | 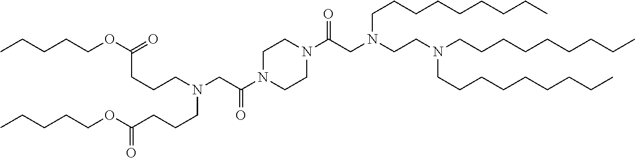 [SMILES: CCCCCCCCCN(CCCCCCCCC)CCN(CCCCCCCCC)CC(=O)N1CCN(C(=O)CN(CCCC(=O)OCCCCC)CCCC(=O)OCCCCC)CC1] |
| VI-73 | 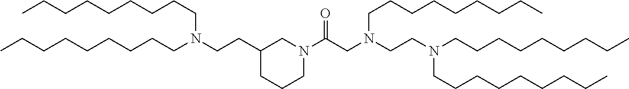 [SMILES: CCCCCCCCCN(CCCCCCCCC)CCC1CCCN(C(=O)CN(CCCCCCCCC)CCN(CCCCCCCCC)CCCCCCCCC)C1] |
| VI-74 | 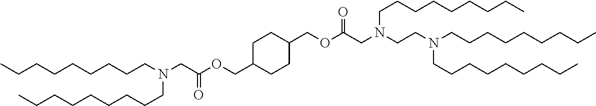 [SMILES: CCCCCCCCCN(CCCCCCCCC)CCN(CCCCCCCCC)CC(=O)OCC1CCC(COC(=O)CN(CCCCCCCCC)CCCCCCCCC)CC1] |
| VI-75 | 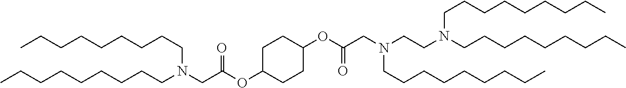 [SMILES: CCCCCCCCCN(CCCCCCCCC)CCN(CCCCCCCCC)CC(=O)OC1CCC(OC(=O)CN(CCCCCCCCC)CCCCCCCCC)CC1] |
| VI-76 | 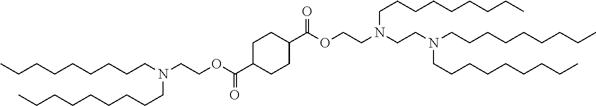 [SMILES: CCCCCCCCCN(CCCCCCCCC)CCOC(=O)C1CCC(C(=O)OCCN(CCCCCCCCC)CCN(CCCCCCCCC)CCCCCCCCC)CC1] |
| VI-77 | 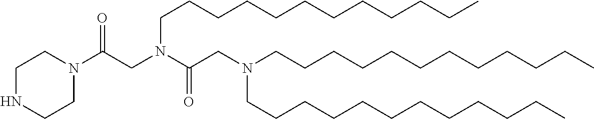 [SMILES: CCCCCCCCCCCCN(CCCCCCCCCCCC)CC(=O)N(CCCCCCCCCCCC)CC(=O)N1CCNCC1] |
| VI-78 | 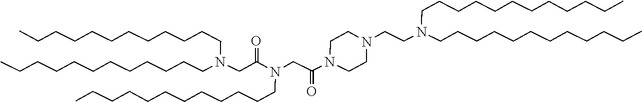 [SMILES: CCCCCCCCCCCCN(CCCCCCCCCCCC)CCN1CCN(C(=O)CN(CCCCCCCCCCCC)C(=O)CN(CCCCCCCCCCCC)CCCCCCCCCCCC)CC1] |
| VI-79 | 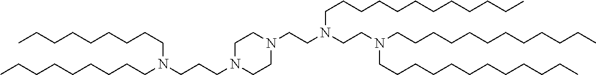 [SMILES: CCCCCCCCCCCCN(CCCCCCCCCCCC)CCN(CCCCCCCCCCCC)CCN1CCN(CCCN(CCCCCCCCC)CCCCCCCCC)CC1] |
| VI-80 | 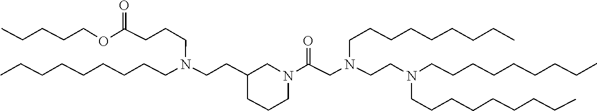 [SMILES: CCCCCCCCCN(CCCC(=O)OCCCCC)CCC1CCCN(C(=O)CN(CCCCCCCCC)CCN(CCCCCCCCC)CCCCCCCCC)C1] |
| VI-81 | 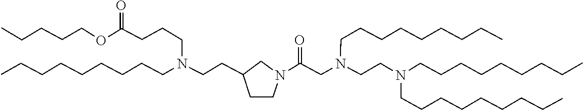 [SMILES: CCCCCCCCCN(CCCC(=O)OCCCCC)CCC1CCN(C(=O)CN(CCCCCCCCC)CCN(CCCCCCCCC)CCCCCCCCC)C1] |
| VI-82 | 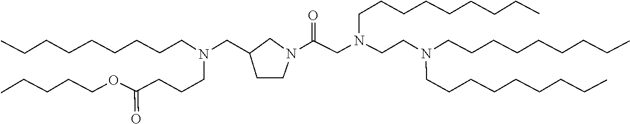 [SMILES: CCCCCCCCCN(CCCCCCCCC)CCN(CCCCCCCCC)CC(=O)N1CCC(CN(CCCCCCCCC)CCCC(=O)OCCCCC)C1] |
| VI-83 | 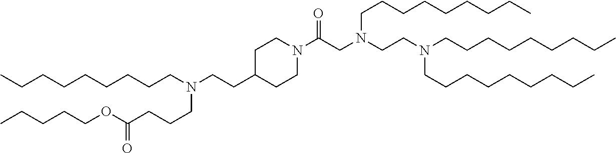 [SMILES: CCCCCCCCCN(CCCC(=O)OCCCCC)CCC1CCN(C(=O)CN(CCCCCCCCC)CCN(CCCCCCCCC)CCCCCCCCC)CC1] |
| VI-84 | 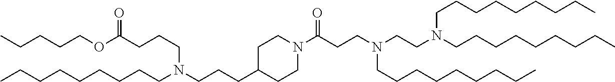 [SMILES: CCCCCCCCCN(CCCC(=O)OCCCCC)CCCC1CCN(C(=O)CCN(CCCCCCCCC)CCN(CCCCCCCCC)CCCCCCCCC)CC1] |
| VI-85 | 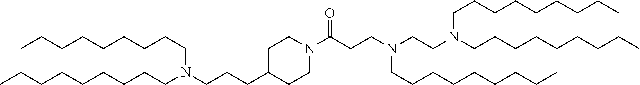 [SMILES: CCCCCCCCCN(CCCCCCCCC)CCCC1CCN(C(=O)CCN(CCCCCCCCC)CCN(CCCCCCCCC)CCCCCCCCC)CC1] |
| VI-86 | 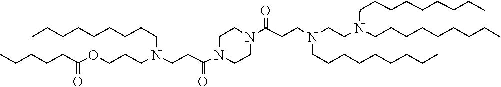 [SMILES: CCCCCCCCCN(CCCOC(=O)CCCCC)CCC(=O)N1CCN(C(=O)CCN(CCCCCCCCC)CCN(CCCCCCCCC)CCCCCCCCC)CC1] |
| VI-87 | 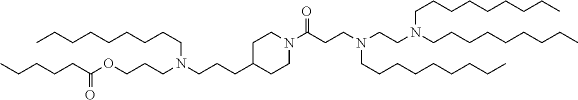 [SMILES: CCCCCCCCCN(CCCOC(=O)CCCCC)CCCC1CCN(C(=O)CCN(CCCCCCCCC)CCN(CCCCCCCCC)CCCCCCCCC)CC1] |
| VI-88 | 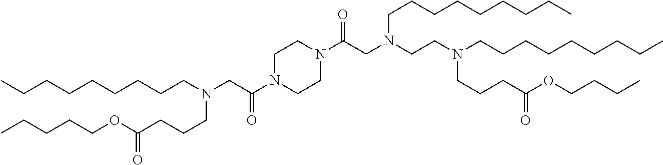 [SMILES: CCCCCCCCCN(CCCC(=O)OCCCC)CCN(CCCCCCCCC)CC(=O)N1CCN(C(=O)CN(CCCCCCCCC)CCCC(=O)OCCCCC)CC1] |
| VI-89 | 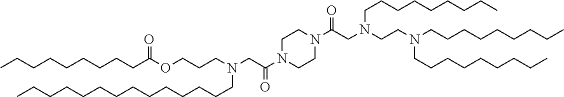 [SMILES: CCCCCCCCCCCCCCN(CCCOC(=O)CCCCCCCCC)CC(=O)N1CCN(C(=O)CN(CCCCCCCCC)CCN(CCCCCCCCC)CCCCCCCCC)CC1] |
| VI-90 | 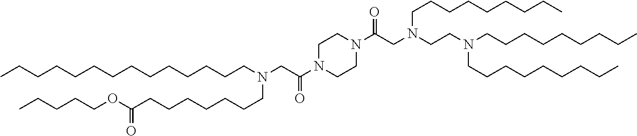 [SMILES: CCCCCCCCCCCCCCN(CCCCCCCC(=O)OCCCCC)CC(=O)N1CCN(C(=O)CN(CCCCCCCCC)CCN(CCCCCCCCC)CCCCCCCCC)CC1] |
| VI-91 | 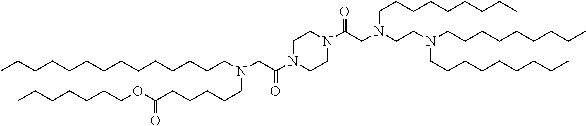 [SMILES: CCCCCCCCCCCCCCN(CCCCCC(=O)OCCCCCCC)CC(=O)N1CCN(C(=O)CN(CCCCCCCCC)CCN(CCCCCCCCC)CCCCCCCCC)CC1] |
| VI-92 | 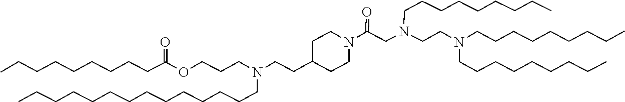 [SMILES: CCCCCCCCCCCCCCN(CCCOC(=O)CCCCCCCCC)CCC1CCN(C(=O)CN(CCCCCCCCC)CCN(CCCCCCCCC)CCCCCCCCC)CC1] |
| VI-93 | 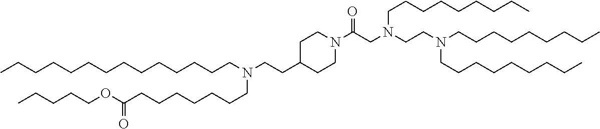 [SMILES: CCCCCCCCCCCCCCN(CCCCCCCC(=O)OCCCCC)CCC1CCN(C(=O)CN(CCCCCCCCC)CCN(CCCCCCCCC)CCCCCCCCC)CC1] |
| VI-94 | 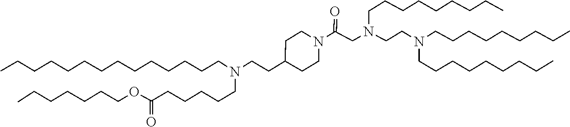 [SMILES: CCCCCCCCCCCCCCN(CCCCCC(=O)OCCCCCCC)CCC1CCN(C(=O)CN(CCCCCCCCC)CCN(CCCCCCCCC)CCCCCCCCC)CC1] |
| VI-95 | 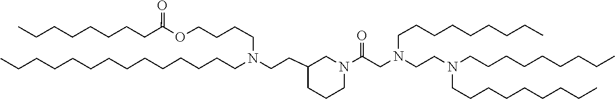 [SMILES: CCCCCCCCCCCCCCN(CCCCOC(=O)CCCCCCCC)CCC1CCCN(C(=O)CN(CCCCCCCCC)CCN(CCCCCCCCC)CCCCCCCCC)C1] |
| VI-96 | 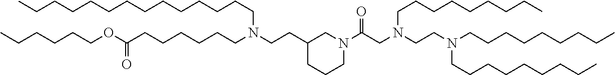 [SMILES: CCCCCCCCCCCCCCN(CCCCCCC(=O)OCCCCCC)CCC1CCCN(C(=O)CN(CCCCCCCCC)CCN(CCCCCCCCC)CCCCCCCCC)C1] |
| VI-97 | 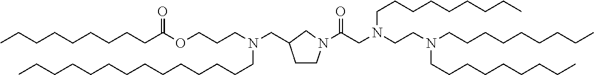 [SMILES: CCCCCCCCCCCCCCN(CCCOC(=O)CCCCCCCCC)CC1CCN(C(=O)CN(CCCCCCCCC)CCN(CCCCCCCCC)CCCCCCCCC)C1] |
| VI-98 | 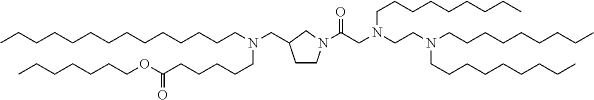 [SMILES: CCCCCCCCCCCCCCN(CCCCCC(=O)OCCCCCCC)CC1CCN(C(=O)CN(CCCCCCCCC)CCN(CCCCCCCCC)CCCCCCCCC)C1] |
| VI-99 | 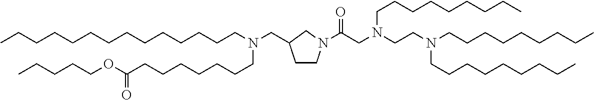 [SMILES: CCCCCCCCCCCCCCN(CCCCCCCC(=O)OCCCCC)CC1CCN(C(=O)CN(CCCCCCCCC)CCN(CCCCCCCCC)CCCCCCCCC)C1] |
| VI-100 | 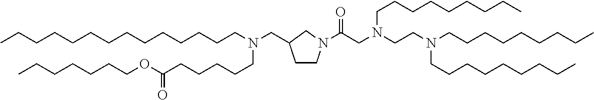 [SMILES: CCCCCCCCCCCCCCN(CCCCCC(=O)OCCCCCCC)CC1CCN(C(=O)CN(CCCCCCCCC)CCN(CCCCCCCCC)CCCCCCCCC)C1] |
| VI-101 | 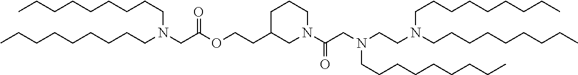 [SMILES: CCCCCCCCCN(CCCCCCCCC)CCN(CCCCCCCCC)CC(=O)N1CCCC(CCOC(=O)CN(CCCCCCCCC)CCCCCCCCC)C1] |
| VI-102 | 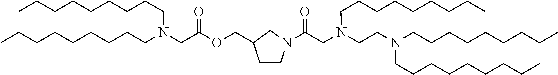 [SMILES: CCCCCCCCCN(CCCCCCCCC)CCN(CCCCCCCCC)CC(=O)N1CCC(COC(=O)CN(CCCCCCCCC)CCCCCCCCC)C1] |
| VI-103 | 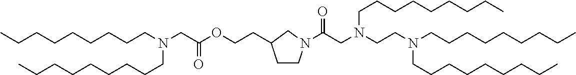 [SMILES: CCCCCCCCCN(CCCCCCCCC)CCN(CCCCCCCCC)CC(=O)N1CCC(CCOC(=O)CN(CCCCCCCCC)CCCCCCCCC)C1] |
| VI-104 | 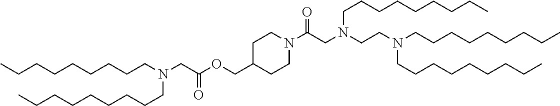 [SMILES: CCCCCCCCCN(CCCCCCCCC)CCN(CCCCCCCCC)CC(=O)N1CCC(COC(=O)CN(CCCCCCCCC)CCCCCCCCC)CC1] |
| VI-105 | 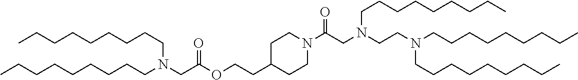 [SMILES: CCCCCCCCCN(CCCCCCCCC)CCN(CCCCCCCCC)CC(=O)N1CCC(CCOC(=O)CN(CCCCCCCCC)CCCCCCCCC)CC1] |
| VI-106 | 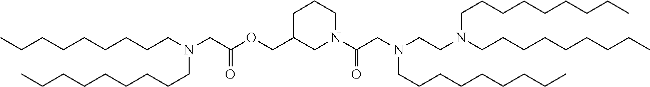 [SMILES: CCCCCCCCCN(CCCCCCCCC)CCN(CCCCCCCCC)CC(=O)N1CCCC(COC(=O)CN(CCCCCCCCC)CCCCCCCCC)C1] |
| VI-107 | 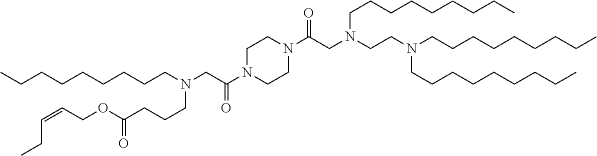 [SMILES: CC/C=C\COC(=O)CCCN(CCCCCCCCC)CC(=O)N1CCN(C(=O)CN(CCCCCCCCC)CCN(CCCCCCCCC)CCCCCCCCC)CC1] |
| VI-108 | 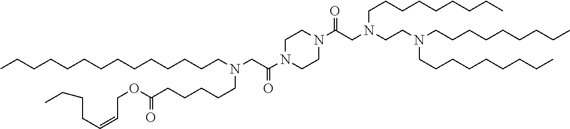 [SMILES: CCCC/C=C\COC(=O)CCCCCN(CCCCCCCCCCCCCC)CC(=O)N1CCN(C(=O)CN(CCCCCCCCC)CCN(CCCCCCCCC)CCCCCCCCC)CC1] |
| VI-109 | 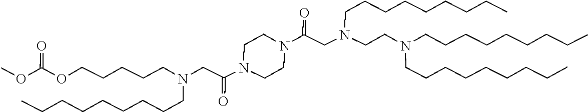 [SMILES: CCCCCCCCCN(CCCCCCCCC)CCN(CCCCCCCCC)CC(=O)N1CCN(C(=O)CN(CCCCCCCCC)CCCCCOC(=O)OC)CC1] |
| VI-110 | 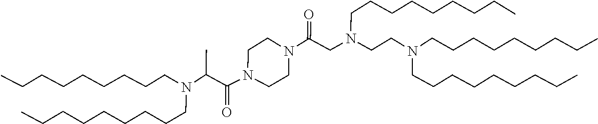 [SMILES: CCCCCCCCCN(CCCCCCCCC)CCN(CCCCCCCCC)CC(=O)N1CCN(C(=O)C(C)N(CCCCCCCCC)CCCCCCCCC)CC1] |
| VI-111 | 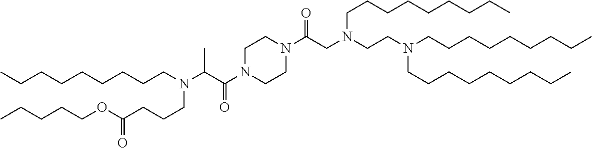 [SMILES: CCCCCCCCCN(CCCCCCCCC)CCN(CCCCCCCCC)CC(=O)N1CCN(C(=O)C(C)N(CCCCCCCCC)CCCC(=O)OCCCCC)CC1] |
| VI-112 | 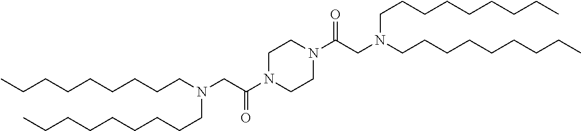 [SMILES: CCCCCCCCCN(CCCCCCCCC)CC(=O)N1CCN(C(=O)CN(CCCCCCCCC)CCCCCCCCC)CC1] |
| VI-113 | 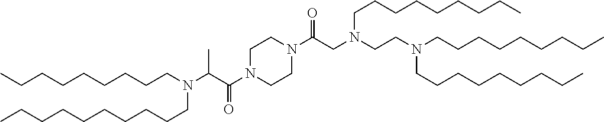 [SMILES: CCCCCCCCCCN(CCCCCCCCC)C(C)C(=O)N1CCN(C(=O)CN(CCCCCCCCC)CCN(CCCCCCCCC)CCCCCCCCC)CC1] |
| VI-114 | 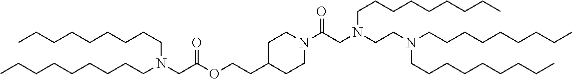 [SMILES: CCCCCCCCCN(CCCCCCCCC)CCN(CCCCCCCCC)CC(=O)N1CCC(CCOC(=O)CN(CCCCCCCCC)CCCCCCCCC)CC1] |
| VI-115 | 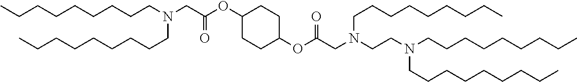 [SMILES: CCCCCCCCCN(CCCCCCCCC)CCN(CCCCCCCCC)CC(=O)OC1CCC(OC(=O)CN(CCCCCCCCC)CCCCCCCCC)CC1] |
| VI-116 | 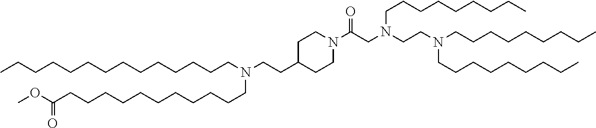 [SMILES: CCCCCCCCCCCCCCN(CCCCCCCCCCCC(=O)OC)CCC1CCN(C(=O)CN(CCCCCCCCC)CCN(CCCCCCCCC)CCCCCCCCC)CC1] |
| VI-117 | 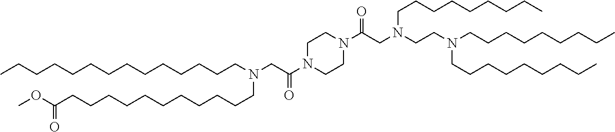 [SMILES: CCCCCCCCCCCCCCN(CCCCCCCCCCCC(=O)OC)CC(=O)N1CCN(C(=O)CN(CCCCCCCCC)CCN(CCCCCCCCC)CCCCCCCCC)CC1] |
| VI-118 | 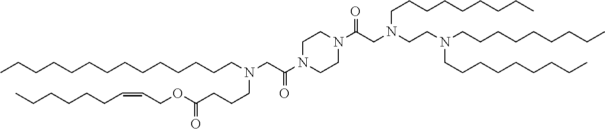 [SMILES: CCCCCC/C=C\COC(=O)CCCN(CCCCCCCCCCCCCC)CC(=O)N1CCN(C(=O)CN(CCCCCCCCC)CCN(CCCCCCCCC)CCCCCCCCC)CC1] |
| VI-119 | 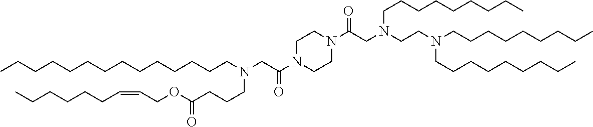 [SMILES: CCCCCC/C=C\COC(=O)CCCN(CCCCCCCCCCCCCC)CC(=O)N1CCN(C(=O)CN(CCCCCCCCC)CCN(CCCCCCCCC)CCCCCCCCC)CC1] |
| VI-120 | 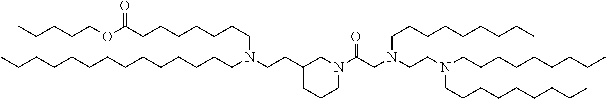 [SMILES: CCCCCCCCCCCCCCN(CCCCCCCC(=O)OCCCCC)CCC1CCCN(C(=O)CN(CCCCCCCCC)CCN(CCCCCCCCC)CCCCCCCCC)C1] |
| VI-121 | 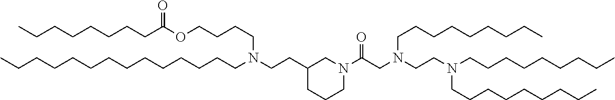 [SMILES: CCCCCCCCCCCCCCN(CCCCOC(=O)CCCCCCCC)CCC1CCCN(C(=O)CN(CCCCCCCCC)CCN(CCCCCCCCC)CCCCCCCCC)C1] |
| VI-122 | 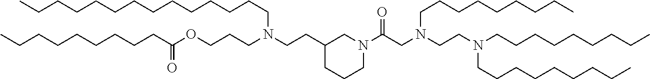 [SMILES: CCCCCCCCCCCCCCN(CCCOC(=O)CCCCCCCCC)CCC1CCCN(C(=O)CN(CCCCCCCCC)CCN(CCCCCCCCC)CCCCCCCCC)C1] |
| VI-123 | 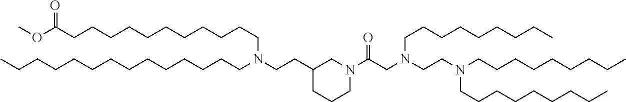 [SMILES: CCCCCCCCCCCCCCN(CCCCCCCCCCCC(=O)OC)CCC1CCCN(C(=O)CN(CCCCCCCCC)CCN(CCCCCCCCC)CCCCCCCCC)C1] |
| VI-124 | 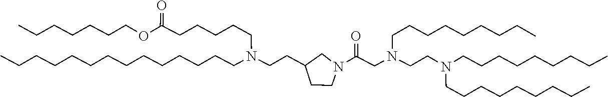 [SMILES: CCCCCCCCCCCCCCN(CCCCCC(=O)OCCCCCCC)CCC1CCN(C(=O)CN(CCCCCCCCC)CCN(CCCCCCCCC)CCCCCCCCC)C1] |
| VI-125 | 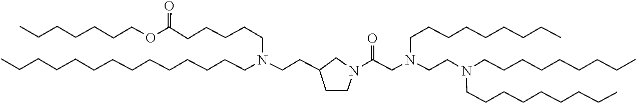 [SMILES: CCCCCCCCCCCCCCN(CCCCCC(=O)OCCCCCCC)CCC1CCN(C(=O)CN(CCCCCCCCC)CCN(CCCCCCCCC)CCCCCCCCC)C1] |
| VI-126 | 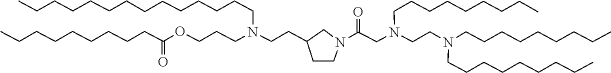 [SMILES: CCCCCCCCCCCCCCN(CCCOC(=O)CCCCCCCCC)CCC1CCN(C(=O)CN(CCCCCCCCC)CCN(CCCCCCCCC)CCCCCCCCC)C1] |
| VI-127 | 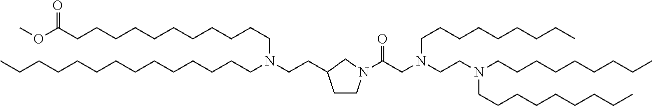 [SMILES: CCCCCCCCCCCCCCN(CCCCCCCCCCCC(=O)OC)CCC1CCN(C(=O)CN(CCCCCCCCC)CCN(CCCCCCCCC)CCCCCCCCC)C1] |
| VI-128 | 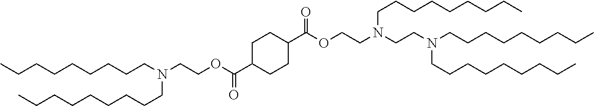 [SMILES: CCCCCCCCCN(CCCCCCCCC)CCOC(=O)C1CCC(C(=O)OCCN(CCCCCCCCC)CCN(CCCCCCCCC)CCCCCCCCC)CC1] |
| VI-129 | 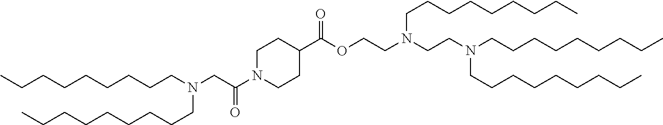 [SMILES: CCCCCCCCCN(CCCCCCCCC)CCN(CCCCCCCCC)CCOC(=O)C1CCN(C(=O)CN(CCCCCCCCC)CCCCCCCCC)CC1] |
| VI-130 | 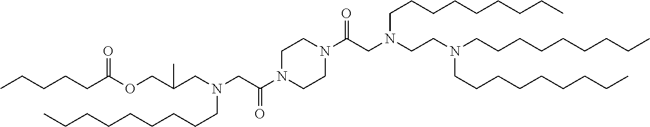 [SMILES: CCCCCCCCCN(CCCCCCCCC)CCN(CCCCCCCCC)CC(=O)N1CCN(C(=O)CN(CCCCCCCCC)CC(C)COC(=O)CCCCC)CC1] |
| VI-131 | 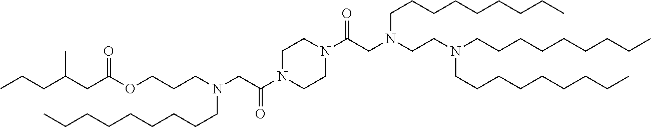 [SMILES: CCCCCCCCCN(CCCCCCCCC)CCN(CCCCCCCCC)CC(=O)N1CCN(C(=O)CN(CCCCCCCCC)CCCOC(=O)CC(C)CCC)CC1] |
| VI-132 | 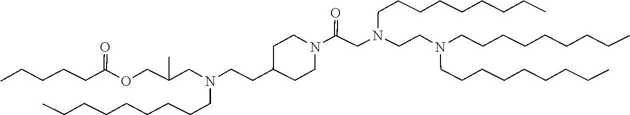 [SMILES: CCCCCCCCCN(CCCCCCCCC)CCN(CCCCCCCCC)CC(=O)N1CCC(CCN(CCCCCCCCC)CC(C)COC(=O)CCCCC)CC1] |
| VI-133 | 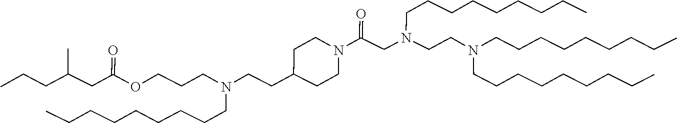 [SMILES: CCCCCCCCCN(CCCOC(=O)CC(C)CCC)CCC1CCN(C(=O)CN(CCCCCCCCC)CCN(CCCCCCCCC)CCCCCCCCC)CC1] |
| VI-134 | 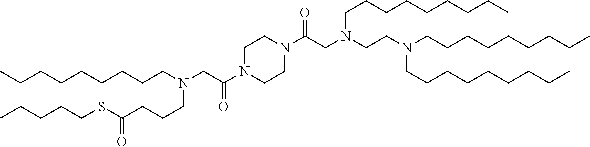 [SMILES: CCCCCCCCCN(CCCCCCCCC)CCN(CCCCCCCCC)CC(=O)N1CCN(C(=O)CN(CCCCCCCCC)CCCC(=O)SCCCCC)CC1] |
| VI-135 | 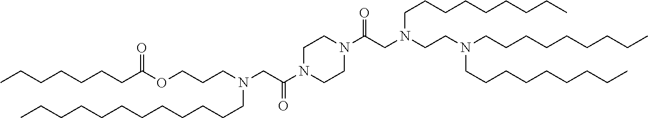 [SMILES: CCCCCCCCCCCCN(CCCOC(=O)CCCCCCC)CC(=O)N1CCN(C(=O)CN(CCCCCCCCC)CCN(CCCCCCCCC)CCCCCCCCC)CC1] |
| VI-136 | 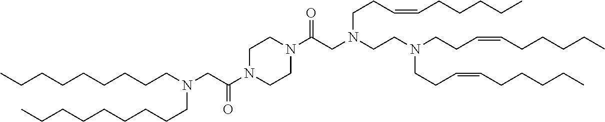 [SMILES: CCCCC/C=C\CCN(CC/C=C\CCCCC)CCN(CC/C=C\CCCCC)CC(=O)N1CCN(C(=O)CN(CCCCCCCCC)CCCCCCCCC)CC1] |
| VI-137 | 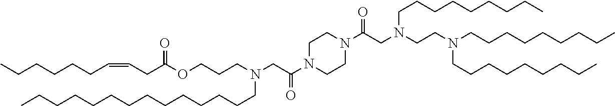 [SMILES: CCCCCC/C=C\CC(=O)OCCCN(CCCCCCCCCCCCCC)CC(=O)N1CCN(C(=O)CN(CCCCCCCCC)CCN(CCCCCCCCC)CCCCCCCCC)CC1] |
| VI-138 | 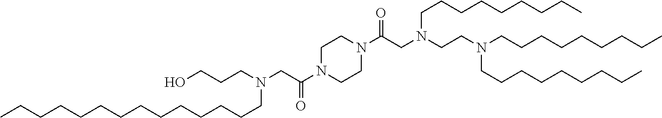 [SMILES: CCCCCCCCCCCCCCN(CCCO)CC(=O)N1CCN(C(=O)CN(CCCCCCCCC)CCN(CCCCCCCCC)CCCCCCCCC)CC1] |
| VI-139 | 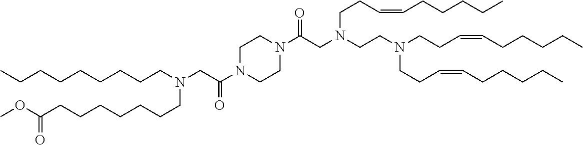 [SMILES: CCCCC/C=C\CCN(CC/C=C\CCCCC)CCN(CC/C=C\CCCCC)CC(=O)N1CCN(C(=O)CN(CCCCCCCCC)CCCCCCCC(=O)OC)CC1] |
| VI-140 | 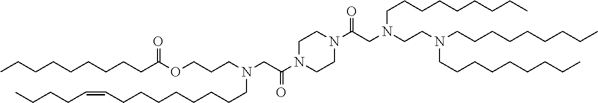 [SMILES: CCCC/C=C\CCCCCCCCN(CCCOC(=O)CCCCCCCCC)CC(=O)N1CCN(C(=O)CN(CCCCCCCCC)CCN(CCCCCCCCC)CCCCCCCCC)CC1] |
| VI-141 | 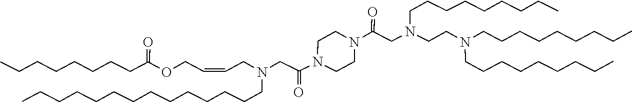 [SMILES: CCCCCCCCCCCCCCN(C/C=C\COC(=O)CCCCCCCC)CC(=O)N1CCN(C(=O)CN(CCCCCCCCC)CCN(CCCCCCCCC)CCCCCCCCC)CC1] |
| VI-142 | 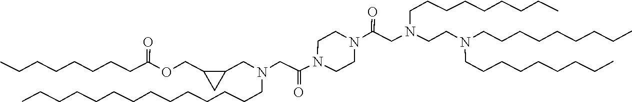 [SMILES: CCCCCCCCCCCCCCN(CC(=O)N1CCN(C(=O)CN(CCCCCCCCC)CCN(CCCCCCCCC)CCCCCCCCC)CC1)CC1CC1COC(=O)CCCCCCCC] |
| VI-143 | 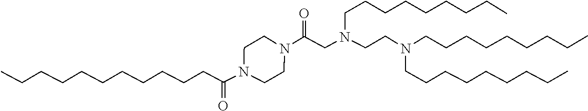 [SMILES: CCCCCCCCCCCC(=O)N1CCN(C(=O)CN(CCCCCCCCC)CCN(CCCCCCCCC)CCCCCCCCC)CC1] |
| VI-144 | 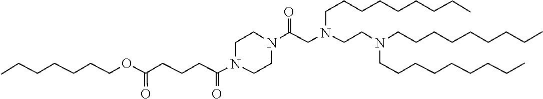 [SMILES: CCCCCCCCCN(CCCCCCCCC)CCN(CCCCCCCCC)CC(=O)N1CCN(C(=O)CCCC(=O)OCCCCCCC)CC1] |
| VI-145 | 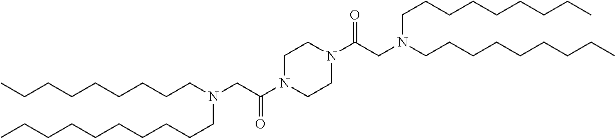 [SMILES: CCCCCCCCCCN(CCCCCCCCC)CC(=O)N1CCN(C(=O)CN(CCCCCCCCC)CCCCCCCCC)CC1] |
| VI-146 | 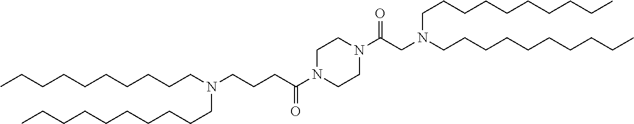 [SMILES: CCCCCCCCCCN(CCCCCCCCCC)CCCC(=O)N1CCN(C(=O)CN(CCCCCCCCCC)CCCCCCCCCC)CC1] |
| VI-147 | 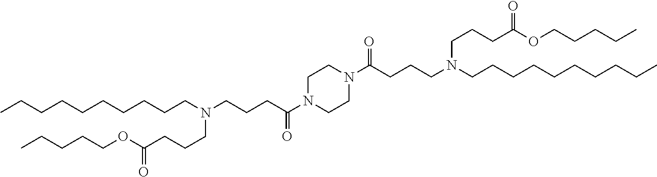 [SMILES: CCCCCCCCCCN(CCCC(=O)OCCCCC)CCCC(=O)N1CCN(C(=O)CCCN(CCCCCCCCCC)CCCC(=O)OCCCCC)CC1] |
| VI-148 | 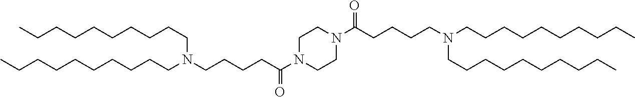 [SMILES: CCCCCCCCCCN(CCCCCCCCCC)CCCCC(=O)N1CCN(C(=O)CCCCN(CCCCCCCCCC)CCCCCCCCCC)CC1] |
| VI-149 | 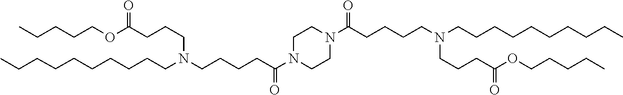 |
| VI-150 | 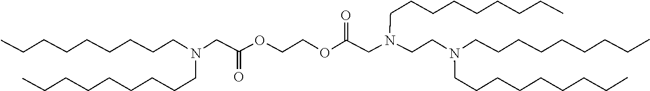 [SMILES: CCCCCCCCCN(CCCCCCCCC)CCN(CCCCCCCCC)CC(=O)OCCOC(=O)CN(CCCCCCCCC)CCCCCCCCC] |
| VI-151 | 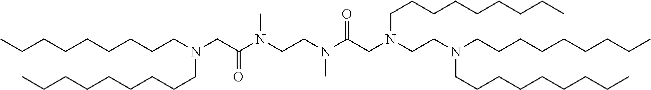 [SMILES: CCCCCCCCCN(CCCCCCCCC)CCN(CCCCCCCCC)CC(=O)N(C)CCN(C)C(=O)CN(CCCCCCCCC)CCCCCCCCC] |
| VI-152 | 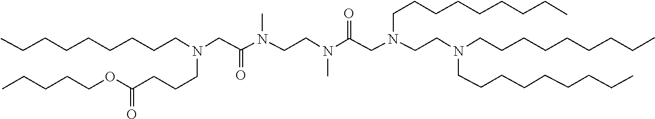 [SMILES: CCCCCCCCCN(CCCCCCCCC)CCN(CCCCCCCCC)CC(=O)N(C)CCN(C)C(=O)CN(CCCCCCCCC)CCCC(=O)OCCCCC] |
| VI-153 | 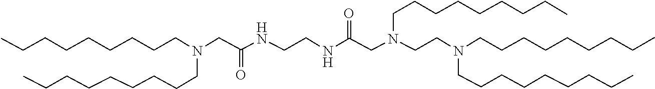 [SMILES: CCCCCCCCCN(CCCCCCCCC)CCN(CCCCCCCCC)CC(=O)NCCNC(=O)CN(CCCCCCCCC)CCCCCCCCC] |
| VI-154 | 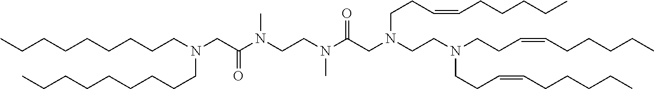 [SMILES: CCCCC/C=C\CCN(CC/C=C\CCCCC)CCN(CC/C=C\CCCCC)CC(=O)N(C)CCN(C)C(=O)CN(CCCCCCCCC)CCCCCCCCC] |
| VI-155 | 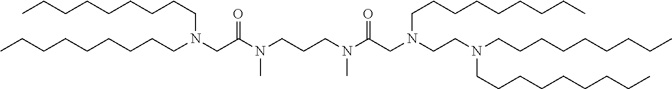 [SMILES: CCCCCCCCCN(CCCCCCCCC)CCN(CCCCCCCCC)CC(=O)N(C)CCCN(C)C(=O)CN(CCCCCCCCC)CCCCCCCCC] |
| VI-156 | 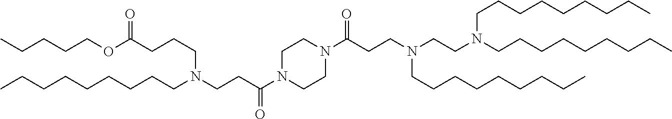 [SMILES: CCCCCCCCCN(CCCC(=O)OCCCCC)CCC(=O)N1CCN(C(=O)CCN(CCCCCCCCC)CCN(CCCCCCCCC)CCCCCCCCC)CC1] |
| VI-157 | 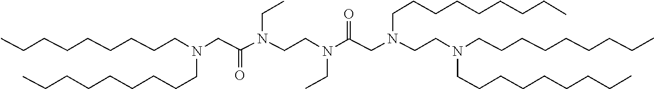 [SMILES: CCCCCCCCCN(CCCCCCCCC)CCN(CCCCCCCCC)CC(=O)N(CC)CCN(CC)C(=O)CN(CCCCCCCCC)CCCCCCCCC] |
| VI-158 | 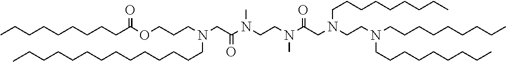 [SMILES: CCCCCCCCCCCCCCN(CCCOC(=O)CCCCCCCCC)CC(=O)N(C)CCN(C)C(=O)CN(CCCCCCCCC)CCN(CCCCCCCCC)CCCCCCCCC] |
| VI-159 | 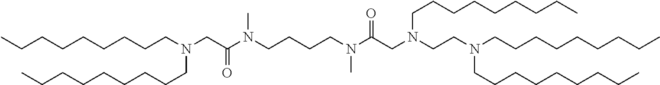 [SMILES: CCCCCCCCCN(CCCCCCCCC)CCN(CCCCCCCCC)CC(=O)N(C)CCCCN(C)C(=O)CN(CCCCCCCCC)CCCCCCCCC] |
| VI-160 | 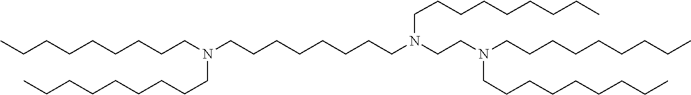 [SMILES: CCCCCCCCCN(CCCCCCCCC)CCCCCCCCN(CCCCCCCCC)CCN(CCCCCCCCC)CCCCCCCCC] |
| VI-161 | 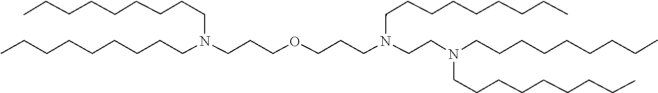 [SMILES: CCCCCCCCCN(CCCCCCCCC)CCCOCCCN(CCCCCCCCC)CCN(CCCCCCCCC)CCCCCCCCC] |
| VI-162 | 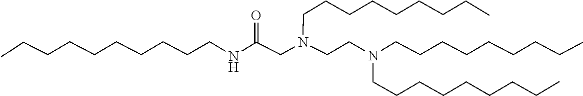 [SMILES: CCCCCCCCCCNC(=O)CN(CCCCCCCCC)CCN(CCCCCCCCC)CCCCCCCCC] |
| VI-163 | 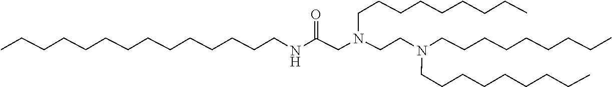 [SMILES: CCCCCCCCCCCCCCNC(=O)CN(CCCCCCCCC)CCN(CCCCCCCCC)CCCCCCCCC] |
| VI-164 | 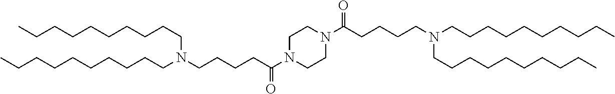 [SMILES: CCCCCCCCCCN(CCCCCCCCCC)CCCCC(=O)N1CCN(C(=O)CCCCN(CCCCCCCCCC)CCCCCCCCCC)CC1] |
| VI-165 | 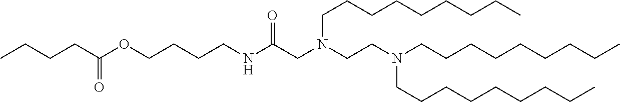 [SMILES: CCCCCCCCCN(CCCCCCCCC)CCN(CCCCCCCCC)CC(=O)NCCCCOC(=O)CCCC] |
| VI-166 | 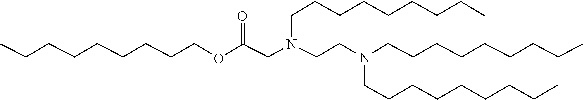 [SMILES: CCCCCCCCCOC(=O)CN(CCCCCCCCC)CCN(CCCCCCCCC)CCCCCCCCC] |
| VI-167 | 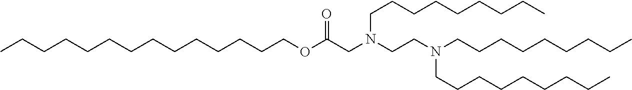 [SMILES: CCCCCCCCCCCCCCOC(=O)CN(CCCCCCCCC)CCN(CCCCCCCCC)CCCCCCCCC] |
| VI-168 | 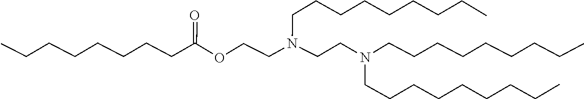 [SMILES: CCCCCCCCCN(CCCCCCCCC)CCN(CCCCCCCCC)CCOC(=O)CCCCCCCC] |
| VI-169 | 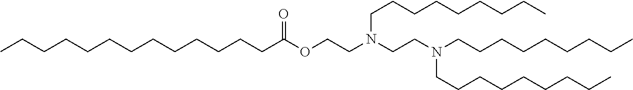 [SMILES: CCCCCCCCCCCCCC(=O)OCCN(CCCCCCCCC)CCN(CCCCCCCCC)CCCCCCCCC] |
| VI-170 | 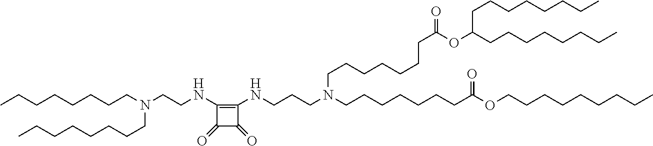 [SMILES: CCCCCCCCCOC(=O)CCCCCCCN(CCCCCCCC(=O)OC(CCCCCCCC)CCCCCCCC)CCCNc1c(NCCN(CCCCCCCC)CCCCCCCC)c(=O)c1=O] |
| VI-171 | 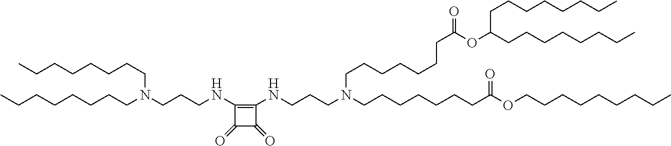 [SMILES: CCCCCCCCCOC(=O)CCCCCCCN(CCCCCCCC(=O)OC(CCCCCCCC)CCCCCCCC)CCCNc1c(NCCCN(CCCCCCCC)CCCCCCCC)c(=O)c1=O] |
| VI-172 | 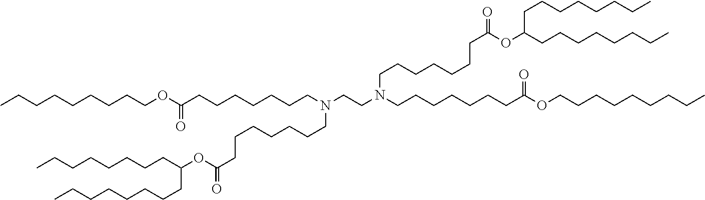 [SMILES: CCCCCCCCCOC(=O)CCCCCCCN(CCCCCCCC(=O)OC(CCCCCCCC)CCCCCCCC)CCN(CCCCCCCC(=O)OCCCCCCCCC)CCCCCCCC(=O)OC(CCCCCCCC)CCCCCCCC] |
| VI-173 | 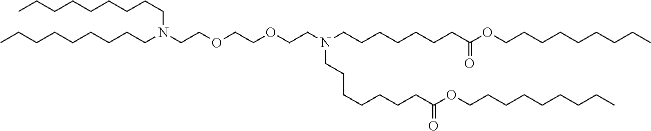 [SMILES: CCCCCCCCCOC(=O)CCCCCCCN(CCCCCCCC(=O)OCCCCCCCCC)CCOCCOCCN(CCCCCCCCC)CCCCCCCCC] |
| VI-174 | 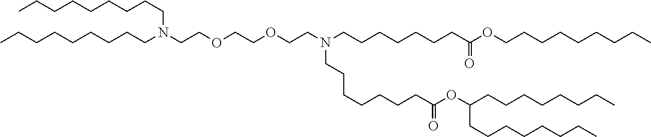 [SMILES: CCCCCCCCCOC(=O)CCCCCCCN(CCCCCCCC(=O)OC(CCCCCCCC)CCCCCCCC)CCOCCOCCN(CCCCCCCCC)CCCCCCCCC] |
| VI-175 | 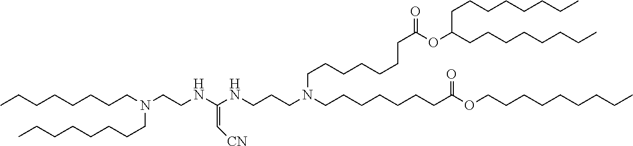 [SMILES: CCCCCCCCCOC(=O)CCCCCCCN(CCCCCCCC(=O)OC(CCCCCCCC)CCCCCCCC)CCCN/C(=C\C#N)NCCN(CCCCCCCC)CCCCCCCC] |
| VI-176 | 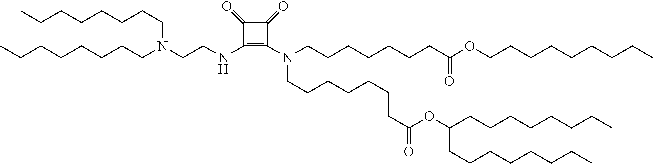 [SMILES: CCCCCCCCCOC(=O)CCCCCCCN(CCCCCCCC(=O)OC(CCCCCCCC)CCCCCCCC)c1c(NCCN(CCCCCCCC)CCCCCCCC)c(=O)c1=O] |
| VI-177 | 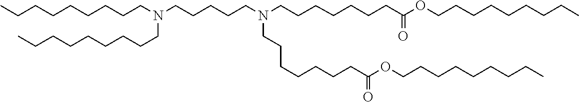 [SMILES: CCCCCCCCCOC(=O)CCCCCCCN(CCCCCCCC(=O)OCCCCCCCCC)CCCCCN(CCCCCCCCC)CCCCCCCCC] |
| VI-178 | 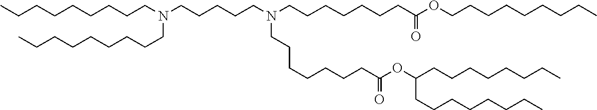 [SMILES: CCCCCCCCCOC(=O)CCCCCCCN(CCCCCCCC(=O)OC(CCCCCCCC)CCCCCCCC)CCCCCN(CCCCCCCCC)CCCCCCCCC] |
| VI-179 | 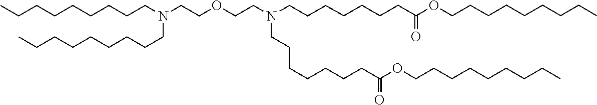 [SMILES: CCCCCCCCCOC(=O)CCCCCCCN(CCCCCCCC(=O)OCCCCCCCCC)CCOCCN(CCCCCCCCC)CCCCCCCCC] |
| VI-180 | 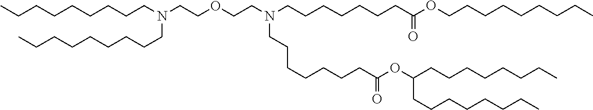 [SMILES: CCCCCCCCCOC(=O)CCCCCCCN(CCCCCCCC(=O)OC(CCCCCCCC)CCCCCCCC)CCOCCN(CCCCCCCCC)CCCCCCCCC] |
| VI-181 | 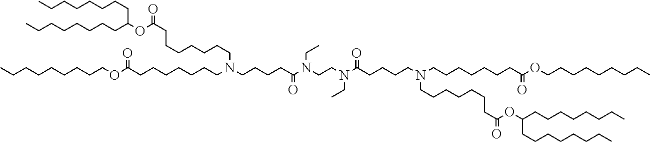 [SMILES: CCCCCCCCCOC(=O)CCCCCCCN(CCCCCCCC(=O)OC(CCCCCCCC)CCCCCCCC)CCCCC(=O)N(CC)CCN(CC)C(=O)CCCCN(CCCCCCCC(=O)OCCCCCCCCC)CCCCCCCC(=O)OC(CCCCCCCC)CCCCCCCC] |
| VI-182 | 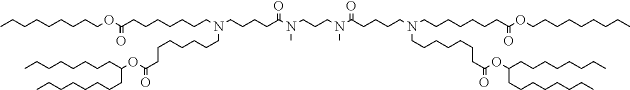 [SMILES: CCCCCCCCCOC(=O)CCCCCCCN(CCCCCCCC(=O)OC(CCCCCCCC)CCCCCCCC)CCCCC(=O)N(C)CCCN(C)C(=O)CCCCN(CCCCCCCC(=O)OCCCCCCCCC)CCCCCCCC(=O)OC(CCCCCCCC)CCCCCCCC] |
| VI-183 | 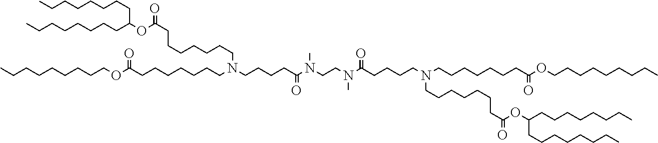 [SMILES: CCCCCCCCCOC(=O)CCCCCCCN(CCCCCCCC(=O)OC(CCCCCCCC)CCCCCCCC)CCCCC(=O)N(C)CCN(C)C(=O)CCCCN(CCCCCCCC(=O)OCCCCCCCCC)CCCCCCCC(=O)OC(CCCCCCCC)CCCCCCCC] |
| VI-184 | 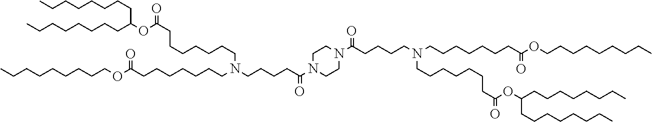 [SMILES: CCCCCCCCCOC(=O)CCCCCCCN(CCCCCCCC(=O)OC(CCCCCCCC)CCCCCCCC)CCCCC(=O)N1CCN(C(=O)CCCCN(CCCCCCCC(=O)OCCCCCCCCC)CCCCCCCC(=O)OC(CCCCCCCC)CCCCCCCC)CC1] |
| VI-185 | 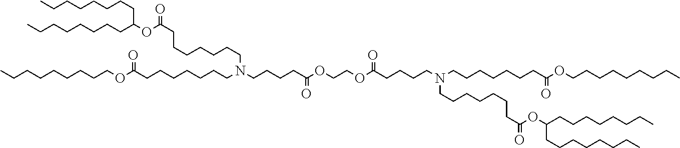 [SMILES: CCCCCCCCCOC(=O)CCCCCCCN(CCCCCCCC(=O)OC(CCCCCCCC)CCCCCCCC)CCCCC(=O)OCCOC(=O)CCCCN(CCCCCCCC(=O)OCCCCCCCCC)CCCCCCCC(=O)OC(CCCCCCCC)CCCCCCCC] |
| VI-186 | 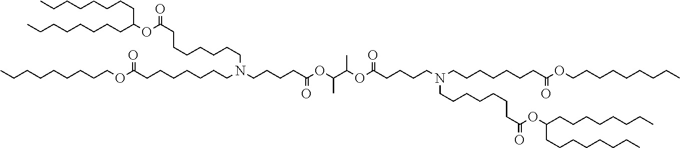 [SMILES: CCCCCCCCCOC(=O)CCCCCCCN(CCCCCCCC(=O)OC(CCCCCCCC)CCCCCCCC)CCCCC(=O)OC(C)C(C)OC(=O)CCCCN(CCCCCCCC(=O)OCCCCCCCCC)CCCCCCCC(=O)OC(CCCCCCCC)CCCCCCCC] |
| VI-187 | 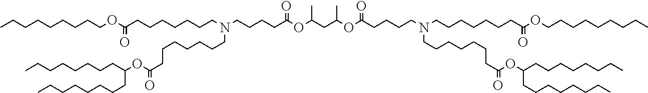 [SMILES: CCCCCCCCCOC(=O)CCCCCCCN(CCCCCCCC(=O)OC(CCCCCCCC)CCCCCCCC)CCCCC(=O)OC(C)CC(C)OC(=O)CCCCN(CCCCCCCC(=O)OCCCCCCCCC)CCCCCCCC(=O)OC(CCCCCCCC)CCCCCCCC] |
| VI-188 | 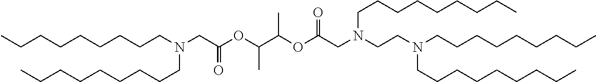 [SMILES: CCCCCCCCCN(CCCCCCCCC)CCN(CCCCCCCCC)CC(=O)OC(C)C(C)OC(=O)CN(CCCCCCCCC)CCCCCCCCC] |
| VI-189 | 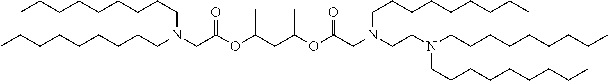 [SMILES: CCCCCCCCCN(CCCCCCCCC)CCN(CCCCCCCCC)CC(=O)OC(C)CC(C)OC(=O)CN(CCCCCCCCC)CCCCCCCCC] |

In some embodiments, the ionizable lipid is a compound selected from:

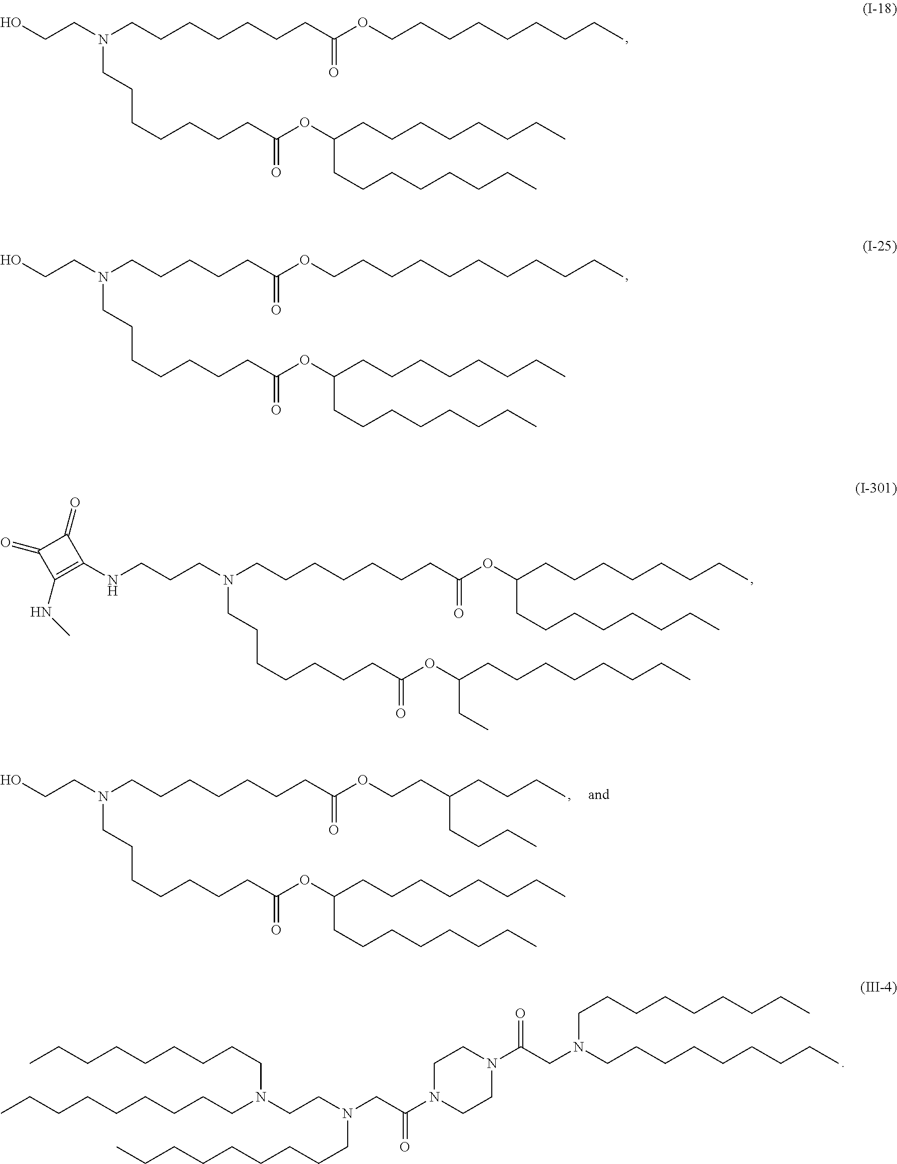

In some embodiments, the ionizable lipid is

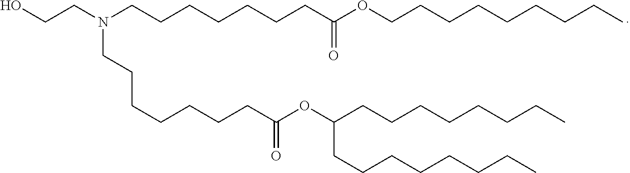 [SMILES: CCCCCCCCCOC(=O)CCCCCCCN(CCO)CCCCCCCC(=O)OC(CCCCCCCC)CCCCCCCC]

In some embodiments, the ionizable lipid is

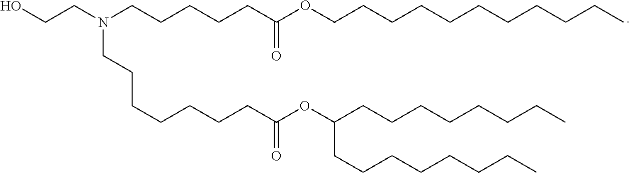 [SMILES: CCCCCCCCCCCOC(=O)CCCCCN(CCO)CCCCCCCC(=O)OC(CCCCCCCC)CCCCCCCC]

In some embodiments, the ionizable lipid is

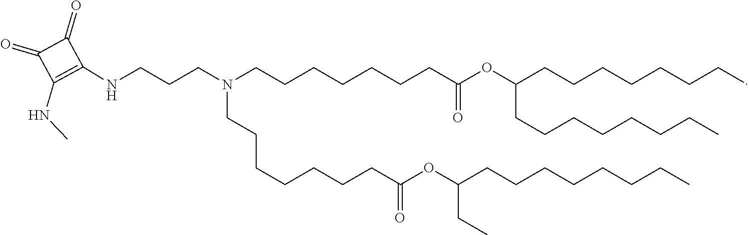

In some embodiments, the ionizable lipid is

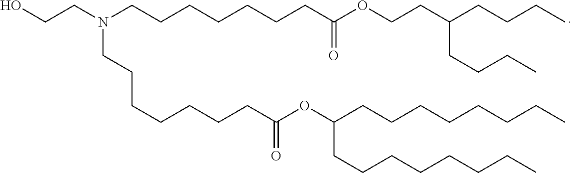 [SMILES: CCCCCCCCC(CCCCCCCC)OC(=O)CCCCCCCN(CCO)CCCCCCCC(=O)OCCC(CCCC)CCCC]

In some embodiments, the ionizable lipid is

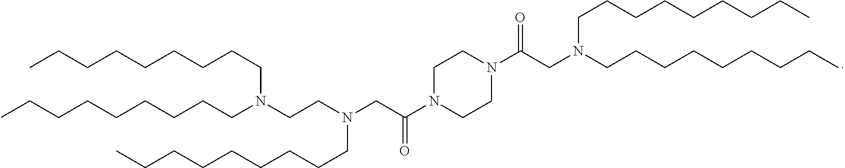 [SMILES: CCCCCCCCCN(CCCCCCCCC)CCN(CCCCCCCCC)CC(=O)N1CCN(C(=O)CN(CCCCCCCCC)CCCCCCCCC)CC1]

Without wishing to be bound by theory, it is understood that an ionizable lipid may have a positive or partial positive charge at physiological pH. Such lipids may be referred to as cationic or ionizable (amino) lipids. Lipids may also be zwitterionic, i.e., neutral molecules having both a positive and a negative charge.

### Polyethylene Glycol (PEG) Lipids

As used herein, the term “PEG lipid” refers to polyethylene glycol (PEG)-modified lipids. Non-limiting examples of PEG lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG lipid includes, but not limited to, 1,2-dimyristoyl-sn-glycerol methoxypolyethylene glycol (PEG-DMG), 1,2-distearoyl-sn-glycero-3-phosphoethanolamine-N-[amino (polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG lipids includes those having lengths of from about C₁₄ to about C₂₂, preferably from about C₁₄ to about C₁₆. In some embodiments, a PEG moiety, for example, an mPEG-NH₂, has a size of about 1000, 2000, 5000, 10,000, 15,000 or 20,000 daltons. In one embodiment, the PEG lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described in International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the PEG-modified lipids are a modified form of PEG DMG. PEG-DMG has the following structure:

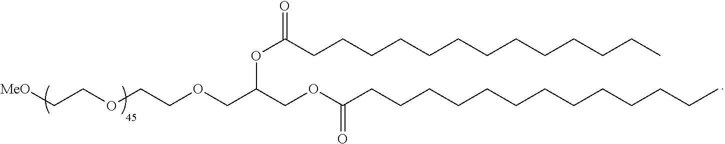

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In some embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In some embodiments, the PEG-OH lipid includes one or more hydroxyl groups on the PEG chain. In some embodiments, a PEG-OH or hydroxy-PEGylated lipid comprises an —OH group at the terminus of the PEG chain. Each possibility represents a separate embodiment of the present invention.

In some embodiments, a PEG lipid useful in the present invention is a compound of Formula (PL-I). Provided herein are compounds of Formula (PL-I):

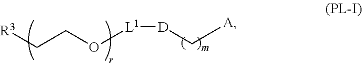

or salts thereof, wherein:
R³ is —OR^{O};
R^{O} is hydrogen, optionally substituted alkyl, or an oxygen-protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, —OC(O), OC(O)O, OC(O)N(R^{N}), NR^{N}C(O)O, or NR^{N}C(O)N(R^{N});
D is absent; or
D is a moiety obtained by click chemistry or a moiety cleavable under physiological conditions;
m is 0, 1, 2, 3, 4, 5, 6, 7, 8, 9, or 10;
A is of the formula:

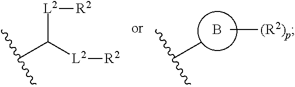

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O) S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen-protecting group;
Ring B is optionally substituted carbocyclyl, optionally substituted heterocyclyl, optionally substituted aryl, or optionally substituted heteroaryl; and
p is 1 or 2.

In some embodiments, the compound of Formula (PL-I) is a PEG-OH lipid (i.e., R³ is —OR^{O}, and R^{O} is hydrogen). In some embodiments, the compound of Formula (PL-I) is of Formula (PL-I-OH):

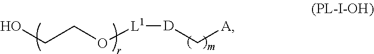

or a salt thereof.

In some embodiments, a PEG lipid useful in the present invention is a PEGylated fatty acid. In some embodiments, a PEG lipid useful in the present invention is a compound of Formula (PL-II). Provided herein are compounds of Formula (PL-II):

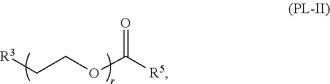

or a salt thereof, wherein:
R³ is —OR^{O};
R^{O} is hydrogen, optionally substituted alkyl or an oxygen-protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), —NR^{N}C(O)O, C(O) S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, —S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂₀; and
each instance of R^{N} is independently hydrogen, optionally substituted alkyl, or a nitrogen-protecting group.

In some embodiments, the compound of Formula (PL-II) is of Formula (PL-II-OH):

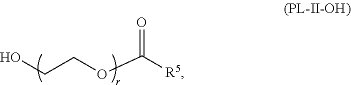

or a salt thereof. In some embodiments, r is 35-55. In some embodiments, r is 45.

In yet other embodiments the compound of Formula (PL-II) is:

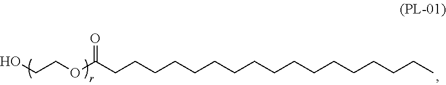

or a salt thereof. In some embodiments, r is 1-100. In some embodiments, r is about 35 to about 55. In some embodiments, r is 35-55. In some embodiments, r is 45.

In one embodiment, the compound of Formula (PL-II) is

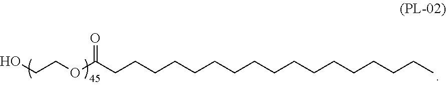

In yet other embodiments, the PEG lipid is PEG1. PEG1 is a plurality of compounds of Formula (PL-01):

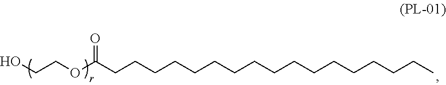

or salts thereof, wherein r is 1-100. In some embodiments, r is about 35 to about 55. In some embodiments, r is 35-55. In some embodiments, r is 45.

In some embodiments, the PEG lipids may be one or more of the PEG lipids described in U.S. Application No. 62/520,530.

### Structural Lipids

As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including, but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the structural lipids may be one or more of the structural lipids described in U.S. Application No. 62/520,530.

### Polynucleotides and Nucleic Acids

In some embodiments, the therapeutic agent is an agent that enhances (i.e., increases, stimulates, or upregulates) protein expression. Non-limiting examples of types of therapeutic agents that can be used for enhancing protein expression include RNAs, mRNAs, dsRNAs, CRISPR/Cas9 technology, ssDNAs and DNAs (e.g., expression vectors). The agent that upregulates protein expression may upregulate expression of a naturally occurring or non-naturally occurring protein (e.g., a chimeric protein that has been modified to improve half life, or one that comprises desirable amino acid changes). Exemplary proteins include intracellular, transmembrane, or secreted proteins, peptides, or polypeptides.

In some embodiments, the therapeutic agent is a DNA therapeutic agent. The DNA molecule can be a double-stranded DNA, a single-stranded DNA (ssDNA), or a molecule that is a partially double-stranded DNA, i.e., has a portion that is double-stranded and a portion that is single-stranded. In some cases, the DNA molecule is triple-stranded or is partially triple-stranded, i.e., has a portion that is triple stranded and a portion that is double stranded. The DNA molecule can be a circular DNA molecule or a linear DNA molecule.

A DNA therapeutic agent can be a DNA molecule that is capable of transferring a gene into a cell, e.g., that encodes and can express a transcript. In other embodiments, the DNA molecule is a synthetic molecule, e.g., a synthetic DNA molecule produced in vitro. In some embodiments, the DNA molecule is a recombinant molecule. Non-limiting exemplary DNA therapeutic agents include plasmid expression vectors and viral expression vectors.

The DNA therapeutic agents described herein, e.g., DNA vectors, can include a variety of different features. The DNA therapeutic agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

In some embodiments, the one or more therapeutic and/or prophylactic agents are a nucleic acid. In some embodiments, the one or more therapeutic and/or prophylactic agents are selected from the group consisting of a ribonucleic acid (RNA) and a deoxyribonucleic acid (DNA).

For example, in some embodiments, when the therapeutic and/or prophylactic agents are a DNA, the DNA is selected from the group consisting of a double-stranded DNA, a single-stranded DNA (ssDNA), a partially double-stranded DNA, a triple stranded DNA, and a partially triple-stranded DNA. In some embodiments, the DNA is selected from the group consisting of a circular DNA, a linear DNA, and mixtures thereof.

In some embodiments, the one or more therapeutic and/or prophylactic agents are selected from the group consisting of a plasmid expression vector, a viral expression vector, and mixtures thereof.

For example, in some embodiments, when the therapeutic and/or prophylactic agents is an RNA, the RNA is selected from the group consisting of a single-stranded RNA, a double-stranded RNA (dsRNA), a partially double-stranded RNA, and mixtures thereof. In some embodiments, the RNA is selected from the group consisting of a circular RNA, a linear RNA, and mixtures thereof.

For example, in some embodiments, when the therapeutic and/or prophylactic agents are an RNA, the RNA is selected from the group consisting of a short interfering RNA (siRNA), an asymmetrical interfering RNA (aiRNA), an RNA interference (RNAi) molecule, a microRNA (miRNA), an antagomir, an antisense RNA, a ribozyme, a Dicer-substrate RNA (dsRNA), a small hairpin RNA (shRNA), a messenger RNA (mRNA), locked nucleic acids (LNAs) and CRISPR/Cas9 technology, and mixtures thereof.

For example, in some embodiments, when the therapeutic and/or prophylactic agents are an RNA, the RNA is selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof.

In some embodiments, the one or more therapeutic and/or prophylactic agents are an mRNA. In some embodiments, the one or more therapeutic and/or prophylactic agents are a modified mRNA (mmRNA).

In some embodiments, the one or more therapeutic and/or prophylactic agents are an mRNA that incorporates a micro-RNA binding site (miR binding site). Further, in some embodiments, an mRNA includes one or more of a stem loop, a chain-terminating nucleoside, a polyA sequence, a polyadenylation signal, and/or a 5′ cap structure.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein, “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900), or thousands (e.g., 1000, 2000, 3000, 4000, 5000, 6000, 7000, 8000, 9000, or 10,000) of base pairs. Any number (e.g., all, some, or none) of nucleobases, nucleosides, or nucleotides may be an analog of a canonical species, substituted, modified, or otherwise non-naturally occurring. In some embodiments, all of a particular nucleobase type may be modified. In some embodiments, all uracils or uridines are modified. When all nucleobases, nucleosides, or nucleotides are modified, e.g., all uracils or uridines, the mRNA can be referred to as “fully modified,” e.g., for uracil or uridine.

In some embodiments, an mRNA, as described herein, may include a 5′ cap structure, a chain-terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,O3′GpppG, m27,O3′GppppG, m27,O2′GppppG, m7Gpppm7G, m73′dGpppG, m27,O3′GpppG, m27,O3′GppppG, and m27,O2′GppppG.

An mRNA may instead or additionally include a chain-terminating nucleoside. For example, a chain-terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2′,3′ dideoxyadenosine, 2′,3′ dideoxyuridine, 2′,3′ dideoxycytosine, 2′,3′ dideoxyguanosine, and 2′,3′ dideoxythymine. In some embodiments, incorporation of a chain-terminating nucleotide into an mRNA, for example at the 3′-terminus, may result in stabilization of the mRNA.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A poly A sequence may also comprise stabilizing nucleotides or analogs. For example, a poly A sequence can include deoxythymidine, e.g., inverted (or reverse linkage) deoxythymidine (dT), as a stabilizing nucleotide or analog. Details on using inverted dT and other stabilizing poly A sequence modifications can be found, for example, in WO2017/049275 A2, the content of which is incorporated herein by reference. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA-binding site. MicroRNA-binding sites (or miR-binding sites) can be used to regulate mRNA expression in various tissues or cell types. In exemplary embodiments, miR-binding sites are engineered into 3′ UTR sequences of an mRNA to regulate, e.g., enhance degradation of mRNA in cells or tissues expressing the cognate miR. Such regulation is useful to regulate or control “off-target” expression in mRNAs, i.e., expression in undesired cells or tissues in vivo. Details on using miR-binding sites can be found, for example, in WO 2017/062513 A2, the content of which is incorporated herein by reference.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (tm5U), 1-taurinomethyl-pseudouridine, 5-taurinomethyl-2-thio-uridine (tm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (mls4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp3U), 1-methyl-3-(3-amino-3-carboxypropyl) pseudouridine (acp3 ψ), 5-(isopentenylaminomethyl) uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include a-thio-adenosine, 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl) adenosine (io6A), 2-methylthio-N6-(cis-hydroxyisopentenyl) adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, a-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2,N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr (p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases). In some embodiments, the modified nucleobase is N1-methylpseudouridine (m1ψ) and the mRNA of the disclosure is fully modified with N1-methylpseudouridine (m1ψ). In some embodiments, N1-methylpseudouridine (m1ψ) represents from 75-100% of the uracils in the mRNA. In some embodiments, N1-methylpseudouridine (m1ψ) represents 100% of the uracils in the mRNA.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, and 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is 1-methyl-pseudouridine (m1ψ), 5-methoxy-uridine (mo5U), 5-methyl-cytidine (m5C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases).

In some embodiments, the mRNA comprises pseudouridine (ψ). In some embodiments, the mRNA comprises pseudouridine (ψ) and 5-methyl-cytidine (m5C). In some embodiments, the mRNA comprises 1-methyl-pseudouridine (m1ψ). In some embodiments, the mRNA comprises 1-methyl-pseudouridine (m1ψ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises N6-methyl-adenosine (m6A). In some embodiments, the mRNA comprises N6-methyl-adenosine (m6A) and 5-methyl-cytidine (m5C).

In some embodiments, an mRNA of the disclosure is uniformly modified (i.e., fully modified, modified through-out the entire sequence) for a particular modification. For example, an mRNA can be uniformly modified with N1-methylpseudouridine (m1ψ) or 5-methyl-cytidine (m5C), meaning that all uridines or all cytosine nucleosides in the mRNA sequence are replaced with N1-methylpseudouridine (m1ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

The mmRNAs of the disclosure can include a combination of modifications to the sugar, the nucleobase, and/or the internucleoside linkage. These combinations can include any one or more modifications described herein.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed, then the naturally occurring ATP, UTP, GTP and/or CTP is used at 100% of the sites of those nucleotides found in the polynucleotide. In this example, all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome-binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, CA) and/or proprietary methods. In some embodiments, the mRNA sequence is optimized using optimization algorithms, e.g., to optimize expression in mammalian cells or enhance mRNA stability.

In some embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including, but not limited to, in vitro transcription (IVT) and synthetic methods. Enzymatic (IVT), solid-phase, liquid-phase, combined synthetic methods, small region synthesis, and ligation methods may be utilized. In some embodiments, mRNAs are made using IVT enzymatic synthesis methods. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In some embodiments, modifications may be on internucleoside linkages, purine or pyrimidine bases, or sugar. In particular embodiments, the modification may be introduced at the terminal of a polynucleotide chain or anywhere else in the polynucleotide chain, with chemical synthesis or with a polymerase enzyme.

Either enzymatic or chemical ligation methods may be used to conjugate polynucleotides or their regions with different functional moieties, such as targeting or delivery agents, fluorescent labels, liquids, nanoparticles, etc. Therapeutic Agents for Reducing Protein Expression

In some embodiments, the therapeutic agent is a therapeutic agent that reduces (i.e., decreases, inhibits, or downregulates) protein expression. Non-limiting examples of types of therapeutic agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

Sensor Sequences and MicroRNA (miRNA) Binding Sites

Sensor sequences include, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. Non-limiting examples of sensor sequences are described in U.S. Publication 2014/0200261, the contents of which are incorporated herein by reference in their entirety.

In some embodiments, a polyribonucleotide (e.g., a ribonucleic acid (RNA), e.g., a messenger RNA (mRNA)) of the disclosure comprising an open reading frame (ORF) encoding a polypeptide further comprises a sensor sequence. In some embodiments, the sensor sequence is a miRNA-binding site.

A miRNA is a 19-25 nucleotide long noncoding RNA that binds to a polyribonucleotide and down-regulates gene expression either by reducing stability or by inhibiting translation of the polyribonucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA-binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA-binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27 (1): 91-105. miRNA profiling of the target cells or tissues can be conducted to determine the presence or absence of miRNA in the cells or tissues. In some embodiments, a polyribonucleotide (e.g., a ribonucleic acid (RNA), e.g., a messenger RNA (mRNA)) of the disclosure comprises one or more microRNA target sequences, microRNA sequences, or microRNA seeds. Such sequences can correspond to any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a polyribonucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polyribonucleotide of the disclosure comprising an ORF encoding a polypeptide further comprises a miRNA-binding site. In exemplary embodiments, a 5′UTR and/or 3′UTR of the polyribonucleotide (e.g., a ribonucleic acid (RNA), e.g., a messenger RNA (mRNA)) comprises a miRNA-binding site.

A miRNA-binding site having sufficient complementarity to a miRNA refers to a degree of complementarity sufficient to facilitate miRNA-mediated regulation of a polyribonucleotide, e.g., miRNA-mediated translational repression or degradation of the polyribonucleotide. In exemplary embodiments of the disclosure, a miRNA-binding site having sufficient complementarity to the miRNA refers to a degree of complementarity sufficient to facilitate miRNA-mediated degradation of the polyribonucleotide, e.g., miRNA-guided RNA-induced silencing complex (RISC)-mediated cleavage of mRNA. The miRNA-binding site can have complementarity to, for example, a 19-25 nucleotide miRNA sequence, to a 19-23 nucleotide miRNA sequence, or to a 22 nucleotide miRNA sequence. A miRNA-binding site can be complementary to only a portion of a miRNA, e.g., to a portion less than 1, 2, 3, or 4 nucleotides of the full length of a naturally occurring miRNA sequence. In some embodiments, the desired regulation is mRNA degradation. In some embodiments, the miRNA-binding site has full or complete complementarity (e.g., full complementarity or complete complementarity over all or a significant portion of the length of a naturally occurring miRNA). In some embodiments, the mRNA degradation has full or complete complementarity.

In some embodiments, a miRNA-binding site includes a sequence that has complementarity (e.g., partial or complete complementarity) with a miRNA seed sequence. In some embodiments, the miRNA-binding site includes a sequence that has complete complementarity with a miRNA seed sequence. In some embodiments, a miRNA-binding site includes a sequence that has complementarity (e.g., partial or complete complementarity) with a miRNA sequence. In some embodiments, the miRNA-binding site includes a sequence that has complete complementarity with a miRNA sequence. In some embodiments, a miRNA-binding site has complete complementarity with a miRNA sequence, but for 1, 2, or 3 nucleotide substitutions, terminal additions, and/or truncations.

In some embodiments, the miRNA-binding site is the same length as the corresponding miRNA. In some embodiments, the miRNA-binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA-binding site is two nucleotides shorter than the corresponding microRNA at the 5′ terminus, the 3′ terminus, or both. The miRNA-binding sites that are shorter than the corresponding miRNAs are still capable of degrading the mRNA incorporating one or more of the miRNA-binding sites or preventing the mRNA from translation.

In some embodiments, the miRNA-binding site binds to the corresponding mature miRNA that is part of an active RISC containing Dicer. In another embodiment, binding of the miRNA-binding site to the corresponding miRNA in RISC degrades the mRNA containing the miRNA-binding site or prevents the mRNA from being translated. In some embodiments, the miRNA-binding site has sufficient complementarity to miRNA so that a RISC complex comprising the miRNA cleaves the polyribonucleotide comprising the miRNA-binding site. In some embodiments, the miRNA-binding site has imperfect complementarity so that a RISC complex comprising the miRNA induces instability in the polyribonucleotide comprising the miRNA-binding site. In another embodiment, the miRNA-binding site has imperfect complementarity so that a RISC complex comprising the miRNA represses transcription of the polyribonucleotide comprising the miRNA-binding site.

In some embodiments, the miRNA-binding site has one, two, three, four, five, six, seven, eight, nine, ten, eleven or twelve mismatch(es) from the corresponding miRNA.

In some embodiments, the miRNA-binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA-binding sites into a polyribonucleotide of the disclosure, the polyribonucleotide can be targeted for degradation or reduced translation, provided the miRNA in question is available. This can reduce off-target effects upon delivery of the polyribonucleotide. In some embodiments, if a polyribonucleotide of the disclosure is not intended to be delivered to a tissue or cell but ends up there, then a miRNA abundant in the tissue or cell can inhibit the expression of the gene of interest if one or multiple binding sites of the miRNA are engineered into the 5′UTR and/or 3′UTR of the polyribonucleotide.

Conversely, miRNA-binding sites can be removed from polyribonucleotide sequences in which they naturally occur in order to increase protein expression in specific tissues. In some embodiments, a binding site for a specific miRNA can be removed from a polyribonucleotide to improve protein expression in tissues or cells containing the miRNA.

In one embodiment, a polyribonucleotide of the disclosure can include at least one miRNA-binding site in the 5′UTR and/or 3′UTR in order to direct cytotoxic or cytoprotective mRNA therapeutics to specific cells such as, but not limited to, normal and/or cancerous cells. In another embodiment, a polyribonucleotide of the disclosure can include two, three, four, five, six, seven, eight, nine, ten, or more miRNA-binding sites in the 5′-UTR and/or 3′-UTR in order to direct cytotoxic or cytoprotective mRNA therapeutics to specific cells such as, but not limited to, normal and/or cancerous cells.

Regulation of expression in multiple tissues can be accomplished through introduction or removal of one or more miRNA-binding sites. The decision of whether to remove or insert a miRNA-binding site can be made based on miRNA expression patterns and/or their profilings in diseases. Identification of miRNAs, miRNA-binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA-binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen-presenting cells (APCs) (e.g., dendritic cells and macrophages), macrophages, monocytes, B lymphocytes, T lymphocytes, granulocytes, natural killer cells, etc. Immune cell-specific miRNAs are involved in immunogenicity, autoimmunity, the immune-response to infection, inflammation, as well as unwanted immune response after gene therapy and tissue/organ transplantation. Immune cells-specific miRNAs also regulate many embodiments of development, proliferation, differentiation and apoptosis of hematopoietic cells (immune cells). In some embodiments, miR-142 and miR-146 are exclusively expressed in immune cells, particularly abundant in myeloid dendritic cells. It has been demonstrated that the immune response to a polyribonucleotide can be shut off by adding miR-142-binding sites to the 3′-UTR of the polyribonucleotide, enabling more stable gene transfer in tissues and cells. miR-142 efficiently degrades exogenous polyribonucleotides in antigen-presenting cells and suppresses cytotoxic elimination of transduced cells (e.g., Annoni A et al., blood, 2009, 114, 5152-5161; Brown B D, et al., Nat med. 2006, 12 (5), 585-591; Brown B D, et al., blood, 2007, 110 (13): 4144-4152, each of which is incorporated herein by reference in its entirety).

An antigen-mediated immune response can refer to an immune response triggered by foreign antigens, which, when entering an organism, are processed by the antigen-presenting cells and displayed on the surface of the antigen-presenting cells. T cells can recognize the presented antigen and induce a cytotoxic elimination of cells that express the antigen.

Introducing a miR-142-binding site into the 5′UTR and/or 3′UTR of a polyribonucleotide of the disclosure can selectively repress gene expression in antigen-presenting cells through miR-142 mediated degradation, limiting antigen presentation in antigen-presenting cells (e.g., dendritic cells) and thereby preventing antigen-mediated immune response after the delivery of the polyribonucleotide. The polyribonucleotide is then stably expressed in target tissues or cells without triggering cytotoxic elimination.

In one embodiment, binding sites for miRNAs that are known to be expressed in immune cells, in particular, antigen-presenting cells, can be engineered into a polyribonucleotide of the disclosure to suppress the expression of the polyribonucleotide in antigen-presenting cells through miRNA-mediated RNA degradation, subduing the antigen-mediated immune response. Expression of the polyribonucleotide is maintained in non-immune cells where the immune cell-specific miRNAs are not expressed. In some embodiments, to prevent an immunogenic reaction against a liver-specific protein, any miR-122-binding site can be removed and a miR-142 (and/or mirR-146)-binding site can be engineered into the 5′UTR and/or 3′UTR of a polyribonucleotide of the disclosure.

To further drive the selective degradation and suppression in APCs and macrophage, a polyribonucleotide of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell-specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1--3p, hsa-let-7f-2--5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116: e118-e127; Vaz C et al., BMC Genomics, 2010, 11, 288, the content of each of which is incorporated herein by reference in its entirety).

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. miRNA-binding sites from any liver-specific miRNA can be introduced to or removed from a polyribonucleotide of the disclosure to regulate expression of the polyribonucleotide in the liver. Liver-specific miRNA-binding sites can be engineered alone or further in combination with immune cell (e.g., APC) miRNA-binding sites in a polyribonucleotide of the disclosure.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. MiRNA-binding sites from any lung-specific miRNA can be introduced to or removed from a polyribonucleotide of the disclosure to regulate expression of the polyribonucleotide in the lung. Lung-specific miRNA-binding sites can be engineered alone or further in combination with immune cell (e.g., APC) miRNA-binding sites in a polyribonucleotide of the disclosure.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. MiRNA-binding sites from any heart-specific microRNA can be introduced to or removed from a polyribonucleotide of the disclosure to regulate expression of the polyribonucleotide in the heart. Heart-specific miRNA-binding sites can be engineered alone or further in combination with immune cell (e.g., APC) miRNA-binding sites in a polyribonucleotide of the disclosure.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. MiRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. MiRNA-binding sites from any CNS-specific miRNA can be introduced to or removed from a polyribonucleotide of the disclosure to regulate expression of the polyribonucleotide in the nervous system. Nervous system-specific miRNA-binding sites can be engineered alone or further in combination with immune cell (e.g., APC) miRNA-binding sites in a polyribonucleotide of the disclosure.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. MiRNA-binding sites from any pancreas-specific miRNA can be introduced to or removed from a polyribonucleotide of the disclosure to regulate expression of the polyribonucleotide in the pancreas. Pancreas-specific miRNA-binding sites can be engineered alone or further in combination with immune cell (e.g., APC) miRNA-binding sites in a polyribonucleotide of the disclosure.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. MiRNA-binding sites from any kidney-specific miRNA can be introduced to or removed from a polyribonucleotide of the disclosure to regulate expression of the polyribonucleotide in the kidney. Kidney-specific miRNA-binding sites can be engineered alone or further in combination with immune cell (e.g., APC) miRNA-binding sites in a polyribonucleotide of the disclosure.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. MiRNA-binding sites from any muscle-specific miRNA can be introduced to or removed from a polyribonucleotide of the disclosure to regulate expression of the polyribonucleotide in the muscle. Muscle-specific miRNA-binding sites can be engineered alone or further in combination with immune cell (e.g., APC) miRNA-binding sites in a polyribonucleotide of the disclosure.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep sequencing analysis (e.g., Voellenkle C et al., RNA, 2012, 18, 472-484, herein incorporated by reference in its entirety). MiRNA-binding sites from any endothelial cell-specific miRNA can be introduced to or removed from a polyribonucleotide of the disclosure to regulate expression of the polyribonucleotide in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. MiRNA-binding sites from any epithelial cell-specific miRNA can be introduced to or removed from a polyribonucleotide of the disclosure to regulate expression of the polyribonucleotide in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13 (5), 757-764; Vidigal J A and Ventura A, Semin Cancer Biol. 2012, 22 (5-6), 428-436; Goff L A et al., PLOS One, 2009, 4: e7192; Morin R D et al., Genome Res, 2008, 18, 610-621; Yoo J K et al., Stem Cells Dev. 2012, 21 (11), 2049-2057, each of which is herein incorporated by reference in its entirety). MiRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-5480-3p, miR-5480-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941, miR-96-3p, miR-96-5p, miR-99b-3p, and miR-99b-5p. Many predicted novel miRNAs are discovered by deep sequencing in human embryonic stem cells (e.g., Morin R D et al., Genome Res, 2008, 18, 610-621; Goff L A et al., PLOS One, 2009, 4: e7192; Bar M et al., Stem cells, 2008, 26, 2496-2505, the content of each of which is incorporated herein by reference in its entirety).

In one embodiment, the binding sites of embryonic stem cell-specific miRNAs can be included in or removed from the 3′UTR of a polyribonucleotide of the disclosure to modulate the development and/or differentiation of embryonic stem cells, to inhibit the senescence of stem cells in a degenerative condition (e.g., degenerative diseases), or to stimulate the senescence and apoptosis of stem cells in a disease condition (e.g., cancer stem cells).

Many miRNA expression studies are conducted to profile the differential expression of miRNAs in various cancer cells/tissues and other diseases. Some miRNAs are abnormally over-expressed in certain cancer cells and others are under-expressed. In some embodiments, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in its entirety).

As a non-limiting example, miRNA-binding sites for miRNAs that are over-expressed in certain cancer and/or tumor cells can be removed from the 3′UTR of a polyribonucleotide of the disclosure, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

MiRNA can also regulate complex biological processes such as angiogenesis (e.g., miR-132) (Anand and Cheresh Curr Opin Hematol 2011 18:171-176). In the polyribonucleotides of the disclosure, miRNA-binding sites that are involved in such processes can be removed or introduced, in order to tailor the expression of the polyribonucleotides to biologically relevant cell types or relevant biological processes. In this context, the polyribonucleotides of the disclosure are defined as auxotrophic polyribonucleotides.

### Peptide/Polypeptide Therapeutic Agents

In some embodiments, the therapeutic agent is a peptide therapeutic agent. In some embodiments, the therapeutic agent is a polypeptide therapeutic agent.

In some embodiments, the peptide or polypeptide is naturally derived, e.g., isolated from a natural source. In other embodiments, the peptide or polypeptide is a synthetic molecule, e.g., a synthetic peptide or polypeptide produced in vitro. In some embodiments, the peptide or polypeptide is a recombinant molecule. In some embodiments, the peptide or polypeptide is a chimeric molecule. In some embodiments, the peptide or polypeptide is a fusion molecule. In some embodiments, the peptide or polypeptide therapeutic agent of the composition is a naturally occurring peptide or polypeptide. In some embodiments, the peptide or polypeptide therapeutic agent of the composition is a modified version of a naturally occurring peptide or polypeptide (e.g., contains less than 3, less than 5, less than 10, less than 15, less than 20, or less than 25 amino substitutions, deletions, or additions compared to its wild type, naturally occurring peptide or polypeptide counterpart).

In some embodiments, the one or more therapeutic and/or prophylactic agents are a polynucleotide or a polypeptide.

### Genome Editing Techniques

In some embodiments, the nucleic acid is suitable for a genome editing technique.

In some embodiments, the genome editing technique is clustered regularly interspaced short palindromic repeats (CRISPR) or transcription activator-like effector nuclease (TALEN).

In some embodiments, the nucleic acid is at least one nucleic acid suitable for a genome editing technique selected from the group consisting of a CRISPR RNA (crRNA), a trans-activating crRNA (tracrRNA), a single guide RNA (sgRNA), and a DNA repair template.

### Vaccines

In some embodiments, the therapeutic and/or prophylactic is a ribonucleic acid (RNA) cancer vaccine of an RNA (e.g., messenger RNA (mRNA)) that can safely direct the body's cellular machinery to produce nearly any cancer protein or fragment thereof of interest. In some embodiments, the RNA is a modified RNA. The RNA vaccines of the present disclosure may be used to induce a balanced immune response against cancers, comprising both cellular and humoral immunity, without risking the possibility of insertional mutagenesis, for example.

The RNA vaccines may be utilized in various settings depending on the prevalence of the cancer or the degree or level of unmet medical need. The RNA vaccines may be utilized to treat and/or prevent a cancer of various stages or degrees of metastasis. The RNA vaccines have superior properties in that they produce much larger antibody titers and produce responses earlier than alternative anti-cancer therapies including cancer vaccines. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines which are manufactured ex vivo and may trigger unwanted cellular responses, the RNA vaccines are presented to the cellular system in a more native fashion.

Some embodiments of the present disclosure provide cancer vaccines that include at least one ribonucleic acid (RNA) polynucleotide having an open reading frame encoding at least one cancer antigenic polypeptide or an immunogenic fragment thereof (e.g., an immunogenic fragment capable of inducing an immune response to cancer). Other embodiments include at least one ribonucleic acid (RNA) polynucleotide having an open reading frame encoding two or more antigens or epitopes capable of inducing an immune response to cancer.

The invention in some embodiments is a vaccine of an mRNA having an open reading frame encoding a cancer antigen and an mRNA having an open reading frame encoding an immune checkpoint modulator. In some embodiments, the immune checkpoint modulator is an inhibitory checkpoint polypeptide. In some embodiments, the inhibitory checkpoint polypeptide is an antibody or fragment thereof that specifically binds to a molecule selected from the group consisting of PD-1, TIM-3, VISTA, A2AR, B7-H3, B7-H4, BTLA, CTLA-4, IDO, KIR and LAG3. The inhibitory checkpoint polypeptide is an anti-CTLA4 or anti-PDl antibody in some embodiments. Optionally the vaccine includes a lipid assembly. In some embodiments a vaccine of an mRNA having an open reading frame encoding a cancer antigen is administered to a subject. In other embodiments a checkpoint inhibitor is administered 3-10 weeks later. In some embodiments the checkpoint inhibitor is administered 4 weeks later.

In other embodiments the invention is a personalized cancer vaccine of an mRNA having an open reading frame encoding at least 2 cancer antigens, wherein the at least 2 cancer antigens are patient-specific cancer antigens, and a lipid nanoparticle carrier. In some embodiments the lipid nanoparticle has a mean diameter of 50-200 nm.

In yet other embodiments, the invention is a personalized cancer vaccine of an mRNA having an open reading frame encoding at least 2 cancer antigens wherein the at least 2 cancer antigens are representative of antigens of a patient. In some embodiments, the antigens of a patient are exosome-identified antigens of the patient. In some embodiments, a single mRNA encodes the cancer antigens. In other embodiments a plurality of mRNA encode the cancer antigens.

Each mRNA may encode 5-10 cancer antigens or a single cancer antigen in other embodiments. In some embodiments, the mRNA encodes 2-100 cancer antigens. In other embodiments, mRNA encodes 10-100, 20-100, 50-100, 100-200, 300-400, 500-600, 600-700, 700-800, 900-1,000, or 1,000-10,000 cancer antigens.

In some embodiments,
a) the mRNA encoding each cancer antigen is interspersed by cleavage sensitive sites;
b) the mRNA encoding each cancer antigen is linked directly to one another without a linker;
c) the mRNA encoding each cancer antigen is linked to one another with a single nucleotide linker;
d) each cancer antigen comprises a 25-35 amino acids and includes a centrally located SNP mutation;
e) at least 30% of the cancer antigens have a highest affinity for class I MHC molecules from the subject;
f) at least 30% of the cancer antigens have a highest affinity for class II MHC molecules from the subject;
g) at least 50% of the cancer antigens have a predicted binding affinity of IC>500 nM for HLA-A, HLA-B and/or DRB 1;
h) the mRNA encodes 20 cancer antigens;
i) 50% of the cancer antigens have a binding affinity for class I MHC and 50% of the cancer antigens have a binding affinity for class II MHC; and/or
j) the mRNA encoding the cancer antigens is arranged such that the cancer antigens are ordered to minimize pseudo-epitopes.

In some embodiments, each cancer antigen comprises 31 amino acids and includes a centrally located SNP mutation with 15 flanking amino acids on each side of the SNP mutation.

In some embodiments, the vaccine is a personalized cancer vaccine and wherein the cancer antigen is a subject specific cancer antigen. In some embodiments, the subject specific cancer antigen may be representative of an exome of a tumor sample of the subject, or of a transcriptome of a tumor sample of the subject. In some embodiments, the subject specific cancer antigen may be representative of an exosome of the subject.

In some embodiments, the open reading frame further encodes one or more traditional cancer antigens. In some embodiments, the traditional cancer antigen is a non-mutated antigen. In some embodiments, the traditional cancer antigen is a mutated antigen.

In some embodiments, the mRNA vaccine further comprises an mRNA having an open reading frame encoding one or more traditional cancer antigens.

In some embodiments, a single mRNA encodes the cancer antigens. In other embodiments, a plurality of mRNA encode the cancer antigens. Each cancer antigen is 10-50 amino acids in length in some embodiments. In other embodiments, each cancer antigen is 15-20 amino acids in length. In other embodiments, the cancer antigen is 20-50, 25-100, 100-200, 200-300, 300-400, 400-500, 500-1,000, or 1,000-10,000 amino acids in length.

In some embodiments, the vaccines further comprise an adjuvant.

Some embodiments of the present disclosure provide a cancer vaccine that includes at least one ribonucleic acid (RNA) polynucleotide having an open reading frame encoding at least one cancer polypeptide, at least one 5′ terminal cap and at least one chemical modification, formulated within a lipid assembly. In some embodiments, a 5′ terminal cap is 7 mG(5′)ppp(5′)NlmpNp.

In some embodiments, at least one chemical modification is selected from pseudouridine, Nl-methylpseudouridine, Nl-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, 5-dihydropseudouridine, 5-methyluridine, methoxyuridine, and 2′-O-methyl uridine. In some embodiments, the extent of incorporation of chemically modified nucleotides has been optimized for improved immune responses to the vaccine formulation.

In some embodiments, a lipid assembly comprises a cationic lipid, a PEG-modified lipid, a sterol, and a non-cationic lipid. In some embodiments, a cationic lipid is an ionizable cationic lipid and the non-cationic lipid is a neutral lipid, and the sterol is a cholesterol. In some embodiments, a cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319).

In some embodiments, the lipid assembly formulation includes an immune potentiator (e.g., TLR agonist) to enhance immunogenicity of the vaccine (formulation).

In some embodiments, 100% of the uracil in the open reading frame have a chemical modification. In some embodiments, a chemical modification is in the 5-position of the uracil. In some embodiments, a chemical modification is a Nl-methyl pseudouridine.

In other embodiments, an mRNA encoding an APC reprograming molecule is included in the vaccine or coadministered with the vaccine. The APC reprograming molecule may be a CIITA, a chaperone protein such as CLIP, HLA-DO, or HLA-DM, a costimulatory molecule such as CD40, CD80, or CD86, a CIITA fragment such as amino acids 26-137 of CIITA or a protein having 80% sequence identity to CIITA.

In other embodiments, a method of eliciting an immune response in a subject by identifying at least 2 cancer antigens from a sample of a subject, wherein the at least 2 cancer antigens include mutations selected from the group consisting of frame-shift mutations and recombinations, and administering an mRNA vaccine having an open reading frame encoding the at least 2 cancer antigens to the subject is provided.

In some embodiments, the cancer antigens are identified from an exosome of the subject. In some embodiments, 2-100 antigens are identified from the exosome. In other embodiments, the mRNA vaccine has an open reading frame encoding the 2-100 antigens. A single mRNA or a plurality of mRNA may encode the antigens.

In some embodiments, the antigens are cancer antigens. The cancer antigens may have mutations selected from point mutations, frame-shift mutations and recombinations. The method may further involve confirming that the cancer antigens are subject specific by exome analysis.

In some embodiments, the method may further involve confirming that the cancer antigens are subject specific by transcriptome analysis.

In some embodiments the method also involves at least one month after the administration of the mRNA vaccine, identifying at least 2 cancer antigens from a sample of the subject to produce a second set of cancer antigens, and administering to the subject an mRNA vaccine having an open reading frame encoding the second set of cancer antigens to the subject.

In other embodiments, the sample of the subject is a tumor sample.

In other embodiments, the invention comprises a method of eliciting an immune response in a subject by identifying at least 2 cancer antigens from a sample of a subject to produce a first set of cancer antigens, administering to the subject an mRNA vaccine having an open reading frame encoding the first set of cancer antigens to the subject, at least one month after the administration of the mRNA vaccine, identifying at least 2 cancer antigens from a sample of a subject to produce a second set of cancer antigens, and administering to the subject an mRNA vaccine having an open reading frame encoding the second set of cancer antigens to the subject.

The mRNA vaccine having an open reading frame encoding second set of antigens, in some embodiments, is administered to the subject 6 months to 1 year after the mRNA vaccine having an open reading frame encoding first set of cancer antigens. In other embodiments, the mRNA vaccine having an open reading frame encoding second set of antigens is administered to the subject 1-2 years after the mRNA vaccine having an open reading frame encoding first set of cancer antigens.

In some embodiments, a single mRNA has an open reading frame encoding the cancer antigens. In other embodiments, a plurality of Mrna encode the antigens. In some embodiments, the second set of cancer antigens includes 2-100 antigens. In other embodiments, the cancer antigens have mutations selected from point mutations, frame-shift mutations, and recombinations.

In other embodiments, the invention comprises a method of eliciting an immune response in a subject, by identifying at least 2 cancer antigens from a sample of a subject, administering an mRNA having an open reading frame encoding the at least 2 cancer antigens to the subject, and administering a cancer therapeutic agent to the subject. In some embodiments, the cancer therapeutic agent is a targeted therapy. The targeted therapy may be a BRAF inhibitor such as vemurafenib (PLX4032) or dabrafenib.

In other embodiments, the cancer therapeutic agent is a T-cell therapeutic agent. The T-cell therapeutic agent may be a checkpoint inhibitor such as an anti-PD-1 antibody or an anti-CTLA-4 antibody. In some embodiments, the anti-PD-1 antibody is BMS-936558 (nivolumab). In other embodiments, the anti-CTLA-4 antibody is ipilimumab. The T-cell therapeutic agent in other embodiments is OX40L. In yet other embodiments, the cancer therapeutic agent is a vaccine comprising a population-based tumor-specific antigen.

In other embodiments, the cancer therapeutic agent is a vaccine comprising an mRNA having an open reading frame encoding one or more traditional cancer antigens.

In some embodiments, the mRNA having an open reading frame encoding the at least 2 cancer antigens is administered to the subject simultaneously with the cancer therapeutic agent. In some embodiments, the mRNA having an open reading frame encoding the at least 2 cancer antigens is administered to the subject before administration of the cancer therapeutic agent. In some embodiments, the mRNA having an open reading frame encoding the at least 2 cancer antigens is administered to the subject after administration of the cancer therapeutic agent.

A method comprising mixing an mRNA having an open reading frame encoding a cancer antigen with a lipid assembly formulation to produce an mRNA cancer vaccine, and administering the mRNA cancer vaccine to a subject within 24 hours of mixing is provided in other embodiments of the invention. In some embodiments, the mRNA cancer vaccine is administered to the subject within 12 hours of mixing. In other embodiments, the mRNA cancer vaccine is administered to the subject within 1 hour of mixing. The mRNA cancer vaccine encodes 2-100 cancer antigens or 10-100 cancer antigens in some embodiments.

In some embodiments, the vaccine is a personalized cancer vaccine and wherein the cancer antigen is a subject specific cancer antigen.

In some embodiments, a single mRNA encodes the cancer antigens. In other embodiments a plurality of mRNA encode the cancer antigens. Each mRNA encodes 5-10 cancer antigens or a single cancer antigen in other embodiments. In yet other embodiments, each cancer antigen is 10-50 amino acids in length or 15-20 amino acids in length.

Further provided herein are uses of cancer vaccines in the manufacture of a medicament for use in a method of inducing an antigen-specific immune response in a subject, the method comprising administering the cancer vaccine to the subject in an amount effective to produce an antigen-specific immune response.

A method of treating cancer in a subject in need thereof by identifying at least 2 cancer antigens from an exosome isolated from the subject; producing, based on the identified antigens, an mRNA vaccine having an open reading frame encoding the antigens; and administering the mRNA vaccine to the subject, wherein the mRNA vaccine induces a tumor-specific immune response in the subject, thereby treating cancer in the subject is provided in other embodiments. The invention in other embodiments is an RNA vaccine preparable according to a method involving identifying at least 2 cancer antigens from an exosome isolated from a subject; producing, based on the identified antigens, an mRNA vaccine having an open reading frame encoding the antigens.

A method of eliciting an immune response in a subject against a cancer antigen is provided in embodiments of the invention. The method involves administering to the subject an RNA vaccine comprising at least one RNA polynucleotide having an open reading frame encoding at least one antigenic polypeptide or an immunogenic fragment thereof, thereby inducing in the subject an immune response specific to the antigenic polypeptide or an immunogenic fragment thereof, wherein the anti-antigenic polypeptide antibody titer in the subject is increased following vaccination relative to anti-antigenic polypeptide antibody titer in a subject vaccinated with a prophylactically effective dose of a traditional vaccine against the cancer. An “anti-antigenic polypeptide antibody” is a serum antibody the binds specifically to the antigenic polypeptide.

A prophylactically effective dose is a therapeutically effective dose that prevents advancement of cancer at a clinically acceptable level. In some embodiments, the therapeutically effective dose is a dose listed in a package insert for the vaccine. A traditional vaccine, as used herein, refers to a vaccine other than the mRNA vaccines of the invention. For instance, a traditional vaccine includes but is not limited to live microorganism vaccines, killed microorganism vaccines, subunit vaccines, protein antigen vaccines, DNA vaccines, etc. In exemplary embodiments, a traditional vaccine is a vaccine that has achieved regulatory approval and/or is registered by a national drug regulatory body, for example, the Food and Drug Administration (FDA) in the United States or the European Medicines Agency (EMA.)

In some embodiments, the anti-antigenic polypeptide antibody titer in the subject is increased 1 log to 10 log following vaccination relative to anti-antigenic polypeptide antibody titer in a subject vaccinated with a prophylactically effective dose of a traditional vaccine against the cancer.

In some embodiments, the anti-antigenic polypeptide antibody titer in the subject is increased 1 log following vaccination relative to anti-antigenic polypeptide antibody titer in a subject vaccinated with a prophylactically effective dose of a traditional vaccine against the cancer.

In some embodiments, the anti-antigenic polypeptide antibody titer in the subject is increased 2 log following vaccination relative to anti-antigenic polypeptide antibody titer in a subject vaccinated with a prophylactically effective dose of a traditional vaccine against the cancer.

In some embodiments, the anti-antigenic polypeptide antibody titer in the subject is increased 3 log following vaccination relative to anti-antigenic polypeptide antibody titer in a subject vaccinated with a prophylactically effective dose of a traditional vaccine against the cancer.

In some embodiments, the anti-antigenic polypeptide antibody titer in the subject is increased 5 log following vaccination relative to anti-antigenic polypeptide antibody titer in a subject vaccinated with a prophylactically effective dose of a traditional vaccine against the or cancer.

In some embodiments, the anti-antigenic polypeptide antibody titer in the subject is increased 10 log following vaccination relative to anti-antigenic polypeptide antibody titer in a subject vaccinated with a prophylactically effective dose of a traditional vaccine against the or cancer.

A method of eliciting an immune response in a subject against a cancer antigen is provided in other embodiments of the invention. The method involves administering to the subject an RNA vaccine comprising at least one RNA polynucleotide having an open reading frame encoding at least one antigenic polypeptide or an immunogenic fragment thereof, thereby inducing in the subject an immune response specific to antigenic polypeptide or an immunogenic fragment thereof, wherein the immune response in the subject is equivalent to an immune response in a subject vaccinated with a traditional vaccine against the cancer antigen at 2 times to 100 times the dosage level relative to the RNA vaccine.

In some embodiments, the immune response in the subject is equivalent to an immune response in a subject vaccinated with a traditional vaccine at twice the dosage level relative to the RNA vaccine.

In some embodiments, the immune response in the subject is equivalent to an immune response in a subject vaccinated with a traditional vaccine at three times the dosage level relative to the RNA vaccine.

In some embodiments, the immune response in the subject is equivalent to an immune response in a subject vaccinated with a traditional vaccine at 4 times the dosage level relative to the RNA vaccine.

In some embodiments, the immune response in the subject is equivalent to an immune response in a subject vaccinated with a traditional vaccine at 5 times the dosage level relative to the RNA vaccine. In some embodiments, the immune response in the subject is equivalent to an immune response in a subject vaccinated with a traditional vaccine at 10 times the dosage level relative to the RNA vaccine.

In some embodiments, the immune response in the subject is equivalent to an immune response in a subject vaccinated with a traditional vaccine at 50 times the dosage level relative to the RNA vaccine.

In some embodiments, the immune response in the subject is equivalent to an immune response in a subject vaccinated with a traditional vaccine at 100 times the dosage level relative to the RNA vaccine.

In some embodiments, the immune response in the subject is equivalent to an immune response in a subject vaccinated with a traditional vaccine at 10 times to 1000 times the dosage level relative to the RNA vaccine.

In some embodiments, the immune response in the subject is equivalent to an immune response in a subject vaccinated with a traditional vaccine at 100 times to 1000 times the dosage level relative to the RNA vaccine.

In other embodiments, the immune response is assessed by determining antibody titer in the subject.

In other embodiments, the invention comprises a method of eliciting an immune response in a subject by administering to the subject an RNA vaccine comprising at least one RNA polynucleotide having an open reading frame encoding at least one cancer antigenic polypeptide or an immunogenic fragment thereof, thereby inducing in the subject an immune response specific to the antigenic polypeptide or an immunogenic fragment thereof, wherein the immune response in the subject is induced 2 days to 10 weeks earlier relative to an immune response induced in a subject vaccinated with a prophylactically effective dose of a traditional vaccine against the cancer antigen. In some embodiments, the immune response in the subject is induced in a subject vaccinated with a prophylactically effective dose of a traditional vaccine at 2 times to 100 times the dosage level relative to the RNA vaccine.

In some embodiments, the immune response in the subject is induced 2 days earlier relative to an immune response induced in a subject vaccinated with a prophylactically effective dose of a traditional vaccine.

In some embodiments, the immune response in the subject is induced 3 days earlier relative to an immune response induced in a subject vaccinated a prophylactically effective dose of a traditional vaccine. In some embodiments, the immune response in the subject is induced 1 week earlier relative to an immune response induced in a subject vaccinated with a prophylactically effective dose of a traditional vaccine.

In some embodiments, the immune response in the subject is induced 2 weeks earlier relative to an immune response induced in a subject vaccinated with a prophylactically effective dose of a traditional vaccine.

In some embodiments, the immune response in the subject is induced 3 weeks earlier relative to an immune response induced in a subject vaccinated with a prophylactically effective dose of a traditional vaccine.

In some embodiments, the immune response in the subject is induced 5 weeks earlier relative to an immune response induced in a subject vaccinated with a prophylactically effective dose of a traditional vaccine.

In some embodiments, the immune response in the subject is induced 10 weeks earlier relative to an immune response induced in a subject vaccinated with a prophylactically effective dose of a traditional vaccine.

A method of eliciting an immune response in a subject against a cancer by administering to the subject a cancer RNA vaccine having an open reading frame encoding a first antigenic polypeptide, wherein the RNA polynucleotide does not include a stabilization element, and wherein an adjuvant is not coformulated or co-administered with the vaccine.

In yet other embodiments, the invention comprises a method of producing an mRNA encoding a concatemeric cancer antigen comprising between 1000 and 3000 nucleotides, the method by
(a) binding a first polynucleotide comprising an open reading frame encoding the concatemeric cancer antigen and a second polynucleotide comprising a 5′-UTR to a polynucleotide conjugated to a solid support;
(b) ligating the 3 ‘-terminus of the second polynucleotide to the 5’-terminus of the first polynucleotide under suitable conditions, wherein the suitable conditions comprise a DNA Ligase, thereby producing a first ligation product;
(c) ligating the 5′ terminus of a third polynucleotide comprising a 3′-UTR to the 3′-terminus of the first ligation product under suitable conditions, wherein the suitable conditions comprise an RNA Ligase, thereby producing a second ligation product; and
(d) releasing the second ligation product from the solid support, thereby producing an mRNA encoding the concatemeric cancer antigen comprising between 1000 and 3000 nucleotides. In some embodiments of any one of the provided compositions or methods, the mRNA encodes one or more recurrent polymorphisms. In some embodiments, the one or more recurrent polymorphisms comprises a recurrent somatic cancer mutation in p53. In some such embodiments, the one or more recurrent somatic cancer mutation in p53 are selected from the group consisting of:
   (1) mutations at the canonical 5′ splice site neighboring codon p.T125;
   (2) mutations at the canonical 5′ splice site neighboring codon p.331;
   (3) mutations at the canonical 3′ splice site neighboring codon p.126; and
   (4) mutations at the canonical 5′ splice site neighboring codon p.224, inducing a cryptic alternative intronic 5′ splice site.

In one embodiment, the invention provides a cancer therapeutic vaccine comprising mRNA encoding an open reading frame (ORF) coding for one or more of neoantigen peptides (1) through (4). In one embodiment, the invention provides the selective administration of a vaccine containing or coding for one or more of peptides (1)-(4), based on the patient's tumor containing any of the above mutations. In one embodiment, the invention provides the selective administration of the vaccine based on the dual criteria of the subject's tumor containing any of the above mutations and the subject's normal HLA type containing the corresponding HLA allele predicted to bind to the resulting neoantigen.

A method for treating a subject with a personalized mRNA cancer vaccine, by isolating a sample from a subject, identifying a set of neoepitopes by analyzing a patient transcriptome and/or a patient exome from the sample to produce a patient specific mutanome, selecting a set of neoepitopes for the vaccine from the mutanome based on MHC binding strength, MHC binding diversity, predicted degree of immunogenicity, low self reactivity, and/or T cell reactivity, preparing the mRNA vaccine to encode the set of neoepitopes and administering the mRNA vaccine to the subject within two months of isolating the sample from the subject is provided in other embodiments of the invention. In some embodiments, the mRNA vaccine is administered to the subject within one month of isolating the sample from the subject.

In other embodiments, the invention comprises a method of identifying a set of neoepitopes for use in a personalized mRNA cancer vaccine having one or more polynucleotides that encode the set of neoepitopes by a) identifying a patient-specific mutanome by analyzing a patient transcriptome and a patient exome, b) selecting a subset of 15-500 neoepitopes from the mutanome using a weighted value for the neoepitopes based on at least three of: an assessment of gene or transcript-level expression in patient RNA-seq; variant call confidence score; RNA-seq allele-specific expression; conservative vs. non-conservative amino acid substitution; position of point mutation (Centering Score for increased TCR engagement); position of point mutation (Anchoring Score for differential HLA binding); Selfness: <100% core epitope homology with patient WES data; HLA-A and -B IC50 for 8mers-1 lmers; HLA-DRB 1 IC50 for 15mers-20mers; promiscuity Score (i.e. number of patient HLAs predicted to bind); HLA-C IC50 for 8mers-1 lmers; HLA-DRB3-5 IC50 for 15mers-20mers; HLA-DQB 1/A1 IC50 for 15mers-20mers; HLA-DPB 1/A1 IC50 for 15mers-20mers; Class I vs Class II proportion; Diversity of patient HLA-A, —B and DRB 1 allotypes covered; proportion of point mutation vs complex epitopes (e.g. frameshifts); and/or pseudo-epitope HLA binding scores, and c) selecting the set of neoepitopes for use in a personalized mRNA cancer vaccine from the subset based on the highest weighted value, wherein the set of neoepitopes comprise 15-40 neoepitopes.

In some embodiments, the nucleic acid vaccines described herein are chemically modified. In other embodiments, the nucleic acid vaccines are unmodified.

Yet other embodiments provide compositions for and methods of vaccinating a subject comprising administering to the subject a nucleic acid vaccine comprising one or more RNA polynucleotides having an open reading frame encoding a first antigenic polypeptide or a concatemeric polypeptide, wherein the RNA polynucleotide does not include a stabilization element, and wherein an adjuvant is not coformulated or co-administered with the vaccine.

In other embodiments, the invention is a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments, the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty-one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100-fold higher level in the local lymph node in comparison with the distal lymph node. In other embodiments, the nucleic acid vaccine is chemically modified, and in other embodiments, the nucleic acid vaccine is not chemically modified.

Embodiments of the invention provide a nucleic acid vaccine comprising one or more RNA polynucleotides having an open reading frame encoding a first antigenic polypeptide or a concatemeric polypeptide, wherein the RNA polynucleotide does not include a stabilization element and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Embodiments of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the invention is a neutralizing antibody titer. In some embodiments, the neutralizing antibody titer is greater than a protein vaccine. In other embodiments, the neutralizing antibody titer produced by the mRNA vaccines of the invention is greater than an adjuvanted protein vaccine. In yet other embodiments, the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

In some embodiments, the vaccines produce prophylactically and/or therapeutically efficacious levels, concentrations and/or titers of antigen-specific antibodies in the blood or serum of a vaccinated subject. As defined herein, the term antibody titer refers to the amount of antigen-specific antibody produces in a subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In some embodiments, antibody titer measurement is expressed as a ratio, such as 1:40, 1:100, etc.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose).

In exemplary embodiments of the invention, antigen-specific antibodies are measured in units of μg/ml or are measured in units of IU/L (International Units per liter) or mlU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml, or >10 μg/ml. In exemplary embodiments of the invention, an efficacious vaccine produces >10 mlU/ml, >20 mlU/ml, >50 mlU/ml, >100 mlU/ml, >200 mlU/ml, >500 mlU/ml, or >1000 mlU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose). In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay. Also provided are nucleic acid vaccines comprising one or more RNA polynucleotides having an open reading frame encoding a first antigenic polypeptide or a concatemeric polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce a neutralizing antibody within one week of a single administration. In some embodiments, the adjuvant is selected from a cationic peptide and an immunostimulatory nucleic acid. In some embodiments, the cationic peptide is protamine.

Embodiments provide nucleic acid vaccines comprising one or more RNA polynucleotides having an open reading frame comprising at least one chemical modification or optionally no nucleotide modification, the open reading frame encoding a first antigenic polypeptide or a concatemeric polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other embodiments provide nucleic acid vaccines comprising one or more RNA polynucleotides having an open reading frame comprising at least one chemical modification or optionally no nucleotide modification, the open reading frame encoding a first antigenic polypeptide or a concatemeric polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Embodiments of the invention also provide a unit of use vaccine, comprising between 10 ug and 400 μg of one or more RNA polynucleotides having an open reading frame comprising at least one chemical modification or optionally no nucleotide modification, the open reading frame encoding a first antigenic polypeptide or a concatemeric polypeptide, and a pharmaceutically acceptable carrier or excipient, formulated for delivery to a human subject. In some embodiments, the vaccine further comprises a cationic lipid assembly.

Embodiments of the invention provide methods of creating, maintaining or restoring antigenic memory to a tumor in an individual or population of individuals comprising administering to said individual or population an antigenic memory booster nucleic acid vaccine comprising (a) at least one RNA polynucleotide, said polynucleotide comprising at least one chemical modification or optionally no nucleotide modification and two or more codon-optimized open reading frames, said open reading frames encoding a set of reference antigenic polypeptides and (b) optionally a pharmaceutically acceptable carrier or excipient. In some embodiments, the vaccine is administered to the individual via a route selected from the group consisting of intramuscular administration, intradermal administration, and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Embodiments of the invention provide methods of vaccinating a subject comprising administering to the subject a single dosage of between 25 μg/kg and 400 μg/kg of a nucleic acid vaccine comprising one or more RNA polynucleotides having an open reading frame encoding a first antigenic polypeptide or a concatemeric polypeptide in an effective amount to vaccinate the subject.

Other embodiments provide nucleic acid vaccines comprising one or more RNA polynucleotides having an open reading frame comprising at least one chemical modification, the open reading frame encoding a first antigenic polypeptide or a concatemeric polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Other embodiments provide nucleic acid vaccines comprising a lipid assembly formulated RNA polynucleotide having an open reading frame comprising no nucleotide modifications (unmodified), the open reading frame encoding a first antigenic polypeptide or a concatemeric polypeptide, wherein the vaccine has at least 10-fold less RNA polynucleotide than is required for an unmodified mRNA vaccine not formulated in a lipid assembly to produce an equivalent antibody titer. In some embodiments, the RNA polynucleotide is present in a dosage of 25-100 micrograms.

In other embodiments, the invention encompasses a method of treating an elderly subject age 60 years or older comprising administering to the subject a nucleic acid vaccine comprising one or more RNA polynucleotides having an open reading frame encoding an antigenic polypeptide or a concatemeric polypeptide in an effective amount to vaccinate the subject.

In other embodiments, the invention encompasses a method of treating a young subject age 17 years or younger comprising administering to the subject a nucleic acid vaccine comprising one or more RNA polynucleotides having an open reading frame encoding an antigenic polypeptide or a concatemeric polypeptide in an effective amount to vaccinate the subject.

In other embodiments, the invention encompasses a method of treating an adult subject comprising administering to the subject a nucleic acid vaccine comprising one or more RNA polynucleotides having an open reading frame encoding an antigenic polypeptide or a concatemeric polypeptide in an effective amount to vaccinate the subject.

In some embodiments, the invention comprises a method of vaccinating a subject with a combination vaccine including at least two nucleic acid sequences encoding antigens wherein the dosage for the vaccine is a combined therapeutic dosage wherein the dosage of each individual nucleic acid encoding an antigen is a sub therapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments, the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject. In some embodiments, the sub therapeutic dosage of each individual nucleic acid encoding an antigen is 1, 2, 3, 4, 5, 6, 7, 8, 9, 10, 11, 12, 13, 14, 15, 16, 17, 18, 19, or 20 micrograms. In other embodiments, the nucleic acid vaccine is chemically modified, and in other embodiments, the nucleic acid vaccine is not chemically modified.

### Other Components

A lipid assembly may include one or more components in addition to those described in the preceding sections. In some embodiments, a lipid assembly may include one or more small hydrophobic molecules such as a vitamin (e.g., vitamin A or vitamin E) or a sterol.

Lipid assemblies may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid assembly. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. In some embodiments, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes, such as polyethylene and polypropylene, polyalkylene glycols, such as poly(ethylene glycol) (PEG), polyalkylene oxides (PEO), polyalkylene terephthalates, such as poly(ethylene terephthalate), polyvinyl alcohols (PVA), polyvinyl ethers, polyvinyl esters, such as poly(vinyl acetate), polyvinyl halides, such as poly(vinyl chloride) (PVC), polyvinylpyrrolidone (PVP), polysiloxanes, polystyrene, polyurethanes, derivatized celluloses, such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho) esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a lipid assembly (e.g., by coating, adsorption, covalent linkage, or other process).

A lipid assembly may also comprise one or more functionalized lipids. In some embodiments, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a lipid assembly may also be conjugated with one or more useful antibodies. Functional groups and conjugates useful in targeted cell delivery, imaging, and membrane permeation are well known in the art.

In addition to these components, lipid assemblies may include any substance useful in pharmaceutical compositions. In some embodiments, the lipid assembly may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art. (See, for example, Remington's The Science and Practice of Pharmacy, 21^{st} Edition, A. R. Gennaro; Lippincott, Williams & Wilkins, Baltimore, MD, 2006.)

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, propylene glycol monostearate, and polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, and methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, and sorbitan monooleate [SPAN®80]), polyoxyethylene esters (e.g., polyoxyethylene monostearate [MYRJR 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, and mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may be selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### Methods of Preparing the Lipid Assemblies

In some embodiments, the present disclosure provides a method of preparing the population of lipid assemblies described herein.

In some embodiments, the method comprises: i) mixing an ionizable lipid, a structural lipid, and a phospholipid, with a first buffer, thereby forming a population of intermediate empty lipid assemblies.

In some embodiments, the method comprises: i) mixing an ionizable lipid, a structural lipid, a phospholipid, and a PEG lipid, with a first buffer, thereby forming a population of intermediate empty lipid assemblies.

In some embodiments, the method further comprises: ii) adding a second buffer to the intermediate empty lipid assemblies, thereby forming a population of empty lipid assemblies.

In some embodiments, the method further comprises: iii) mixing a therapeutic agent (e.g., a nucleic acid) with the empty-lipid assemblies, thereby forming a population of loaded lipid assemblies.

In some embodiments, the method further comprises processing the empty lipid assemblies or the loaded lipid assemblies.

In some embodiments, the step of processing comprises:
a) adding a cryoprotectant to the empty lipid assemblies or the loaded lipid assemblies;
b) lyophilizing the empty lipid assemblies or the loaded lipid assemblies;
c) storing the lyophilized empty lipid assemblies or the lyophilized loaded lipid assemblies; and/or
d) adding a buffering solution to the lyophilized empty lipid assemblies or the lyophilized loaded lipid assemblies.

Suitable methods for preparing the population of lipid assemblies described herein are also described in PCT Application Publication No. WO/2020/160397, WO/2021/155274, and WO/2022/032087, each of which is incorporated herein by reference.

### Pharmaceutical Compositions

In some embodiments, the present disclosure provides a pharmaceutical composition, comprising the population of lipid assemblies described herein, and one or more pharmaceutically acceptable carriers or excipients.

In some embodiments, the pharmaceutical composition is free of therapeutic agent (e.g., RNA).

In some embodiments, the pharmaceutical composition comprises a therapeutic agent (e.g., RNA).

Pharmaceutical compositions may include one or more lipid assemblies. In some embodiments, a pharmaceutical composition may include one or more lipid assemblies including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a lipid assembly in the formulation of the disclosure. An excipient or accessory ingredient may be incompatible with a component of a lipid assembly of the formulation if its combination with the component or lipid assembly may result in any undesirable biological effect or otherwise deleterious effect.

In some embodiments, one or more excipients or accessory ingredients may make up greater than 50% of the total mass or volume of a pharmaceutical composition including a lipid assembly. In some embodiments, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid assemblies, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition comprises between 0.1% and 100% (wt/wt) of one or more lipid assemblies. As another example, a pharmaceutical composition comprises between 0.1% and 15% (wt/vol) of one or more amphiphilic polymers (e.g., 0.5%, 1%, 2.5%, 5%, 10%, or 12.5% w/v).

In some embodiments, the lipid assemblies and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid assemblies is a solution or solid (e.g., via lyophilization) that is refrigerated for storage and/or shipment at, for example, about −20° C., −30° C., −40° C., −50° C., −60° C., −70° C., or −80° C. In some embodiments, the disclosure also relates to a method of increasing stability of the lipid assemblies and by storing the lipid assemblies and/or pharmaceutical compositions thereof at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.).

Lipid assemblies and/or pharmaceutical compositions including one or more lipid assemblies may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of lipid assemblies and pharmaceutical compositions including lipid assemblies are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more lipid assemblies may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., lipid assembly). The amount of the active ingredient is generally equal to the dosage of the active ingredient which would be administered to a subject and/or a convenient fraction of such a dosage, such as, for example, one-half or one-third of such a dosage.

Pharmaceutical compositions may be prepared in a variety of forms suitable for a variety of routes and methods of administration. In some embodiments, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms comprise inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents, such as wetting agents, emulsifying and suspending agents, sweetening, flavoring, and/or perfuming agents. In some embodiments for parenteral administration, compositions are mixed with solubilizing agents, such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients, such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, films, powders, and granules. In such solid dosage forms, an active ingredient is mixed with at least one inert, pharmaceutically acceptable excipient, such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay and silicates), and lubricants (e.g., talc, calcium stearate, magnesium stearate, solid polyethylene glycols, and sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells, such as enteric coatings and other coatings well known in the pharmaceutical formulating art. They may optionally comprise opacifying agents and can be of a composition that they release the active ingredient(s) only. In some embodiments, the solid compositions may optionally comprise opacifying agents and can be of a composition that they release the active ingredient(s) in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices, such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations, such as liniments, lotions, oil in water and/or water in oil emulsions, such as creams, ointments and/or pastes, and/or solutions and/or suspensions. Topically administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container, such as a device comprising the active ingredient dissolved and/or suspended in a low-boiling propellant in a sealed container. Dry powder compositions may include a solid fine powder diluent, such as sugar, and are conveniently provided in a unit dose form.

Low-boiling propellants generally include liquid propellants having a boiling point of below 65° F. at atmospheric pressure. Generally, the propellant may constitute 50% to 99.9% (wt/wt) of the composition, and active ingredient may constitute 0.1% to 20% (wt/wt) of the composition. A propellant may further comprise additional ingredients, such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent, such as saccharin sodium, a volatile oil, a buffering agent, a surface-active agent, and/or a preservative, such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e., by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may comprise, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically administrable formulations which are useful include those which comprise the active ingredient in microcrystalline form and/or in a liposomal preparation. Ear drops and/or eye drops are contemplated as being within the scope of this present disclosure.

### Methods of Producing Polypeptides in Cells

The present disclosure provides methods of producing a polypeptide of interest in a mammalian cell. Methods of producing polypeptides involve contacting a cell with a formulation of the disclosure comprising a lipid assembly including an mRNA encoding the polypeptide of interest. Upon contacting the cell with the lipid assembly, the mRNA may be taken up and translated in the cell to produce the polypeptide of interest.

In general, the step of contacting a mammalian cell with a lipid assembly including an mRNA encoding a polypeptide of interest may be performed in vivo, ex vivo, in culture, or in vitro. The amount of lipid assembly contacted with a cell, and/or the amount of mRNA therein, may depend on the type of cell or tissue being contacted, the means of administration, the physiochemical characteristics of the lipid assembly and the mRNA (e.g., size, charge, and chemical composition) therein, and other factors. In general, an effective amount of the lipid assembly will allow for efficient polypeptide production in the cell. Metrics for efficiency may include polypeptide translation (indicated by polypeptide expression), level of mRNA degradation, and immune response indicators.

The step of contacting a lipid assembly including an mRNA with a cell may involve or cause transfection. A phospholipid including in the lipid component of a lipid assembly may facilitate transfection and/or increase transfection efficiency, for example, by interacting and/or fusing with a cellular or intracellular membrane. Transfection may allow for the translation of the mRNA within the cell.

In some embodiments, the lipid assemblies described herein may be used therapeutically. For example, an mRNA included in a lipid assembly may encode a therapeutic polypeptide (e.g., in a translatable region) and produce the therapeutic polypeptide upon contacting and/or entry (e.g., transfection) into a cell. In other embodiments, an mRNA included in a lipid assembly may encode a polypeptide that may improve or increase the immunity of a subject. In some embodiments, an mRNA may encode a granulocyte-colony stimulating factor or trastuzumab.

In some embodiments, an mRNA included in a lipid assembly may encode a recombinant polypeptide that may replace one or more polypeptides that may be substantially absent in a cell contacted with the lipid assembly. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment, such as the nucleus, or may be engineered for secretion from the cell or for translocation to the plasma membrane of the cell.

In some embodiments, contacting a cell with a lipid assembly including an mRNA may reduce the innate immune response of a cell to an exogenous nucleic acid. A cell may be contacted with a first lipid assembly including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

### Methods of Delivering Therapeutic Agents to Cells and Organs

In some embodiments, the present disclosure provides a method of delivering a therapeutic agent to a cell in a subject, comprising administering to the subject the population of lipid assemblies or pharmaceutical composition described herein.

In some embodiments, the present disclosure provides the population of lipid assemblies or pharmaceutical composition described herein for use in delivering a therapeutic agent to a cell in a subject.

In some embodiments, the present disclosure provides use of the population of lipid assemblies or composition described herein in the manufacture of a medicament for delivering a therapeutic agent to a cell in a subject.

In some embodiments, the cell is a hematopoietic stem and progenitor cell (HSPC).

In some embodiments, the present disclosure provides a method of delivering a therapeutic agent to a hematopoietic stem and progenitor cell (HSPC) in a subject, comprising administering to the subject the population of lipid assemblies or pharmaceutical composition described herein.

In some embodiments, the present disclosure provides the population of lipid assemblies or pharmaceutical composition described herein for use in delivering a therapeutic agent to a hematopoietic stem and progenitor cell (HSPC) in a subject.

In some embodiments, the present disclosure provides use of the population of lipid assemblies or composition described herein in the manufacture of a medicament for delivering a therapeutic agent to a hematopoietic stem and progenitor cell (HSPC) in a subject.

In some embodiments, the subject is human.

The present disclosure provides methods of delivering a therapeutic and/or prophylactic, such as a nucleic acid, to a mammalian cell or organ. Delivery of a therapeutic and/or prophylactic to a cell involves administering a formulation of the disclosure that comprises a lipid assembly including the therapeutic and/or prophylactic, such as a nucleic acid, to a subject, where administration of the composition involves contacting the cell with the composition. In some embodiments, a protein, cytotoxic agent, radioactive ion, chemotherapeutic agent, or nucleic acid (such as an RNA, e.g., mRNA) may be delivered to a cell or organ. In the instance that a therapeutic and/or prophylactic is an mRNA, upon contacting a cell with the lipid assembly, a translatable mRNA may be translated in the cell to produce a polypeptide of interest. However, mRNAs that are substantially not translatable may also be delivered to cells. Substantially non-translatable mRNAs may be useful as vaccines and/or may sequester translational components of a cell to reduce expression of other species in the cell.

In some embodiments, a lipid assembly may target a particular type or class of cells (e.g., cells of a particular organ or system thereof). In some embodiments, a lipid assembly including a therapeutic and/or prophylactic of interest may be specifically delivered to a mammalian liver, kidney, spleen, femur, or lung. Specific delivery to a particular class of cells, an organ, or a system or group thereof implies that a higher proportion of lipid assemblies including a therapeutic and/or prophylactic are delivered to the destination (e.g., tissue) of interest relative to other destinations, e.g., upon administration of a lipid assembly to a mammal. In some embodiments, specific delivery may result in a greater than 2-fold, 5-fold, 10-fold, 15-fold, or 20-fold increase in the amount of therapeutic and/or prophylactic per 1 g of tissue of the targeted destination (e.g., tissue of interest, such as a liver) as compared to another destination (e.g., the spleen). In some embodiments, the tissue of interest is selected from the group consisting of a liver, kidney, a lung, a spleen, a femur, vascular endothelium in vessels (e.g., intra-coronary or intra-femoral) or kidney, and tumor tissue (e.g., via intratumoral injection).

As another example of targeted or specific delivery, an mRNA that encodes a protein-binding partner (e.g., an antibody or functional fragment thereof, a scaffold protein, or a peptide) or a receptor on a cell surface may be included in a lipid assembly. An mRNA may additionally or instead be used to direct the synthesis and extracellular localization of lipids, carbohydrates, or other biological moieties. Alternatively, other therapeutics and/or prophylactics or elements (e.g., lipids or ligands) of a lipid assembly may be selected based on their affinity for particular receptors (e.g., low density lipoprotein receptors) such that a lipid assembly may more readily interact with a target cell population including the receptors. In some embodiments, ligands may include, but are not limited to, members of a specific binding pair, antibodies, monoclonal antibodies, Fv fragments, single chain Fv (scFv) fragments, Fab′ fragments, F(ab′)2 fragments, single domain antibodies, camelized antibodies and fragments thereof, humanized antibodies and fragments thereof, and multivalent versions thereof; multivalent binding reagents including mono- or bi-specific antibodies, such as disulfide stabilized Fv fragments, scFv tandems, diabodies, tribodies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

A ligand can be selected, e.g., by a person skilled in the biological arts, based on the desired localization or function of the cell.

In some embodiments, a lipid assembly may target hepatocytes. Apolipoproteins, such as apolipoprotein E (apoE), have been shown to associate with neutral or near neutral lipid-containing lipid assemblies in the body, and are known to associate with receptors, such as low-density lipoprotein receptors (LDLRs) found on the surface of hepatocytes. Thus, a lipid assembly including a lipid component with a neutral or near neutral charge that is administered to a subject may acquire apoE in a subject's body and may subsequently deliver a therapeutic and/or prophylactic (e.g., an RNA) to hepatocytes including LDLRs in a targeted manner.

### Methods of Treating Diseases and Disorders

In some embodiments, the present disclosure provides a method of treating or preventing a disease or disorder, the method comprising administering to a subject in need thereof the population of lipid assemblies or pharmaceutical composition described herein (e.g., in a therapeutically effective amount).

In some embodiments, the present disclosure provides the population of lipid assemblies or pharmaceutical composition described herein for use in treating or preventing a disease or disorder in a subject.

In some embodiments, the present disclosure provides use of the population of lipid assemblies or pharmaceutical composition described herein in the manufacture of a medicament for treating or preventing a disease or disorder.

In some embodiments, the population of lipid assemblies or pharmaceutical composition is administered parenterally.

In some embodiments, the population of lipid assemblies or pharmaceutical composition is administered intramuscularly, intradermally, subcutaneously, and/or intravenously.

Lipid assemblies may be useful for treating a disease, disorder, or condition. In particular, such compositions may be useful in treating a disease, disorder, or condition characterized by missing or aberrant protein or polypeptide activity. In some embodiments, a formulation of the disclosure that comprises a lipid assembly including an mRNA encoding a missing or aberrant polypeptide may be administered or delivered to a cell. Subsequent translation of the mRNA may produce the polypeptide, thereby reducing or eliminating an issue caused by the absence of or aberrant activity caused by the polypeptide. Because translation may occur rapidly, the methods and compositions may be useful in the treatment of acute diseases, disorders, or conditions, such as sepsis, stroke, and myocardial infarction. A therapeutic and/or prophylactic included in a lipid assembly may also be capable of altering the rate of transcription of a given species, thereby affecting gene expression.

The disclosure provides methods involving administering lipid assemblies including one or more therapeutic and/or prophylactic agents, such as a nucleic acid, and pharmaceutical compositions including the same. The terms therapeutic and prophylactic can be used interchangeably herein with respect to features and embodiments of the present disclosure. Therapeutic compositions, or imaging, diagnostic, or prophylactic compositions thereof, may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject; the purpose of the administration; the particular composition; the mode of administration; and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment. The specific therapeutically effective, prophylactically effective, or otherwise appropriate dose level (e.g., for imaging) for any particular patient will depend upon a variety of factors including the severity and identity of a disorder being treated, if any; the one or more therapeutics and/or prophylactics employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A lipid assembly including one or more therapeutics and/or prophylactics, such as a nucleic acid, may be administered by any route. In some embodiments, compositions, including prophylactic, diagnostic, or imaging compositions including one or more lipid assemblies described herein, are administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, topical (e.g., by powders, ointments, creams, gels, lotions, and/or drops), mucosal, nasal, buccal, enteral, intravitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery or administration of compositions described herein by any appropriate route taking into consideration likely advances in the sciences of drug delivery. In general, the most appropriate route of administration will depend upon a variety of factors including the nature of the lipid assembly including one or more therapeutics and/or prophylactics (e.g., its stability in various bodily environments such as the bloodstream and gastrointestinal tract), the condition of the patient (e.g., whether the patient is able to tolerate particular routes of administration), etc.

Lipid assemblies including one or more therapeutics and/or prophylactics, such as a nucleic acid, may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. In some embodiments, one or more lipid assemblies including one or more different therapeutics and/or prophylactics may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects, such as infusion related reactions).

A lipid assembly may be used in combination with an agent to increase the effectiveness and/or therapeutic window of the composition. Such an agent may be, for example, an anti-inflammatory compound, a steroid (e.g., a corticosteroid), a statin, an estradiol, a BTK inhibitor, an S1P1 agonist, a glucocorticoid receptor modulator (GRM), or an anti-histamine. In some embodiments, a lipid assembly may be used in combination with dexamethasone, methotrexate, acetaminophen, an H1 receptor blocker, or an H2 receptor blocker. In some embodiments, a method of treating a subject in need thereof or of delivering a therapeutic and/or prophylactic to a subject (e.g., a mammal) may involve pre-treating the subject with one or more agents prior to administering a lipid assembly. In some embodiments, a subject may be pre-treated with a useful amount (e.g., 10 mg, 20 mg, 30 mg, 40 mg, 50 mg, 60 mg, 70 mg, 80 mg, 90 mg, 100 mg, or any other useful amount) of dexamethasone, methotrexate, acetaminophen, an H1 receptor blocker, or an H2 receptor blocker. Pre-treatment may occur 24 or fewer hours (e.g., 24 hours, 20 hours, 16 hours, 12 hours, 8 hours, 4 hours, 2 hours, 1 hour, 50 minutes, 40 minutes, 30 minutes, 20 minutes, or 10 minutes) before administration of the lipid assembly and may occur one, two, or more times in, for example, increasing dosage amounts.

### Biological Assays

Lipids, lipid assemblies, populations of lipid assemblies, and pharmaceutical compositions described herein can be characterized using a variety of assays known to those skilled in the art to determine whether the compounds have biological activity. For example, the lipids, lipid assemblies, populations of lipid assemblies, and pharmaceutical compositions can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay is described in the Examples herein.

In some embodiments, the biological assay is a transfection assay. In some embodiments, the biological assays is a bone marrow transfection assay. In some embodiments, the assay is a spleen cell transfection assay. In some embodiments, the assay is an immune cell transfection assay.

### Definitions

As used herein, the term “lipid assembly” or “lipid assemblies” refers to a composition having a structure assembled from one or more types of lipids (e.g., of ionizable lipids, structural lipids, phospholipids, and PEG lipids). The assembled one or more lipids may form a lipid single layer, a lipid bilayer, or a combination thereof. In some embodiments, the lipid assembly comprises a lipid nanoparticle, a liposome, or a combination thereof. In some embodiments, the lipid assembly has a size of about 500 nm or less, about 450 nm or less, about 400 nm or less, about 350 nm or less, about 300 nm or less, about 250 nm or less, about 200 nm or less, about 150 nm or less, or about 100 nm or less. In some embodiments, the lipid assembly has a size ranging from about 1 nm to about 100 nm. In some embodiments, about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, or about 95% or greater of the surface area of the lipid assemblies comprises a lipid bilayer. In some embodiments, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, or about 5% or less of the surface area of the lipid assemblies comprises a lipid bilayer.

As used herein, the term “lipid nanoparticle” or “LNP” refers to a nanoparticle comprising one or more lipids. In some embodiments, the LNP has a size of about 500 nm or less, about 450 nm or less, about 400 nm or less, about 350 nm or less, about 300 nm or less, about 250 nm or less, about 200 nm or less, about 150 nm or less, or about 100 nm or less. In some embodiments, the LNP has a size ranging from about 1 nm to about 100 nm.

As used herein, the term “liposome” refers to a composite having at least one lipid bilayer. In some embodiments, the liposome has a size of about 500 nm or less, about 450 nm or less, about 400 nm or less, about 350 nm or less, about 300 nm or less, about 250 nm or less, about 200 nm or less, about 150 nm or less, or about 100 nm or less. In some embodiments, the liposome has a size ranging from about 1 nm to about 100 nm.

As used herein, the term “total lipids” refers to the collection of ionizable lipids, structural lipids, and phospholipids, and PEG lipids (to the extent of their existence) in a given composition (e.g., a population of lipid assemblies). In some embodiments, when a population of lipid assemblies is free of PEG lipid, the total lipids in the population is the total amount of the ionizable lipid, the structural lipid, and the phospholipid in the population. In some embodiments, when a population of lipid assemblies comprises a PEG lipid, the total lipids in the population is the total amount of the ionizable lipid, the structural lipid, the phospholipid, and the PEG lipid in the population.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. In some embodiments, C₁₈ alkenyl may include one or more double bonds. A C₁₈ alkenyl group including two double bonds may be a linoleyl group. Unless otherwise specified, an alkenyl group described herein refers to both unsubstituted and substituted alkenyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2-dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. In some embodiments, M and M′ can be selected from the non-limiting group consisting of optionally substituted phenyl, oxazole, and thiazole. In the formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR —OC(O)R), an aldehyde (e.g., —C(O)H), a carbonyl (e.g., —C(O)R, alternatively represented by C═O), an acyl halide (e.g., —C(O)X, in which X is a halide selected from bromide, fluoride, chloride, and iodide), a carbonate (e.g., —OC(O)OR), an alkoxy (e.g., —OR), an acetal (e.g., —C(OR)₂R″″, in which each OR is alkoxy groups that can be the same or different and R″″ is an alkyl or alkenyl group), a phosphate (e.g., P(O)₄³⁻), a thiol (e.g., —SH), a sulfoxide (e.g., —S(O)R), a sulfinic acid (e.g., —S(O)OH), a sulfonic acid (e.g., —S(O)₂OH), a thial (e.g., —C(S)H), a sulfate (e.g., S(O)₄²⁻), a sulfonyl (e.g., —S(O)₂—), an amide (e.g., —C(O)NR₂ or —N(R)C(O)R), an azido (e.g., —N₃), a nitro (e.g., —NO₂), a cyano (e.g., —CN), an isocyano (e.g., —NC), an acyloxy (e.g., —OC(O)R), an amino (e.g., —NR₂, —NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH₂, —N(R)S(O)₂R, —N(H) S(O)₂R, —N(R)S(O)₂H, or —N(H) S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. In some embodiments, a C₁₋₆ alkyl group may be further substituted with one, two, three, four, five, or six substituents as described herein.

As used herein, the terms “approximately” and “about,” as applied to one or more values of interest, refer to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, when used in the context of an amount of a given compound in a lipid component of a lipid assembly, “about” may mean +/−10% of the recited value. For instance, a lipid assembly including a lipid component having about 40% of a given compound may include 30-50% of the compound.

As used herein, the term “compound” is meant to include all isomers and isotopes of the structure depicted. “Isotopes” refers to atoms having the same atomic number but different mass numbers resulting from a different number of neutrons in the nuclei. In some embodiments, isotopes of hydrogen include tritium and deuterium. Further, a compound, salt, or complex of the present disclosure can be prepared in combination with solvent or water molecules to form solvates and hydrates by routine methods.

As used herein, the term “upon” intends to refer to the time point being after an action happens. For example, “upon administration” refers to the time point being after the action of administration.

As used herein, the term “contacting” means establishing a physical connection between two or more entities. In some embodiments, contacting a mammalian cell with a lipid assembly means that the mammalian cell and a nanoparticle are made to share a physical connection. Methods of contacting cells with external entities both in vivo and ex vivo are well known in the biological arts. In some embodiments, contacting a lipid assembly and a mammalian cell disposed within a mammal may be performed by varied routes of administration (e.g., intravenous, intramuscular, intradermal, and subcutaneous) and may involve varied amounts of lipid assemblies. Moreover, more than one mammalian cell may be contacted by a lipid assembly.

As used herein, the term “comparable method” refers to a method with comparable parameters or steps, as of the method being compared (e.g., producing the lipid assembly formulation of the present disclosure). In some embodiments, the “comparable method” is a method with one or more of steps i), ia), iaa), ib), ii), iia), iib), iic), iid), and iie) of the method being compared. In some embodiments, the “comparable method” is a method without one or more of steps i), ia), iaa), ib), ii), iia), iib), iic), iid), and iie) of the method being compared. In some embodiments, the “comparable method” is a method without one or more of steps ia) and ib) of the method being compared. In some embodiments, the “comparable method” is a method employing a water-soluble salt of a nucleic acid. In some embodiments, the “comparable method” is a method employing an organic solution that does not comprise an organic solvent-soluble nucleic acid. In some embodiments, the “comparable method” is a method comprising processing the lipid assembly prior to administering the lipid assembly formulation.

As used herein, the term “delivering” means providing an entity to a destination. In some embodiments, delivering a therapeutic and/or prophylactic to a subject may involve administering a lipid assembly including the therapeutic and/or prophylactic to the subject (e.g., by an intravenous, intramuscular, intradermal, or subcutaneous route). Administration of a lipid assembly to a mammal or mammalian cell may involve contacting one or more cells with the lipid assembly.

As used herein, the term “enhanced delivery” means delivery of more (e.g., at least 1.5-fold more, at least 2-fold more, at least 3-fold more, at least 4-fold more, at least 5-fold more, at least 6-fold more, at least 7-fold more, at least 8-fold more, at least 9-fold more, or at least 10-fold more) of a therapeutic and/or prophylactic by a nanoparticle to a target tissue of interest (e.g., mammalian liver) compared to the level of delivery of a therapeutic and/or prophylactic by a control nanoparticle to a target tissue of interest (e.g., MC3, KC2, or DLinDMA). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. It will be understood that the enhanced delivery of a nanoparticle to a target tissue need not be determined in a subject being treated; it may be determined in a surrogate such as an animal model (e.g., a rat model).

As used herein, the term “specific delivery,” “specifically deliver,” or “specifically delivering” means delivery of more (e.g., at least 1.5-fold more, at least 2-fold more, at least 3-fold more, at least 4-fold more, at least 5-fold more, at least 6-fold more, at least 7-fold more, at least 8-fold more, at least 9-fold more, or at least 10-fold more) of a therapeutic and/or prophylactic by a nanoparticle to a target tissue of interest (e.g., mammalian liver) compared to an off-target tissue (e.g., mammalian spleen). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. In some embodiments, for renovascular targeting, a therapeutic and/or prophylactic is specifically provided to a mammalian kidney as compared to the liver and spleen if 1.5-fold, 2-fold, 3-fold, 5-fold, 10-fold, 15 fold, or 20 fold more therapeutic and/or prophylactic per 1 g of tissue is delivered to a kidney compared to that delivered to the liver or spleen following systemic administration of the therapeutic and/or prophylactic. It will be understood that the ability of a nanoparticle to specifically deliver to a target tissue need not be determined in a subject being treated; it may be determined in a surrogate such as an animal model (e.g., a rat model).

As used herein, “encapsulation efficiency” refers to the amount of a therapeutic and/or prophylactic that becomes part of a nanoparticle composition, relative to the initial tatai amount of therapeutic and/or prophylactic used in the preparation of a nanoparticle composition. For example, if 97 mg of therapeutic and/or prophylactic are encapsulated in a nanoparticle composition out of a tatai 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, “encapsulation,” “encapsulated,” “loaded,” and “associated” may refer to complete, substantial, or partial enclosure, confinement, surrounding, or encasement. As used herein, “encapsulation” or “association” may refer to the process of confining an individual nucleic acid molecule within a nanoparticle and/or establishing a physiochemical relationship between an individual nucleic acid molecule and a nanoparticle. As used herein, an “empty nanoparticle” may refer to a nanoparticle that is substantially free of a therapeutic or prophylactic agent. As used herein, an “empty nanoparticle” or an “empty lipid nanoparticle” may refer to a nanoparticle that is substantially free of a nucleic acid. As used herein, an “empty nanoparticle” or an “empty lipid nanoparticle” may refer to a nanoparticle that is substantially free of a nucleotide or a polypeptide. As used herein, an “empty nanoparticle” or an “empty lipid nanoparticle” may refer to a nanoparticle that consists substantially of only lipid components. As used herein, a “loaded nanoparticle” or a “loaded lipid nanoparticle” (also referred to as a “full nanoparticle” or a “full lipid nanoparticle”) may refer to a nanoparticle comprising the components of the empty nanoparticle, and a therapeutic or prophylactic agent. As used herein, a “loaded nanoparticle” or a “loaded lipid nanoparticle” (also referred to as a “full nanoparticle” or a “full lipid nanoparticle”) may refer to a nanoparticle comprising the components of the empty nanoparticle, and a nucleotide or polypeptide. As used herein, a “loaded nanoparticle” or a “loaded lipid nanoparticle” (also referred to as a “full nanoparticle” or a “full lipid nanoparticle”) may refer to a nanoparticle comprising the components of the empty nanoparticle, and a nucleic acid.

As used herein, “expression” of a nucleic acid sequence refers to translation of an mRNA into a polypeptide or protein and/or post-translational modification of a polypeptide or protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers (i.e., geometric E/Z isomers) or diastereomers (e.g., enantiomers (i.e., (+) or (−)) or cis/trans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known.

As used herein, a “lipid component” is that component of a lipid assembly that includes one or more lipids. In some embodiments, the lipid component may include one or more cationic/ionizable, PEGylated, structural, or other lipids, such as phospholipids.

As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. In some embodiments, two nucleosides of a cap analog may be linked at their 5′ positions by a triphosphate group or by a chain including two phosphate moieties and a boranophosphate moiety.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, “modified” means non-natural. In some embodiments, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. In some embodiments, a modified nucleobase species may include one or more substitutions that are not naturally occurring.

As used herein, the “N:P ratio” is the molar ratio of ionizable (in the physiological pH range) nitrogen atoms in a lipid to phosphate groups in an RNA, e.g., in a lipid assembly including a lipid component and an RNA.

As used herein, “naturally occurring” means existing in nature without artificial aid.

As used herein, “patient” refers to a subject who may seek or be in need of treatment, requires treatment, is receiving treatment, or will receive treatment, or a subject who is under care by a trained professional for a particular disease or condition.

As used herein, a “PEG lipid” or “PEGylated lipid” refers to a lipid comprising a polyethylene glycol component.

As used herein, a “polymeric lipid” refers to a lipid comprising repeating subunits in its chemical structure. In some embodiments, the polymeric lipid is a lipid comprising a polymer component. In some embodiments, the polymeric lipid is a PEG lipid. In some embodiments, the polymeric lipid is not a PEG lipid. In some embodiments, the polymeric lipid is Brij or OH-PEG-stearate.

The phrase “pharmaceutically acceptable” is used herein to refer to those compounds, materials, composition, and/or dosage forms which are, within the scope of sound medical judgment, suitable for use in contact with the tissues of human beings and animals without excessive toxicity, irritation, allergic response, or other problems or complication, commensurate with a reasonable benefit/risk ratio.

The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example, anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to, ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. In some embodiments, the nonaqueous media are ether, ethyl acetate, ethanol, isopropanol, or acetonitrile. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. In some embodiments, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, the “polydispersity index,” or “PDI,” is a ratio that describes the homogeneity of the particle size distribution of a system. A small value, e.g., less than 0.3, indicates a narrow particle size distribution.

As used herein, an amphiphilic “polymer” is an amphiphilic compound that comprises an oligomer or a polymer. In some embodiments, an amphiphilic polymer can comprise an oligomer fragment, such as two or more PEG monomer units. In some embodiments, an amphiphilic polymer described herein can be PS 20.

Unless indicated otherwise, and as one of ordinary skill in the art would understand, the number of repeating units indicated in the structure of a polymer refers to the average number of repeating units (a.k.a., average degree of polymerization). For example, a PEG lipid of the following structure

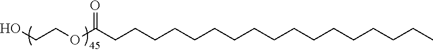

refers to a plurality of polymers with an average chain length of 45 ethylene glycol units. E.g., in some embodiments, r is an integer from about 35 to about 55.

As used herein, the term “polypeptide” or “polypeptide of interest” refers to a polymer of amino acid residues typically joined by peptide bonds that can be produced naturally (e.g., isolated or purified) or synthetically.

As used herein, an “RNA” refers to a ribonucleic acid that may be naturally or non-naturally occurring. In some embodiments, an RNA may include modified and/or non-naturally occurring components such as one or more nucleobases, nucleosides, nucleotides, or linkers. An RNA may include a cap structure, a chain-terminating nucleoside, a stem loop, a polyA sequence, and/or a polyadenylation signal. An RNA may have a nucleotide sequence encoding a polypeptide of interest. In some embodiments, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

As used herein, a “single unit dose” is a dose of any therapeutic administered in one dose/at one time/single route/single point of contact, i.e., single administration event.

As used herein, a “split dose” is the division of a single unit dose or total daily dose into two or more doses.

As used herein, a “total daily dose” is an amount given or prescribed in a 24-hour period. It may be administered as a single unit dose.

As used herein, the term “subject” refers to any organism to which a composition or formulation in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, “targeted cells” refers to any one or more cells of interest. The cells may be found in vitro, in vivo, in situ, or in the tissue or organ of an organism. The organism may be an animal. In some embodiments, the organism is a mammal. In some embodiments, the organism is a human. In some embodiments, the organism is a patient.

As used herein, “target tissue” refers to any one or more tissue types of interest in which the delivery of a therapeutic and/or prophylactic would result in a desired biological and/or pharmacological effect. Examples of target tissues of interest include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, the term “transfection” refers to the introduction of a species (e.g., an RNA) into a cell. Transfection may occur, for example, in vitro, ex vivo, or in vivo.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. In some embodiments, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “zeta potential” refers to the surface charge of colloidal dispersions. The magnitude of the zeta potential indicates the degree of electrostatic repulsion between adjacent, similarly charged particles in the dispersion. Zeta potential can be measured on a Wyatt Technologies Mobius Zeta Potential instrument. This instrument characterizes the mobility and zeta potential by the principle of “Massively Parallel Phase Analysis Light Scattering” or MP-PALS. This measurement is more sensitive and less stress-inducing than ISO Method 13099-1:2012 which only uses one angle of detection and required higher voltage for operation. In some embodiments, the zeta potential of the herein described empty lipid nanoparticle compositions lipid is measured using an instrument employing the principle of MP-PALS. Zeta potential can be measured on a Malvern Zetasizer (Nano ZS).

It is understood that some properties of lipid assemblies disclosed herein may be characterized by capillary zone electrophoresis (CZE). Capillary zone electrophoresis (CZE) refers to a separation technique which uses high voltage across a capillary to separate charged species based on their electrophoretic mobility. In some embodiments, the CZE is conducted with an acetate buffer (e.g., 50 mM sodium acetate at pH 5). In some embodiments, the CZE is conducted with a reverse voltage of about 10 kV across a 75 um capillary of 20 cm effective length. In some embodiments, the capillary is coated with polyethyleneimine.

The term “mobility peak,” as used herein, refers to a peak representing the distribution of a substance (e.g., a population of lipid assemblies) as measured by CZE. In some embodiments, the intensity of the mobility peak is detected by scattered light. It is understood that the intensity of the peak may indicate the amount of the portion of the substance at the position of the peak. In some embodiments, the position of the peak is calculated against a neutral reference standard (e.g., DMSO) being characterized by a mobility peak at 0, and a charged reference standard (e.g., benzylamine) being characterized by a mobility peak at 1.0. In some embodiments, a population of lipid assemblies may exhibit more than one peaks as measured by CZE, and unless indicated otherwise, the mobility peak refers to the peak having the greatest peak area among the more than one peaks.

The term “free of,” as used herein, means not comprising the referenced component. For example, when a population, solution, or formulation is described as being “free of PEG lipid,” the population, solution, or formulation does not comprise PEG lipid (e.g., does not comprise a PEG lipid described herein (e.g., does not comprise PEG-DMG)).

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all, of the group members are present in, employed in, or otherwise relevant to a given product or process.

It is also noted that the term “comprising” is intended to be open and permits but does not require the inclusion of additional elements or steps. When the term “comprising” is used herein, the terms “consisting essentially of” and “consisting of” are thus also encompassed and disclosed. Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein.

All cited sources, for example, references, publications, patent applications, databases, database entries, and art cited herein, are incorporated into this application by reference, even if not expressly stated in the citation. In case of conflicting statements of a cited source and the instant application, the statement in the instant application shall control.

The disclosure having been described, the following examples are offered by way of illustration and not limitation.

### EXEMPLARY EMBODIMENTS

Embodiment I-1. A population of lipid assemblies comprising a phosphatidylserine phospholipid, an ionizable lipid, and a structural lipid, wherein the phosphatidylserine phospholipid is of Formula (PL-I):

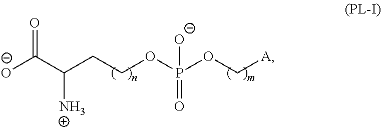

or a salt thereof, wherein:
n is 0, 1, 2, 3, 4, 5, 6, 7, 8, 9, or 10;
m is 0, 1, 2, 3, 4, 5, 6, 7, 8, 9, or 10;
A is of the formula:

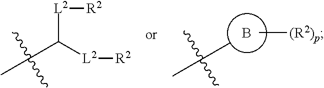

each instance of L² is independently a bond or an optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently cholesterol, optionally substituted cycloalkyl, optionally substituted C₁₋₃₀ alkyl, optionally substituted C₁₋₃₀ alkenyl, or optionally substituted C₁₋₃₀ alkynyl; optionally wherein one or more methylene units of the optionally substituted C₁₋₃₀ alkyl, optionally substituted C₁₋₃₀ alkenyl, or optionally substituted C₁₋₃₀ alkynyl are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

Embodiment I-2. The population of lipid assemblies of embodiment I-1, wherein the phosphatidylserine phospholipid is of Formula (PL-Ia):

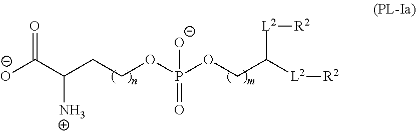

or a salt thereof.

Embodiment I-3. The population of lipid assemblies of embodiments I-1 and I-2, wherein n is 0 and m is 1.

Embodiment I-4. The population of lipid assemblies of any of the preceding embodiments, wherein the phosphatidylserine phospholipid is of Formula (PL-Ib):

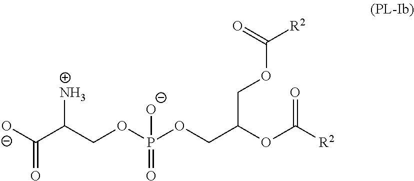

or a salt thereof.

Embodiment I-5. The population of lipid assemblies of any of the preceding embodiments, wherein each instance of R² is independently C₁₋₃₀, C₉₋₂₁, C₁₁₋₁₉, or C₁₃₋₁₇ alkyl or C₁₋₃₀, C₉₋₂₁, C₁₁₋₁₉, or C₁₃₋₁₇ alkenyl.

Embodiment I-6. The population of lipid assemblies of any of the preceding embodiments, wherein the phosphatidylserine phospholipid is

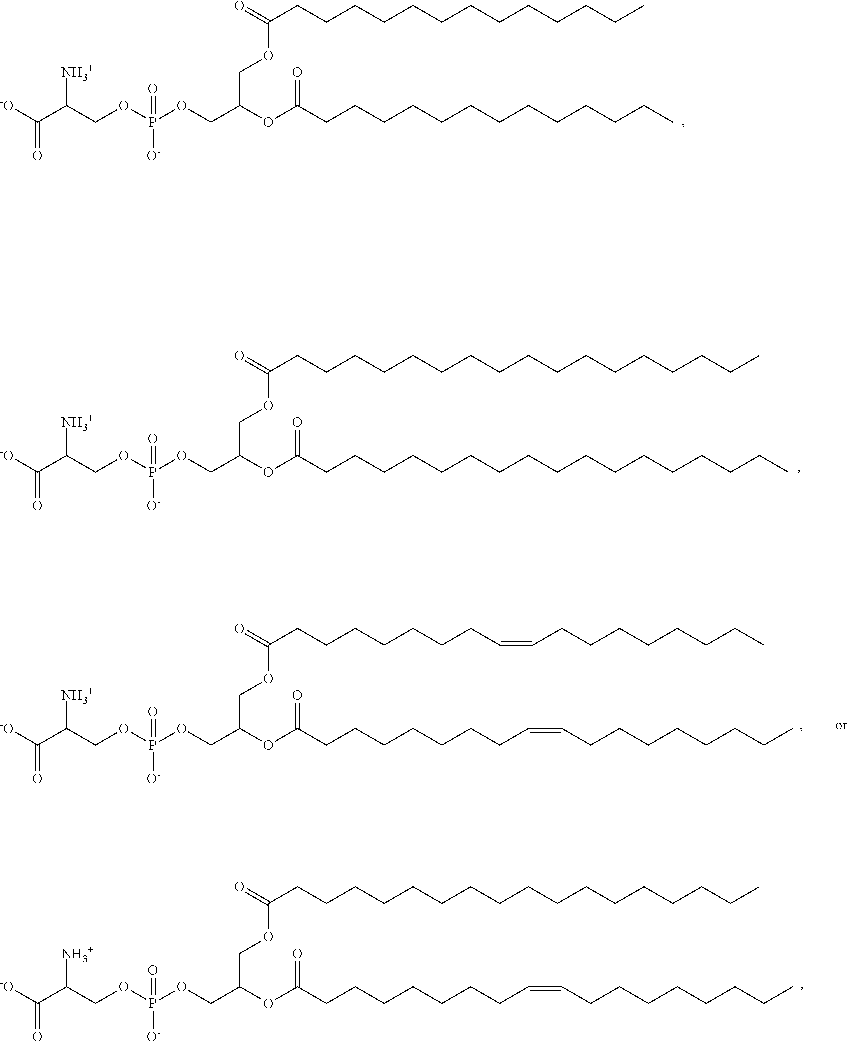

or a salt thereof.

Embodiment I-7. The population of lipid assemblies of any of the preceding embodiments, comprising between about 1 mol % to about 10 mol %, about 1 mol % to about 8 mol %, about 1 mol % to about 5 mol %, or about 2 mol % to about 5 mol % of the phosphatidylserine phospholipid.

Embodiment I-8. The population of lipid assemblies of any of the preceding embodiments, comprising about 1 mol %, about 2 mol %, about 3 mol %, about 4 mol %, or about 5 mol % of the phosphatidylserine phospholipid.

Embodiment I-9. The population of lipid assemblies of any of the preceding embodiments, comprising about 30 mol % to about 50 mol %, about 30 mol % to about 45 mol %, about 30 mol % to about 40 mol %, about 35 mol % to about 45 mol %, or about 35 mol % to about 40 mol % of the ionizable lipid.

Embodiment I-10. The population of lipid assemblies of any of the preceding embodiments, comprising about 30 mol %, about 35 mol %, about 40 mol %, about 45 mol %, or about 50 mol % of the ionizable lipid.

Embodiment I-11. The population of lipid assemblies of any of the preceding embodiments, wherein the ionizable lipid is compound 301 or compound 22.

Embodiment I-12. The population of lipid assemblies of any of the preceding embodiments, comprising about 15 mol % to about 45 mol %, about 20 mol % to about 45 mol %, about 25 mol % to about 45 mol %, about 30 mol % to about 45 mol %, about 35 mol % to about 45 mol %, or about 40 mol % to about 45 mol % of the structural lipid.

Embodiment I-13. The population of lipid assemblies of any of the preceding embodiments, comprising about 15 mol %, about 20 mol %, about 25 mol %, about 30 mol %, about 35 mol %, about 40 mol %, or about 45 mol % of the structural lipid.

Embodiment I-14. The population of lipid assemblies of any of the preceding embodiments, wherein the structural lipid is cholesterol.

Embodiment I-15. The population of lipid assemblies of any of the preceding embodiments, further comprising a second phospholipid.

Embodiment I-16. The population of lipid assemblies of embodiment I-15, comprising about 10 mol % to about 30 mol %, 10 mol % to about 25 mol %, 10 mol % to about 20 mol %, about 15 mol % to about 25 mol %, or about 15 mol % to about 20 mol % of the second phospholipid.

Embodiment I-17. The population of lipid assemblies of embodiments I-15 and I-16, comprising about 10 mol %, about 15 mol %, about 18 mol %, about 20 mol %, about 22 mol %, about 25 mol %, or about 30 mol % of the second phospholipid.

Embodiment I-18. The population of lipid assemblies of any of embodiments I-15 to I-17, wherein the second phospholipid is DSPC, DOPE, DOPC, POPE, or POPC.

Embodiment I-19. The population of lipid assemblies of any of the preceding embodiments, being free of PEG lipid.

Embodiment I-20. The population of lipid assemblies of any of embodiments I-1 to I-18, further comprising a PEG lipid.

Embodiment I-21. The population of lipid assemblies of embodiment I-20, comprising about 0.5 mol % to about 10 mol %, about 0.5 mol % to about 5 mol %, about 1 mol % to about 5 mol %, or about 1 mol % to about 3 mol % of the PEG lipid.

Embodiment I-22. The population of lipid assemblies of embodiments I-20 and I-21, comprising about 0.5 mol %, about 1 mol %, about 2 mol %, about 3 mol %, about 4 mol %, or about 5 mol % of the PEG lipid.

Embodiment I-23. The population of lipid assemblies of any of embodiments I-19 to I-22, wherein the PEG lipid is PL-02.

Embodiment I-24. The population of lipid assemblies of any of the preceding embodiments, having a pH value lower than the pKa value of the ionizable lipid.

Embodiment I-25. The population of lipid assemblies of any of the preceding embodiments, having a pH value of about 4.0±2.0, about 4.0±1.5, about 4.0±1.4, about 4.0±1.3, about 4.0±1.2, about 4.0±1.1, about 4.0±1.0, about 4.0±0.9, about 4.0±0.8, about 4.0±0.7, about 4.0±0.6, about 4.0±0.5, about 4.0±0.4, about 4.0±0.3, about 4.0±0.2, or about 4.0±0.1.

Embodiment I-26. The population of lipid assemblies of any of the preceding embodiments, having a pH value of about 5.0±2.0, about 5.0±1.5, about 5.0±1.4, about 5.0±1.3, about 5.0±1.2, about 5.0±1.1, about 5.0±1.0, about 5.0±0.9, about 5.0±0.8, about 5.0±0.7, about 5.0±0.6, about 5.0±0.5, about 5.0±0.4, about 5.0±0.3, about 5.0±0.2, or about 5.0±0.1.

Embodiment I-27. The population of lipid assemblies of any of embodiments I-1 to I-23, having a pH value higher than the pKa value of the ionizable lipid.

Embodiment I-28. The population of lipid assemblies of any of embodiments I-1 to I-23 and I-27, having a pH value of about 8.0±2.0, about 8.0±1.5, about 8.0±1.4, about 8.0±1.3, about 8.0±1.2, about 8.0±1.1, about 8.0±1.0, about 8.0±0.9, about 8.0±0.8, about 8.0±0.7, about 8.0±0.6, about 8.0±0.5, about 8.0±0.4, about 8.0±0.3, about 8.0±0.2, or about 8.0±0.1.

Embodiment I-29. The population of lipid assemblies of any of embodiments I-1 to I-23 and I-27, having a pH value of about 9.0±3.0, about 9.0±2.0, about 9.0±1.5, about 9.0±1.4, about 9.0±1.3, about 9.0±1.2, about 9.0±1.1, about 9.0±1.0, about 9.0±0.9, about 9.0±0.8, about 9.0±0.7, about 9.0±0.6, about 9.0±0.5, about 9.0±0.4, about 9.0±0.3, about 9.0±0.2, or about 9.0±0.1.

Embodiment I-30. The population of lipid assemblies of any of embodiments I-1 to I-23 and I-27, having a pH value of about 12.0±±2.0, about 12.0±1.5, about 12.0±1.4, about 12.0±1.3, about 12.0±1.2, about 12.0±1.1, about 12.0±1.0, about 12.0±0.9, about 12.0±0.8, about 12.0±0.7, about 12.0±0.6, about 12.0±0.5, about 12.0±0.4, about 12.0±0.3, about 12.0±0.2, or about 12.0±0.1.

Embodiment I-31. The population of lipid assemblies of any preceding embodiments, having a zeta potential between about −40 mV to about −5 mV, about −30 mV to about −5 mV, about −20m V to about −5 mV, about −40 mV to about −10 mV, about −30 mV to about −10 mv, or about −20m V to about −10 mV when measured in 0.1N PBS at pH 7.5.

Embodiment I-32. The population of lipid assemblies of any of the preceding embodiments, being free of therapeutic agent.

Embodiment I-33. The population of lipid assemblies of any of embodiments I-1 to I-31, further comprising a therapeutic agent.

Embodiment I-34. The population of lipid assemblies of embodiment I-33, wherein the therapeutic agent is an RNA.

Embodiment I-35. A pharmaceutical composition comprising the population of lipid assemblies of any of embodiments I-1 to I-34 and one or more pharmaceutically acceptable carriers or excipients.

Embodiment I-36. A method of preparing the population of lipid assemblies of any of embodiments I-1 to I-34.

Embodiment I-37. A method of preparing the pharmaceutical composition of embodiment I-35.

Embodiment I-38. A method of delivering a therapeutic agent to a hematopoietic stem and progenitor cell (HSPC) in a subject, comprising administering to the subject the population of lipid assemblies or the pharmaceutical composition of any of embodiments I-1 to I-35.

Embodiment I-39. The population of lipid assemblies or the pharmaceutical composition of any of embodiments I-1 to I-35 for use in delivering a therapeutic agent to a hematopoietic stem and progenitor cell (HSPC) in a subject.

Embodiment I-40. Use of the population of lipid assemblies or the pharmaceutical composition of any of embodiments I-1 to I-35 in the manufacture of a medicament for delivering a therapeutic agent to a hematopoietic stem and progenitor cell (HSPC) in a subject.

Embodiment I-41. The method, population, pharmaceutical composition, or use of any of embodiments I-38 to I-40, wherein the HSPC is in the spleen of the subject.

Embodiment I-42. The method, population, pharmaceutical composition, or use of any of embodiments I-38 to I-40, wherein the HSPC is in the bone marrow of the subject.

Embodiment I-43. A method of treating or preventing a disease or disorder, the method comprising administering to a subject in need thereof the population of lipid assemblies or the pharmaceutical composition of any of embodiments I-1 to I-35.

Embodiment I-44. The population of lipid assemblies or pharmaceutical composition of any of embodiments I-1 to I-35 for use in treating or preventing a disease or disorder in a subject.

Embodiment I-45. Use of the population of lipid assemblies or the pharmaceutical composition of any of embodiments I-1 to I-35 in the manufacture of a medicament for treating or preventing a disease or disorder in a subject.

Embodiment I-46. The method, population, pharmaceutical composition, or use of any of embodiments I-43 to I-45, wherein the disease or disorder is a spleen disease or a spleen disorder.

Embodiment I-47. The method, population, pharmaceutical composition, or use of any of embodiments I-43 to I-45, wherein the disease or disorder is a bone marrow disease or a bone marrow disorder.

Embodiment I-48. The method, population, pharmaceutical composition, or use of any of embodiments I-38 to I-47, wherein the subject is human.

Embodiment I-49. A process of preparing a nanoparticle, the process comprising contacting an aqueous stream comprising a phosphatidylserine lipid with an organic stream comprising an ionizable lipid and a structural lipid, thereby obtaining a mixed product.

Embodiment I-50. The process of embodiment I-49, wherein the organic stream further comprises a PEG lipid.

Embodiment I-51. The process of embodiment I-49 or I-50, wherein the pH of the aqueous stream is lower than the pKa of the ionizable lipid.

Embodiment I-52. The process of any of embodiments I-49 to I-51, wherein the process further comprises subjecting the mixed product to an in-line dilution.

Embodiment I-53. The process of embodiment I-52, wherein the in-line dilution lowers an organic solvent content of the mixed product.

Embodiment I-54. The process of embodiment I-52 or I-53, wherein the in-line dilution raises the pH of the mixed product above the pKa of the ionizable lipid.

Embodiment I-55. The process of any of embodiments I-49 to I-51, wherein the phosphatidylserine phospholipid is DMPS.

Embodiment I-56. The process of any of embodiments I-49 to I-51, wherein the ionizable lipid is Compound 22 or Compound 301.

Embodiment I-57. A nanoparticle prepared by the process of any of embodiments I-49 to I-56.

### EXAMPLES

### LNP Examples

### LNP Example 1—Example Processes for Making LNP Samples

LNPs were prepared by co-precipitation reactions in accordance with the following example protocols.

Empty Lipid Nanoparticles (eLNPs): General procedure—An example LNP sample was formed by first manufacturing an empty LNP (eLNP) followed by mRNA loading. To form the eLNPs, an acetate buffer stream (pH 4, 45 mM) was mixed with an ethanol stream containing all the lipids (including 0.5% PEG) at a ratio of 7:3. The mixed product was subjected to an in-line dilution that simultaneously lowered ethanol content (12.5%) and raised the pH to 5. This material was then passed on to the tangential flow filtration (TFF) setup to concentrate the eLNPs and exchange into pH 5, 5 mM acetate.

Loading LNPs: General Procedure—The eLNPs were subjected to a freeze thaw and diluted (pH 5, 5 mM acetate, 20% sucrose) before being mixed with the mRNA (pH 3.75, 30 mM acetate) at a ratio of 2:3. The concentration of the eLNPs and mRNA were adjusted to meet an N/P 3.4. LNPs were stored in 20 mM Tris, 8% sucrose and concentrated via centrifugal filters or TFF.

Phosphatidylserine Empty Lipid Nanoparticles (PS eLNPs): Process A-A″: These 5-component LNPs were manufactured via a novel process to improve incorporation of the anionic phospholipid (phosphotidylserine, PS). The first stage in this process is to manufacture an empty LNP (eLNP) that contains the PS lipid. The eLNPs were precipitated above the pKa of the ionizable lipid (Compound 22 or 301) using an aqueous stream containing PS and an organic stream containing the other lipids. The mixed product was met with an additional aqueous stream to lower organic solvent content prior to buffer exchange via desalting columns or Tangential Flow Filtration (TFF). Post exchange, the PS eLNPs received a sucrose spike to enable frozen storage.

Loading PS LNPs: For mRNA encapsulation, the PS eLNPs were diluted in an aqueous buffer and mixed with a second aqueous stream containing the mRNA of interest. The resulting pH of the mixed product was optimized depending on the ionizable lipid. The mixed product was then neutralized with Tris Sucrose followed by a final spike of the surface polymer (PL-02) to a total of 3%. Lastly, the LNPs were concentrated further and characterized to meet specs prior to in vivo dosing.

Samples comprising Compound 22 and DMPS were made via Process A. Samples comprising Compound 301 and DMPS were made via Process A′.

### LNP Example 2—Example LNP Samples

Tables X-1, X-2, and X-3 present exemplary LNP compositions and their constituent components, their physical characteristics, and the results of experiments concerning their transfection efficacy.

### BIOLOGICAL EXAMPLES

### Biological Example 1—Transfection Data

Table X-3 summarizes transfection data from select bone marrow and splenic HSPC and immune cell subsets at 24 h post LNP injection (0.5-1 mpk) in C57Bl/6 mice. Consistently high transfection (>60%) and protein expression (gMFI, not included) were observed in bone marrow hematopoietic stem and progenitor cells (HSPCs, gated as Lineage-Scal+cKit+LSK) and true hematopoietic stem cells (HSC). Additionally, all immune cell subsets in the splenic compartment including macrophages, monocytes, neutrophils, and lymphocytes were highly transfected using these LNP.

### Biological Example 1a—Bone Marrow (BM) Study Methodology

Mice were dosed at 0.5 mg/kg with LNP/mRNA diluted in sterile PBS or tris/sucrose buffer by i.v. tail vein injection. For mouse tissue collection, LNP-treated or vehicle-treated naïve mice were euthanized using CO2, and tissues/organs (spleen and BM) were harvested and processed into single cell suspensions for flow cytometric analysis.

For BM cell isolation, femur and tibia bones were collected and stripped of muscle tissue and either flushed out with 15 ml PBS using a 28G needle and syringe, or placed in a mortar and crushed with pestle to generate a cell suspension. The ensuing cell suspension was filtered through a 70 μm cell strainer and then centrifuged (300 g, 5 min). Resulting cell pellet was subjected to RBC lysis using ACK buffer for 2 min, diluted with PBS, filtered, and centrifuged. After centrifugation, the cell pellet was resuspended in FACS buffer and counted using the AO/PI method on the Cellaca MX. Approximately, 20×10⁶ cells were stained at a concentration of 40×10⁶/ml. For flow cytometry staining, cell suspensions were first stained with fixable viability dyes (Thermo Fisher Scientific) in PBS for 30 min on ice. Viability staining was stopped by adding FACS buffer and the ensuing cell suspension centrifuged. Cells were resuspended in FACS buffer and blocked with CD16/32 (clones 93, 2.4G2), FcgRIV (clone 9E9), and/or rat serum (2%; Stem Cell Technologies) for 5 min on ice prior to adding fluorescent antibodies (BioLegend, BD, or Thermo Fisher Scientific) for cell surface staining. For mouse BM, cells were stained for 20 min on ice, with fluorescent antibodies specific to lineage markers such as CD11b, CD4, CD5, CD8a, Ter119, Gr1, and B220, as well as antibodies specific for stem cell markers CD117/c-Kit (clone 2B8), Sca-1 (clone D7), CD150 (clone TC15-12F12.2), CD48 (clone HM48.1), and CD135 (clone A2F10), and antibody directed against the anti-OX40L reporter protein. Cell staining was stopped by adding FACS buffer and the ensuing cell suspension washed three times at 300 g centrifugation for 5 min. Cells were resuspended in FACS buffer and filtered prior to flow cytometry acquisition. Cells were run on a BD flow cytometer (Fortessa or Fusion) and analyzed using FlowJo software (BD).

### Biological Example 1b—Spleen Study Methodology

Immune cells from murine spleens were analyzed for mOX40L reporter expression at 24 hours post LNP dosing using flow cytometry. Whole spleens were harvested and dissociated using the gentleMACS Octo Dissociator in a GentleMACS C tube containing 5 mL of RPMI1640 medium. The dissociator was programmed with the vendor's “m_spleen_01” program. After dissociation, spleens were filtered over a 70 μM filter into a 50 mL conical and centrifuged at 1500 rpm for 5 min at 4° C. After centrifugation, the supernatant was removed and cell pellets were resuspended in 2 mL of Ammonium-Chloride-Potassium (ACK) Lysing Buffer, incubating for 2-3 minutes for lysis of red blood cells. The ACK reaction was then quenched with 10 mL of FACS Buffer (1×PBS with 1% BSA and 2 mM EDTA) before centrifugation at 1500 rpm for 5 min at 4° C. After removal of the supernatant, the cell pellet was resuspended in FACS buffer and cells were counted using the AO/PI method on the Cellaca MX. 2-4 million cells per spleen were plated in a 96-well V-bottom plate for flow cytometry staining. Cells were first stained at 4° C. in the dark for 20 minutes with 100 μL viability dye (Invitrogen, Cat. #L34957A) at a dilution of 1:1000 in PBS. Viability was washed off by centrifugation at 1500 rpm for 5 min at 4° C. Cells were then stained at 4° C. in the dark for 20 minutes with Fc Block (Mouse CD16.2 FcgRIV Fc Block and Mouse CD16/32 Fc Block) at 1:200 and the standard mouse mature immune cell phenotyping panel (Table Y-1). After staining, washing by centrifugation was repeated at 1500 rpm for 5 min at 4° C. for a total of four times before proceeding to flow cytometry acquisition with a BD LSRFortessa or BD FACSymphony A3. Major immune cell populations were identified by the markers in Table Y-2.

TABLE Y-1

| | | | | Catalog | Dilu- |
|---|---|---|---|---|---|
| Marker | Dye | Clone | Vendor | # | tion |
| CD86 | BUV396 | GL1 | BD | 564199 | 200 |
| B220 | AF700 | RA-6B2 | BD | 564662 | 400 |
| CD11c | BV421 | N418 | Biolegend | 117330 | 400 |
| Ly6G | BV605 | 1A8 | Biolegend | 127639 | 800 |
| TCRb | BV650 | 145-2C11 | BD | 564378 | 600 |
| F4/80 | BV711 | BM8 | Biolegend | 123147 | 800 |
| MHC-II | BV786 | M5/114-15.2 | Biolegend | 107645 | 400 |
| Ly6c | AF488 | HK1.4 | Biolegend | 128022 | 800 |
| CD11b | PerCP-Cy5.5 | M1/70 | Biolegend | 101228 | 400 |
| CD4 | PeCy7 | GK1.5 | Biolegend | 100422 | 400 |
| NK1.1 | AF647 | PK136 | Biolegend | 108720 | 800 |
| CD8a | APC-Cy7 | 53-6.7 | Biolegend | 100714 | 800 |
| mOX40L | PE | RM134L | Invitrogen | 12-5905-83 | 400 |

TABLE Y-2

| Population | Phenotype |
|---|---|
| Macrophage | Live, singlet, CD11b+ CD11c− F4/80+ |
| Neutrophil | Live, singlet, F4/80− Ly6G+ |
| cDC1 | Live, singlet, MHC II+ CD11c+ CD8a+ |
| cDC2 | Live, singlet, MHC II+ CD11c+ CD8a− |
| NK cells | Live, singlet, TCRb− F4/80− B220− CD11c− NK1.1+ |
| CD4 T cells | Live, singlet, B220− CD11b− TCRb+ CD4+ CD8− |
| CD8 T cells | Live, singlet, B220− CD11b− TCRb+ CD8+ CD4− |
| Ly6C-hi | Live, singlet, CD11b+ Ly6C+ Ly6G− CD11c− B220− |
| monocytes | TCRb− |
| Ly6C-lo | Live, singlet, CD11b+ Ly6Clo Ly6G− CD11c− B220− |
| monocytes | TCRb− |
| B cells | Live, singlet, CD11b− CD11c− TCRb− B220+ |
| Undefined | Live, singlet, CD11c− CD11b− TCRb− B220− Ly6C− |
| | Ly6G− |

TABLE X-1
LNP Sample Components

| | | | | Ionizable | Ionizable | | | | |
|---|---|---|---|---|---|---|---|---|---|
| | | | Dose | Lipid | Lipid | DSPC | DMPS | Chol | PL-02 |
| Sample # | Process, N/P and Dose | N/P | (mpk) | Identify | (mol %) | (mol %) | (mol %) | (mol %) | (mol %) |
| Buffer | N/A | | | | | | | | |
| Crtl. | | | | | | | | | |
| 1 | DMPS Post-Added | 4.9 | 0.5 | Compound 22 | 42.1 | 9.9 | 10 | 35 | 3 |
| 2 | DMPS Post-Added | 4.9 | 0.5 | Compound 22 | 41.2 | 9.7 | 10.0 | 34.2 | 5 |
| 3 | DMPS eLNP (1.5% PEG) | 4.9 | 0.5 | Compound 22 | 42.1 | 9.9 | 10 | 35 | 3 |
| 4 | DMPS eLNP (3% PEG) | 4.9 | 0.5 | Compound 22 | 42.1 | 9.9 | 10 | 35 | 3 |
| 5 | DMPS eLNP (1% PEG) | 4.9 | 0.5 | Compound 301 | 42.1 | 9.9 | 10 | 35 | 3 |
| 6 | DMPS eLNP (1% PEG) | 3.4 | 0.5 | Compound 301 | 42.1 | 9.9 | 10 | 35 | 3 |
| 7 | DMPS eLNP (1% PEG) | 3.4 | 0.5 | Compound 301 | 38.2 | 11.8 | 7.8 | 39.2 | 3 |
| 8 | DMPS eLNP (1% PEG) | 3.4 | 0.5 | Compound 301 | 38.2 | 19.6 | 0 | 39.2 | 3 |
| 9 | DMPS eLNP (1% PEG) | 3.4 | 0.5 | Compound 301 | 47 | 23.5 | 9.8 | 16.7 | 3 |
| 10 | DMPS eLNP (1% PEG) | 3.4 | 0.5 | Compound 301 | 47 | 33.3 | 0 | 16.7 | 3 |
| 11 | DMPS eLNP (1% PEG) | 3.4 | 0.5 | Compound 301 | 31.3 | 24.4 | 6.3 | 35 | 3 |
| 12 | DMPS eLNP (1% PEG) | 3.4 | 0.5 | Compound 301 | 35.3 | 9.1 | 10.4 | 42.1 | 3 |
| 13 | DMPS eLNP (1% PEG) | 3.4 | 0.5 | Compound 301 | 43.5 | 12.3 | 10.8 | 30.4 | 3 |
| 14 | DMPS eLNP (1% PEG) | 3.4 | 0.5 | Compound 301 | 30.6 | 12.5 | 7.6 | 46.2 | 3 |
| 15 | DMPS eLNP (1% PEG) | 3.4 | 0.5 | Compound 301 | 43.8 | 16.8 | 1.5 | 34.9 | 3 |
| 16 | DMPS eLNP (1% PEG) | 3.4 | 0.5 | Compound 301 | 42.1 | 9.9 | 10 | 35 | 3 |
| 17 | DMPS eLNP (1% PEG) | 3.4 | 0.5 | Compound 301 | 30.3 | 11.3 | 12 | 43.5 | 3 |
| 18 | DMPS eLNP (1% PEG) | 3.4 | 0.5 | Compound 301 | 41.6 | 0.2 | 15.3 | 39.9 | 3 |
| 19 | DMPS eLNP (1% PEG) | 3.4 | 0.5 | Compound 301 | 33.5 | 13.1 | 18.2 | 32.2 | 3 |
| 20 | DMPS eLNP (1% PEG) | 3.4 | 0.5 | Compound 301 | 46.8 | 10.1 | 7.8 | 32.2 | 3 |
| 21 | DMPS eLNP (1% PEG) | 3.4 | 0.5 | Compound 301 | 32.8 | 16.3 | 4.8 | 43 | 3 |
| 22 | DMPS eLNP (1% PEG) | 3.4 | 0.5 | Compound 301 | 39.5 | 23.7 | 1.7 | 32.2 | 3 |
| 23 | DMPS eLNP (1% PEG) | 3.4 | 0.5 | Compound 301 | 35.6 | 21.3 | 9.7 | 30.4 | 3 |
| 24 | DMPS eLNP (1% PEG) | 3.4 | 0.5 | Compound 301 | 39.7 | 18.7 | 4.1 | 34.5 | 3 |
| 25 | DMPS eLNP (1% PEG) | 3.4 | 0.5 | Compound 301 | 43.8 | 24 | 7.1 | 22.1 | 3 |
| 26 | I-18 Control | 4.9 | 0.5 | I-18 | 48 | 11 | 0 | 39 | 3 |
| 27 | DMPS eLNP (1% PEG) | 4.9 | 0.5 | Compound 22 | 39.7 | 18.7 | 4.1 | 34.5 | 3 |
| 28 | DMPS eLNP (1% PEG) | 3.4 | 0.5 | Compound 22 | 39.7 | 18.7 | 4.1 | 34.5 | 3 |
| 29 | DMPS eLNP (1% PEG) | 3.4 | 0.5 | Compound 301 | 42.1 | 9.9 | 10 | 35 | 3 |
| 30 | DMPS eLNP (1% PEG) | 3.4 | 0.5 | Compound 301 | 39.7 | 18.7 | 4.1 | 34.5 | 3 |
| 31 | DMPS eLNP (1% PEG) | 3.4 | 1.0 | Compound 301 | 42.1 | 9.9 | 10 | 35 | 3 |
| 32 | DMPS eLNP (1% PEG) | 4.9 | 1.0 | Compound 22 | 39.7 | 18.7 | 4.1 | 34.5 | 3 |
| 33 | DMPS eLNP (1% PEG) | 3.4 | 1.0 | Compound 301 | 39.7 | 18.7 | 4.1 | 34.5 | 1 |
| 34 | DMPS eLNP (1% PEG) | 3.4 | 1.0 | Compound 301 | 43.8 | 24 | 7.1 | 22.1 | 3 |
| 35 | DMPS Post-Added | 4.9 | 0.5 | Compound 22 | 42.1 | 9.9 | 10 | 35 | 3 |
| 36 | DMPS eLNP (1% PEG) | 4.9 | 0.5 | Compound 22 | 42.1 | 9.9 | 10 | 35 | 3 |
| 37 | DMPS eLNP (1% PEG) | 3.4 | 0.5 | Compound 301 | 42.1 | 9.9 | 10 | 35 | 3 |
| 38 | DMPS eLNP (1% PEG) | 4.9 | 0.5 | Compound 22 | 42.1 | 9.9 | 10 | 35 | 3 |

TABLE X-2
LNP Study Description and Physical Characterization

| | Ionizable | | | | Zeta Potential |
|---|---|---|---|---|---|
| | Lipid | D | | | (Mean ± Stdev, |
| Sample # | Identify | (nm) | PDI | % EE | m V) |
| Buffer Crtl. | N/A | N/A | | | |
| — | Compound 22 | 90 | 0.238 | 94.2 | −22.44 ± 24.14 |
| 2 | Compound 22 | 91.6 | 0.184 | 92.7 | −12.5 ± 11.85 |
| 3 | Compound 22 | 67.3 | 0.14 | 96.5 | −7.64 ± 10.01 |
| 4 | Compound 22 | 58.3 | 0.173 | 93.2 | −10.32 ± 12.89 |
| 5 | Compound 301 | 96.8 | 0.034 | 94.9 | −7.05 ± 9.18 |
| 6 | Compound 301 | 97.5 | 0.1 | 95.7 | −6.74 ± 9.22 |
| 7 | Compound 301 | 121.9 | 0.121 | 95.3 | −5.23 ± 7.78 |
| 8 | Compound 301 | 98.7 | 0.112 | 95.5 | −1.99 ± 10 |
| 9 | Compound 301 | 117.6 | 0.147 | 66.5 | −11.5 ± 9.18 |
| 10 | Compound 301 | 147.1 | 0.191 | 65 | −4.83 ± 8.55 |
| 11 | Compound 301 | 117.5 | 0.16 | 92.9 | −6.78 ± 8.2 |
| 12 | Compound 301 | 112.5 | 0.121 | 94.5 | −5.86 ± 8.49 |
| 13 | Compound 301 | 112 | 0.083 | 90.8 | −8.01 ± 8.64 |
| 14 | Compound 301 | 120.9 | 0.109 | 95.6 | −6.27 ± 7.78 |
| 15 | Compound 301 | 81.7 | 0.163 | 95.3 | −3.74 ± 9.35 |
| 16 | Compound 301 | 97.5 | 0.1 | 95.7 | −6.74 ± 9.22 |
| 17 | Compound 301 | 130.1 | 0.119 | 93.8 | −11.05 ± 10.62 |
| 18 | Compound 301 | 125.6 | 0.061 | 70.5 | −14.97 ± 9.7 |
| 19 | Compound 301 | 149.7 | 0.092 | 79.54 | −15.83 ± 8.13 |
| 20 | Compound 301 | 107.4 | 0.106 | 95 | −5.6 ± 9.27 |
| 21 | Compound 301 | 113.6 | 0.125 | 97 | −5.42 ± 9.91 |
| 22 | Compound 301 | 89.3 | 0.148 | 96.9 | −3.27 ± 10.2 |
| 23 | Compound 301 | 114.2 | 0.117 | 93.2 | −10.7 ± 11 |
| 24 | Compound 301 | 96 | 0.087 | 97.5 | −5.11 ± 9.57 |
| 25 | Compound 301 | 99.1 | 0.11 | 91.8 | −7.33 ± 7.95 |
| 26 | I-18 | 68.40 | 0.09 | 93.30 | N/A |
| 27 | Compound 22 | 74.20 | 0.13 | 93.60 | −4.52 ± 10.70 |
| 28 | Compound 22 | 78.20 | 0.10 | 95.90 | −4.01 ± 11.50 |
| 29 | Compound 301 | 102.40 | 0.09 | 96.20 | −5.02 ± 8.36 |
| 30 | Compound 301 | 108.50 | 0.06 | 95.40 | −13.25 ± 10.22 |
| 31 | Compound 301 | 97.50 | 0.06 | 93.80 | −6.65 ± 9.88 |
| 32 | Compound 22 | 74.23 | 0.13 | 93.60 | −4.52 ± 10.7 |
| 33 | Compound 301 | 108.50 | 0.06 | 95.40 | −13.25 ± 10.22 |
| 34 | Compound 301 | 99.1 | 0.11 | 91.8 | −7.33 ± 7.95 |
| 35 | Compound 22 | 71.3 | 0.256 | 94.7 | −27.29 ± 18.87 |
| 36 | Compound 22 | 79.5 | 0.148 | 97.8 | −6.8 ± 10.45 |
| 37 | Compound 301 | 97.50 | 0.06 | 93.80 | −6.65 ± 9.88 |
| 38 | Compound 22 | 89.76 | 0.07 | 90 | −6.27 ± 9.39 |

TABLE X-3
LNP Study Description and Activity Data

| | | | Bone | Bone | | Splenic | Splenic |
|---|---|---|---|---|---|---|---|
| | Ionizable | | Marrow | Marrow | Splenic | Infl | Ptrl |
| | Lipid | Dose | HSC | LSK | Macrophages | Monocytes | Monocytes |
| Sample # | Identify | (mpk) | % mOX40L Mean ± StDev | | | | |
| Buffer | | 0 | 0.69 ± 0.6 | 0.69 ± 0.42 | 1.14 ± 0.82 | 0.88 ± 0.5 | 0.98 ± 0.52 |
| Crtl. | | | | | | | |
| 1 | Cmpd. 22 | 0.5 | 90.53 ± 2.68 | 91.2 ± 2.2 | 99.88 ± 0.13 | 89.88 ± 2.15 | 88.9 ± 2.65 |
| 2 | Cmpd. 22 | 0.5 | 92 ± 1.72 | 91.83 ± 1.56 | 99.38 ± 0.15 | 89.43 ± 6.59 | 83.83 ± 8.06 |
| 3 | Cmpd. 22 | 0.5 | 90.48 ± 3.29 | 88.28 ± 2.67 | 99.23 ± 0.33 | 82.9 ± 2.99 | 77.58 ± 3.5 |
| 4 | Cmpd. 22 | 0.5 | 85.33 ± 5.02 | 84.75 ± 2.56 | 98.78 ± 0.6 | 70.68 ± 5.87 | 62.53 ± 8.5 |
| 5 | Cmpd. 301 | 0.5 | 84.9 ± 1.93 | 86.66 ± 1.54 | 99.06 ± 0.08 | 77.06 ± 2.43 | 46.62 ± 0.68 |
| 6 | Cmpd. 301 | 0.5 | 85.33 ± 2.53 | 87.65 ± 1.79 | 96.75 ± 0.96 | 72.63 ± 7.93 | 64.9 ± 6.72 |
| 7 | Cmpd. 301 | 0.5 | 85.4 ± 1.5 | 88.63 ± 1.23 | 98.15 ± 0.8 | 84.95 ± 1.62 | 72.35 ± 1.36 |
| 8 | Cmpd. 301 | 0.5 | 31.3 ± 5.9 | 47.73 ± 7.95 | 98.13 ± 0.59 | 53.43 ± 3.2 | 64.75 ± 1.91 |
| 9 | Cmpd. 301 | 0.5 | 51.4 ± 9.95 | 68.98 ± 6.97 | 98.65 ± 0.34 | 76.53 ± 1.8 | 65.7 ± 4.91 |
| 10 | Cmpd. 301 | 0.5 | 21.58 ± 7.58 | 37.25 ± 9.08 | 96.7 ± 1.98 | 42.98 ± 5.59 | 47.25 ± 6.2 |
| 11 | Cmpd. 301 | 0.5 | 66.65 ± 8.26 | 79.95 ± 6.53 | 98.93 ± 0.52 | 83.48 ± 3.8 | 73.6 ± 4.91 |
| 12 | Cmpd. 301 | 0.5 | 86.63 ± 3.39 | 90.03 ± 1.61 | 97.7 ± 0.89 | 76.65 ± 3.84 | 64.38 ± 6.7 |
| 13 | Cmpd. 301 | 0.5 | 75.53 ± 1.87 | 82.6 ± 3.12 | 97 ± 1.03 | 81.9 ± 1.13 | 69.2 ± 4.59 |
| 14 | Cmpd. 301 | 0.5 | 87.88 ± 1.9 | 91.18 ± 1.91 | 98.03 ± 0.94 | 81.85 ± 1.93 | 67.08 ± 3.33 |
| 15 | Cmpd. 301 | 0.5 | 74.03 ± 9.1 | 83.9 ± 4.55 | 99.53 ± 0.18 | 84 ± 2.72 | 68.08 ± 2.31 |
| 16 | Cmpd. 301 | 0.5 | 77.95 ± 6.03 | 86.15 ± 3.29 | 90.23 ± 1.07 | 60.83 ± 5.77 | 60.15 ± 6.37 |
| 17 | Cmpd. 301 | 0.5 | 77.63 ± 5.78 | 81.5 ± 5.3 | 89.03 ± 2.54 | 63.25 ± 8.93 | 60.03 ± 9.25 |
| 18 | Cmpd. 301 | 0.5 | 27.33 ± 9.45 | 36.88 ± 13.09 | 72.28 ± 2.87 | 35.23 ± 4.75 | 20.1 ± 4.37 |
| 19 | Cmpd. 301 | 0.5 | 61.03 ± 4.79 | 71.1 ± 3.75 | 91.93 ± 1.83 | 55.33 ± 3.24 | 43.88 ± 4.57 |
| 20 | Cmpd. 301 | 0.5 | 74.4 ± 7.51 | 83.95 ± 5.28 | 93.5 ± 1.32 | 70.53 ± 4.67 | 67.08 ± 3.29 |
| 21 | Cmpd. 301 | 0.5 | 89.85 ± 1.02 | 92.53 ± 0.91 | 96.93 ± 0.88 | 82.53 ± 0.56 | 70.08 ± 2.74 |
| 22 | Cmpd. 301 | 0.5 | 71.85 ± 10.64 | 81.38 ± 6.7 | 97.7 ± 1.06 | 63.43 ± 10.43 | 68.88 ± 4.63 |
| 23 | Cmpd. 301 | 0.5 | 86.73 ± 4.43 | 90.48 ± 1.83 | 95.13 ± 0.71 | 77.43 ± 1.9 | 70.93 ± 1.61 |
| 24 | Cmpd. 301 | 0.5 | 89.98 ± 2.37 | 92.9 ± 0.87 | 97.98 ± 0.48 | 87.18 ± 1.35 | 77.08 ± 2.61 |
| 25 | Cmpd. 301 | 0.5 | 84.83 ± 6.03 | 90.3 ± 2.94 | 97.13 ± 0.55 | 82.23 ± 1.67 | 67.58 ± 3.57 |
| 26 | I-18 | 0.5 | 10.15 ± 2.6 | 11.2 ± 2.81 | 66.9 ± 12.34 | 13.75 ± 5.09 | 17.35 ± 4.91 |
| 27 | Cmpd. 22 | 0.5 | 86.48 ± 6.38 | 84.78 ± 5.02 | 90.48 ± 2.36 | 74.53 ± 5.9 | 62.93 ± 4.8 |
| 28 | Cmpd. 22 | 0.5 | 83.8 ± 3.74 | 84.93 ± 2.9 | 90.28 ± 1.69 | 73.38 ± 4.86 | 59.93 ± 3.63 |
| 29 | Cmpd. 301 | 0.5 | 88.93 ± 5.24 | 89.5 ± 3.15 | 83.23 ± 4.74 | 74.2 ± 10.75 | 64.93 ± 5.07 |
| 30 | Cmpd. 301 | 0.5 | 86.25 ± 1.24 | 90.4 ± 0.94 | 79.45 ± 8.42 | 81.6 ± 0.76 | 75.35 ± 0.84 |
| 31 | Cmpd. 301 | 1 | 93.75 ± 0.45 | 90.8 ± 1.7 | 74.45 ± 0.05 | 43.9 ± 2.7 | 46.4 ± 2.5 |
| 32 | Cmpd. 22 | 1 | 95.05 ± 0.05 | 91.4 ± 2 | 95.45 ± 0.25 | 79.4 ± 1.3 | 68.25 ± 1.05 |
| 33 | Cmpd. 301 | 1 | 92.6 ± 0 | 86.7 ± 1.7 | 89.05 ± 1.05 | 66.85 ± 4.25 | 62.3 ± 2.6 |
| 34 | Cmpd. 301 | 1 | 79.5 ± 12.5 | 79.5 ± 5.6 | 74.35 ± 9.75 | 48.1 ± 5.3 | 39.75 ± 9.25 |
| 35 | Cmpd. 22 | 0.5 | 80.24 ± 11.76 | 82.34 ± 9.56 | 99.4 ± 0.39 | 72.66 ± 7.76 | 67.74 ± 15.19 |
| 36 | Cmpd. 22 | 0.5 | 86.88 ± 7.48 | 85.77 ± 7.62 | 99.24 ± 0.46 | 75.79 ± 10.45 | 65.12 ± 17.18 |
| 37 | Cmpd. 301 | 0.5 | 75.57 ± 11.23 | 81.36 ± 7.86 | 99.45 ± 0.38 | 77.79 ± 4.31 | 67.88 ± 22.24 |
| 38 | Cmpd. 22 | 0.5 | 75.83 ± 12.63 | 80.52 ± 8.92 | 89.51 ± 12.49 | 81.38 ± 6.92 | 76.93 ± 13.83 |

| | | | Splenic | Splenic | |
|---|---|---|---|---|---|
| | Splenic | Splenic | CD8 | CD4 | Splenic |
| | Neutrophils | B cells | T cells | T cells | NK cells |
| Sample # | % mOX40L Mean ± StDev | | | | |
| Buffer | 0.82 ± 0.54 | 0.76 ± 0.84 | 0.61 ± 0.34 | 0.71 ± 0.43 | 0.71 ± 0.56 |
| Crtl. | | | | | |
| 1 | 71.65 ± 4.68 | 27.33 ± 4.11 | 15.88 ± 1.22 | 8.53 ± 1.16 | 82.05 ± 3.7 |
| 2 | 82.05 ± 11.34 | 29.65 ± 4.44 | 15.53 ± 3.48 | 16.2 ± 2.45 | 68.775 ± 8.3 |
| 3 | 73.78 ± 4.69 | 32.9 ± 3.04 | 20.85 ± 2.01 | 16.55 ± 1.99 | 58.9 ± 8.14 |
| 4 | 73.28 ± 1.52 | 23.2 ± 2.05 | 12.2 ± 1.64 | 13.88 ± 1.29 | 43.475 ± 4.13 |
| 5 | 34.32 ± 2.65 | 30.22 ± 2.33 | 12.56 ± 0.95 | 7.21 ± 0.86 | 34.32 ± 2.65 |
| 6 | 23.18 ± 4.13 | 29.08 ± 5.64 | 18.73 ± 4.26 | 14.38 ± 2.28 | 58.9 ± 8.23 |
| 7 | 29.4 ± 2.27 | 35.4 ± 1.7 | 22.53 ± 0.83 | 16.15 ± 0.52 | 67.225 ± 2.45 |
| 8 | 11.58 ± 1.96 | 12.23 ± 1.04 | 7.94 ± 0.66 | 6.65 ± 0.42 | 33.55 ± 4.7 |
| 9 | 26.9 ± 2.27 | 27.18 ± 1.98 | 14.98 ± 1.94 | 13.23 + 2.01 | 59.55 ± 4.65 |
| 10 | 8.13 ± 0.79 | 9.75 ± 0.72 | 6.8 ± 0.65 | 6.1 ± 0.41 | 28.7 ± 4.23 |
| 11 | 34.73 ± 2.35 | 30.9 ± 5.45 | 20.13 ± 2.41 | 15.98 + 1.64 | 71.925 ± 4.98 |
| 12 | 21.13 ± 2 | 26.3 ± 1.39 | 17 ± 1.48 | 13 ± 0.65 | 56.6 ± 3.42 |
| 13 | 22.35 ± 1.76 | 27.88 ± 0.8 | 18.58 ± 2.84 | 15.08 + 1.64 | 59.5 ± 1.39 |
| 14 | 23.2 ± 4.04 | 27.5 ± 3.98 | 18.35 ± 2.83 | 13.7 ± 1.62 | 60.7 ± 5.23 |
| 15 | 36.55 ± 3.85 | 30.93 ± 3.9 | 17.7 ± 1.37 | 14.23 ± 1.13 | 66.25 ± 3.32 |
| 16 | 17.48 ± 4.32 | 23.08 ± 4.6 | 16.68 ± 3.55 | 13.7 ± 4.23 | 43.7 ± 7.77 |
| 17 | 15.23 ± 5.52 | 21.13 ± 4.18 | 16.28 ± 1.98 | 11.88 ± 1.93 | 45.325 ± 10.1 |
| 18 | 10.69 ± 1.95 | 10.05 ± 2.02 | 7.17 ± 0.95 | 6.59 ± 0.92 | 23.175 ± 3.36 |
| 19 | 17.78 ± 2.33 | 18.25 ± 4.59 | 11 ± 1.05 | 9.45 ± 0.9 | 36.475 ± 4.22 |
| 20 | 18.35 ± 1.42 | 21.78 ± 1.68 | 13.73 ± 1.68 | 11.31 ± 1.44 | 52.45 ± 3.7 |
| 21 | 23.05 ± 3.21 | 37.23 ± 7.26 | 19.65 ± 2.46 | 13.48 ± 1.17 | 64.775 ± 3.85 |
| 22 | 17.4 ± 1.71 | 19.55 ± 2.67 | 12.65 ± 1.06 | 12.1 ± 0.85 | 44.775 ± 7.34 |
| 23 | 15.88 ± 1.34 | 21.18 ± 3.22 | 14.43 ± 2.37 | 13.53 ± 1.25 | 53.175 ± 2.74 |
| 24 | 31.48 ± 2.69 | 35.55 ± 10.29 | 20.15 ± 3.64 | 17.65 ± 1.76 | 71.075 ± 3.02 |
| 25 | 15.65 ± 2.15 | 22.4 ± 1.8 | 15.15 ± 0.87 | 13.95 ± 0.67 | 57.35 ± 1.27 |
| 26 | 4.14 ± 1.83 | 4.57 ± 0.58 | 3.16 ± 0.92 | 2.39 ± 0.69 | 9.5625 ± 2.29 |
| 27 | 47.63 ± 8.89 | 32.98 ± 2.71 | 16.88 ± 1.53 | 11.95 ± 1.21 | 60.15 ± 5.85 |
| 28 | 51.13 ± 3.53 | 41.33 ± 6.85 | 23.95 ± 7.51 | 14.85 ± 4.16 | 60.625 ± 3.61 |
| 29 | 31.13 ± 4.98 | 34.53 ± 7.01 | 24.43 ± 6.87 | 16.38 ± 5.48 | 61.75 ± 5.56 |
| 30 | 41.05 ± 5.26 | 35.73 ± 2.24 | 20.15 ± 1.84 | 13.6 ± 0.89 | 70.9 ± 1.39 |
| 31 | 18.85 ± 3.15 | 24.3 ± 4.8 | 14.6 ± 2.3 | 7.47 ± 1.14 | 47.15 ± 5.65 |
| 32 | 48.55 ± 0.65 | 45 ± 3.4 | 27.45 ± 0.75 | 12.5 ± 0.4 | 80.5 ± 2.6 |
| 33 | 35.5 ± 1.3 | 33.3 ± 0.8 | 18.2 ± 0.8 | 9.08 ± 0.38 | 65.35 ± 4.95 |
| 34 | 18.3 ± 7.5 | 24.05 ± 1.35 | 15.95 ± 3.25 | 8.49 ± 1.13 | 54.4 ± 12.5 |
| 35 | 57.36 ± 17.44 | 35.03 ± 5.07 | 22.65 ± 6.9 | 12.62 ± 1.39 | 41.34 ± 26.22 |
| 36 | 56.41 ± 20.58 | 41.28 ± 5.14 | 20.77 ± 3.32 | 14.97 ± 1.99 | 22.06 ± 20.08 |
| 37 | 45.45 ± 10.44 | 33.36 ± 6.51 | 20.42 ± 9.86 | 9.13 ± 2.84 | 50.78 ± 14.59 |
| 38 | 51.68 ± 10.58 | 29.67 ± 4.29 | 21.66 ± 4.21 | 11.26 ± 1.23 | 62.43 ± 6.59 |

### EQUIVALENTS

The details of one or more embodiments of the invention are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the invention to the precise form disclosed, but by the claims appended hereto.

What is claimed is:

1. A lipid assembly comprising a phosphatidylserine phospholipid, an ionizable lipid, and a structural lipid, wherein the phosphatidylserine phospholipid is of Formula (PL-I): 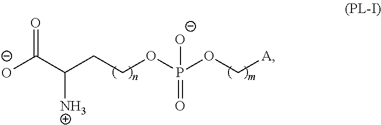
or a salt thereof, wherein:
n is 0, 1, 2, 3, 4, 5, 6, 7, 8, 9, or 10;
m is 0, 1, 2, 3, 4, 5, 6, 7, 8, 9, or 10;
A is of the formula: 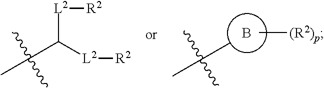
each instance of L² is independently a bond or an optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently cholesterol, optionally substituted cycloalkyl, optionally substituted C₁₋₃₀ alkyl, optionally substituted C₁₋₃₀ alkenyl, or optionally substituted C₁₋₃₀ alkynyl; optionally wherein one or more methylene units of the optionally substituted C₁₋₃₀ alkyl, optionally substituted C₁₋₃₀ alkenyl, or optionally substituted C₁₋₃₀ alkynyl are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

2. A population of lipid assemblies comprising a phosphatidylserine phospholipid, an ionizable lipid, and a structural lipid, wherein the phosphatidylserine phospholipid is of Formula (PL-I): 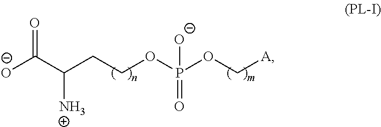
or a salt thereof, wherein:
n is 0, 1, 2, 3, 4, 5, 6, 7, 8, 9, or 10;
m is 0, 1, 2, 3, 4, 5, 6, 7, 8, 9, or 10;
A is of the formula: 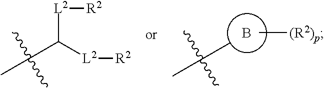
each instance of L² is independently a bond or an optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently cholesterol, optionally substituted cycloalkyl, optionally substituted C₁₋₃₀ alkyl, optionally substituted C₁₋₃₀ alkenyl, or optionally substituted C₁₋₃₀ alkynyl; optionally wherein one or more methylene units of the optionally substituted C₁₋₃₀ alkyl, optionally substituted C₁₋₃₀ alkenyl, or optionally substituted C₁₋₃₀ alkynyl are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

3. The lipid assembly or the population of lipid assemblies of claim 1 or 2, wherein the phosphatidylserine phospholipid is of Formula (PL-Ia): 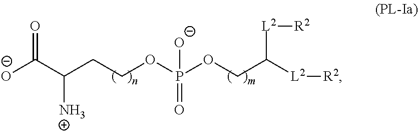
or a salt thereof.

4. The lipid assembly or the population of lipid assemblies of any one of claims 1 to 3, wherein n is 0 and m is 1.

5. The lipid assembly or the population of lipid assemblies of any one of the preceding claims, wherein the phosphatidylserine phospholipid is of Formula (PL-Ib): 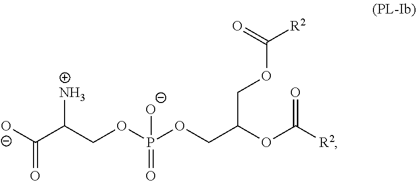
or a salt thereof.

6. The lipid assembly or the population of lipid assemblies of any one of the preceding claims, wherein each instance of R² is independently C₁₋₃₀, C₉₋₂₁, C₁₁₋₁₉, or C₁₃₋₁₇ alkyl or C₁₋₃₀, C₉₋₂₁, C₁₁₋₁₉, or C₁₃₋₁₇ alkenyl.

7. The lipid assembly or the population of lipid assemblies of any one of the preceding claims, wherein the phosphatidylserine phospholipid is 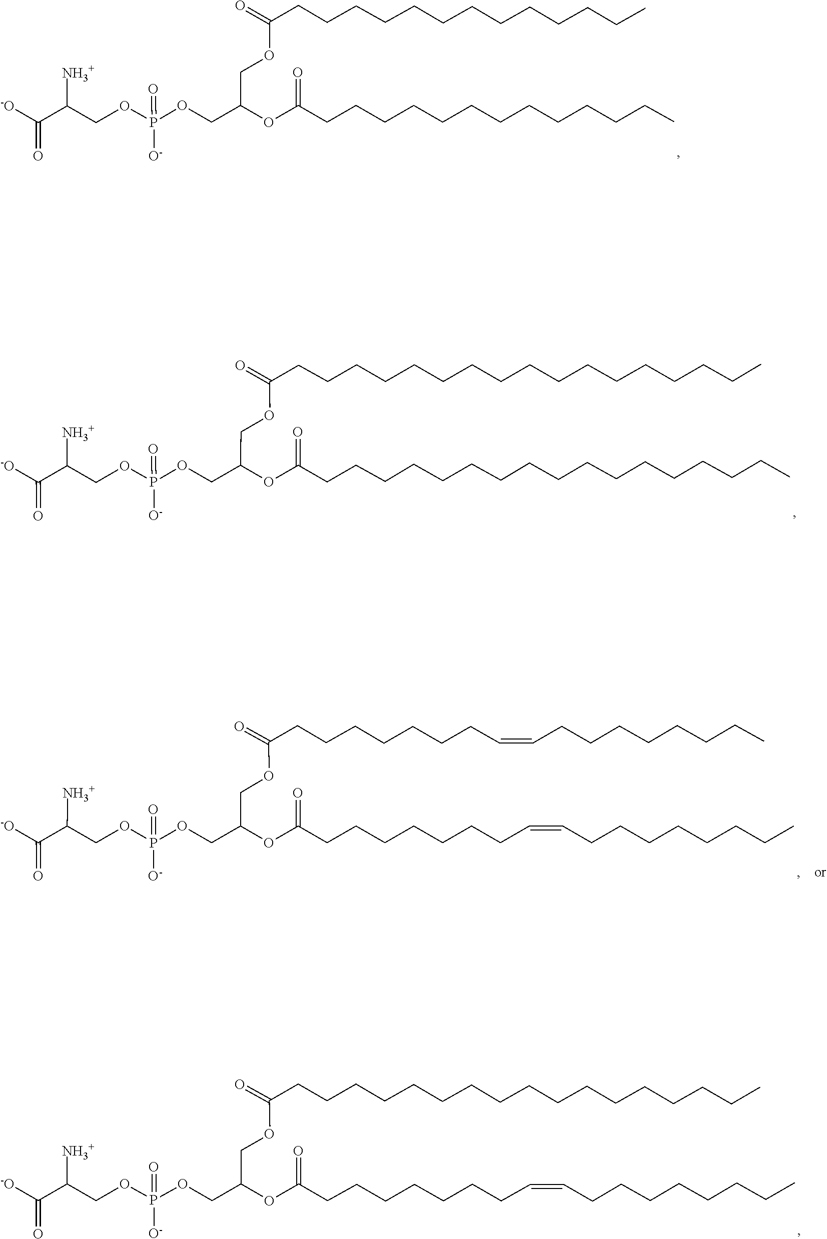
or a salt thereof.

8. The lipid assembly or the population of lipid assemblies of any one of the preceding claims, comprising between about 1 mol % to about 10 mol %, about 1 mol % to about 8 mol %, about 1 mol % to about 5 mol %, or about 2 mol % to about 5 mol % of the phosphatidylserine phospholipid; and/or
comprising about 1 mol %, about 2 mol %, about 3 mol %, about 4 mol %, or about 5 mol % of the phosphatidylserine phospholipid.

9. The lipid assembly or the population of lipid assemblies of any one of the preceding claims, comprising about 30 mol % to about 50 mol %, about 30 mol % to about 45 mol %, about 30 mol % to about 40 mol %, about 35 mol % to about 45 mol %, or about 35 mol % to about 40 mol % of the ionizable lipid; and/or
comprising about 30 mol %, about 35 mol %, about 40 mol %, about 45 mol %, or about 50 mol % of the ionizable lipid; and/or
wherein the ionizable lipid is compound 301 or compound 22.

10. The lipid assembly or the population of lipid assemblies of any one of the preceding claims, comprising about 15 mol % to about 45 mol %, about 20 mol % to about 45 mol %, about 25 mol % to about 45 mol %, about 30 mol % to about 45 mol %, about 35 mol % to about 45 mol %, or about 40 mol % to about 45 mol % of the structural lipid; and/or
comprising about 15 mol %, about 20 mol %, about 25 mol %, about 30 mol %, about 35 mol %, about 40 mol %, or about 45 mol % of the structural lipid; and/or
wherein the structural lipid is cholesterol.

11. The lipid assembly or the population of lipid assemblies of any one of the preceding claims, further comprising a second phospholipid; optionally:
comprising about 10 mol % to about 30 mol %, 10 mol % to about 25 mol %, 10 mol % to about 20 mol %, about 15 mol % to about 25 mol %, or about 15 mol % to about 20 mol % of the second phospholipid; and/or:
comprising about 10 mol %, about 15 mol %, about 18 mol %, about 20 mol %, about 22 mol %, about 25 mol %, or about 30 mol % of the second phospholipid; and/or
wherein the second phospholipid is DSPC, DOPE, DOPC, POPE, or POPC.

12. The lipid assembly or the population of lipid assemblies of any one of the preceding claims, being free of PEG lipid.

13. The lipid assembly or the population of lipid assemblies of any one of claims 1 to 11, further comprising a PEG lipid; optionally:
comprising about 0.5 mol % to about 10 mol %, about 0.5 mol % to about 5 mol %, about 1 mol % to about 5 mol %, or about 1 mol % to about 3 mol % of the PEG lipid; and/or
comprising about 0.5 mol %, about 1 mol %, about 2 mol %, about 3 mol %, about 4 mol %, or about 5 mol % of the PEG lipid; and/or
wherein the PEG lipid is PL-02.

14. The lipid assembly or the population of lipid assemblies of any one of the preceding claims, having a pH value lower than the pKa value of the ionizable lipid; and/or
having a pH value of about 4.0±2.0, about 4.0±1.5, about 4.0±1.4, about 4.0±1.3, about 4.0±1.2, about 4.0±1.1, about 4.0±1.0, about 4.0±0.9, about 4.0±0.8, about 4.0±0.7, about 4.0±0.6, about 4.0±0.5, about 4.0±0.4, about 4.0±0.3, about 4.0±0.2, or about 4.0±0.1; or
having a pH value of about 5.0±2.0, about 5.0±1.5, about 5.0±1.4, about 5.0±1.3, about 5.0±1.2, about 5.0±1.1, about 5.0±1.0, about 5.0±0.9, about 5.0±0.8, about 5.0±0.7, about 5.0±0.6, about 5.0±0.5, about 5.0±0.4, about 5.0±0.3, about 5.0±0.2, or about 5.0±0.1.

15. The lipid assembly or the population of lipid assemblies of any one of claims 1 to 13, having a pH value higher than the pKa value of the ionizable lipid; and/or
having a pH value of about 8.0±2.0, about 8.0±1.5, about 8.0±1.4, about 8.0±1.3, about 8.0±1.2, about 8.0±1.1, about 8.0±1.0, about 8.0±0.9, about 8.0±0.8, about 8.0±0.7, about 8.0±0.6, about 8.0±0.5, about 8.0±0.4, about 8.0±0.3, about 8.0±0.2, or about 8.0±0.1; or
having a pH value of about 9.0±3.0, about 9.0±2.0, about 9.0±1.5, about 9.0±1.4, about 9.0±1.3, about 9.0±1.2, about 9.0±1.1, about 9.0±1.0, about 9.0±0.9, about 9.0±0.8, about 9.0±0.7, about 9.0±0.6, about 9.0±0.5, about 9.0±0.4, about 9.0±0.3, about 9.0±0.2, or about 9.0±0.1; or
having a pH value of about 12.0±2.0, about 12.0±1.5, about 12.0±1.4, about 12.0±1.3, about 12.0±1.2, about 12.0±1.1, about 12.0±1.0, about 12.0±0.9, about 12.0±0.8, about 12.0±0.7, about 12.0±0.6, about 12.0±0.5, about 12.0±0.4, about 12.0±0.3, about 12.0±0.2, or about 12.0±0.1.

16. The lipid assembly or the population of lipid assemblies of any preceding claim, having a zeta potential between about −40 mV to about −5 mV, about −30 mV to about −5 mV, about −20 mV to about −5 mV, about −40 mV to about −10 mV, about −30 mV to about −10 mv, or about −20 mV to about −10 mV when measured in 0.1N PBS at pH 7.5.

17. The lipid assembly or the population of lipid assemblies of any one of the preceding claims, being free of therapeutic agent.

18. The lipid assembly or the population of lipid assemblies of any one of claims 1 to 16, further comprising a therapeutic agent; optionally wherein the therapeutic agent is an RNA.

19. A pharmaceutical composition comprising the lipid assembly or the population of lipid assemblies of any one of claims 1 to 18 and one or more pharmaceutically acceptable carriers or excipients.

20. A method of preparing the lipid assembly or the population population of lipid assemblies of any one of claims 1 to 18.

21. A method of preparing the pharmaceutical composition of claim 19.

22. A method of delivering a therapeutic agent to a hematopoietic stem and progenitor cell (HSPC) in a subject, comprising administering to the subject the lipid assembly, the population of lipid assemblies, or the pharmaceutical composition of any one of claims 1 to 19.

23. The lipid assembly, the population of lipid assemblies, or the pharmaceutical composition of any one of claims 1 to 19 for use in delivering a therapeutic agent to a hematopoietic stem and progenitor cell (HSPC) in a subject.

24. Use of lipid assembly, the population of lipid assemblies, or the pharmaceutical composition of any one of claims 1 to 19 in the manufacture of a medicament for delivering a therapeutic agent to a hematopoietic stem and progenitor cell (HSPC) in a subject.

25. The method, lipid assembly, population, pharmaceutical composition, or use of any one of claims 22 to 24, wherein:
the HSPC is in the spleen of the subject; or
the HSPC is in the bone marrow of the subject.

26. A method of treating or preventing a disease or disorder, the method comprising administering to a subject in need thereof the lipid assembly, the population of lipid assemblies, or the pharmaceutical composition of any one of claims 1 to 19.

27. The lipid assembly, the population of lipid assemblies, or the pharmaceutical composition of any one of claims 1 to 19 for use in treating or preventing a disease or disorder in a subject.

28. Use of the lipid assembly, the population of lipid assemblies, or the pharmaceutical composition of any one of claims 1 to 19 in the manufacture of a medicament for treating or preventing a disease or disorder in a subject.

29. The method, lipid assembly, population, pharmaceutical composition, or use of any one of claims 26 to 28, wherein:
the subject is human; and/or
the disease or disorder is a spleen disease or a spleen disorder; or
the disease or disorder is a bone marrow disease or a bone marrow disorder.

30. A process of preparing a nanoparticle, the process comprising contacting an aqueous stream comprising a phosphatidylserine lipid with an organic stream comprising an ionizable lipid and a structural lipid, thereby obtaining a mixed product; optionally:
wherein the organic stream further comprises a PEG lipid; and/or
wherein the pH of the aqueous stream is lower than the pKa of the ionizable lipid.

31. The process of claim 30, wherein the process further comprises subjecting the mixed product to an in-line dilution; optionally:
wherein the in-line dilution lowers an organic solvent content of the mixed product; and/or
wherein the in-line dilution raises the pH of the mixed product above the pKa of the ionizable lipid.

32. The process of claim 30 or 31, wherein the phosphatidylserine phospholipid is DMPS; and/or
wherein the ionizable lipid is Compound 22 or Compound 301.

33. A nanoparticle prepared by the process of any one of claims 30 to 32.
